(19) United States
(12) Patent Application Publication
(10) Pub. No.: US 2025/0282714 A1
(43) Pub. Date: Sep. 11, 2025

(54) COMPOSITIONS FOR AND METHODS OF MODULATING RNA STABILITY

(71) Applicant: Duke University, Durham, NC (US)

(72) Inventors: Amanda Hargrove, Durham, NC (US); Martina Zafferani, Durham, NC (US)

(21) Appl. No.: 18/849,279

(22) PCT Filed: Mar. 23, 2023
(86) PCT No.: PCT/US2023/064879

§ 371 Date: Sep. 20, 2024

Related U.S. Application Data
(60) Provisional application No. 63/322,902, filed on Mar. 23, 2022.

(51) Int. Cl. C07C 257/18; A61K 31/655; A61P 35/00; C07C 317/32; C07D 211/34; C07D 211/58; C07D 213/58; C07D 213/78; C07D 295/135; C07D 295/155; C07D 307/54; C07D 401/14

(52) U.S. Cl. CPC C07C 257/18; A61K 31/655; A61P 35/00; C07C 317/32; C07D 211/34; C07D 211/58; C07D 213/58; C07D 213/78; C07D 295/135; C07D 295/155; C07D 307/54; C07D 401/14; C07C 2601/02

(57) ABSTRACT

Disclosed herein are DMZ-derived compositions for and methods of modulating the expression and/or function of disease associated and/or diseasing causing secondary RNA structures and tertiary RNA structures.

### I. CROSS-REFERENCE TO RELATED APPLICATION

This application claims the benefit of priority to U.S. Provisional Patent Application No. 63/322,902 filed 23 Mar. 2022, which is incorporated by reference herein in its entirety.

### STATEMENT REGARDING FEDERALLY SPONSORED RESEARCH OR DEVELOPMENT

This invention was made with government support under R35GM124785 awarded by the National Institute of General Medical Sciences. The government has certain rights in the invention.

### II. REFERENCE TO THE SEQUENCE LISTING

The Sequence Listing submitted 23 Mar. 2023 as an XML file named “23_2070_WO_Sequence_Listing”, created on 23 Mar. 2023 and having a size of 109 KB is hereby incorporated by reference pursuant to 37 C.F.R. § 1.52 (e) (5).

### III. BACKGROUND

The rapidly increasing characterization of RNA tertiary structures has revealed their pervasiveness and active roles in human diseases. Small molecule-mediated modulation of RNA tertiary structures constitutes an attractive avenue for the development of tools for both therapeutically targeting and/or uncovering the pathways associated with these RNA motifs. This potential has been highlighted by targeting of the triple helix present at the 3′-end of the non-coding RNA MALAT1, a transcript implicated in several human diseases. This triplex has been reported to decrease the transcript susceptibility to degradation and promote its cellular accumulation. While small molecules have been shown to bind and impact the stability of the MALAT1 triple helix, the small molecule properties that lead to these structural modulations are not well understood.

Thus, there remains an unmet need to test the applicability of previously identified RNA-targeted chemical space to more complex RNAs and to develop new methods to better understand these molecular recognition events. Moreover, as it relates to human pathology, there remains an unmet need to develop techniques and/or strategies to modulate the expression and/or function of disease associated and/or diseasing causing RNA secondary and tertiary structures.

### IV. BRIEF DESCRIPTION OF THE FIGURES

FIG. 1A-FIG. 1C show the design and synthesis of the diminazene focused library.

FIG. 1A shows the representation of MALAT1 triple helix base pairing (left) and crystal structure (right). The triple helix is formed by the recruitment of the genomically encoded poly(A) tail (purple) to a uridine-rich stem loop (green) (adapted from Brown A, et al. (2014) Nat Struct Mol Biol. 21(7):633-640).

FIG. 1B is the envelope diagram of principal moments of inertia calculated for the 90-member theoretical diminazene library. The 21 small molecules selected using the Kennard Stone algorithm are outlined in red.

FIG. 1C shows the synthetic scheme designed for synthesis of biscyano substituted scaffolds and coupling of commercially available amines as subunits (R).

FIG. 2A-FIG. 2C show that an indicator displacement assay (IDA) allowed evaluation of the affinity of the entire library against the MALAT1 triple helix.

FIG. 2A is a schematic of fluorescence-displacement assays. RiboGreen™ has measurable fluorescence signal when bound to RNA. Upon small molecule binding to the RNA, the fluorophore is displaced, and the fluorescence signal is quenched (left). Hypothetical curves of strong binders (green) and weak or non-binders (blue) where % displacement is plotted as a function of small molecule concentration (right).

FIG. 2B show the subunits chosen to create the theoretical 90-member library. Subunits selected for synthesis are in red.

FIG. 2C shows the relative affinity expressed as CD₅₀ (small molecule concentration needed to achieve 50% of competitive displacement) obtained via small molecule titrations using the optimized IDA assay with Ribogreen (0.5 mM) and the MALAT1 triplex (0.17 mM) in 20 mM HEPES-KOH, 52 mM KCl, 0.1 mM MgCl₂ at pH=7.4. Molecules labeled as out of range (O.R.) indicate small molecules with CD₅₀ outside of the range of small molecule concentrations (>500 mM) that lead to minimal dye displacement.

FIG. 3A-FIG. 3B show the cheminformatic trend analysis and QSAR model generation reveal affinity-based trends.

FIG. 3A is an envelope diagram of the synthesized diminazene small molecules. Small molecules that exhibited highest affinity for the triplex clustered in the rod-like sub-triangle (red). As the affinity of the small molecules decreases, the density of the ligands in the rod sub-triangle decreases (orange), with weak and non-binders being the most disc and sphere-like ligands (grey).

FIG. 3B is a plot of the best model obtained via QSAR for the CD₅₀ calculated for all 21 small molecules.

FIG. 4A-FIG. 4B show the validation of indicator displacement assay (IDA) utilizing isothermal titration calorimetry (ITC).

FIG. 4A shows affinity as measured via isothermal titration calorimetry in three independent replicates for small molecules soluble with less than or equal to 3% DMSO in 20 mM Tris-HCl, 25 mM NaCl, 3 mM MgCl₂ buffer at pH=7.4.

FIG. 4B shows the correlation between affinity measured via indicator displacement assay (IDA) and ITC.

FIG. 5A-FIG. 5C show the evaluation of the effect of the focused library on the thermal stability of the MALAT1 triplex via differential scanning fluorimetry (DSF).

FIG. 5A shows the difference (4) in melting temperature between the MALAT1 triplex (5 mM) with small molecules (5 mM) compared to the DMSO vehicle. Difference in the first but not the second peak of the melting curves supports binding in the triplex tract and not the upper stem of the MALAT1 construct.

FIG. 5B shows the overlap of representative melting curves of the MALAT1 triple helix (blue) and the isolated upper stem (orange) are in line with the second peak of the triplex bi-phasic melting curve representing the upper stem.

FIG. 5C is a plot of the best model obtained via QSAR for the ΔTm₁ calculated for all 21 small molecules.

FIG. 6A-FIG. 6D show the design and optimization of RNase A assay allows evaluation of the effect of the small molecule library on the triple helix enzymatic degradation profile.

FIG. 6A is a schematic of the RNase A assay; small molecules that stabilize base-paired regions reduce the amount of ssRNA tracts that the enzyme can cleave, resulting in a decrease of enzymatic degradation over time.

FIG. 6B shows that five time points were collected for each small molecule (2 mM) and triplex (0.2 mM) reaction, allowing the plotting of a degradation curve. The 5-minute time-point was chosen to compare small molecules assuming it is in the linear phase of decay of both stabilizing and destabilizing small molecules. Amount of RNA degraded over time was assessed by denaturing gel electrophoresis, Diamond staining, and Image J quantification.

FIG. 6C shows bar-graphs representing the fold-change of RNA degradation at the 5-minute time point for all the small molecules. RNA degradation is normalized to the same time point of DMSO as a control. All small molecules were evaluated in three independent experiments. A 5% DMSO control was included for every set of small molecules loaded on the gel.

FIG. 6D shows plot of the predictive model yielded by QSAR analysis. Stabilizers (orange) and destabilizers (blue) present separately in the graph.

FIG. 7 shows a suite of powerful orthogonal biophysical tools for the evaluation of small molecule:RNA triplex interactions that generate predictive models and will allow small molecule interrogation of the growing body of disease-associated RNA triple helice.

FIG. 8A shows an envelope diagram representing 3D shape diversity of the 90-member theoretical library of diminazene analogues (blue). A sub-set of 20 chosen for synthetic investigation using the Kennard Stone algorithm (pink). FIG. 8B shows unreactive and unstable small molecule candidates were then substituted with small molecules closest in the envelope diagram and afforded a total of 20 small molecule analogues and the Berenil® scaffold (purple).

FIG. 9 shows the coupling conditions attempted for R-19, R-25, R-27, and R-28 that did not yield any product formation.

FIG. 10 shows the MALAT1 triple helix construct run on Small RNA chip on Agilent bioanalyzer. Construct size is within 25% confidence value of sizing for the gel chip.

FIG. 11 shows a cartoon representation of constructs used for DSF selectivity experiments.

FIG. 12A-FIG. 12U show the small molecule affinity (CD₅₀) determined via RiboGreen™ IDA for the library as screened in three independent replicates in a 16-point titration.

FIG. 13A-FIG. 13Z show the melting profiles of the MALAT1 triple helix with equimolar small molecule. The entire diminazene (DMZ) library was screened in three independent replicates. Two small molecules previously published and identified as destabilizers showed little to no effect on the thermal stability of the triplex, thereby indicating that DSF might not be a suitable assay to identify destabilizing small molecules.

FIG. 14 shows the correlation between the difference in melting temperature of the first peak of the bi-phasic melting curve of the triplex (ΔTm1) and IDA-derived small molecule affinity (Log(CD₅₀)). The small molecules with the highest affinity for the MALAT1 triple helix are also the ones that cause the largest shifts in thermal stability. Spearman's correlation −0.89 (p-value <0.0001).

FIG. 15 shows the dose-dependent increase in thermal stability of the MALAT1 triple helix as measured by DSF. Shown is a representative set of small molecules identified as the best stabilizers.

FIG. 16A-FIG. 16F show the melting profiles of the MALAT1 upper stem and triple helix (top) and of the upper stem with equimolar small molecule (bottom). A sub-set of DMZ small molecules were screened in three independent replicates. Small molecules were chosen for testing based on their stabilization of ΔTm1 but not ΔTm2 of the MALAT1 triple helix. No small molecules caused a significant shift in the melting profile of the upper stem, indicating selective interactions with the triplex construct.

FIG. 17A-FIG. 17E show the melting profiles of the MALAT1 stem loop proxy and triple helix (top) and of the stem loop proxy with equimolar small molecule (bottom). A sub-set of DMZ small molecules were screened in three independent replicates. Small molecules were chosen for testing based on their stabilization of ΔTm1 but not ΔTm2 of the MALAT1 triple helix. No small molecules caused a significant shift in the melting profile of the stem loop proxy, indicating selective interactions with the triplex construct.

FIG. 18A-FIG. 18W show the RNase A enzymatic degradation assay of the entire diminazene (DMZ) library and two controls, a stabilizer (DPFp20) and a destabilizer (SM5). The two controls exhibit the same effects observed via RNase R enzymatic degradation study, thereby validating the fitness of the new, faster RNase A assay. A total of five time-points were collected for each small molecules in three independent replicates and the time point of 5 minutes was chosen to classify small molecules as stabilizers or destabilizers as it is in the linear phase of decay.

FIG. 19A shows representative 15% TBE denaturing gel of DMSO RNase A enzymatic degradation assay with ladder. At the zero time-point no degradation of the triplex is observed. As time progresses the triplex is degraded is shorter fragments. A short dsDNA loading control (10 bp band) was added to each loaded sample to normalize the intensity of the bands to account for loading and pipetting errors (left).

FIG. 19B shows imaging Cy5 reveals 5′ fragments, leading to identification of potential RNase A cleavage sites (right).

FIG. 20A-FIG. 20H show the melting profiles of RNase A enzyme with (orange) and without (black) small molecule measured via differential scanning fluorimetry (DSF) in three independent replicates.

FIG. 21A-FIG. 21C show the controls performed with MALAT1 triple helix and small molecules for ITC. FIG. 21A shows 10 μM MALAT1 triple helix control in buffer (20 mM Tris-HCl, 25 mM NaCl, 3 mM MgCl₂ pH=7.4). FIG. 21B shows 10 μM MALAT1 triple helix control in buffer (20 mM Tris-HCl, 25 mM NaCl, 3 mM MgCl₂ pH=7.4) and 3% DMSO. FIG. 21C shows representative 5 mM small molecule control in the syringe and buffer in the sample cell.

FIG. 22A-FIG. 22J show the MALAT1 ITC titrations across 3 independent replicates for various small molecules.

FIG. 23A-FIG. 23B show MTT experiments with LNCaP cells at 48 hrs (FIG. 23A) and 72 hours (FIG. 23B).

### V. BRIEF SUMMARY

Disclosed herein is a compound comprising a small molecule having a diminazene scaffold. Disclosed herein is a compound comprising a small molecule having a diminazene scaffold and a diamidine moiety.

Disclosed herein is a compound of formula

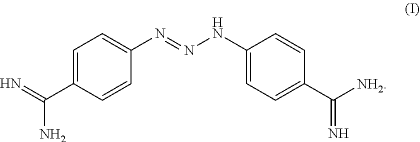 [SMILES: N=C(N)c1ccc(/N=N/Nc2ccc(C(=N)N)cc2)cc1]

Disclosed herein is a compound of formula

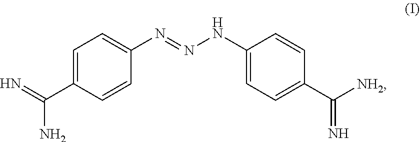 [SMILES: N=C(N)c1ccc(/N=N/Nc2ccc(C(=N)N)cc2)cc1]

having one or more of ortho, para, and/or meta substitutions.

Disclosed herein is a compound identified as DMZ-P1, DMZ-P5, DMZ-P8, DMZ-P9, DMZ-P13, DMZ-P14, DMZ-P17, DMZ-O1, DMZ-O2, DMZ-O4, DMZ-O5, DMZ-O6, DMZ-M1, DMZ-M3, DMZ-M4, DMZ-M7, DMZ-M10, DMZ-M15, DMZ-M22, DMZ-M24, DMZ-N-Me-M9, DMZ-N-Me-mPy-P13, DMZ-mPy-P5-2HCl, DMZ-mPy-P13, DMZ-mF-P5, DMZ-P5-mono, DMZ-mF-P13, DMZ-mF-P13-mono, DMZ-P29, DMZ-P29-mono, DMZ-P0-P5, DMZ-P30, DMZ-P30-mono, DMZ-mMe-P5, DMZ-mMe-P5-mono, DMZ-mMe-P13, DMZ-mMe-P13-mono, DMZ-N-Me-P5, DMZ-N-Me-P13, DMZ-P31, DMZ-P0-P32, DMZ-oMe-P5, DMZ-oMe-P5-mono, DMZ-M5, DMZ-M5-mono, DMZ-M13, Aniline-P13, DMZ-P0-P13, DMZ-M4-P5, DMZ-M4-P13, DMZ-P4, DMZ-P4-mono, DMZ-P5-P13, DMZ-P33, DMZ-P33-mono, DMZ-P2, DMZ-P2-mono, DMZ-P32, DMZ-P35, DMZ-oMe-P13, DMZ-N-Et-P13, DMZ-P34, DMZ-N-Me-M13, DMZ-N-Me-mPy-P13, DMZ-N-Et-P5, DMZ-N-Me-P36, DMZ-N-Me-P36-mono, DMZ-P37, DMZ-P37-mono, DMZ-N-nPr-P13, DMZ-N-Me-P38, DMZ-N-Me-M9, or a pharmaceutically acceptable salt, a hydrate, a prodrug, an ester, or a derivative thereof.

Disclosed herein is a composition comprising a disclosed compound, and one or more carriers and/or excipients.

Disclosed herein is a composition comprising a disclosed compound comprising a small molecule having a diminazene scaffold, and one or more carriers and/or excipients.

Disclosed herein is a composition comprising a disclosed compound comprising a small molecule having a diminazene scaffold and a diamidine moiety, and one or more carriers and/or excipients.

Disclosed herein is a composition comprising DMZ-P1, DMZ-P5, DMZ-P8, DMZ-P9, DMZ-P13, DMZ-P14, DMZ-P17, DMZ-O1, DMZ-O2, DMZ-O4, DMZ-O5, DMZ-O6, DMZ-M1, DMZ-M3, DMZ-M4, DMZ-M7, DMZ-M10, DMZ-M15, DMZ-M22, DMZ-M24, DMZ-N-Me-M9, DMZ-N-Me-mPy-P13, DMZ-mPy-P5-2HCl, DMZ-mPy-P13, DMZ-mF-P5, DMZ-P5-mono, DMZ-mF-P13, DMZ-mF-P13-mono, DMZ-P29, DMZ-P29-mono, DMZ-P0-P5, DMZ-P30, DMZ-P30-mono, DMZ-mMe-P5, DMZ-mMe-P5-mono, DMZ-mMe-P13, DMZ-mMe-P13-mono, DMZ-N-Me-P5, DMZ-N-Me-P13, DMZ-P31, DMZ-P0-P32, DMZ-oMe-P5, DMZ-oMe-P5-mono, DMZ-M5, DMZ-M5-mono, DMZ-M13, Aniline-P13, DMZ-P0-P13, DMZ-M4-P5, DMZ-M4-P13, DMZ-P4, DMZ-P4-mono, DMZ-P5-P13, DMZ-P33, DMZ-P33-mono, DMZ-P2, DMZ-P2-mono, DMZ-P32, DMZ-P35, DMZ-oMe-P13, DMZ-N-Et-P13, DMZ-P34, DMZ-N-Me-M13, DMZ-N-Me-mPy-P13, DMZ-N-Et-P5, DMZ-N-Me-P36, DMZ-N-Me-P36-mono, DMZ-P37, DMZ-P37-mono, DMZ-N-nPr-P13, DMZ-N-Me-P38, DMZ-N-Me-M9, a pharmaceutically acceptable salt, a hydrate, a prodrug, an ester, or a derivative thereof, or any combination thereof.

Disclosed herein is a pharmaceutical formulation comprising a disclosed compound and one or more pharmaceutically acceptable carriers. Disclosed herein is pharmaceutical formulation comprising a disclosed composition and one or more pharmaceutically acceptable carriers. Disclosed herein is a pharmaceutical formulation comprising one or more disclosed compounds and one or more pharmaceutically acceptable carriers. Disclosed herein is pharmaceutical formulation comprising one or more disclosed compositions and one or more pharmaceutically acceptable carriers.

Disclosed herein is a library comprising one or more disclosed small molecules having a diminazene scaffold. Disclosed herein is a library comprising one or more disclosed shape diverse small molecules having a diminazene scaffold. Disclosed herein is library comprising one or more disclosed small molecules having a diminazene scaffold and a diamidine moiety.

Disclosed herein is a method of treating a subject in need thereof, the method comprising administering to a subject one or more disclosed compounds. Disclosed herein is a method of treating a subject in need thereof, the method comprising administering to a subject one or more disclosed compositions. Disclosed herein is a method of treating a subject in need thereof, the method comprising administering to a subject one or more disclosed pharmaceutical formulations.

Disclosed herein is a method of treating a subject in need thereof, the method comprising administering to a subject one or more disclosed compounds, and treating a disease, condition, or disorder in the subject. Disclosed herein is a method of treating a subject in need thereof, the method comprising administering to a subject one or more disclosed compositions, and treating a disease, condition, or disorder in the subject. Disclosed herein is a method of treating a subject in need thereof, the method comprising administering to a subject one or more disclosed pharmaceutical formulations, and treating a disease, condition, or disorder in the subject.

Disclosed herein is a method of modulating one or more secondary RNA structures and/or tertiary RNA structures, the method comprising contacting one or more RNA structures with one or more disclosed compounds, disclosed compositions, disclosed pharmaceutical formulations, or any combination thereof.

Disclosed herein is a method of assessing a library of small molecules that target RNA, the method comprising using a multi-dimensional approach comprising designing and optimizing affinity-based and structural stability-based assays.

Disclosed herein is a method of assessing a library of shape diverse small molecule analogs that modulate a targeted RNA or a targeted RNA structure, the method comprising choosing a candidate small molecule; generating a theoretical library of shape diverse small molecule analogs based on the candidate small molecule; evaluating the three-dimensional shape of one or more shape diverse small molecule analogs of the theoretical library; selecting one or more shape diverse small molecules for synthesis; synthesizing one or more shape diverse small molecule analogs in the theoretic library; and characterizing the one or more synthesized shape diverse small molecule analogs.

### VI. DETAILED DESCRIPTION

The present disclosure describes formulations, compounded compositions, kits, capsules, containers, and/or methods thereof. It is to be understood that the inventive aspects of which are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

### A. Relevant Definitions

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

This disclosure describes inventive concepts with reference to specific examples. However, the intent is to cover all modifications, equivalents, and alternatives of the inventive concepts that are consistent with this disclosure.

As used in the specification and the appended claims, the singular forms “a”, “an”, and “the” include plural referents unless the context clearly dictates otherwise.

The phrase “consisting essentially of” limits the scope of a claim to the recited components in a composition or the recited steps in a method as well as those that do not materially affect the basic and novel characteristic or characteristics of the claimed composition or claimed method. The phrase “consisting of” excludes any component, step, or element that is not recited in the claim. The phrase “comprising” is synonymous with “including”, “containing”, or “characterized by”, and is inclusive or open-ended. “Comprising” does not exclude additional, unrecited components or steps.

As used herein, when referring to any numerical value, the term “about” means a value falling within a range that is ±10% of the stated value.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight component X and 5 parts by weight component Y, X and Y are present at a weight ratio of 2:5, and are present in such ratio regardless of whether additional components are contained in the compound.

As used herein, the terms “optional” or “optionally” means that the subsequently described event or circumstance can or cannot occur, and that the description includes instances where said event or circumstance occurs and instances where it does not. In an aspect, a disclosed method can optionally comprise one or more additional steps, such as, for example, repeating an administering step or altering an administering step.

As used herein, the term “subject” refers to the target of administration, e.g., a human being. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig, or rodent. The term does not denote a particular age or sex, and thus, adult and child subjects, as well as fetuses, whether male or female, are intended to be covered. In an aspect, a subject can be a human patient. In an aspect, a subject can have a disease, condition, or disorder caused by, related to, and/or exacerbated by the presence of, expression of, and/or activity of one or more secondary RNA structures, one or more tertiary RNA structures, or any combination thereof.

The term “biological sample” as used herein includes, but is not limited to, a sample containing tissues, cells, and/or biological fluids isolated from a subject. Examples of biological samples include, but are not limited to, tissues, cells, biopsies, blood, lymph, serum, plasma, urine, saliva, mucus, and tears. In an aspect, the biological sample can be a biopsy (such as a tumor biopsy). A biological sample can be obtained directly from a subject (e.g., by blood or tissue sampling) or from a third party (e.g., received from an intermediary, such as a healthcare provider or lab technician).

As used herein, the term “diagnosed” means having been subjected to an examination by a person of skill, for example, a physician, and found to have a condition that can be diagnosed or treated by one or more of the disclosed compounds, disclosed compositions, disclosed pharmaceutical formulations, or any combination thereof, or by one or more of the disclosed methods. For example, “diagnosed with a disease, condition, or disorder caused by, related to, and/or exacerbated by the presence of, expression of, and/or activity of one or more secondary RNA structures, one or more tertiary RNA structures, or any combination thereof” means having been subjected to an examination by a person of skill, for example, a physician, and found to have a condition that can be treated by one or more of the disclosed compounds, disclosed compositions, disclosed pharmaceutical formulations, or any combination thereof, or by one or more of the disclosed methods. For example, “suspected of having a disease, condition, or disorder caused by, related to, and/or exacerbated by the presence of, expression of, and/or activity of one or more secondary RNA structures, one or more tertiary RNA structures, or any combination thereof” can mean having been subjected to an examination by a person of skill, for example, a physician, and found to have a condition that can likely be treated by one or more of the disclosed compounds, disclosed compositions, disclosed pharmaceutical formulations, or any combination thereof, or by one or more of the disclosed methods. In an aspect, an examination can be physical, can involve various tests (e.g., blood tests, genotyping, biopsies, etc.) and assays (e.g., enzymatic assay), or a combination thereof.

A “patient” can refer to a subject that has been diagnosed with or is suspected of having a disease, condition, or disorder caused by, related to, and/or exacerbated by the presence of, expression of, and/or activity of one or more secondary RNA structures, one or more tertiary RNA structures, or any combination thereof. In an aspect, a patient can refer to a subject that has been diagnosed with or is suspected of having a disease, condition, or disorder caused by, related to, and/or exacerbated by the presence of, expression of, and/or activity of one or more secondary RNA structures, one or more tertiary RNA structures, or any combination thereof and is seeking treatment or receiving treatment.

As used herein, the phrase “identified to be in need of treatment for a disorder,” or the like, refers to selection of a subject based upon need for treatment of the disorder. For example, a subject can be identified as having a need for treatment of a disorder (e.g., such as a disease, condition, or disorder caused by, related to, and/or exacerbated by the presence of, expression of, and/or activity of one or more secondary RNA structures, one or more tertiary RNA structures, or any combination thereof) based upon an earlier diagnosis by a person of skill and thereafter subjected to treatment for the disorder (e.g., a disease, condition, or disorder caused by, related to, and/or exacerbated by the presence of, expression of, and/or activity of one or more secondary RNA structures, one or more tertiary RNA structures, or any combination thereof). In an aspect, the identification can be performed by a person different from the person making the diagnosis. In an aspect, the administration can be performed by one who performed the diagnosis.

As used herein, “inhibit,” “inhibiting”, and “inhibition” mean to diminish or decrease an activity, level, response, expression, condition, severity, disease, or other biological parameter. This can include, but is not limited to, the complete ablation of the activity, level, response, expression, condition, severity, disease, or other biological parameter. This can also include, for example, a 10% inhibition or reduction in the activity, level, response, condition, severity, disease, or other biological parameter as compared to the native or control level (e.g., a subject not having a disease, condition, or disorder caused by, related to, and/or exacerbated by the presence of, expression of, and/or activity of one or more secondary RNA structures, one or more tertiary RNA structures, or any combination thereof). Thus, in an aspect, the inhibition or reduction can be a 10%, 20%, 30%, 40%, 50%, 60%, 70%, 80%, 90%, 100%, or any amount of reduction in between as compared to native or control levels. In an aspect, the inhibition or reduction can be 10-20%, 20-30%, 30-40%, 40-50%, 50-60%, 60-70%, 70-80%, 80-90%, or 90-100% as compared to native or control levels. In an aspect, the inhibition or reduction can be 0-25%, 25-50%, 50-75%, or 75-100% as compared to native or control levels. In an aspect, a native or control level can be a pre-disease or pre-disorder level.

The words “treat” or “treating” or “treatment” include palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder (such as a disease, condition, or disorder caused by, related to, and/or exacerbated by the presence of, expression of, and/or activity of one or more secondary RNA structures, one or more tertiary RNA structures, or any combination thereof). In an aspect, the terms cover any treatment of a subject, including a mammal (e.g., a human), and includes: (i) preventing the undesired physiological change, disease, pathological condition, or disorder from occurring in a subject that can be predisposed to the disease but has not yet been diagnosed as having it; (ii) inhibiting the physiological change, disease, pathological condition, or disorder, i.e., arresting its development; or (iii) relieving the physiological change, disease, pathological condition, or disorder, i.e., causing regression of the disease. For example, in an aspect, treating a disease, condition, or disorder caused by, related to, and/or exacerbated by the presence of, expression of, and/or activity of one or more secondary RNA structures, one or more tertiary RNA structures, or any combination thereof can reduce the severity of an established disease in a subject by 1%-100% as compared to a control (such as, for example, an individual not having a disease, condition, or disorder caused by, related to, and/or exacerbated by the presence of, expression of, and/or activity of one or more secondary RNA structures, one or more tertiary RNA structures, or any combination thereof). In an aspect, treating can refer to a 1%, 2%, 3%, 4%, 5%, 6%, 7%, 8%, 9%, 10%, 20%, 30%, 40%, 50%, 60%, 70%, 80%, 90%, or 100% reduction in the severity of a disease, condition, or disorder caused by, related to, and/or exacerbated by the presence of, expression of, and/or activity of one or more secondary RNA structures, one or more tertiary RNA structures, or any combination thereof. For example, treating a disease, condition, or disorder caused by, related to, and/or exacerbated by the presence of, expression of, and/or activity of one or more secondary RNA structures, one or more tertiary RNA structures, or any combination thereof can reduce one or more symptoms of a disease, condition, or disorder caused by, related to, and/or exacerbated by the presence of, expression of, and/or activity of one or more secondary RNA structures, one or more tertiary RNA structures, or any combination thereof in a subject by 1%-100% as compared to a control (such as, for example, an individual not having a disease, condition, or disorder caused by, related to, and/or exacerbated by the presence of, expression of, and/or activity of one or more secondary RNA structures, one or more tertiary RNA structures, or any combination thereof). In an aspect, treating can refer to 1%, 2%, 3%, 4%, 5%, 6%, 7%, 8%, 9%, 10%, 20%, 30%, 40%, 50%, 60%, 70%, 80%, 90%, 100% reduction of one or more symptoms of an established disease, condition, or disorder caused by, related to, and/or exacerbated by the presence of, expression of, and/or activity of one or more secondary RNA structures, one or more tertiary RNA structures, or any combination thereof. It is understood that treatment does not necessarily refer to a cure or complete ablation or eradication of a disease, condition, or disorder caused by, related to, and/or exacerbated by the presence of, expression of, and/or activity of one or more secondary RNA structures, one or more tertiary RNA structures, or any combination thereof. However, in an aspect, treatment can refer to a cure or complete ablation or eradication of a disease, condition, or disorder caused by, related to, and/or exacerbated by the presence of, expression of, and/or activity of one or more secondary RNA structures, one or more tertiary RNA structures, or any combination thereof.

As used herein, a “biomarker” refers to a defined characteristic that is measured as an indicator of normal biological processes, pathogenic processes, or response to an exposure of intervention. In an aspect, a biomarker can be diagnostic (i.e., detects or classifies a pathological condition), prognostic (i.e., predicts the probability of disease occurrence or progression), pharmacodynamic/responsive (i.e., identifies a change in response to a therapeutic intervention), predictive (i.e., predicts how an individual or subject might respond to a particular intervention or event). In an aspect, a biomarker can be diagnostic, prognostic, pharmacodynamic/responsive, and/or predictive at the same time. In an aspect, a biomarker can be diagnostic, prognostic, pharmacodynamic/responsive, and/or predictive at different times (e.g., first a biomarker can be diagnostic and then later, the same biomarker can be prognostic, pharmacodynamic/responsive, and/or predictive). A biomarker can be an objective measure that can be linked to a clinical outcome assessment. A biomarker can be used by the skilled person to make a clinical decision based on its context of use.

As used herein, “peptide,” “polypeptide,” and “protein” are used interchangeably, and refer to a compound comprised of amino acid residues covalently linked by peptide bonds. A protein must contain at least two amino acids and there is no limitation on the maximum number of amino acids that can comprise a protein's sequence. The term “peptide” can refer to a short chain of amino acids including, for example, natural peptides, recombinant peptides, synthetic peptides, or any combination thereof. Proteins and peptides can include, for example, biologically active fragments, substantially homologous polypeptides, oligopeptides, homodimers, heterodimers, variants of polypeptides, modified polypeptides, derivatives, analogs, and fusion proteins, among others.

“Nucleic acid” or “oligonucleotide” or “polynucleotide” as used herein means at least two nucleotides covalently linked together. The depiction of a single strand can also define the sequence of the complementary strand. Thus, a nucleic acid can encompass the complementary strand of a depicted single strand. Many variants of a nucleic acid can be used for the same purpose as a given nucleic acid. Thus, a nucleic acid can encompass substantially identical nucleic acids and complements thereof. A single strand can provide a probe that can hybridize to a target sequence under stringent hybridization conditions. Thus, a nucleic acid can encompass a probe that hybridizes under stringent hybridization conditions. A nucleic acid can be single-stranded, or double-stranded, or can contain portions of both double-stranded and single-stranded sequence. The nucleic acid can be DNA, both genomic and cDNA, RNA, or a hybrid, where the nucleic acid can contain combinations of deoxyribo- and ribo-nucleotides, and combinations of bases including uracil, adenine, thymine, cytosine, guanine, inosine, xanthine hypoxanthine, isocytosine and isoguanine. Nucleic acids can be obtained by chemical synthesis methods or by recombinant methods. Also as used herein, the terms “nucleic acid,” “nucleic acid molecule,” “nucleic acid construct,” “nucleotide sequence”, and “polynucleotide” can refer to RNA or DNA that is linear or branched, single or double stranded, or a hybrid thereof. The term can encompass RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made. A “synthetic” nucleic acid or polynucleotide, as used herein, refers to a nucleic acid or polynucleotide that is not found in nature but is constructed by the hand of man and therefore is not a product of nature.

A “polynucleotide” is a sequence of nucleotide bases, and may be RNA, DNA, or DNA-RNA hybrid sequences (including both naturally occurring and non-naturally occurring nucleotides).

A “fragment” or “portion” of a nucleotide sequence can be understood to mean a nucleotide sequence of reduced length relative (e.g., reduced by 1, 2, 3, 4, 5, 6, 7, 8, 9, 10, 11, 12, 13, 14, 15, 16, 17, 18, 19, 20 or more nucleotides) to a reference nucleic acid or nucleotide sequence and comprising, consisting essentially of, or consisting of a nucleotide sequence of contiguous nucleotides identical or almost identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical) to the reference nucleic acid or nucleotide sequence. Such a nucleic acid fragment or portion according to the disclosure can be, where appropriate, included in a larger polynucleotide of which it is a constituent. In an aspect, a fragment or portion of a nucleotide sequence or nucleic acid sequence can comprise the sequence encoding an exon having one or more mutations. In an aspect, a fragment or portion of a nucleotide sequence or nucleic acid sequence can comprise a target of interest.

A “fragment” or “portion” of an amino acid sequence can be understood to mean an amino acid sequence of reduced length relative (e.g., reduced by 1, 2, 3, 4, 5, 6, 7, 8, 9, 10, 11, 12, 13, 14, 15, 16, 17, 18, 19, 20, 30, 40, 50, 60, 70, 80, 90, 100, 150, 200, 250, or more amino acids) to a reference amino acid sequence and comprising, consisting essentially of, or consisting of an amino acid sequence of contiguous amino acids identical or almost identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical) to the reference amino acid sequence. Such an amino acid fragment or portion according to the disclosure can be, where appropriate, included in a larger amino acid sequence of which it is a constituent.

“Complement” or “complementary” as used herein means a nucleic acid can mean Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

As used herein, the term “prevent” or “preventing” or “prevention” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. In an aspect, preventing progression of a disease, condition, or disorder caused by, related to, and/or exacerbated by the presence of, expression of, and/or activity of one or more secondary RNA structures, one or more tertiary RNA structures, or any combination thereof is intended. The words “prevent” and “preventing” and “prevention” also refer to prophylactic or preventative measures for protecting or precluding a subject (e.g., an individual) not having a disease, condition, or disorder caused by, related to, and/or exacerbated by the presence of, expression of, and/or activity of one or more secondary RNA structures, one or more tertiary RNA structures, or any combination thereof or a elated complication from progressing to that complication.

As used herein, the terms “administering” and “administration” refer to any method of providing one or more of the disclosed compounds, disclosed compositions, disclosed pharmaceutical formulations, or any combination thereof to a subject. Such methods are well known to those skilled in the art and include, but are not limited to, the following routes: oral administration, transdermal administration, administration by inhalation, nasal administration, topical administration, in utero administration, intrahepatic administration, intravaginal administration, ophthalmic administration, intraaural administration, otic administration, intracerebral administration, rectal administration, sublingual administration, buccal administration, and parenteral administration, including injectable such as intravenous administration, intra-CSF administration, intra-arterial administration, intramuscular administration, and subcutaneous administration. Administration can also include hepatic intra-arterial administration or administration through the hepatic portal vein (HPV). Administration of a disclosed therapeutic agent, a disclosed pharmaceutical composition, or a combination thereof can comprise administration directly into the CNS (e.g., intraparenchymal, intracerebroventriular, inthrathecal cisternal, intrathecal (lumbar), deep gray matter delivery, convection-enhanced delivery to deep gray matter) or the PNS. Administration can be continuous or intermittent.

In an aspect, a “therapeutic agent” can be a “biologically active agent” or “biologic active agent” or “bioactive agent”, which refers to an agent that is capable of providing a local or systemic biological, physiological, or therapeutic effect in the biological system to which it is applied. For example, the bioactive agent can act to control infection or inflammation, enhance cell growth and tissue regeneration, control tumor growth, act as an analgesic, promote anti-cell attachment, and enhance bone growth, among other functions. Other suitable bioactive agents can include anti-viral agents, vaccines, hormones, antibodies (including active antibody fragments sFv, Fv, and Fab fragments), aptamers, peptide mimetics, functional nucleic acids, therapeutic proteins, peptides, or nucleic acids. Other bioactive agents include prodrugs, which are agents that are not biologically active when administered but, upon administration to a subject are converted to bioactive agents through metabolism or some other mechanism. Additionally, any of the compositions of the invention can contain combinations of two or more bioactive agents. It is understood that a biologically active agent can be used in connection with administration to various subjects, for example, to humans (i.e., medical administration) or to animals (i.e., veterinary administration). As used herein, the recitation of a biologically active agent inherently encompasses the pharmaceutically acceptable salts thereof.

In an aspect, a “therapeutic agent” can be any agent that effects a desired clinical outcome in a subject having a disease, condition, or disorder caused by, related to, and/or exacerbated by the presence of, expression of, and/or activity of one or more secondary RNA structures, one or more tertiary RNA structures, or any combination thereof, suspected of having a disease, condition, or disorder caused by, related to, and/or exacerbated by the presence of, expression of, and/or activity of one or more secondary RNA structures, one or more tertiary RNA structures, or any combination thereof, and/or likely to develop or acquire a disease, condition, or disorder caused by, related to, and/or exacerbated by the presence of, expression of, and/or activity of one or more secondary RNA structures, one or more tertiary RNA structures, or any combination thereof. In an aspect, a disclosed therapeutic agent can be an oligonucleotide therapeutic agent. A disclosed oligonucleotide therapeutic agent can comprise a single-stranded or double-stranded DNA, iRNA, shRNA, siRNA, mRNA, non-coding RNA (ncRNA), an antisense molecule, miRNA, a morpholino, a peptide-nucleic acid (PNA), or an analog or conjugate thereof. In an aspect, a disclosed oligonucleotide therapeutic agent can be an ASO or an RNAi. In an aspect, a disclosed oligonucleotide therapeutic agent can comprise one or more modifications at any position applicable.

By “determining the amount” is meant both an absolute quantification of a particular analyte (e.g., a disclosed secondary RNA structure and/or a disclosed tertiary RNA structure) or a determination of the relative abundance of a particular analyte (e.g., an amount as compared to a control amount). The phrase includes both direct or indirect measurements of abundance (e.g., individual mRNA transcripts may be quantified or the amount of amplification of an mRNA sequence under certain conditions for a certain period may be used a surrogate for individual transcript quantification) or both.

As used herein, “modifying the method” can comprise modifying or changing one or more features or aspects of one or more steps of a disclosed method. For example, in an aspect, a method can be altered by changing the amount of one or more of the disclosed compounds, disclosed compositions, disclosed pharmaceutical formulations, or any combination thereof administered to a subject, or by changing the frequency of administration of one or more of the disclosed compounds, disclosed compositions, disclosed pharmaceutical formulations, or any combination thereof to a subject, by changing the duration of time one or more of the disclosed compounds, disclosed compositions, disclosed pharmaceutical formulations, or any combination thereof are administered to a subject, or by substituting for one or more of the disclosed components and/or reagents with a similar or equivalent component and/or reagent. The same applies to all the disclosed compounds, disclosed compositions, disclosed pharmaceutical formulations, or any combination thereof.

In an aspect, a therapeutic agent can be a “drug” or a “vaccine” and means a molecule, group of molecules, complex or substance administered to an organism for diagnostic, therapeutic, preventative medical, or veterinary purposes. This term includes externally and internally administered topical, localized and systemic human and animal pharmaceuticals, treatments, remedies, nutraceuticals, cosmeceuticals, biologicals, devices, diagnostics and contraceptives, including preparations useful in clinical and veterinary screening, prevention, prophylaxis, healing, wellness, detection, imaging, diagnosis, therapy, surgery, monitoring, cosmetics, prosthetics, forensics and the like. This term may also be used in reference to agriceutical, workplace, military, industrial and environmental therapeutics or remedies comprising selected molecules or selected nucleic acid sequences capable of recognizing cellular receptors, membrane receptors, hormone receptors, therapeutic receptors, microbes, viruses or selected targets comprising or capable of contacting plants, animals and/or humans. Examples include but are not limited to a radiosensitizer, the combination of a radiosensitizer and a chemotherapeutic, a steroid, a xanthine, a beta-2-agonist bronchodilator, an anti-inflammatory agent, an analgesic agent, a calcium antagonist, an angiotensin-converting enzyme inhibitors, a beta-blocker, a centrally active alpha-agonist, an alpha-1-antagonist, carbonic anhydrase inhibitors, prostaglandin analogs, a combination of an alpha agonist and a beta blocker, a combination of a carbonic anhydrase inhibitor and a beta blocker, an anticholinergic/antispasmodic agent, a vasopressin analogue, an antiarrhythmic agent, an antiparkinsonian agent, an antiangina/antihypertensive agent, an anticoagulant agent, an antiplatelet agent, a sedative, an ansiolytic agent, a peptidic agent, a biopolymeric agent, an antineoplastic agent, a laxative, an antidiarrheal agent, an antimicrobial agent, an antifungal agent, or a vaccine. In a further aspect, the pharmaceutically active agent can be coumarin, albumin, bromolidine, steroids such as betamethasone, dexamethasone, methylprednisolone, prednisolone, prednisone, triamcinolone, budesonide, hydrocortisone, and pharmaceutically acceptable hydrocortisone derivatives; xanthines such as theophylline and doxophylline; beta-2-agonist bronchodilators such as salbutamol, fenterol, clenbuterol, bambuterol, salmeterol, fenoterol; anti-inflammatory agents, including antiasthmatic anti-inflammatory agents, antiarthritis anti-inflammatory agents, and non-steroidal anti-inflammatory agents, examples of which include but are not limited to sulfides, mesalamine, budesonide, salazopyrin, diclofenac, pharmaceutically acceptable diclofenac salts, nimesulide, naproxene, acetaminophen, ibuprofen, ketoprofen and piroxicam; analgesic agents such as salicylates; calcium channel blockers such as nifedipine, amlodipine, and nicardipine; angiotensin-converting enzyme inhibitors such as captopril, benazepril hydrochloride, fosinopril sodium, trandolapril, ramipril, lisinopril, enalapril, quinapril hydrochloride, and moexipril hydrochloride; beta-blockers (i.e., beta adrenergic blocking agents) such as sotalol hydrochloride, timolol maleate, timol hemihydrate, levobunolol hydrochloride, esmolol hydrochloride, carteolol, propanolol hydrochloride, betaxolol hydrochloride, penbutolol sulfate, metoprolol tartrate, metoprolol succinate, acebutolol hydrochloride, atenolol, pindolol, and bisoprolol fumarate; centrally active alpha-2-agonists (i.e., alpha adrenergic receptor agonist) such as clonidine, brimonidine tartrate, and apraclonidine hydrochloride; alpha-1-antagonists such as doxazosin and prazosin; anticholinergic/antispasmodic agents such as dicyclomine hydrochloride, scopolamine hydrobromide, glycopyrrolate, clidinium bromide, flavoxate, and oxybutynin; vasopressin analogues such as vasopressin and desmopressin; prostaglandin analogs such as latanoprost, travoprost, and bimatoprost; cholinergics (i.e., acetylcholine receptor agonists) such as pilocarpine hydrochloride and carbachol; glutamate receptor agonists such as the N-methyl D-aspartate receptor agonist memantine; anti-Vascular endothelial growth factor (VEGF) aptamers such as pegaptanib; anti-VEGF antibodies (including but not limited to anti-VEGF-A antibodies) such as ranibizumab and bevacizumab; carbonic anhydrase inhibitors such as methazolamide, brinzolamide, dorzolamide hydrochloride, and acetazolamide; antiarrhythmic agents such as quinidine, lidocaine, tocainide hydrochloride, mexiletine hydrochloride, digoxin, verapamil hydrochloride, propafenone hydrochloride, flecaimide acetate, procainamide hydrochloride, moricizine hydrochloride, and diisopyramide phosphate; antiparkinsonian agents, such as dopamine, L-Dopa/Carbidopa, selegiline, dihydroergocryptine, pergolide, lisuride, apomorphine, and bromocryptine; antiangina agents and antihypertensive agents such as isosorbide mononitrate, isosorbide dinitrate, propranolol, atenolol and verapamil; anticoagulant and antiplatelet agents such as coumadin, warfarin, acetylsalicylic acid, and ticlopidine; sedatives such as benzodiazapines and barbiturates; ansiolytic agents such as lorazepam, bromazepam, and diazepam; peptidic and biopolymeric agents such as calcitonin, leuprolide and other LHRH agonists, hirudin, cyclosporin, insulin, somatostatin, protirelin, interferon, desmopressin, somatotropin, thymopentin, pidotimod, erythropoietin, interleukins, melatonin, granulocyte/macrophage-CSF, and heparin; antineoplastic agents such as etoposide, etoposide phosphate, cyclophosphamide, methotrexate, 5-fluorouracil, vincristine, doxorubicin, cisplatin, hydroxyurea, leucovorin calcium, tamoxifen, flutamide, asparaginase, altretamine, mitotane, and procarbazine hydrochloride; laxatives such as senna concentrate, casanthranol, bisacodyl, and sodium picosulphate; antidiarrheal agents such as difenoxine hydrochloride, loperamide hydrochloride, furazolidone, diphenoxylate hydrochloride, and microorganisms; vaccines such as bacterial and viral vaccines; antimicrobial agents such as penicillins, cephalosporins, and macrolides, antifungal agents such as imidazolic and triazolic derivatives; and nucleic acids such as DNA sequences encoding for biological proteins, and antisense oligonucleotides. It is understood that a pharmaceutically active agent can be used in connection with administration to various subjects, for example, to humans (i.e., medical administration) or to animals (i.e., veterinary administration). As used herein, the recitation of a pharmaceutically active agent inherently encompasses the pharmaceutically acceptable salts thereof.

“Sequence identity” and “sequence similarity” can be determined by alignment of two peptide or two nucleotide sequences using global or local alignment algorithms. Sequences may then be referred to as “substantially identical” or “essentially similar” when they are optimally aligned. For example, sequence similarity or identity can be determined by searching against databases such as FASTA, BLAST, etc., but hits should be retrieved and aligned pairwise to compare sequence identity. Two proteins or two protein domains, or two nucleic acid sequences can have “substantial sequence identity” if the percentage sequence identity is at least 70%, 75%, 80%, 85%, 90%, 95%, 98%, 99% or more, preferably 90%, 95%, 98%, 99% or more. Such sequences are also referred to as “variants” herein, e.g., other variants of glycogen branching enzymes and amylases. Sequences with substantial sequence identity do not necessarily have the same length and may differ in length. For example, sequences that have the same nucleotide sequence but of which one has additional nucleotides on the 3′- and/or 5′-side are 100% identical.

In an aspect, the skilled person can determine an efficacious dose, an efficacious schedule, and an efficacious route of administration for one or more of the disclosed compounds, disclosed compositions, disclosed pharmaceutical formulations, or any combination thereof so as to treat or prevent a disease, condition, or disorder caused by, related to, and/or exacerbated by the presence of, expression of, and/or activity of one or more secondary RNA structures, one or more tertiary RNA structures, or any combination thereof. In an aspect, the skilled person can also alter, change, or modify an aspect of an administering step to improve efficacy of one or more of the disclosed compounds, disclosed compositions, disclosed pharmaceutical formulations, or any combination thereof. In an aspect, the skilled person can determine an efficacious dose, an efficacious schedule, and an efficacious route of administration for any of the disclosed compounds, disclosed compositions, disclosed pharmaceutical formulations, or any combination thereof.

As used herein, “modifying the method” can comprise modifying or changing one or more features or aspects of one or more steps of a disclosed method. For example, in an aspect, a method can be altered by changing the amount of one or more of the disclosed compounds, disclosed compositions, disclosed pharmaceutical formulations, or any combination thereof administered to a subject, or by changing the frequency of administration of one or more of the disclosed compounds, disclosed compositions, disclosed pharmaceutical formulations, or any combination thereof, or by changing the duration of time that the one or more of the disclosed compounds, disclosed compositions, disclosed pharmaceutical formulations, or any combination thereof are administered to a subject.

As used herein, “isolated” refers to a nucleic acid molecule or a nucleic acid sequence that has been substantially separated, produced apart from, or purified away from other biological components in the cell or tissue of an organism in which the component occurs, such as other cells, chromosomal and extrachromosomal DNA and RNA, and proteins. Nucleic acids and proteins that have been “isolated” include nucleic acids and proteins purified by standard purification methods. The term also embraces nucleic acids and proteins prepared by recombinant expression in a host cell as well as chemically synthesized nucleic acids and proteins. Isolated proteins or nucleic acids, or cells containing such, in some examples are at least 50% pure, such as at least 75%, at least 80%, at least 90%, at least 95%, at least 98%, or at least 100% pure.

As used herein, “concurrently” means (1) simultaneously in time, or (2) at different times during the course of a common treatment schedule.

The term “contacting” as used herein refers to bringing one or more of the disclosed compounds, disclosed compositions, disclosed pharmaceutical formulations, or any combination thereof together with a target area or intended target area in such a manner that the one or more of the disclosed compounds, disclosed compositions, disclosed pharmaceutical formulations, or any combination thereof exert an effect on the intended target or targeted area either directly or indirectly. A target area or intended target area can be one or more of a subject's organs (e.g., lungs, heart, liver, kidney, brain, etc.). In an aspect, a target area or intended target area can be any cell or any organ affected by one or more disease-associated RNAs or disease relevant RNAs. In an aspect, a target area or intended target area can be the brain or various neuronal populations. In an aspect, a target area or intended target area can be any cell or any organ infected by an over-expression or an under-expression of one or more disease-associated RNAs or disease relevant RNAs.

As used herein, “determining” can refer to measuring or ascertaining the expression and/or activity of one or more disease-associated RNAs or disease relevant RNAs. Methods and techniques used to determine the expression and/or activity of one or more disease-associated RNAs or disease relevant RNAs are typically known to the medical arts. For example, the art is familiar with the ways to identify and/or diagnose the presence, severity, or both of a disease, condition, or disorder caused by, related to, and/or exacerbated by the presence of, expression of, and/or activity of one or more secondary RNA structures, one or more tertiary RNA structures, or any combination thereof. In an aspect, “determining” can also refer to measuring or ascertaining the level of one or more proteins or peptides in a bio sample, or measuring or ascertaining the level or one or more RNAs or miRNAs in a bio sample. Methods and techniques for determining the expression and/or activity level of relevant proteins, peptides, mRNA, DNA, or any combination thereof known to the art and are disclosed herein.

As used herein, “effective amount” and “amount effective” can refer to an amount that is sufficient to achieve the desired result such as, for example, the treatment and/or prevention of a disease, condition, or disorder caused by, related to, and/or exacerbated by the presence of, expression of, and/or activity of one or more secondary RNA structures, one or more tertiary RNA structures, or any combination thereof. As used herein, the terms “effective amount” and “amount effective” can refer to an amount that is sufficient to achieve the desired an effect on an undesired condition (e.g., a disease, condition, or disorder caused by, related to, and/or exacerbated by the presence of, expression of, and/or activity of one or more secondary RNA structures, one or more tertiary RNA structures, or any combination thereof). For example, a “therapeutically effective amount” refers to an amount that is sufficient to achieve the desired therapeutic result or to have an effect on undesired symptoms, but is generally insufficient to cause adverse side effects. In an aspect, “therapeutically effective amount” means an amount of the disclosed compounds, disclosed compositions, disclosed pharmaceutical formulations, or any combination thereof that (i) treats the particular disease, condition, or disorder (e.g., a disease, condition, or disorder caused by, related to, and/or exacerbated by the presence of, expression of, and/or activity of one or more secondary RNA structures, one or more tertiary RNA structures, or any combination thereof), (ii) attenuates, ameliorates, or eliminates one or more symptoms of the particular disease, condition, or disorder (e.g., a disease, condition, or disorder caused by, related to, and/or exacerbated by the presence of, expression of, and/or activity of one or more secondary RNA structures, one or more tertiary RNA structures, or any combination thereof), or (iii) delays the onset of one or more symptoms of the particular disease, condition, or disorder described herein (e.g., a disease, condition, or disorder caused by, related to, and/or exacerbated by the presence of, expression of, and/or activity of one or more secondary RNA structures, one or more tertiary RNA structures, or any combination thereof). The specific therapeutically effective dose level for any particular patient will depend upon a variety of factors including the disorder being treated and the severity of the disorder; the disclosed compounds, disclosed compositions, disclosed pharmaceutical formulations, or any combination thereof employed; the disclosed methods employed; the age, body weight, general health, sex and diet of the patient; the time of administration; the route of administration; the rate of excretion of the disclosed compounds, disclosed compositions, disclosed pharmaceutical formulations, or any combination thereof employed; the duration of the treatment; drugs used in combination or coincidental with the disclosed compounds, disclosed compositions, disclosed pharmaceutical formulations, or any combination thereof employed, and other like factors well known in the medical arts. For example, it is well within the skill of the art to start doses of the disclosed compounds, disclosed compositions, disclosed pharmaceutical formulations, or any combination thereof at levels lower than those required to achieve the desired therapeutic effect and to gradually increase the dosage until the desired effect is achieved. If desired, then the effective daily dose can be divided into multiple doses for purposes of administration. Consequently, a single dose of the disclosed compounds, disclosed compositions, disclosed pharmaceutical formulations, or any combination thereof can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition, such as, for example, a disease, condition, or disorder caused by, related to, and/or exacerbated by the presence of, expression of, and/or activity of one or more secondary RNA structures, one or more tertiary RNA structures, or any combination thereof.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents, or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. In an aspect, a pharmaceutical carrier employed can be a solid, liquid, or gas. In an aspect, examples of solid carriers can include lactose, terra alba, sucrose, talc, gelatin, agar, pectin, acacia, magnesium stearate, and stearic acid. In an aspect, examples of liquid carriers can include sugar syrup, peanut oil, olive oil, and water. In an aspect, examples of gaseous carriers can include carbon dioxide and nitrogen. In preparing a disclosed composition for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “excipient” refers to an inert substance which is commonly used as a diluent, vehicle, preservative, binder, or stabilizing agent, and includes, but is not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). See, also, for reference, Remington's Pharmaceutical Sciences, (1990) Mack Publishing Co., Easton, Pa., which is hereby incorporated by reference in its entirety.

As used herein, the term “package insert” is used to refer to instructions customarily included in commercial packages of therapeutic products, that contain information about the indications, usage, dosage, administration, contraindications and/or warnings concerning the use of such therapeutic products.

As used herein, the term “in combination” in the context of the administration of one or more of the disclosed compounds, disclosed compositions, disclosed pharmaceutical formulations, or any combination thereof includes the use of more than one therapy (e.g., additional therapeutic agents). Administration “in combination with” one or more additional therapeutic agents includes simultaneous (e.g., concurrent) and consecutive administration in any order. The use of the term “in combination” does not restrict the order in which therapies are administered to a subject. By way of non-limiting example, a first therapy (e.g., one or more of the disclosed compounds, disclosed compositions, disclosed pharmaceutical formulations, or any combination thereof) may be administered prior to (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 8 weeks, 8 weeks, 9 weeks, 10 weeks, 11 weeks, or 12 weeks), concurrently, or after (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 7 weeks, 8 weeks, 9 weeks, 10 weeks, 11 weeks, or 12 weeks or longer) the administration of a second therapy (e.g., one or more of the disclosed compounds, disclosed compositions, disclosed pharmaceutical formulations, or any combination thereof or one or more additional therapeutic agents) to a subject having or diagnosed with a disease, condition, or disorder caused by, related to, and/or exacerbated by the presence of, expression of, and/or activity of one or more secondary RNA structures, one or more tertiary RNA structures, or any combination thereof.

Disclosed are the components to be used to prepare the disclosed compounds, disclosed compositions, disclosed pharmaceutical formulations, or any combination thereof as well the disclosed compounds, disclosed compositions, disclosed pharmaceutical formulations, or any combination thereof used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific aspects or combination of aspects of the disclosed methods.

### B. RNA Tertiary Structures

RNA tertiary structure is defined as the arrangement and interaction of secondary structure building blocks in three-dimensional (3D) space. RNA tertiary structure is defined as the three-dimensional arrangement of RNA building blocks, which include helical duplexes, triple-stranded structures, and other components that are held together through connections collectively termed RNA tertiary interactions. Biophysical techniques are being used to elucidate the driving forces for tertiary structure formation and the mechanisms for its stabilization.

RNA tertiary folding is promoted by maximization of base stacking, much like the hydrophobic effect that drives protein folding. RNA folding also requires electrostatic stabilization, both through charge screening and site binding of metals, and it is enhanced by desolvation of the phosphate backbone. A major determinant for overall tertiary RNA architecture is local conformation in secondary-structure junctions, which are regions from which two or more duplexes project. At junctions and other structures, such as pseudoknots and kissing loops, adjacent helices stack on one another, and these coaxial stacks play a major role in dictating the overall architectural form of an RNA molecule. In addition to RNA junction topology, a second determinant for RNA tertiary structure is the formation of sequence-specific interactions. Networks of triple helices, tetraloop receptor interactions, and other sequence-specific contacts establish the framework for the overall tertiary fold. The third determinant of tertiary structure is the formation of stabilizing stacking and backbone interactions, and many are not sequence specific. For example, ribose zippers allow 2′-hydroxyl groups on different RNA strands to form networks of interdigitated hydrogen bonds, serving to seal strands together and thereby stabilize adjacent substructures. These motifs often require monovalent and divalent cations, which can interact diffusely or through chelation to specific RNA functional groups.

### Stacking and Coaxial Helices

Stacking of the aromatic nucleic acid bases is one of the most important driving forces during formation of RNA structure. In general, an RNA molecule will attempt to maximize base stacking, particularly at helical termini. If two helices are next to each other (e.g., separated by a phosphodiester linkage), their terminal base pairs will stack, and the helices will become colinear, resulting in a “coaxially stacked” substructure. The earliest glimpses of nucleic acid tertiary structure, in the form of tRNA crystal structures, revealed that coaxial stacking of helices determines the overall molecular shape. Each of the four helices in the “cloverleaf” tRNA secondary structure chooses a stacking partner, and these pairs of helices stack end-on-end, forming two long helices that ultimately arrange themselves through tertiary interactions. Numerous subsequent studies have shown that the choice of stacking partners among sets of helices is a critical determinant of RNA structural fate. The coaxial stacking of helices at junctions is thermodynamically favorable, and the free energy gained is sequence dependent, closely following the trends observed for nearest neighbor interactions in formation of RNA secondary structure. From 0.5 to 3.0 kcal/mol of free energy is released upon formation of a coaxial stack, and these energetic differences can influence choice of coaxial stacking partners at a junction.

Tertiary structures such as kissing loops and pseudoknots are composed of coaxially stacked helices and therefore represent RNA structures that are almost completely dictated by the forces driving coaxial stacking. At RNA junctions, duplexes immediately next to each other tend to coaxially stack, to minimize folding free energy. However, determining which helices are actually adjacent can be difficult because “linker” nucleotides are not always flexible. Rather, linker nucleotides often form noncanonical base pairs that extend the helical terminus, thereby presenting an alternative interface with other helices. For example, A G, C C, and G U pairs are common at helical termini, where they serve as energetically favorable interfaces for coaxial stacks. Although the role of sequence is central, coaxial stacking choice can be strongly dictated by ionic conditions and by the topological constraints of intervening junctions.

### Kissing Loops

Long-range base pairings between hairpin stem loops are known as “kissing” loops. These interactions are mediated by loop nucleotides that interact through complementary Watson Crick base pairs. Kissing loops have been found in many RNAs, including tRNA, mRNA, and rRNA. Stable loop-loop interactions can occur with as few as two base pairs between the loops. Kissing loop interactions are commonly used by retroviruses to initiate dimerization of genomic RNA. The HIV-1 dimerization initiation site (DIS) is a well-studied example of this interaction. The self-complementary loop forms a stable interaction involving six base pairs that coaxially stack within a continuous helix that contains bulged purines.

### Kink Turns

Structural Features. Kink turns (or K-turns) were identified during analysis of the large ribosomal subunit crystal structure, and they have been found in a diversity of RNAs. The K-turn is a helix loop helix motif that bends the RNA helical axis by ˜120°, resulting in a close juxtaposition of helical minor grooves. One helix is canonical, whereas the other is noncanonical with tandem G-A base pairs. The bend is initiated by a three-nucleotide loop that is typically purine-rich. The severe kink in the loop is facilitated by the G-A pairs, which form cross-strand stacking interactions that twist the backbone in a characteristic manner. The adenines of the tandem G-A pairs stack on each other and participate in A-minor interactions across the junction.

K-turns are often bound and stabilized by proteins. In isolation, the K-turn is in equilibrium between the kinked form and a more extended conformation. The kinked population is stabilized by high concentrations of metal ions, but it remains in equilibrium with an extended form. K-turns are not believed to provide a thermodynamic driving force for RNA tertiary folding, but rather require cooperation from surrounding proteins or RNA tertiary structure to stabilize them. The “hook turn” is a recurrent motif that was originally characterized during crystallographic investigations of a loop E motif from the 5S rRNA of a purple sulfur bacterium. This RNA, which undergoes a sudden reverse in direction stabilized by a sheared G-A pair and a reverse-Hoogsteen pair, was found to be recurrent by scanning structures of the ribosome using the motif-finding algorithm Primos. The same algorithm was also used to identify the constituent K-turns and S-turns within the ribosome, revealing that the S-turn occurs in two distinct forms.

### Pseudoknots

Pseudoknots are RNA tertiary structures that arise from energetically favorable long-range interactions between loops and hairpins that are stabilized by coaxial stacking of the newly formed RNA helices. Such structures have been found and mapped across many species and RNAs, from human telomerase RNA to viral genomes, with biological functions that are still being elucidated. Notably, pseudoknots have been found to be essential for viral replication in a number of viruses, including severe acute respiratory syndrome coronavirus-2 (SARS-COV), where their main function is in 1 programmed ribosomal frameshifting. Frameshifting is a desirable target in some viruses as essential proteins encoded by the secondary open reading frame (ORF1b) region are normally out of frame, thus requiring a re-arrangement of the ribosome to proceed with elongation and translation. In coronaviruses, re-arrangement takes place through the presence of a slippery site, a linker region, and the downstream regulatory pseudoknot. While normal ribosome positioning starts at the zero position of the slippery site, the encounter between the ribosome and the pseudoknot tertiary structure causes the ribosome to pause and re-arrange at the 1 position, which puts ORF1b in frame and allows for ribosomal elongation upon pseudoknot unwinding. Mutational studies as well as antisense peptide nucleic acid (PNA) targeting of the pseudoknot revealed its potential as an antiviral target to dramatically decrease replication and virion production.

### Triple-Stranded RNA Structures

Base triples occur frequently in RNA tertiary structure. For example, there are 27 base triples in the 50S ribosomal subunit, and 10 triples in the 30S subunit. The number of possible base triples constrained by at least three hydrogen bonds is 840. In the Tetrahymena intron, the active site contains a sandwich of four base triples. Triple-stranded RNA structures contain a Watson Crick base-paired duplex that hydrogen bonds to a third strand. While some triples occur in the minor groove (A-minor motifs, vide supra), base triples can also occur within the narrow major groove of RNA. Base triples that involve a third strand in the major groove often utilize the “Hoogsteen” face of purines. One example of such an interaction is observed in the telomerase pseudoknot, which has a loop that lies in the helical major groove and forms a series of Hoogsteen base triples. Similarly, a series of stacked major groove triples comprises the conserved “catalytic triplex” within the group II intron active site, where it plays a key role in supporting reaction chemistry. A similar triplex network is hypothesized to exist within the active site of the eukaryotic spliceosome.

Hoogsteen triples are most commonly U-A·U, where the adenine N7 accepts a hydrogen bond from the U imino proton. The base triple C-G·C+ is isosteric with U-A·U but requires protonation of the third strand C in order to form a hydrogen bond with the guanine N7. The SAM-II riboswitch bound to S-adenosylmethionine contains a pseudoknot with an extended major-groove triple helix.

Another example was recently identified within the Kaposi's sarcoma-associated herpesvirus, which produces a highly abundant noncoding RNA, the polyadenylated nuclear (PAN) RNA. PAN contains an expression and nuclear retention element (ENE) that prevents degradation of its message. The ENE contains an internal loop is made up of uracils, which clamp onto the poly-A tail of the message, thereby forming a triple stranded U-A· U structure.

### Triple Helices

Base triples occur frequently in RNA and usually result from the insertion of a third single RNA strand into a duplex in the minor groove, via A-minor interactions, or in the major groove through Hoogsteen H-bonds. Base triples can be found as part of other tertiary and quaternary structures to enhance their stability, such as within pseudoknots or ribosomal subunits. Stand-alone triple helices have recently been mapped within several non-coding RNAs (ncRNAs) and are linked to an increase in the half-life of the transcript by protecting the transcript from degradation. Despite their clear biological relevance, biophysical and structural methods for their characterization in vitro and in vivo are still limited. Triple helices have mainly been structurally investigated through NMR, FRET experiments, ultraviolet (UV)-visible, and X-ray diffraction, which have limitations in the throughput and/or reflection of the conformer population.

### G-Quadruplexes

Initially discovered in DNA decades ago, G4s are folding structures formed within a G-rich strand or between two G-rich strands. Generally, a tetrad of guanines arranges co-planarly via Hoogsteen H-bond interactions further stabilized by interactions with a cation. Computational and experimental methods have helped map G4 structures in the promoter region of many eukaryotic and prokaryotic genes, with the most notable one being telomeric DNA. G4 overall structure depends on strand polarity, orientation of connecting loops, and cation stabilization, thereby making it a diverse topological family. Recent studies have supported the existence of G4s in several ncRNA, though these differ from DNA G4s as they can be transient and highly dynamic. Remarkably, RNA G4s have showed higher stability than their DNA counterparts due to the added H-bonds of the 20′-OH, as well as stronger stacking of the G-quartets. These key differences ultimately distinguish RNA G4s unique topological features, allowing for small molecule discrimination between RNA and DNA.

### Tetraloop-Receptor Motifs

Tetraloop-receptor motifs are among the most common types of long-range RNA tertiary interaction. They have been observed in almost every large RNA crystal structure and have even been employed in the fabrication of RNA nanostructures. In every case, this motif involves a terminal hairpin loop that contains a signature sequence, and known examples of interacting tetraloops are the GNRA and GANC tetraloops. While other conserved tetraloops are known (such as UNCG and CUYG), these serve as stable caps for hairpin termini and do not typically function as tertiary interaction partners.

There are several types of receptor motifs for tetraloops, and they differ in their level of structural complexity. The most complex type of receptors are highly conserved internal loop motifs, such as the “11-nucleotide motif”, which specifically recognizes GAAA tetraloops, and the IC3 motif, which has a more relaxed specificity for GNRA tetraloops. The interaction between the GAAA tetraloop and the 11-nucleotide receptor is surprisingly stable.

Molecular interactions between GAAA tetraloops and the 11-nucleotide receptor are extensive and include two major components: (1) The second adenine of the GAAA tetraloop stacks on an A-A platform that is formed within the receptor loop. Note that in some instances, the A-A platform can be A-C. The second adenine of the tetraloop also forms supporting hydrogen bonds to the receptor. (2) The remaining bases of the tetraloop engage in a network of hydrogen bonds with G C base pairs in the adjacent receptor helix.

A simpler class of receptors are tandem GC pairs, which represent the first type of tetraloop-receptor interaction to be visualized crystallographically. The interaction first appeared as a set of crystal contacts reported during structural studies of the hammerhead ribozyme. Later dubbed “A-minor motifs” (vide supra), the G C pairs within the minor groove of these receptors form hydrogen bonds with base and sugar substituents on tetraloop nucleotides 3 and 4. Similar variations on this subfamily were reported within group I introns, and they have subsequently been observed in many RNA crystal structures. By contrast, the GANC tetraloops interact exclusively through base stacking with their cognate receptors, which are simple extrahelical bulged purines. Occurring only in group IIC introns, these tetraloop-receptor interactions are highly conserved.

### RNA Quadruplex Structures

Repeated stretches of guanine-rich sequences can form quadruple base pairs that stack on each other to form quadruplexes. Telomeric DNA contains such guanine repeats, and it has recently been discovered that telomeres are transcribed into a large noncoding RNA called TERRA (telomeric repeat-containing RNA). TERRA RNA has been implicated in regulation of telomere length, through telomerase inhibition and chromatin remodeling. The crystal structure of a quadruplex derived from human TERRA has been solved. Guanine quadruplexes are stabilized by potassium ions, which are chelated in the center of the quadruplex by the guanine oxygens. Electrospray mass spectrometry reveals that TERRA quadruplexes form stable multimers. An emerging body of evidence suggests that RNA quadruplex structures play an important role in regulating translation of mRNA and splicing of some pre-mRNAs. Such RNA quadruplexes are recognized by the fragile X mental retardation protein (FMRP), which acts as a repressor of translation.

### Ribose and the 20′-Hydroxyl Group: Key Components of RNA Tertiary Structure

The 20′-hydroxyl group of the RNA backbone is a stabilizing component in many tertiary interactions, since it can make two hydrogen bonds, acting as both hydrogen-bond donor and acceptor. The prevalence of 20′-OH contacts was evident from crystal structures of tRNA, and their thermodynamic contribution to RNA tertiary structure was revealed through studies of the Tetrahymena ribozyme. Many subsequent biochemical and structural studies revealed the importance of 20′-hydroxyl groups in ribozymes and other large RNA molecules, where they contribute to many motifs, including the A-minor interaction. Structural studies show that 20′-OH groups usually form networks of interactions, forming arrays with different types of morphologies. The most common type of 20′-OH array is the ribose zipper motif, which brings two backbone strands into close proximity through interdigitated 20′-OH interactions, thereby stabilizing neighboring structural features. Ribose zippers were first reported in early studies of ribozyme structure, where they play a key role in supporting core architecture. Inspection of solved structures shows that groups of ribose zippers tend to surround and buttress more sequence-specific types of tertiary interactions, such as tetraloop-receptor interactions, kissing loops, and S-turns. In this way, ribose zippers provide the “glue” that maintains stability of entire tertiary substructures. A single 20-hydroxyl group within a tertiary interaction network may contribute 1-2 kcal/mol of interaction free energy, so the stabilizing influence of a ribose zipper is likely to be considerable. While most ribose zipper motifs would seem to be sequence independent (all nucleotides contain a ribose), some types of ribose zipper motifs are found in specific sequence contexts, and their formation may be directed by the surrounding structural environment.

### C. MALAT1

One example of a biologically relevant triple helix is the one present at the 30-end of the metastasis-associated lung adenocarcinoma transcript 1 (MALAT1). Metastasis Associated Lung Adenocarcinoma Transcript 1 (MALAT1) is also known as Nuclear Enriched Abundant Transcript 2 (NEAT2) and was first identified in a microarray screen of tumors from patients with non-small cell lung cancer. MALAT1 was found to be upregulated in the tumors with a higher propensity to metastasize. The MALAT1 gene is encoded on human chromosome 11q13.1 and mouse chromosome 19qA. It is in a gene dense region with a very high syntenic evolutionary conservation.

This lncRNA gained attention as a therapeutic target after it was found to be overexpressed in several cancer types, and it has now been implicated in other disease-related pathways. Specifically, the 8.7-kb long immature MALAT1 transcript undergoes a multiple-step processing, including the cleavage of a 30-end 61-nucleotide (nt) tRNA-like structure. The process yields the blunt formation of a 30-end triple helix that has been associated with nuclear accumulation of the transcript and as an essential element for protection against degradation. A reported crystal structure obtained from a truncated version of the wild-type (WT) triple helix revealed 9 U,A-U base triples resulting from the major groove insertion of a genomically encoded A-rich 30-tail, interrupted by an essential+C,G-C triple and a C-G doublet (Brown et al., 2014). Recently, two notable examples of small molecule targeting have been reported for the MALAT1 triple helix utilizing a focused library and high-throughput approach, respectively.

MALAT1 exhibits a remarkable sequence conservation with greater than 50% overall conservation in vertebrates and greater than 80% conservation at the 3′ end of the transcript. This is one of the key distinguishing features of MALAT1 as very few lncRNAs show such a high level of evolutionary conservation. Less than 10% of all lncRNAs show exonic as well as structural conservation equivalent to that of protein coding genes. The MALAT1 transcript has been confirmed as a non-coding RNA as it exhibits low protein-coding potential using two independent coding potential calculating algorithms CPC2 and CPAT.

Human MALAT1 is ˜8.7 knt long, whereas the mouse RNA is 6.7 knt long. It is transcribed by RNA polymerase II and its promoter has an accessible open chromatin architecture, which has been shown in several high-throughput studies and DNAse sensitivity assays. The steady state expression level of MALAT1 is very high and is comparable to highly transcribed housekeeping genes, such as B-Actin. Further, MALAT1 is ubiquitously expressed across all tissues with an average median expression of about 150 TPM (transcripts per million) with highest expression in ovaries with a median expression of 287 TPM. The abundance of MALAT1 in cells is likely the consequence of strong promoter activity coupled with increased stability of the transcribed RNA.

MALAT1 was originally classified as an intron-less transcript with a genomically-encoded poly A tract. However, with a number of deep sequencing e orts, several alternatively spliced isoforms and transcripts with alternative transcription start sites have been identified that are expressed during different physiological states such as cancer. The MALAT1 primary transcript is processed to yield the well characterized nuclear retained MALAT1 transcript, and from its 3′ end a tRNA-like small RNA. The biogenesis of the small RNA is mediated by the tRNA processing machinery, RNase P and RNase Z. The 61-nucleotide tRNA-like MALAT1-associated small cytoplasmic RNA (mascRNA) is exported to the cytoplasm. The resultant 3′ end of the nuclear MALAT1 transcript post-processing is not polyadenylated, however, it contains a genomically-encoded poly(A)-rich stretch which pairs with an upstream U-rich region and then adopts a unique triple helical confirmation. This triple helical structure was first identified in the PAN (polyadenylated nuclear) RNA produced by the human oncogenic Kaposi sarcoma-associated-herpesvirus (KSHV) PAN RNA. The only other human or mouse RNA that exhibits such a structure is the ˜20 knt Men-(NEAT1_2) RNA. The triple helical structure has been shown to confer stability and nuclear localization to MALAT1 in the absence of a true poly(A) tail and has been shown to bind several RNA binding proteins (RBPs) including METTL16 which is an m⁶A RNA methyl-transferase.

Taken together, the MALAT1 locus displays remarkable evolutionarily conserved secondary and tertiary structural features and an unusual 3′ end processing mechanism. It is not fully apparent whether the full length MALAT1 RNA with its 3′ end triplex structure, the processed tRNA-like RNA, and the natural antisense RNA have a concerted mechanism of action or if each component derived from this interesting locus has a disparate function. Further, high-throughput chemical mapping experiments have highlighted extensive epi-transcriptomic changes in the MALAT1 transcript, for example, m⁶A, pseudouridylation and 5-methyl cytosine. It has been shown that the addition of m⁶A at the A2577 position could destabilize the hairpin stem of MALAT1, making it accessible for RNA-binding proteins such as HNRNPC. Additional detailed molecular studies elucidating the transcriptional and post-transcriptional regulation of the MALAT1 locus will address these issues and allow us to further understand the regulation and function of the MALAT1 locus.

### D. SCHLAP1

SWI/SNF Complex Antagonist Associated with Prostate Cancer 1 (SCHLAP1) is an RNA gene, and is affiliated with the lncRNA class. Diseases associated with SCHLAP1 include prostate disease and prostate cancer. SCHLAP1 is also identified as HGNC 48603, NCBI Entrez Gene 101669767, Ensembl ENSG00000281131, and OMIM 615568. Chromosome locus associated with prostate-1 (SChLAP1) is one of the most important lncRNAs. It is located in the nucleus. SChLAP1 is highly overexpressed in a subset of prostate cancers and is associated with lethal disease. However, little is known about the exact mechanism of SChLAP1 in prostate cancer development.

### E. NEAT1

Nuclear Paraspeckle Assembly Transcript 1 (NEAT1) is an RNA gene and is affiliated with the lncRNA class. NEAT1 RNA, a highly abundant 4 kb ncRNA, is retained in nuclei in approximately 10 to 20 large foci that are completely coincident with paraspeckles, nuclear domains implicated in mRNA nuclear retention. Diseases associated with NEAT1 include Dengue Disease and Gastric Adenocarcinoma. Among its related pathways are Extrafollicular B cell activation by SARS-CoV-2. NEAT1 is also identified as HGNC 30815, NCBI Entrez Gene 283131, Ensembl ENSG00000245532, and OMIM 612769.

### F. LMO7

LMO7 (LIM Domain 7) is a protein coding gene. This gene encodes a protein containing a calponin homology (CH) domain, a PDZ domain, and a LIM domain, and may be involved in protein-protein interactions. Several alternatively spliced transcript variants encoding different isoforms have been found for this gene, however, the full-length nature of some variants is not known. Diseases associated with LMO7 include Townes-Brocks Syndrome and Hyperalphalipoproteinemia 1. Among its related pathways are Class I MHC mediated antigen processing and presentation and Signaling by ALK in cancer. Gene Ontology (GO) annotations related to this gene include ubiquitin-protein transferase activity and actinin binding. LMO7 is also identified as HGNC 6646, NCBI Entrez Gene 4008, Ensembl ENSG00000136153, OMIM 604362, and UniProtKB/Swiss-Prot Q8WWI1.

### G. Inventive Compounds

Disclosed herein are one or more compounds made by a method of assessing a library of shape diverse small molecules. Disclosed herein are one or more compounds made by a disclosed method.

Disclosed herein is a compound comprising a small molecule having a diminazene (DMZ) scaffold. Disclosed herein is a compound comprising a small molecule having a diminazene scaffold and a diamidine moiety.

Disclosed herein is a compound of formula

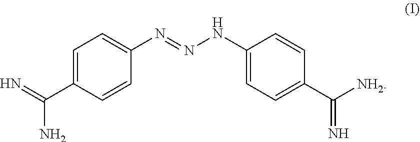 [SMILES: N=C(N)c1ccc(/N=N/Nc2ccc(C(=N)N)cc2)cc1]

Disclosed herein is a compound of formula

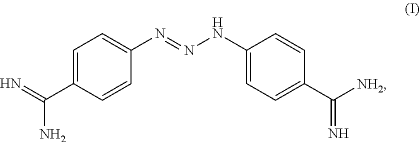 [SMILES: N=C(N)c1ccc(/N=N/Nc2ccc(C(=N)N)cc2)cc1]

having one or more of ortho, para, and/or meta substitutions.

In an aspect, a disclosed ortho, para, and/or meta substitution can comprise any one of R0-R28 or a combination of any one of R0-R28.

In an aspect, R0 can be

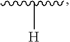

### R1 can be

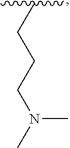

### R2 can be

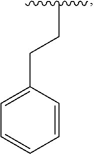

### R3 can be

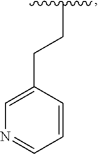

### R4 can be

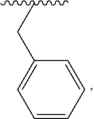 [SMILES: *Cc1ccccc1]

### R5 can be

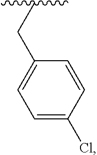 [SMILES: *Cc1ccc(Cl)cc1]

### R6 can be

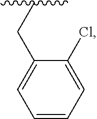 [SMILES: *Cc1ccccc1Cl]

### R7 can be

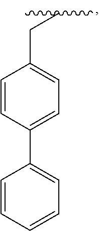 [SMILES: *Cc1ccc(-c2ccccc2)cc1]

### R8 can be

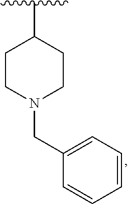 [SMILES: *C1CCN(Cc2ccccc2)CC1]

### R9 can be

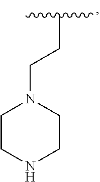 [SMILES: *CCN1CCNCC1]

### R10 can be

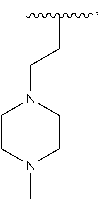 [SMILES: *CCN1CCN(C)CC1]

### R11 can be

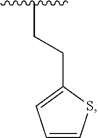 [SMILES: *CCc1cccs1]

### R12 can be

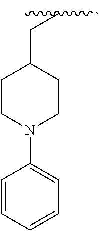 [SMILES: *CC1CCN(c2ccccc2)CC1]

### R13 can be

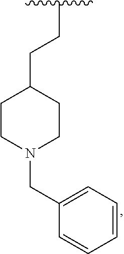 [SMILES: *CCC1CCN(Cc2ccccc2)CC1]

### R14 can be

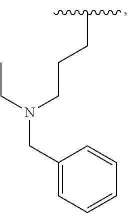 [SMILES: *CCCN(CC)Cc1ccccc1]

### R15 can be

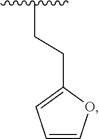 [SMILES: *CCc1ccco1]

### R16 can be

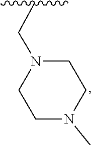 [SMILES: *CN1CCN(C)CC1]

### R17 can be

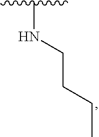 [SMILES: *NCCCC]

### R18 can be

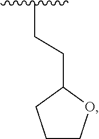 [SMILES: *CCC1CCCO1]

### R19 can be

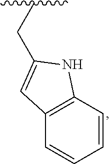 [SMILES: *Cc1cc2ccccc2[nH]1]

### R20 can be

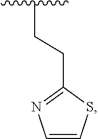 [SMILES: *CCc1nccs1]

### R21 can be

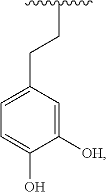 [SMILES: *CCc1ccc(O)c(O)c1]

### R22 can be

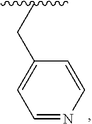 [SMILES: *Cc1ccncc1]

### R23 can be

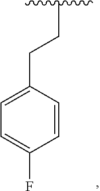 [SMILES: *CCc1ccc(F)cc1]

### R24 can be

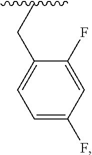 [SMILES: *Cc1ccc(F)cc1F]

### R25 can be

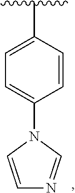 [SMILES: *c1ccc(-n2ccnc2)cc1]

### R26 can be

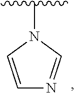 [SMILES: *n1ccnc1]

### R27 can be

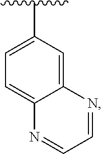 [SMILES: *c1ccc2nccnc2c1]

### and R28 can be

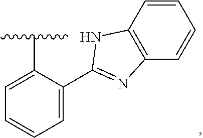 [SMILES: *c1ccccc1-c1nc2ccccc2[nH]1]

or any combination thereof.

Disclosed herein is a compound of formula

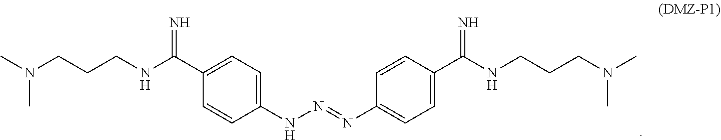

Disclosed herein is a compound of formula

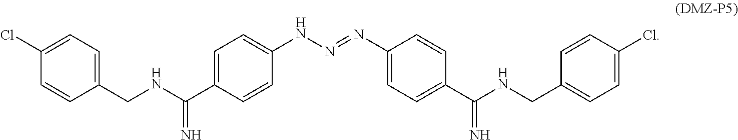 [SMILES: N=C(NCc1ccc(Cl)cc1)c1ccc(/N=N/Nc2ccc(C(=N)NCc3ccc(Cl)cc3)cc2)cc1]

Disclosed herein is a compound of formula

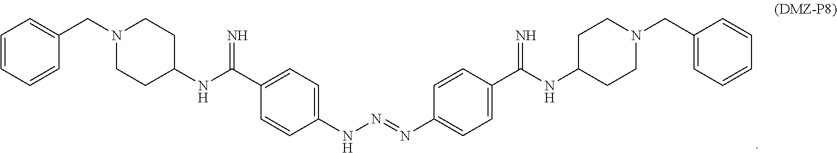 [SMILES: N=C(NC1CCN(Cc2ccccc2)CC1)c1ccc(/N=N/Nc2ccc(C(=N)NC3CCN(Cc4ccccc4)CC3)cc2)cc1]

Disclosed herein is a compound of formula

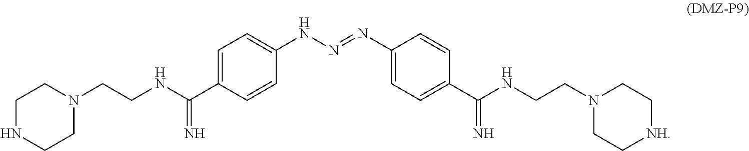 [SMILES: N=C(NCCN1CCNCC1)c1ccc(/N=N/Nc2ccc(C(=N)NCCN3CCNCC3)cc2)cc1]

Disclosed herein is a compound of formula

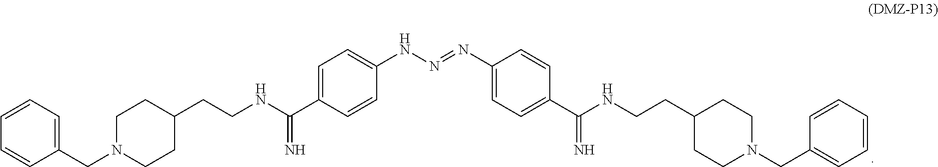 [SMILES: N=C(NCCC1CCN(Cc2ccccc2)CC1)c1ccc(/N=N/Nc2ccc(C(=N)NCCC3CCN(Cc4ccccc4)CC3)cc2)cc1]

Disclosed herein is a compound of formula

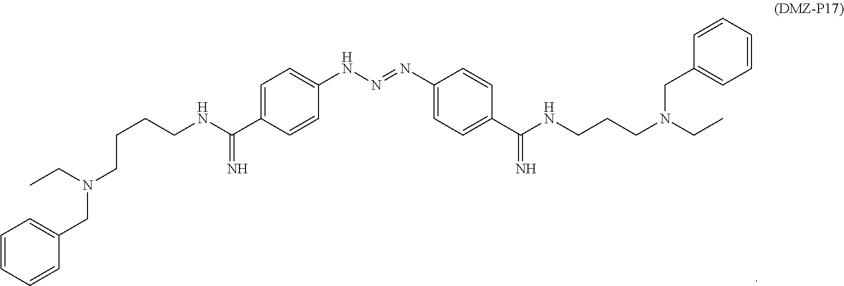 [SMILES: CCN(CCCCNC(=N)c1ccc(N/N=N/c2ccc(C(=N)NCCCN(CC)Cc3ccccc3)cc2)cc1)Cc1ccccc1]

Disclosed herein is a compound of formula

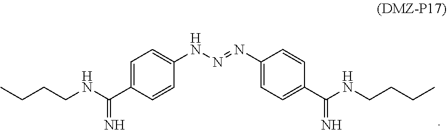 [SMILES: CCCCNC(=N)c1ccc(/N=N/Nc2ccc(C(=N)NCCCC)cc2)cc1]

Disclosed herein is a compound of formula

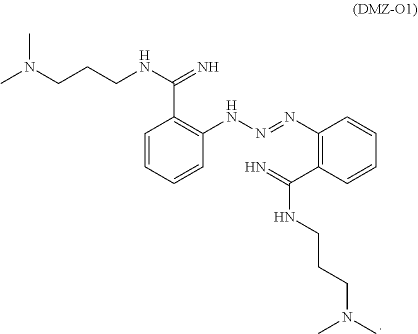

Disclosed herein is a compound of formula

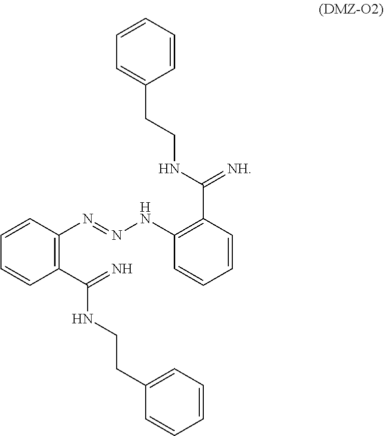 [SMILES: N=C(NCCc1ccccc1)c1ccccc1/N=N/Nc1ccccc1C(=N)NCCc1ccccc1]

Disclosed herein is a compound of formula

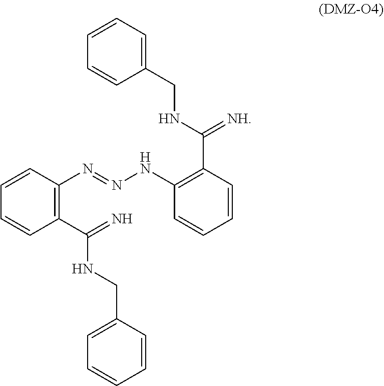 [SMILES: N=C(NCc1ccccc1)c1ccccc1/N=N/Nc1ccccc1C(=N)NCc1ccccc1]

Disclosed herein is a compound of formula

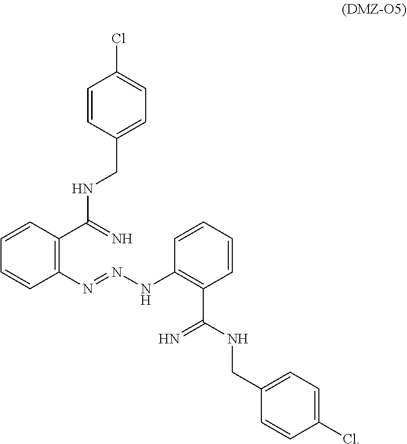 [SMILES: N=C(NCc1ccc(Cl)cc1)c1ccccc1/N=N/Nc1ccccc1C(=N)NCc1ccc(Cl)cc1]

Disclosed herein is a compound of formula

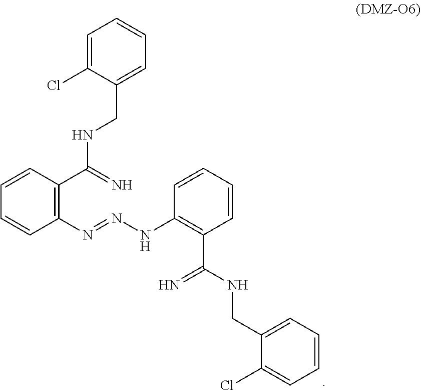 [SMILES: N=C(NCc1ccccc1Cl)c1ccccc1/N=N/Nc1ccccc1C(=N)NCc1ccccc1Cl]

Disclosed herein is a compound of formula

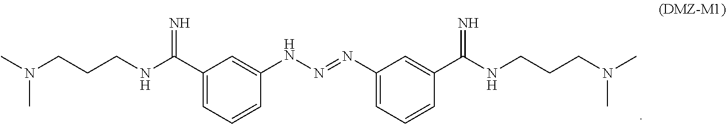

Disclosed herein is a compound of formula

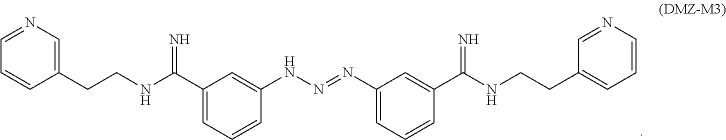 [SMILES: N=C(NCCc1cccnc1)c1cccc(/N=N/Nc2cccc(C(=N)NCCc3cccnc3)c2)c1]

Disclosed herein is a compound of formula

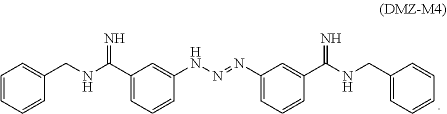 [SMILES: N=C(NCc1ccccc1)c1cccc(/N=N/Nc2cccc(C(=N)NCc3ccccc3)c2)c1]

Disclosed herein is a compound of formula

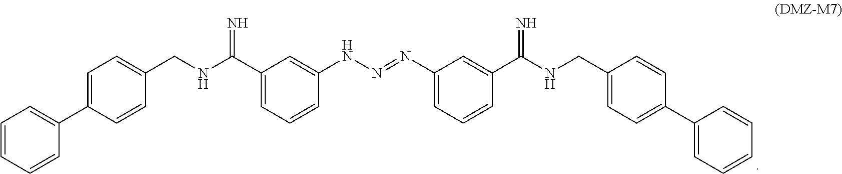 [SMILES: N=C(NCc1ccc(-c2ccccc2)cc1)c1cccc(/N=N/Nc2cccc(C(=N)NCc3ccc(-c4ccccc4)cc3)c2)c1]

Disclosed herein is a compound of formula

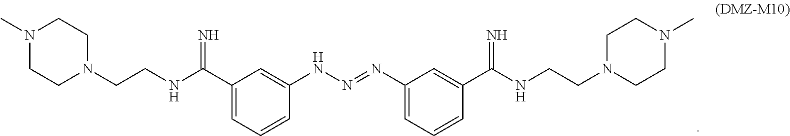

Disclosed herein is a compound of formula

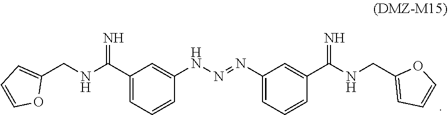 [SMILES: N=C(NCc1ccco1)c1cccc(/N=N/Nc2cccc(C(=N)NCc3ccco3)c2)c1]

Disclosed herein is a compound of formula

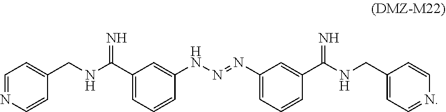 [SMILES: N=C(NCc1ccncc1)c1cccc(/N=N/Nc2cccc(C(=N)NCc3ccncc3)c2)c1]

Disclosed herein is a compound of formula

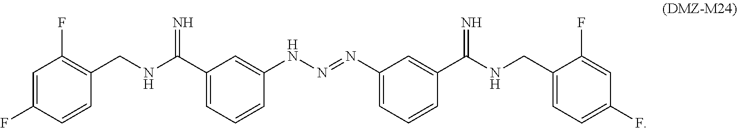 [SMILES: N=C(NCc1ccc(F)cc1F)c1cccc(/N=N/Nc2cccc(C(=N)NCc3ccc(F)cc3F)c2)c1]

Disclosed herein is a compound of formula

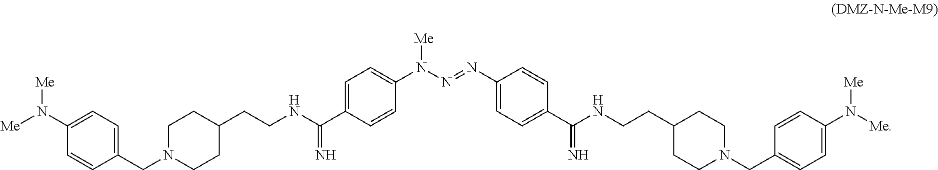 [SMILES: CN(C)c1ccc(CN2CCC(CCNC(=N)c3ccc(/N=N/N(C)c4ccc(C(=N)NCCC5CCN(Cc6ccc(N(C)C)cc6)CC5)cc4)cc3)CC2)cc1]

Disclosed herein is a compound of formula

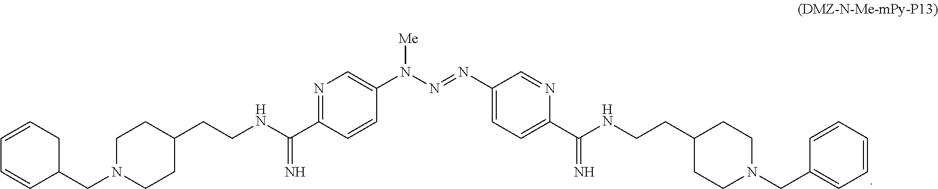 [SMILES: CN(/N=N/c1ccc(C(=N)NCCC2CCN(Cc3ccccc3)CC2)nc1)c1ccc(C(=N)NCCC2CCN(CC3C=CC=CC3)CC2)nc1]

Disclosed herein is a compound of formula

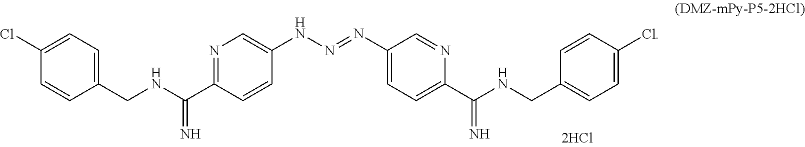 [SMILES: N=C(NCc1ccc(Cl)cc1)c1ccc(/N=N/Nc2ccc(C(=N)NCc3ccc(Cl)cc3)nc2)cn1]

Disclosed herein is a compound of formula

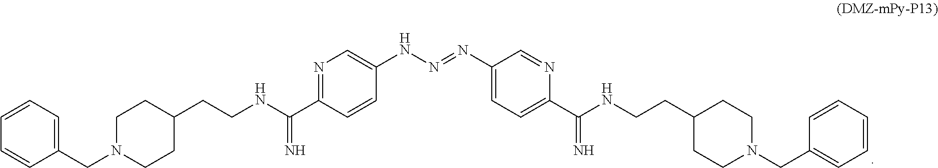 [SMILES: N=C(NCCC1CCN(Cc2ccccc2)CC1)c1ccc(/N=N/Nc2ccc(C(=N)NCCC3CCN(Cc4ccccc4)CC3)nc2)cn1]

Disclosed herein is a compound of formula

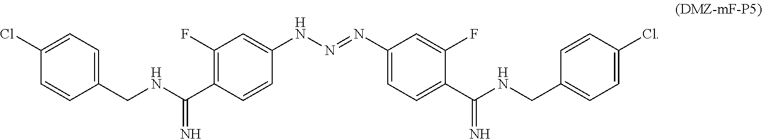 [SMILES: N=C(NCc1ccc(Cl)cc1)c1ccc(/N=N/Nc2ccc(C(=N)NCc3ccc(Cl)cc3)c(F)c2)cc1F]

Disclosed herein is a compound of formula

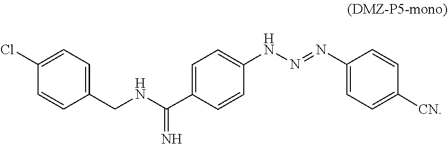 [SMILES: N#Cc1ccc(/N=N/Nc2ccc(C(=N)NCc3ccc(Cl)cc3)cc2)cc1]

Disclosed herein is a compound of formula

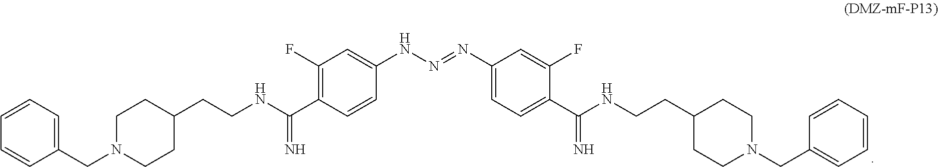 [SMILES: N=C(NCCC1CCN(Cc2ccccc2)CC1)c1ccc(/N=N/Nc2ccc(C(=N)NCCC3CCN(Cc4ccccc4)CC3)c(F)c2)cc1F]

Disclosed herein is a compound of formula

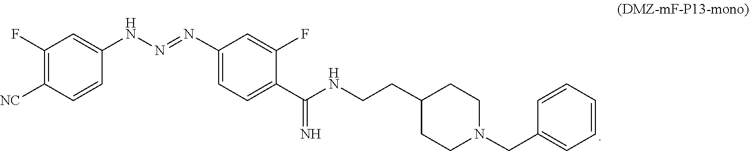 [SMILES: N#Cc1ccc(N/N=N/c2ccc(C(=N)NCCC3CCN(Cc4ccccc4)CC3)c(F)c2)cc1F]

Disclosed herein is a compound of formula

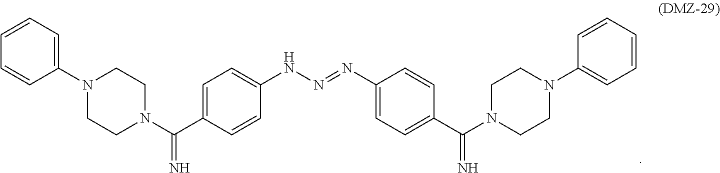 [SMILES: N=C(c1ccc(/N=N/Nc2ccc(C(=N)N3CCN(c4ccccc4)CC3)cc2)cc1)N1CCN(c2ccccc2)CC1]

Disclosed herein is a compound of formula

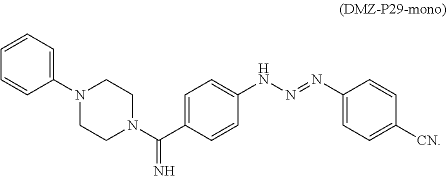 [SMILES: N#Cc1ccc(/N=N/Nc2ccc(C(=N)N3CCN(c4ccccc4)CC3)cc2)cc1]

Disclosed herein is a compound of formula

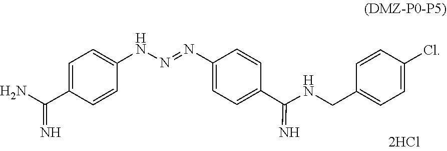 [SMILES: N=C(N)c1ccc(N/N=N/c2ccc(C(=N)NCc3ccc(Cl)cc3)cc2)cc1]

Disclosed herein is a compound of formula

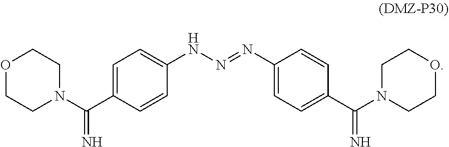 [SMILES: N=C(c1ccc(/N=N/Nc2ccc(C(=N)N3CCOCC3)cc2)cc1)N1CCOCC1]

Disclosed herein is a compound of formula

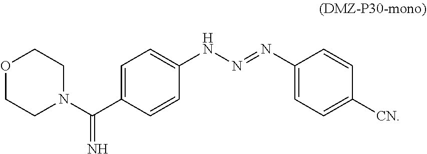 [SMILES: N#Cc1ccc(/N=N/Nc2ccc(C(=N)N3CCOCC3)cc2)cc1]

Disclosed herein is a compound of formula

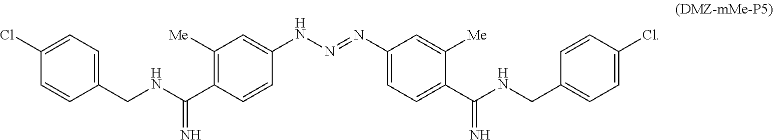 [SMILES: Cc1cc(/N=N/Nc2ccc(C(=N)NCc3ccc(Cl)cc3)c(C)c2)ccc1C(=N)NCc1ccc(Cl)cc1]

Disclosed herein is a compound of formula

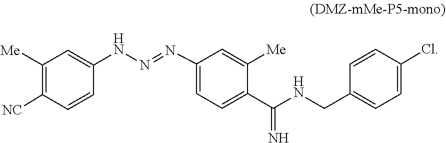 [SMILES: Cc1cc(N/N=N/c2ccc(C(=N)NCc3ccc(Cl)cc3)c(C)c2)ccc1C#N]

Disclosed herein is a compound of formula

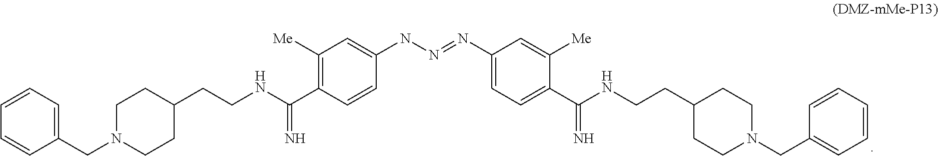 [SMILES: Cc1cc(/N=N/Nc2ccc(C(=N)NCCC3CCN(Cc4ccccc4)CC3)c(C)c2)ccc1C(=N)NCCC1CCN(Cc2ccccc2)CC1]

Disclosed herein is a compound of formula

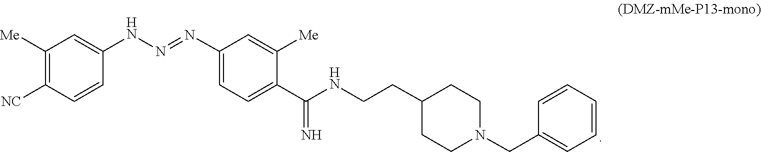 [SMILES: Cc1cc(N/N=N/c2ccc(C(=N)NCCC3CCN(Cc4ccccc4)CC3)c(C)c2)ccc1C#N]

Disclosed herein is a compound of formula

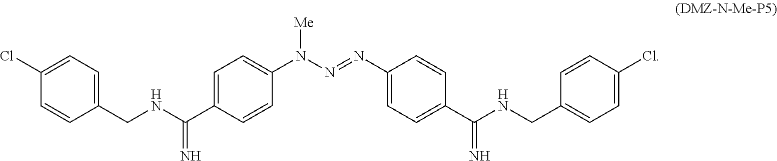 [SMILES: CN(/N=N/c1ccc(C(=N)NCc2ccc(Cl)cc2)cc1)c1ccc(C(=N)NCc2ccc(Cl)cc2)cc1]

Disclosed herein is a compound of formula

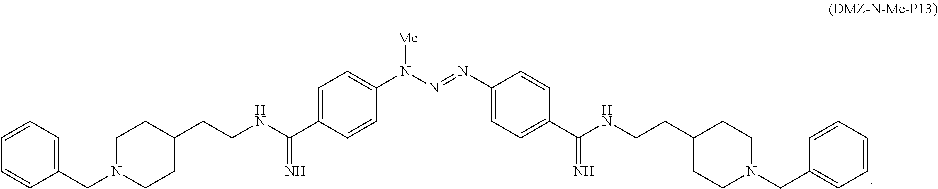 [SMILES: CN(/N=N/c1ccc(C(=N)NCCC2CCN(Cc3ccccc3)CC2)cc1)c1ccc(C(=N)NCCC2CCN(Cc3ccccc3)CC2)cc1]

Disclosed herein is a compound of formula

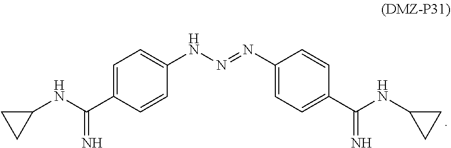 [SMILES: N=C(NC1CC1)c1ccc(/N=N/Nc2ccc(C(=N)NC3CC3)cc2)cc1]

Disclosed herein is a compound of formula

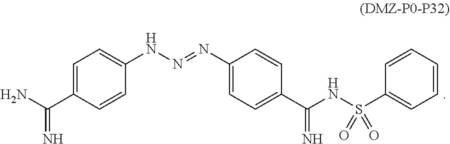 [SMILES: N=C(N)c1ccc(N/N=N/c2ccc(C(=N)NS(=O)(=O)c3ccccc3)cc2)cc1]

Disclosed herein is a compound of formula

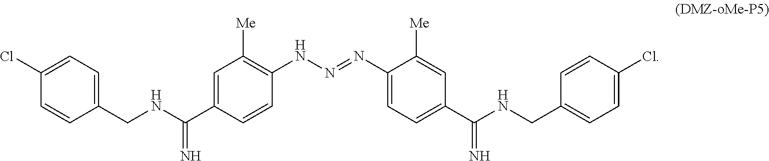 [SMILES: Cc1cc(C(=N)NCc2ccc(Cl)cc2)ccc1/N=N/Nc1ccc(C(=N)NCc2ccc(Cl)cc2)cc1C]

Disclosed herein is a compound of formula

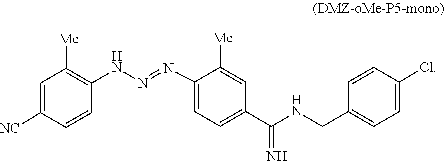 [SMILES: Cc1cc(C(=N)NCc2ccc(Cl)cc2)ccc1/N=N/Nc1ccc(C#N)cc1C]

Disclosed herein is a compound of formula

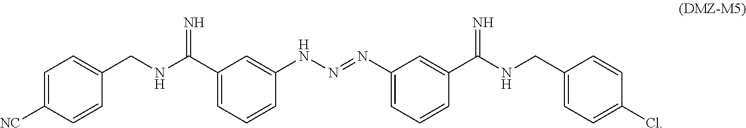 [SMILES: N#Cc1ccc(CNC(=N)c2cccc(N/N=N/c3cccc(C(=N)NCc4ccc(Cl)cc4)c3)c2)cc1]

Disclosed herein is a compound of formula

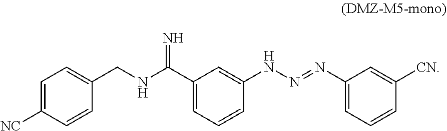 [SMILES: N#Cc1ccc(CNC(=N)c2cccc(N/N=N/c3cccc(C#N)c3)c2)cc1]

Disclosed herein is a compound of formula

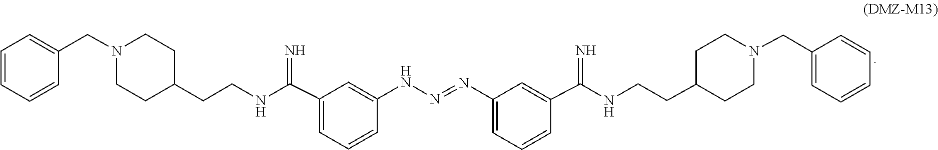 [SMILES: N=C(NCCC1CCN(Cc2ccccc2)CC1)c1cccc(/N=N/Nc2cccc(C(=N)NCCC3CCN(Cc4ccccc4)CC3)c2)c1]

Disclosed herein is a compound of formula

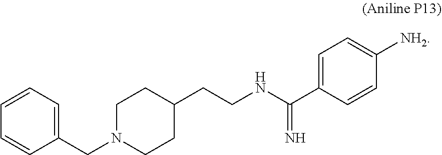 [SMILES: N=C(NCCC1CCN(Cc2ccccc2)CC1)c1ccc(N)cc1]

Disclosed herein is a compound of formula

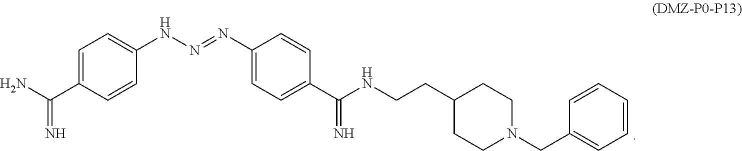 [SMILES: N=C(N)c1ccc(N/N=N/c2ccc(C(=N)NCCC3CCN(Cc4ccccc4)CC3)cc2)cc1]

Disclosed herein is a compound of formula

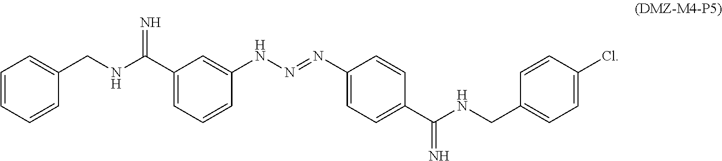 [SMILES: N=C(NCc1ccc(Cl)cc1)c1ccc(/N=N/Nc2cccc(C(=N)NCc3ccccc3)c2)cc1]

Disclosed herein is a compound of formula

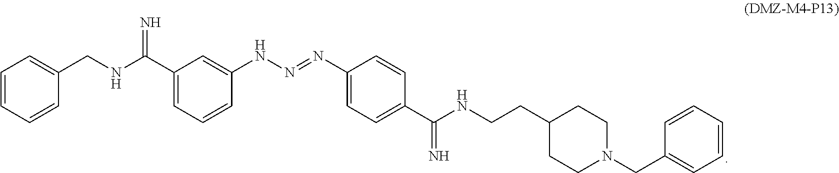 [SMILES: N=C(NCCC1CCN(Cc2ccccc2)CC1)c1ccc(/N=N/Nc2cccc(C(=N)NCc3ccccc3)c2)cc1]

Disclosed herein is a compound of formula

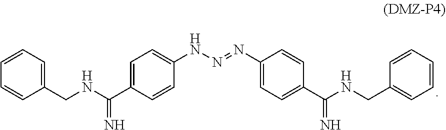 [SMILES: N=C(NCc1ccccc1)c1ccc(/N=N/Nc2ccc(C(=N)NCc3ccccc3)cc2)cc1]

Disclosed herein is a compound of formula

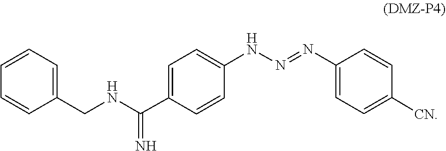 [SMILES: N#Cc1ccc(/N=N/Nc2ccc(C(=N)NCc3ccccc3)cc2)cc1]

Disclosed herein is a compound of formula

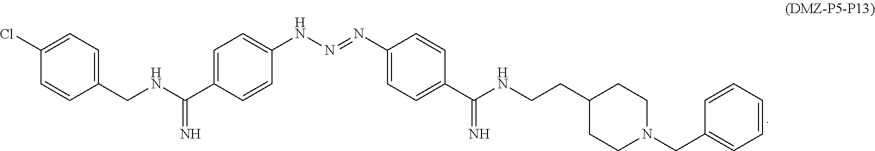 [SMILES: N=C(NCCC1CCN(Cc2ccccc2)CC1)c1ccc(/N=N/Nc2ccc(C(=N)NCc3ccc(Cl)cc3)cc2)cc1]

Disclosed herein is a compound of formula

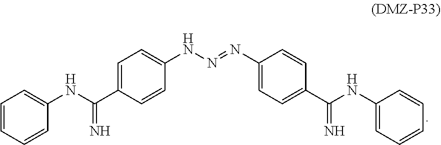 [SMILES: N=C(Nc1ccccc1)c1ccc(/N=N/Nc2ccc(C(=N)Nc3ccccc3)cc2)cc1]

Disclosed herein is a compound of formula

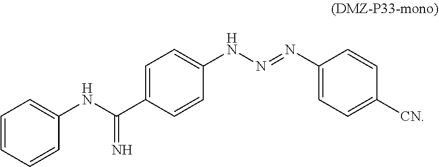 [SMILES: N#Cc1ccc(/N=N/Nc2ccc(C(=N)Nc3ccccc3)cc2)cc1]

Disclosed herein is a compound of formula

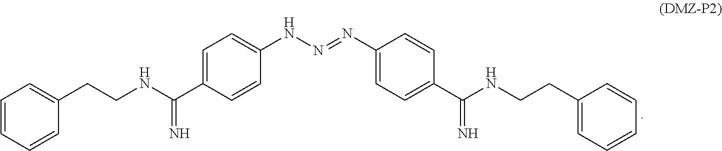 [SMILES: N=C(NCCc1ccccc1)c1ccc(/N=N/Nc2ccc(C(=N)NCCc3ccccc3)cc2)cc1]

Disclosed herein is a compound of formula

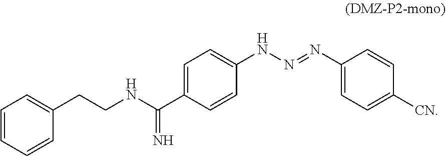 [SMILES: N#Cc1ccc(/N=N/Nc2ccc(C(=N)NCCc3ccccc3)cc2)cc1]

Disclosed herein is a compound of formula

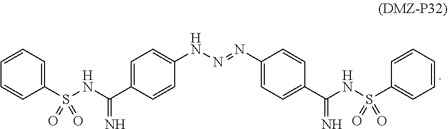 [SMILES: N=C(NS(=O)(=O)c1ccccc1)c1ccc(/N=N/Nc2ccc(C(=N)NS(=O)(=O)c3ccccc3)cc2)cc1]

Disclosed herein is a compound of formula

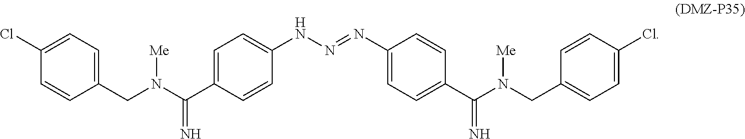 [SMILES: CN(Cc1ccc(Cl)cc1)C(=N)c1ccc(/N=N/Nc2ccc(C(=N)N(C)Cc3ccc(Cl)cc3)cc2)cc1]

Disclosed herein is a compound of formula

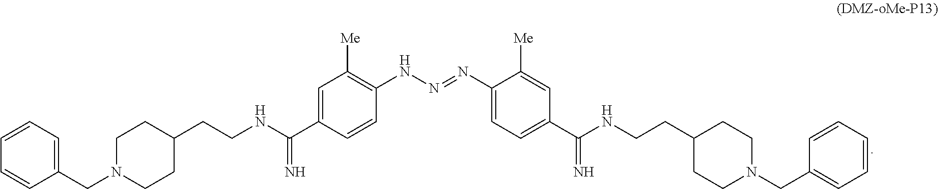 [SMILES: Cc1cc(C(=N)NCCC2CCN(Cc3ccccc3)CC2)ccc1/N=N/Nc1ccc(C(=N)NCCC2CCN(Cc3ccccc3)CC2)cc1C]

Disclosed herein is a compound of formula

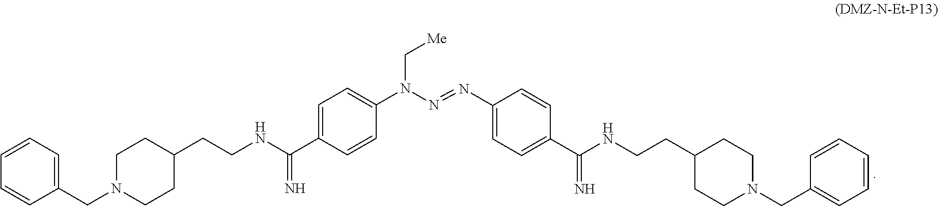 [SMILES: CCN(/N=N/c1ccc(C(=N)NCCC2CCN(Cc3ccccc3)CC2)cc1)c1ccc(C(=N)NCCC2CCN(Cc3ccccc3)CC2)cc1]

Disclosed herein is a compound of formula

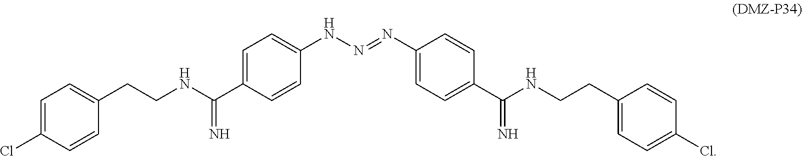 [SMILES: N=C(NCCc1ccc(Cl)cc1)c1ccc(/N=N/Nc2ccc(C(=N)NCCc3ccc(Cl)cc3)cc2)cc1]

Disclosed herein is a compound of formula

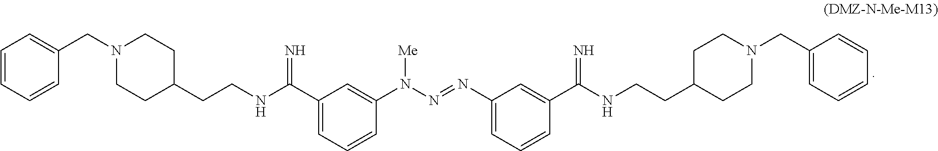 [SMILES: CN(/N=N/c1cccc(C(=N)NCCC2CCN(Cc3ccccc3)CC2)c1)c1cccc(C(=N)NCCC2CCN(Cc3ccccc3)CC2)c1]

Disclosed herein is a compound of formula

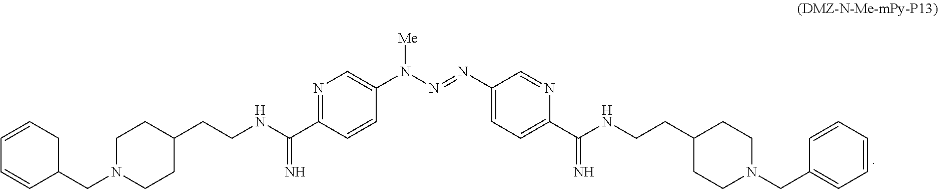 [SMILES: CN(/N=N/c1ccc(C(=N)NCCC2CCN(Cc3ccccc3)CC2)nc1)c1ccc(C(=N)NCCC2CCN(CC3C=CC=CC3)CC2)nc1]

Disclosed herein is a compound of formula

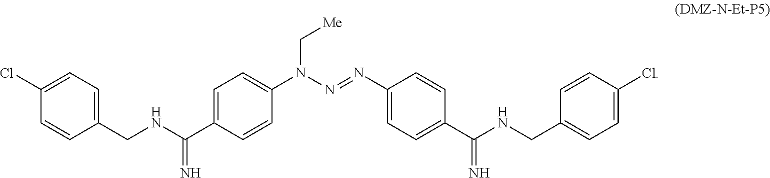 [SMILES: CCN(/N=N/c1ccc(C(=N)NCc2ccc(Cl)cc2)cc1)c1ccc(C(=N)NCc2ccc(Cl)cc2)cc1]

Disclosed herein is a compound of formula

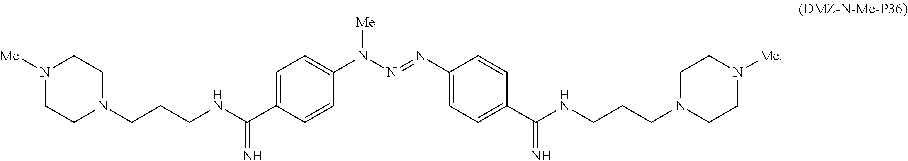 [SMILES: CN1CCN(CCCNC(=N)c2ccc(/N=N/N(C)c3ccc(C(=N)NCCCN4CCN(C)CC4)cc3)cc2)CC1]

Disclosed herein is a compound of formula

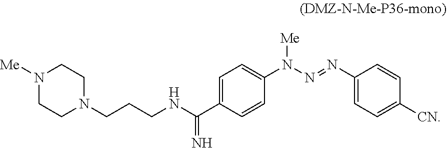 [SMILES: CN1CCN(CCCNC(=N)c2ccc(N(C)/N=N/c3ccc(C#N)cc3)cc2)CC1]

Disclosed herein is a compound of formula

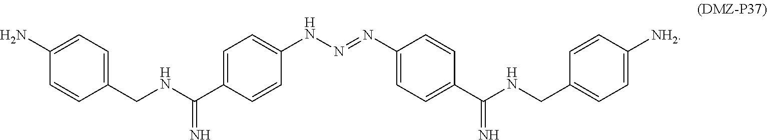 [SMILES: N=C(NCc1ccc(N)cc1)c1ccc(/N=N/Nc2ccc(C(=N)NCc3ccc(N)cc3)cc2)cc1]

Disclosed herein is a compound of formula

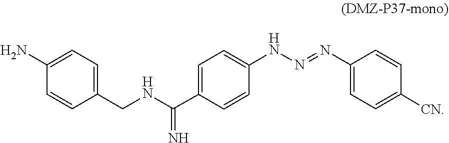 [SMILES: N#Cc1ccc(/N=N/Nc2ccc(C(=N)NCc3ccc(N)cc3)cc2)cc1]

Disclosed herein is a compound of formula

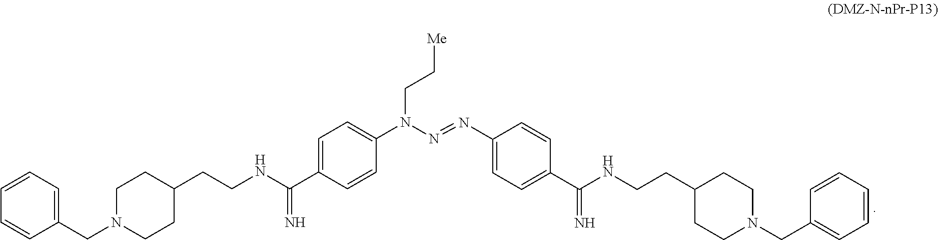 [SMILES: CCCN(/N=N/c1ccc(C(=N)NCCC2CCN(Cc3ccccc3)CC2)cc1)c1ccc(C(=N)NCCC2CCN(Cc3ccccc3)CC2)cc1]

Disclosed herein is a compound of formula

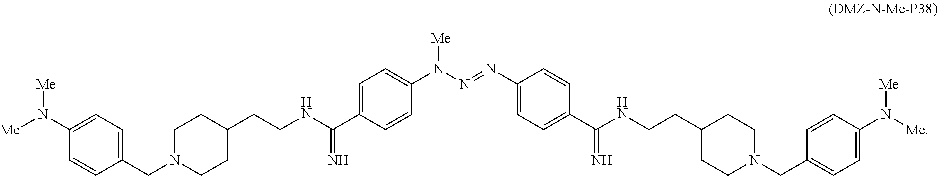 [SMILES: CN(C)c1ccc(CN2CCC(CCNC(=N)c3ccc(/N=N/N(C)c4ccc(C(=N)NCCC5CCN(Cc6ccc(N(C)C)cc6)CC5)cc4)cc3)CC2)cc1]

Disclosed herein is a compound of formula

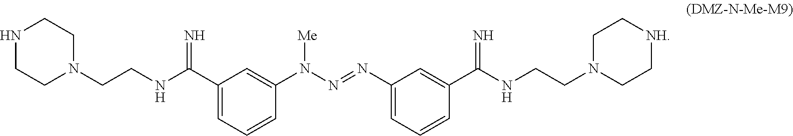 [SMILES: CN(/N=N/c1cccc(C(=N)NCCN2CCNCC2)c1)c1cccc(C(=N)NCCN2CCNCC2)c1]

Disclosed herein is a compound referred to as DMZ-P1, DMZ-P5, DMZ-P8, DMZ-P9, DMZ-P13, DMZ-P14, DMZ-P17, DMZ-O1, DMZ-O2, DMZ-O4, DMZ-O5, DMZ-O6, DMZ-M1, DMZ-M3, DMZ-M4, DMZ-M7, DMZ-M10, DMZ-M15, DMZ-M22, DMZ-M24, DMZ-N-Me-M9, DMZ-N-Me-mPy-P13, DMZ-mPy-P5-2HCl, DMZ-mPy-P13, DMZ-mF-P5, DMZ-P5-mono, DMZ-mF-P13, DMZ-mF-P13-mono, DMZ-P29, DMZ-P29-mono, DMZ-P0-P5, DMZ-P30, DMZ-P30-mono, DMZ-mMe-P5, DMZ-mMe-P5-mono, DMZ-mMe-P13, DMZ-mMe-P13-mono, DMZ-N-Me-P5, DMZ-N-Me-P13, DMZ-P31, DMZ-P0-P32, DMZ-oMe-P5, DMZ-oMe-P5-mono, DMZ-M5, DMZ-M5-mono, DMZ-M13, Aniline-P13, DMZ-P0-P13, DMZ-M4-P5, DMZ-M4-P13, DMZ-P4, DMZ-P4-mono, DMZ-P5-P13, DMZ-P33, DMZ-P33-mono, DMZ-P2, DMZ-P2-mono, DMZ-P32, DMZ-P35, DMZ-oMe-P13, DMZ-N-Et-P13, DMZ-P34, DMZ-N-Me-M13, DMZ-N-Me-mPy-P13, DMZ-N-Et-P5, DMZ-N-Me-P36, DMZ-N-Me-P36-mono, DMZ-P37, DMZ-P37-mono, DMZ-N-nPr-P13, DMZ-N-Me-P38, DMZ-N-Me-M9, or a pharmaceutically acceptable salt, a hydrate, a prodrug, an ester, or a derivative thereof.

In an aspect, a disclosed DMZ compound can be a pharmaceutical acceptable salt, a hydrate, a prodrug, an ester, or a derivative thereof.

In an aspect, DMZ-P8, DMZ-P9, DMZ-P13, DMZ-P17, DMZ-O1, DMZ-O4, DMZ-O6, DMZ-M1, DMZ-M3, DMZ-M7, DMZ-M10, DMZ-NMe-P38, DMZ-NMe-mPy-P1, or any combination thereof can be destabilizing. In an aspect, DMZ-P0, DMZ-P1, DMZ-P5, DMZ-P14, DMZ-O2, DMZ-O5, DMZ-M4, DMZ-M15, DMZ-M22, or any combination thereof can be stabilizing.

In an aspect, a disclosed compound can target a disclosed RNA or a disclosed RNA structure. In an aspect, a disclosed RNA or a disclosed RNA structure can comprise a functionally important RNA.

In an aspect, a disclosed compound can target a bulge, a S-turn, an internal loop, a 3-multiloop, a hairpin loop, a pseudoknot, an apical loop, a kissing loop, a coaxial helix, a stacking helix, a two-way junction, a three-way junction, a four-way junction, or any combination thereof.

In an aspect, a disclosed compound can target a GNAA tetraloop, a GNGA loop, a T-loop, a C-loop, a E-loop, a Sarcin-ricin loop, a Kink-turn, a Reverse kink-turn, a Hook-turn, a Tandem shear, a Tetraloop-receptor motifs, an interacting tetraloop (e.g., GNRA, GANC, and GAAA tetraloops), a triple-stranded RNA, or any combination thereof.

In an aspect, a disclosed compound can decrease the expression and/or level of a disclosed secondary RNA structure. In an aspect, a disclosed compound can increase the expression and/or level of a disclosed secondary RNA structure. In an aspect, a disclosed compound can modulate a disclosed secondary RNA structure. In an aspect, a disclosed compound can stabilize a disclosed secondary RNA structure. In an aspect, a disclosed compound can destabilize a disclosed secondary RNA structure.

In an aspect, stabilizing a disclosed secondary RNA structure can comprise measuring less degradation of the disclosed secondary RNA structure when subjected to an enzymatic assay in the presence of the disclosed compound than a control or reference level of degradation. In an aspect, stabilizing a disclosed secondary RNA structure can comprise measuring less degradation of the disclosed secondary RNA structure when subjected to an enzymatic assay in the presence of the disclosed compound than the level of degradation in a control or reference sample.

In an aspect, destabilizing a disclosed secondary RNA structure can comprise measuring more degradation of the disclosed secondary RNA structure when subjected to an enzymatic assay in the presence of the disclosed compound than a control or reference level of degradation. In an aspect, destabilizing a disclosed secondary RNA structure can comprise measuring more degradation of the disclosed secondary RNA structure when subjected to an enzymatic assay in the presence of the disclosed compound than the level of degradation in a control or reference sample.

In an aspect, a disclosed compound can destabilize a disclosed secondary RNA structure, can destabilize a disclosed secondary RNA structure, can be toxic to one or more cell types, can disrupt and/or impair the functionality of one or more cell types, can initiate, induce, promote, elicit, hasten, and/or cause the death of a cancer cell or a tumor cell, can reduce and/or minimize the likelihood of metastasis, or any combination thereof.

In an aspect, a disclosed compound can decrease the expression and/or level of a disclosed tertiary RNA structure. In an aspect, a disclosed compound can increase the expression and/or level of a disclosed tertiary RNA structure. In an aspect, a disclosed compound can modulate a disclosed tertiary RNA structure. In an aspect, a disclosed compound can stabilize a disclosed tertiary RNA structure. In an aspect, a disclosed compound can destabilize a disclosed tertiary RNA structure.

In an aspect, stabilizing a disclosed tertiary RNA structure can comprise measuring less degradation of the disclosed tertiary RNA structure when subjected to an enzymatic assay in the presence of the disclosed compound than a control or reference level of degradation. In an aspect, stabilizing a disclosed tertiary RNA structure can comprise measuring less degradation of the disclosed tertiary RNA structure when subjected to an enzymatic assay in the presence of the disclosed compound than the level of degradation in a control or reference sample.

In an aspect, destabilizing a disclosed tertiary RNA structure can comprise measuring more degradation of the disclosed tertiary RNA structure when subjected to an enzymatic assay in the presence of the disclosed compound than a control or reference level of degradation. In an aspect, destabilizing a disclosed tertiary RNA structure can comprise measuring more degradation of the disclosed tertiary RNA structure when subjected to an enzymatic assay in the presence of the disclosed compound than the level of degradation in a control or reference sample.

In an aspect, a disclosed compound can destabilize a disclosed tertiary RNA structure, can destabilize a disclosed tertiary RNA structure, can be toxic to one or more cell types, can disrupt and/or impair the functionality of one or more cell types, can initiate, induce, promote, elicit, hasten, and/or cause the death of a cancer cell or a tumor cell, can reduce and/or minimize the likelihood of metastasis, or any combination thereof.

In an aspect, a disclosed compound can decrease the expression and/or level of a disclosed MALAT1 triple helix, a disclosed NEAT1 triplex, a disclosed PAN triplex, or any combination thereof. In an aspect, a disclosed compound can decrease the expression and/or level of a disclosed lncRNA. In an aspect, a disclosed compound can decrease the expression and/or level of a disclosed lncRNA. In an aspect, a disclosed compound can decrease the expression and/or level of a disclosed SARS-CoV-2 pseudoknot, a disclosed HIV packaging signal, a disclosed complex junction in the long noncoding RNA SChLAP1, and combinations thereof. In an aspect, a disclosed compound can decrease the expression and/or level of a disclosed LMO7 functional secondary and/or tertiary RNA structure (including splice sites), a disclosed AR functional secondary and/or tertiary RNA structure (including splice sites), or any combinations thereof. In an aspect, a disclosed compound can decrease the expression and/or level of a disclosed Kaposi's sarcoma-associated herpesvirus (KSHV) polyadenylated nuclear (PAN) 3′-end triple helix, a disclosed Kaposi's sarcoma-associated herpesvirus (KSHV) polyadenylated nuclear (PAN) functional secondary structures and/or tertiary structures, a disclosed SARS-CoV-2 5′-untranslated region structure and/or 3′-untranslated region, a disclosed SARS-CoV-2 5′-untranslated region structure can comprise a frameshifting pseudoknot, a disclosed HIV element (e.g., a trans-activation response (TAR) element, a Rev response element (RRE), EESV, a frameshift site, a packing signal (interaction between (w) stem-loop 3 (SL3) RNA and Gag), or any combination thereof), a disclosed secondary structure or a disclosed tertiary structure in a poliovirus, a rhinovirus, an encephalomyocarditis virus, a foot-and-mouth disease virus, a Kaposi's sarcoma-associated herpesvirus, a hepatitis A virus, or a hepatitis C virus, a disclosed secondary or tertiary RNA structure in a coronavirus, a flavivirus, alphaviruses, a picornavirus, or a positive sense RNA virus, or any combinations thereof. In an aspect, a disclosed coronavirus can be 229E (alpha coronavirus), NL63 (alpha coronavirus), OC43 (beta coronavirus), HKU1 (beta coronavirus), MERS-COV (beta coronavirus), SARS-COV (beta coronavirus), and SARS-CoV-2, or a combination thereof.

In an aspect, a disclosed compound can increase the expression and/or level of a disclosed MALAT1 triple helix, a disclosed NEAT1 triplex, a disclosed PAN triplex, or any combination thereof. In an aspect, a disclosed compound can increase the expression and/or level of a disclosed lncRNA. In an aspect, a disclosed compound can increase the expression and/or level of a disclosed SARS-CoV-2 pseudoknot, a disclosed HIV packaging signal, a disclosed complex junction in the long noncoding RNA SCHLAP1, and combinations thereof. In an aspect, a disclosed compound can increase the expression and/or level of a disclosed LMO7 functional secondary and/or tertiary RNA structure (including splice sites), a disclosed AR functional secondary and/or tertiary RNA structure (including splice sites), or any combinations thereof. In an aspect, a disclosed compound can increase the expression and/or level of a disclosed Kaposi's sarcoma-associated herpesvirus (KSHV) polyadenylated nuclear (PAN) 3′-end triple helix, a disclosed Kaposi's sarcoma-associated herpesvirus (KSHV) polyadenylated nuclear (PAN) functional secondary structures and/or tertiary structures, a disclosed SARS-CoV-2 5′-untranslated region structure and/or 3′-untranslated region, a disclosed SARS-CoV-2 5′-untranslated region structure can comprise a frameshifting pseudoknot, a disclosed HIV element (e.g., a trans-activation response (TAR) element, a Rev response element (RRE), EESV, a frameshift site, a packing signal (interaction between (w) stem-loop 3 (SL3) RNA and Gag), or any combination thereof), a disclosed secondary structure or a disclosed tertiary structure in a poliovirus, a rhinovirus, an encephalomyocarditis virus, a foot-and-mouth disease virus, a Kaposi's sarcoma-associated herpesvirus, a hepatitis A virus, or a hepatitis C virus, a disclosed secondary or tertiary RNA structure in a coronavirus, a flavivirus, alphaviruses, a picornavirus, or a positive sense RNA virus, or any combinations thereof.

In an aspect, a disclosed compound can modulate a disclosed MALAT1 triple helix, a disclosed NEAT1 triplex, a disclosed PAN triplex, or any combination thereof. In an aspect, a disclosed compound can modulate a disclosed lncRNA. In an aspect, a disclosed compound can modulate a disclosed SARS-CoV-2 pseudoknot, a disclosed HIV packaging signal, a disclosed complex junction in the long noncoding RNA SChLAP1, and combinations thereof. In an aspect, a disclosed compound can modulate a disclosed LMO7 functional secondary and/or tertiary RNA structure (including splice sites), a disclosed AR functional secondary and/or tertiary RNA structure (including splice sites), or any combinations thereof. In an aspect, a disclosed compound can modulate a disclosed Kaposi's sarcoma-associated herpesvirus (KSHV) polyadenylated nuclear (PAN) 3′-end triple helix, a disclosed Kaposi's sarcoma-associated herpesvirus (KSHV) polyadenylated nuclear (PAN) functional secondary structures and/or tertiary structures, a disclosed SARS-CoV-2 5′-untranslated region structure and/or 3′-untranslated region, a disclosed SARS-CoV-2 5′-untranslated region structure can comprise a frameshifting pseudoknot, a disclosed HIV element (e.g., a trans-activation response (TAR) element, a Rev response element (RRE), EESV, a frameshift site, a packing signal (interaction between (w) stem-loop 3 (SL3) RNA and Gag), or any combination thereof), a disclosed secondary structure or a disclosed tertiary structure in a poliovirus, a rhinovirus, an encephalomyocarditis virus, a foot-and-mouth disease virus, a Kaposi's sarcoma-associated herpesvirus, a hepatitis A virus, or a hepatitis C virus, a disclosed secondary or tertiary RNA structure in a coronavirus, a flavivirus, alphaviruses, a picornavirus, or a positive sense RNA virus, or any combinations thereof.

In an aspect, a disclosed compound can stabilize a disclosed MALAT1 triple helix, a disclosed NEAT1 triplex, a disclosed PAN triplex, or any combination thereof. In an aspect, a disclosed compound can stabilize a disclosed lncRNA. In an aspect, a disclosed compound can stabilize a disclosed SARS-CoV-2 pseudoknot, a disclosed HIV packaging signal, a disclosed complex junction in the long noncoding RNA SChLAP1, and combinations thereof. In an aspect, a disclosed compound can stabilize a disclosed LMO7 functional secondary and/or tertiary RNA structure (including splice sites), a disclosed AR functional secondary and/or tertiary RNA structure (including splice sites), or any combinations thereof. In an aspect, a disclosed compound can stabilize a disclosed Kaposi's sarcoma-associated herpesvirus (KSHV) polyadenylated nuclear (PAN) 3′-end triple helix, a disclosed Kaposi's sarcoma-associated herpesvirus (KSHV) polyadenylated nuclear (PAN) functional secondary structures and/or tertiary structures, a disclosed SARS-CoV-2 5′-untranslated region structure and/or 3′-untranslated region, a disclosed SARS-CoV-2 5′-untranslated region structure can comprise a frameshifting pseudoknot, a disclosed HIV element (e.g., a trans-activation response (TAR) element, a Rev response element (RRE), EESV, a frameshift site, a packing signal (interaction between (w) stem-loop 3 (SL3) RNA and Gag), or any combination thereof), a disclosed secondary structure or a disclosed tertiary structure in a poliovirus, a rhinovirus, an encephalomyocarditis virus, a foot-and-mouth disease virus, a Kaposi's sarcoma-associated herpesvirus, a hepatitis A virus, or a hepatitis C virus, a disclosed secondary or tertiary RNA structure in a coronavirus, a flavivirus, alphaviruses, a picornavirus, or a positive sense RNA virus, or any combinations thereof.

In an aspect, a disclosed compound can destabilize a disclosed MALAT1 triple helix, a disclosed NEAT1 triplex, a disclosed PAN triplex, or any combination thereof. In an aspect, a disclosed compound can destabilize a disclosed lncRNA. In an aspect, a disclosed compound can destabilize a disclosed SARS-CoV-2 pseudoknot, a disclosed HIV packaging signal, a disclosed complex junction in the long noncoding RNA SChLAP1, and combinations thereof. In an aspect, a disclosed compound can destabilize a disclosed LMO7 functional secondary and/or tertiary RNA structure (including splice sites), a disclosed AR functional secondary and/or tertiary RNA structure (including splice sites), or any combinations thereof. In an aspect, a disclosed compound can destabilize a disclosed Kaposi's sarcoma-associated herpesvirus (KSHV) polyadenylated nuclear (PAN) 3′-end triple helix, a disclosed Kaposi's sarcoma-associated herpesvirus (KSHV) polyadenylated nuclear (PAN) functional secondary structures and/or tertiary structures, a disclosed SARS-CoV-2 5′-untranslated region structure and/or 3′-untranslated region, a disclosed SARS-CoV-2 5′-untranslated region structure can comprise a frameshifting pseudoknot, a disclosed HIV element (e.g., a trans-activation response (TAR) element, a Rev response element (RRE), EESV, a frameshift site, a packing signal (interaction between (w) stem-loop 3 (SL3) RNA and Gag), or any combination thereof), a disclosed secondary structure or a disclosed tertiary structure in a poliovirus, a rhinovirus, an encephalomyocarditis virus, a foot-and-mouth disease virus, a Kaposi's sarcoma-associated herpesvirus, a hepatitis A virus, or a hepatitis C virus, a disclosed secondary or tertiary RNA structure in a coronavirus, a flavivirus, alphaviruses, a picornavirus, or a positive sense RNA virus, or any combinations thereof.

In an aspect, stabilizing a disclosed MALAT1 triple helix, a disclosed NEAT1 triplex, a disclosed PAN triplex, or any combination thereof can comprise measuring less degradation of the disclosed MALAT1 triple helix, the disclosed NEAT1 triplex, the disclosed PAN triplex, or any combination thereof when subjected to an enzymatic assay in the presence of the disclosed compound than a control or reference level of degradation. In an aspect, stabilizing a disclosed lncRNA can comprise measuring less degradation of the lncRNA when subjected to an enzymatic assay in the presence of the disclosed compound than a control or reference level of degradation. In an aspect, stabilizing a disclosed HIV packaging signal, a disclosed complex junction in the long noncoding RNA SChLAP1, and combinations thereof can comprise measuring less degradation of the disclosed HIV packaging signal, a disclosed complex junction in the long noncoding RNA SChLAP1, and combinations thereof when subjected to an enzymatic assay in the presence of the disclosed compound than a control or reference level of degradation.

In an aspect, stabilizing a LMO7 functional secondary and/or tertiary RNA structure (including splice sites), an AR functional secondary and/or tertiary RNA structure (including splice sites), or any combinations thereof can comprise measuring less degradation of the disclosed LMO7 functional secondary and/or tertiary RNA structure (including splice sites), a the disclosed AR functional secondary and/or tertiary RNA structure (including splice sites), or any combinations thereof when subjected to an enzymatic assay in the presence of the disclosed compound than a control or reference level of degradation. In an aspect, stabilizing a disclosed Kaposi's sarcoma-associated herpesvirus (KSHV) polyadenylated nuclear (PAN) 3′-end triple helix, a disclosed Kaposi's sarcoma-associated herpesvirus (KSHV) polyadenylated nuclear (PAN) functional secondary structures and/or tertiary structures, a disclosed SARS-CoV-2 5′-untranslated region structure and/or 3′-untranslated region, a disclosed SARS-CoV-2 5′-untranslated region structure can comprise a frameshifting pseudoknot, a disclosed HIV element (e.g., a trans-activation response (TAR) element, a Rev response element (RRE), EESV, a frameshift site, a packing signal (interaction between (w) stem-loop 3 (SL3) RNA and Gag), or any combination thereof), a disclosed secondary structure or a disclosed tertiary structure in a poliovirus, a rhinovirus, an encephalomyocarditis virus, a foot-and-mouth disease virus, a Kaposi's sarcoma-associated herpesvirus, a hepatitis A virus, or a hepatitis C virus, a disclosed secondary or tertiary RNA structure in a coronavirus, a flavivirus, alphaviruses, a picornavirus, or a positive sense RNA virus, or any combinations thereof can comprise measuring less degradation of the disclosed Kaposi's sarcoma-associated herpesvirus (KSHV) polyadenylated nuclear (PAN) 3′-end triple helix, the disclosed Kaposi's sarcoma-associated herpesvirus (KSHV) polyadenylated nuclear (PAN) functional secondary structures and/or tertiary structures, the disclosed SARS-CoV-2 5′-untranslated region structure and/or 3′-untranslated region, the disclosed SARS-CoV-2 5′-untranslated region structure can comprise a frameshifting pseudoknot, the disclosed HIV element (e.g., a trans-activation response (TAR) element, the Rev response element (RRE), EESV, the frameshift site, the packing signal (interaction between (ψ) stem-loop 3 (SL3) RNA and Gag), or any combination thereof), the disclosed secondary structure or a disclosed tertiary structure in a poliovirus, a rhinovirus, an encephalomyocarditis virus, a foot-and-mouth disease virus, a Kaposi's sarcoma-associated herpesvirus, a hepatitis A virus, or a hepatitis C virus, the disclosed secondary or tertiary RNA structure in a coronavirus, a flavivirus, alphaviruses, a picornavirus, or a positive sense RNA virus, or any combinations thereof when subjected to an enzymatic assay in the presence of the disclosed compound than a control or reference level of degradation.

In an aspect, destabilizing a disclosed MALAT1 triple helix, a disclosed NEAT1 triplex, a disclosed PAN triplex, or any combination thereof can comprise measuring more degradation of the disclosed MALAT1 triple helix, the disclosed NEAT1 triplex, the disclosed PAN triplex, or any combination thereof when subjected to an enzymatic assay in the presence of the disclosed compound than a control or reference level of degradation. In an aspect, destabilizing a disclosed lncRNA can comprise measuring more degradation of the lncRNA when subjected to an enzymatic assay in the presence of the disclosed compound than a control or reference level of degradation. In an aspect, destabilizing a disclosed HIV packaging signal, a disclosed complex junction in the long noncoding RNA SChLAP1, and combinations thereof can comprise measuring more degradation of the disclosed HIV packaging signal, a disclosed complex junction in the long noncoding RNA SChLAP1, and combinations thereof when subjected to an enzymatic assay in the presence of the disclosed compound than a control or reference level of degradation.

In an aspect, destabilizing a LMO7 functional secondary and/or tertiary RNA structure (including splice sites), an AR functional secondary and/or tertiary RNA structure (including splice sites), or any combinations thereof can comprise measuring more degradation of the disclosed LMO7 functional secondary and/or tertiary RNA structure (including splice sites), a the disclosed AR functional secondary and/or tertiary RNA structure (including splice sites), or any combinations thereof when subjected to an enzymatic assay in the presence of the disclosed compound than a control or reference level of degradation. In an aspect, destabilizing a disclosed Kaposi's sarcoma-associated herpesvirus (KSHV) polyadenylated nuclear (PAN) 3′-end triple helix, a disclosed Kaposi's sarcoma-associated herpesvirus (KSHV) polyadenylated nuclear (PAN) functional secondary structures and/or tertiary structures, a disclosed SARS-CoV-2 5′-untranslated region structure and/or 3′-untranslated region, a disclosed SARS-CoV-2 5′-untranslated region structure can comprise a frameshifting pseudoknot, a disclosed HIV element (e.g., a trans-activation response (TAR) element, a Rev response element (RRE), EESV, a frameshift site, a packing signal (interaction between (w) stem-loop 3 (SL3) RNA and Gag), or any combination thereof), a disclosed secondary structure or a disclosed tertiary structure in a poliovirus, a rhinovirus, an encephalomyocarditis virus, a foot-and-mouth disease virus, a Kaposi's sarcoma-associated herpesvirus, a hepatitis A virus, or a hepatitis C virus, a disclosed secondary or tertiary RNA structure in a coronavirus, a flavivirus, alphaviruses, a picornavirus, or a positive sense RNA virus, or any combinations thereof can comprise measuring more degradation of the disclosed Kaposi's sarcoma-associated herpesvirus (KSHV) polyadenylated nuclear (PAN) 3′-end triple helix, the disclosed Kaposi's sarcoma-associated herpesvirus (KSHV) polyadenylated nuclear (PAN) functional secondary structures and/or tertiary structures, the disclosed SARS-CoV-2 5′-untranslated region structure and/or 3′-untranslated region, the disclosed SARS-CoV-2 5′-untranslated region structure can comprise a frameshifting pseudoknot, the disclosed HIV element (e.g., a trans-activation response (TAR) element, the Rev response element (RRE), EESV, the frameshift site, the packing signal (interaction between (w) stem-loop 3 (SL3) RNA and Gag), or any combination thereof), the disclosed secondary structure or a disclosed tertiary structure in a poliovirus, a rhinovirus, an encephalomyocarditis virus, a foot-and-mouth disease virus, a Kaposi's sarcoma-associated herpesvirus, a hepatitis A virus, or a hepatitis C virus, the disclosed secondary or tertiary RNA structure in a coronavirus, a flavivirus, alphaviruses, a picornavirus, or a positive sense RNA virus, or any combinations thereof when subjected to an enzymatic assay in the presence of the disclosed compound than a control or reference level of degradation.

In an aspect, stabilizing a disclosed MALAT1 triple helix, a disclosed NEAT1 triplex, a disclosed PAN triplex, or any combination thereof can comprise measuring less degradation of the disclosed MALAT1 triple helix, the disclosed NEAT1 triplex, the disclosed PAN triplex, or any combination thereof when subjected to an enzymatic assay in the presence of the disclosed compound than the level of degradation in a control or reference sample. In an aspect, stabilizing a disclosed lncRNA thereof can comprise measuring less degradation of the disclosed lncRNA when subjected to an enzymatic assay in the presence of the disclosed compound than the level of degradation in a control or reference sample. In an aspect, stabilizing a disclosed HIV packaging signal, a disclosed complex junction in the long noncoding RNA SChLAP1, and combinations thereof can comprise measuring less degradation of the disclosed HIV packaging signal, a disclosed complex junction in the long noncoding RNA SChLAP1, and combinations thereof when subjected to an enzymatic assay in the presence of the disclosed compound than the level of degradation in a control or reference sample. In an aspect, stabilizing a LMO7 functional secondary and/or tertiary RNA structure (including splice sites), an AR functional secondary and/or tertiary RNA structure (including splice sites), or any combinations thereof can comprise measuring less degradation of the disclosed LMO7 functional secondary and/or tertiary RNA structure (including splice sites), a the disclosed AR functional secondary and/or tertiary RNA structure (including splice sites), or any combinations thereof when subjected to an enzymatic assay in the presence of the disclosed compound than the level of degradation in a control or reference sample. In an aspect, stabilizing a disclosed Kaposi's sarcoma-associated herpesvirus (KSHV) polyadenylated nuclear (PAN) 3′-end triple helix, a disclosed Kaposi's sarcoma-associated herpesvirus (KSHV) polyadenylated nuclear (PAN) functional secondary structures and/or tertiary structures, a disclosed SARS-CoV-2 5′-untranslated region structure and/or 3′-untranslated region, a disclosed SARS-CoV-2 5′-untranslated region structure can comprise a frameshifting pseudoknot, a disclosed HIV element (e.g., a trans-activation response (TAR) element, a Rev response element (RRE), EESV, a frameshift site, a packing signal (interaction between (w) stem-loop 3 (SL3) RNA and Gag), or any combination thereof), a disclosed secondary structure or a disclosed tertiary structure in a poliovirus, a rhinovirus, an encephalomyocarditis virus, a foot-and-mouth disease virus, a Kaposi's sarcoma-associated herpesvirus, a hepatitis A virus, or a hepatitis C virus, a disclosed secondary or tertiary RNA structure in a coronavirus, a flavivirus, alphaviruses, a picornavirus, or a positive sense RNA virus, or any combinations thereof can comprise measuring less degradation of the disclosed Kaposi's sarcoma-associated herpesvirus (KSHV) polyadenylated nuclear (PAN) 3′-end triple helix, the disclosed Kaposi's sarcoma-associated herpesvirus (KSHV) polyadenylated nuclear (PAN) functional secondary structures and/or tertiary structures, the disclosed SARS-CoV-2 5′-untranslated region structure and/or 3′-untranslated region, the disclosed SARS-CoV-2 5′-untranslated region structure can comprise a frameshifting pseudoknot, the disclosed HIV element (e.g., a trans-activation response (TAR) element, the Rev response element (RRE), EESV, the frameshift site, the packing signal (interaction between (w) stem-loop 3 (SL3) RNA and Gag), or any combination thereof), the disclosed secondary structure or a disclosed tertiary structure in a poliovirus, a rhinovirus, an encephalomyocarditis virus, a foot-and-mouth disease virus, a Kaposi's sarcoma-associated herpesvirus, a hepatitis A virus, or a hepatitis C virus, the disclosed secondary or tertiary RNA structure in a coronavirus, a flavivirus, alphaviruses, a picornavirus, or a positive sense RNA virus, or any combinations thereof when subjected to an enzymatic assay in the presence of the disclosed compound than the level of degradation in a control or reference sample.

In an aspect, destabilizing a disclosed MALAT1 triple helix, a disclosed NEAT1 triplex, a disclosed PAN triplex, or any combination thereof can comprise measuring more degradation of the disclosed MALAT1 triple helix, the disclosed NEAT1 triplex, the disclosed PAN triplex, or any combination thereof when subjected to an enzymatic assay in the presence of the disclosed compound than the level of degradation in a control or reference sample. In an aspect, destabilizing a disclosed lncRNA thereof can comprise measuring more degradation of the disclosed lncRNA when subjected to an enzymatic assay in the presence of the disclosed compound than the level of degradation in a control or reference sample. In an aspect, destabilizing a disclosed HIV packaging signal, a disclosed complex junction in the long noncoding RNA SChLAP1, and combinations thereof can comprise measuring more degradation of the disclosed HIV packaging signal, a disclosed complex junction in the long noncoding RNA SChLAP1, and combinations thereof when subjected to an enzymatic assay in the presence of the disclosed compound than the level of degradation in a control or reference sample. In an aspect, destabilizing a LMO7 functional secondary and/or tertiary RNA structure (including splice sites), an AR functional secondary and/or tertiary RNA structure (including splice sites), or any combinations thereof can comprise measuring more degradation of the disclosed LMO7 functional secondary and/or tertiary RNA structure (including splice sites), a the disclosed AR functional secondary and/or tertiary RNA structure (including splice sites), or any combinations thereof when subjected to an enzymatic assay in the presence of the disclosed compound than the level of degradation in a control or reference sample. In an aspect, destabilizing a disclosed Kaposi's sarcoma-associated herpesvirus (KSHV) polyadenylated nuclear (PAN) 3′-end triple helix, a disclosed Kaposi's sarcoma-associated herpesvirus (KSHV) polyadenylated nuclear (PAN) functional secondary structures and/or tertiary structures, a disclosed SARS-CoV-2 5′-untranslated region structure and/or 3′-untranslated region, a disclosed SARS-CoV-2 5′-untranslated region structure can comprise a frameshifting pseudoknot, a disclosed HIV element (e.g., a trans-activation response (TAR) element, a Rev response element (RRE), EESV, a frameshift site, a packing signal (interaction between (w) stem-loop 3 (SL3) RNA and Gag), or any combination thereof), a disclosed secondary structure or a disclosed tertiary structure in a poliovirus, a rhinovirus, an encephalomyocarditis virus, a foot-and-mouth disease virus, a Kaposi's sarcoma-associated herpesvirus, a hepatitis A virus, or a hepatitis C virus, a disclosed secondary or tertiary RNA structure in a coronavirus, a flavivirus, alphaviruses, a picornavirus, or a positive sense RNA virus, or any combinations thereof can comprise measuring more degradation of the disclosed Kaposi's sarcoma-associated herpesvirus (KSHV) polyadenylated nuclear (PAN) 3′-end triple helix, the disclosed Kaposi's sarcoma-associated herpesvirus (KSHV) polyadenylated nuclear (PAN) functional secondary structures and/or tertiary structures, the disclosed SARS-CoV-2 5′-untranslated region structure and/or 3′-untranslated region, the disclosed SARS-CoV-2 5′-untranslated region structure can comprise a frameshifting pseudoknot, the disclosed HIV element (e.g., a trans-activation response (TAR) element, the Rev response element (RRE), EESV, the frameshift site, the packing signal (interaction between (w) stem-loop 3 (SL3) RNA and Gag), or any combination thereof), the disclosed secondary structure or a disclosed tertiary structure in a poliovirus, a rhinovirus, an encephalomyocarditis virus, a foot-and-mouth disease virus, a Kaposi's sarcoma-associated herpesvirus, a hepatitis A virus, or a hepatitis C virus, the disclosed secondary or tertiary RNA structure in a coronavirus, a flavivirus, alphaviruses, a picornavirus, or a positive sense RNA virus, or any combinations thereof when subjected to an enzymatic assay in the presence of the disclosed compound than the level of degradation in a control or reference sample.

In an aspect, a disclosed lncRNA can comprise any disclosed lncRNA (e.g., for example, those listed in one or more databases such as LincSNP, LncVar, LncRNADisease, EVLncRNAs, CLC, Lnc2Cancer, LncBook, NONCODE, LncRNAWiki, or any combination thereof).

In an aspect, a disclosed lncRNA can comprise ALAL-1, ANRIL, ATXN80S, CCAT2, CCR5AS, CUPID1/2, DA125942, DBE-T, DIRC3, DISC2, FALI, Gas5, HOTAIR, HULC, H19, IPW, IFNG-AS, Lnc-NR2F1, Lnc13, LINC00237, LINC00305, Linc-HELLP, LOC285194, MIAT, MIR2052HG, NEAT1, OVAL, PCAN-R1, PCAN-R2, PCAT1, PCAT1, PRAL, PTCSC3, PVT1, RMRP, RMST, RUNXOR, SAMMSON, SNHG17, or any combination thereof.

In an aspect, modulating a disclosed lncRNA can comprise stabilizing a disclosed lncRNA. In an aspect, modulating a disclosed lncRNA can comprise destabilizing a disclosed lncRNA. In an aspect, stabilizing a disclosed lncRNA can comprise measuring less degradation of a disclosed lncRNA when subjected to an enzymatic assay in the presence of the disclosed compound than a control or reference level of degradation. In an aspect, stabilizing a disclosed lncRNA can comprise measuring less degradation of a disclosed lncRNA when subjected to an enzymatic assay in the presence of the disclosed compound than the level of degradation in a control or reference sample. In an aspect, destabilizing a disclosed lncRNA can comprise measuring more degradation of a disclosed lncRNA when subjected to an enzymatic assay in the presence of the disclosed compound than a control or reference level of degradation. In an aspect, destabilizing a disclosed lncRNA can comprise measuring more degradation of a disclosed lncRNA when subjected to an enzymatic assay in the presence of the disclosed compound than the level of degradation in a control or reference sample. In an aspect, a disclosed lncRNA can comprise any disclosed lncRNA. In an aspect, modulating a disclosed lncRNA can comprise stabilizing a disclosed lncRNA. In an aspect, modulating a disclosed lncRNA can comprise destabilizing a disclosed lncRNA. In an aspect, a disclosed compound can decrease the expression and/or level of a disclosed lncRNA.

In an aspect, a disclosed enzymatic assay can comprise using an RNase. In an aspect, a disclosed enzymatic assay can comprise using one or more RNases. In an aspect, a disclosed RNase can comprise an endoribonuclease or an exoribonuclease. In an aspect, a disclosed endoribonuclease can comprise RNase A, RNase P, RNase H, RNase I, RNase III, RNase T1, RNase T2, RNase U2, RNase V1, RNase PhyM, RNase V, or any combination thereof. In an aspect, a disclosed RNase can comprise RNase A. In an aspect, a disclosed exoribonuclease can comprise RNase PH, RNase II, RNase R, RNase D, RNA T, or any combination thereof.

In an aspect, a disclosed control or reference level of degradation can be obtained. In an aspect, obtaining the control or reference level of degradation can comprise subjecting the disclosed tertiary RNA structure to a RNase in the absence of the compound. In an aspect, a disclosed level of degradation in a control or reference sample can be obtained. In an aspect, a disclosed level of degradation in a control or reference sample can be obtained by subjecting the tertiary RNA structure to a RNase in the absence of the disclosed compound. In an aspect, a disclosed control or reference level of degradation can be obtained. In an aspect, a disclosed control or reference level of degradation can be obtained by subjecting the RNA triplex to a RNase in the absence of the compound. In an aspect, a disclosed level of degradation in a control or reference sample can be obtained. In an aspect, a disclosed level of degradation in a control or reference sample can be obtained subjecting the RNA triplex to a RNase in the absence of the disclosed compound.

In an aspect, a disclosed tertiary RNA structure can comprise one or more functionally important RNAs. In an aspect, disclosed functionally important RNAs can comprise human RNAs, viral RNAs, bacterial RNAs, fungal RNAs, yeast RNAs, or RNAs of an organism. In an aspect, a disclosed targeted RNA or a disclosed targeted RNA structure can comprise a bulge, a S-turn, an internal loop, a 3-multiloop, a hairpin loop, a pseudoknot, an apical loop, a kissing loop, a coaxial helix, a stacking helix, a two-way junction, a three-way junction, a four-way junction, or any combination thereof. In an aspect, a disclosed targeted RNA or a disclosed targeted RNA structure can comprise a GNAA tetraloop, a GNGA loop, a T-loop, a C-loop, a E-loop, a Sarcin-ricin loop, a Kink-turn, a Reverse kink-turn, a Hook-turn, a Tandem shear, a Tetraloop-receptor motifs, an interacting tetraloop (e.g., GNRA, GANC, and GAAA tetraloops), a triple-stranded RNA, or any combination thereof.

In an aspect, a disclosed tertiary RNA structure can comprise one or more disease-associated RNAs or disease relevant RNAs. In an aspect, a disclosed tertiary RNA structure can comprise a disclosed tertiary RNA structure. In an aspect, a disclosed tertiary RNA structure can comprise a NEAT1 triplex, a MALAT1 triplex, a PAN triplex, or any combination thereof. In an aspect, a disclosed tertiary RNA structure can comprise a SARS-CoV-2 pseudoknot, the HIV packaging signal, a complex junction in the long noncoding RNA SChLAP1, or any combination thereof. In an aspect, a disclosed tertiary RNA structure can comprise a MALAT1 triplex. In an aspect, a disclosed tertiary RNA structure can comprise a RNA triplex. In an aspect, a disclosed RNA triplex can comprise one or more functionally important RNAs. In an aspect, a disclosed RNA triplex com can comprise one or more disease-associated RNA triplexes or one or more disease relevant RNA triplexes. In an aspect, a disclosed RNA triplex can comprise a NEAT1 triplex, a MALAT1 triplex, a PAN triplex, or any combination thereof. In an aspect, a disclosed RNA triplex can comprise a MALAT1 triplex. In an aspect, a disclosed tertiary RNA structure can comprise LMO7 tertiary RNA structure (including splice sites), an AR tertiary RNA structure (including splice sites), or any combination thereof. In an aspect, a disclosed secondary RNA structure can comprise LMO7 tertiary RNA structure (including splice sites), an AR secondary RNA structure (including splice sites), or any combination thereof.

In an aspect, a disclosed secondary structure and/or a disclosed tertiary structure can comprise a disclosed Kaposi's sarcoma-associated herpesvirus (KSHV) polyadenylated nuclear (PAN) 3′-end triple helix, a disclosed Kaposi's sarcoma-associated herpesvirus (KSHV) polyadenylated nuclear (PAN) functional secondary structures and/or tertiary structures, a disclosed SARS-CoV-2 5′-untranslated region structure and/or 3′-untranslated region, a disclosed SARS-CoV-2 5′-untranslated region structure can comprise a frameshifting pseudoknot, a disclosed HIV element (e.g., a trans-activation response (TAR) element, a Rev response element (RRE), EESV, a frameshift site, a packing signal (interaction between (w) stem-loop 3 (SL3) RNA and Gag), or any combination thereof), a disclosed secondary structure or a disclosed tertiary structure in a poliovirus, a rhinovirus, an encephalomyocarditis virus, a foot-and-mouth disease virus, a Kaposi's sarcoma-associated herpesvirus, a hepatitis A virus, or a hepatitis C virus, a disclosed secondary or tertiary RNA structure in a coronavirus, a flavivirus, alphaviruses, a picornavirus, or a positive sense RNA virus, or any combinations thereof.

In an aspect, a disclosed compound can physically disturb and/or disrupt and/or interrupt the binding of one or more disclosed secondary RNA structures, one or more tertiary RNA structures, or any combination thereof to another structure or molecule. In an aspect, a disclosed compound can physically prevent the binding of one or more disclosed secondary RNA structures, one or more tertiary RNA structures, or any combination thereof to another structure or molecule. In an aspect, a disclosed compound can be toxic to one or more cell types. In an aspect, a disclosed compound can disrupt and/or impair the functionality of one or more cell types. In an aspect, a disclosed cell type can comprise cancer cells or tumor cells.

In an aspect, disclosed cancer cells or disclosed tumor cells can comprise cells associated with adenocarcinoma of the ileum; adenocarcinoma; AIDS associated leukemias and adult T-cell leukemia lymphoma; basal cell carcinoma; basocellular cancer; bile duct cancer; biliary tract cancer; bladder cancer; brain cancer including glioblastomas and medulloblastomas; breast cancer; carcinosarcoma; cervical cancer; choriocarcinoma; clear cell renal cell carcinoma; colon cancer; embryonal carcinoma; embryonic testicular cancer; endometrial cancer; esophageal cancer; gastric cancer; head and neck cancer; hematological neoplasms (e.g., acute lymphocytic and myelogenous leukemia); hepatic cancer; intraepithelial neoplasms (e.g., Bowen's disease and Paget's disease); Kaposi's sarcoma; kidney cancer; liver cancer; lung cancer; lymphomas including Hodgkin's disease and lymphocytic lymphomas; malignant melanoma; malignant pleomorphic adenoma; melanoma; multiple myeloma; neuroblastomas; non-small cell lung cancer (NSCLC); oral cancer (e.g., squamous cell carcinoma); ovarian adenocarcinoma; ovarian cancer (e.g., including those arising and/or affect epithelial cells; stromal cells; germ cells and mesenchymal cells); ovarian teratocarcinoma; pancreatic cancer; papillary carcinoma; papillary renal cell carcinoma; placental choriocarcinoma; prostate cancer; rectal cancer; renal cell carcinoma; renal cancer including adenocarcinoma and Wilms tumor; sarcomas (e.g., leiomyosarcoma, rhabdomyosarcoma, liposarcoma, fibrosarcoma, osteosarcoma, etc.); skin cancer; small bowel adenocarcinoma; small bowel cancer; small cell lung cancer (SCLC); squamous cell carcinoma; squamous cell lung carcinoma; synovial sarcoma; teratocarcinoma; testicular embryonal carcinoma; testicular seminoma; testicular teratoma; testicular cancer including germinal tumors (seminoma, non-seminoma[teratomas; choriocarcinomas]), stromal tumors and germ cell tumors); thyroid cancer including thyroid adenocarcinoma and medullar carcinoma; transitional cell carcinoma; uterine cancer; or any combination thereof.

In an aspect, a disclosed compound can initiate, induce, promote, elicit, hasten, and/or cause the death of the cancer cell or tumor cell. In an aspect, a disclosed compound can reduce and/or minimize the likelihood of metastasis. In an aspect, cell death can be apoptotic and/or necrotic.

### H. Inventive Compositions

Disclosed herein are compositions comprising one or more compounds made by a method of assessing a library of shape diverse small molecules. Disclosed herein are compositions comprising one or more compounds made by a method of assessing a library of shape diverse small molecules. Disclosed herein are compositions comprising one or more compounds made by a disclosed method. Disclosed herein is a composition comprising a disclosed compound, and one or more carriers and/or excipients. Disclosed herein is a composition comprising a disclosed compound comprising a small molecule having a diminazene scaffold, and one or more carriers and/or excipients.

Disclosed herein is a composition comprising DMZ-P1, DMZ-P5, DMZ-P8, DMZ-P9, DMZ-P13, DMZ-P14, DMZ-P17, DMZ-O1, DMZ-O2, DMZ-O4, DMZ-O5, DMZ-O6, DMZ-M1, DMZ-M3, DMZ-M4, DMZ-M7, DMZ-M10, DMZ-M15, DMZ-M22, DMZ-M24, DMZ-N-Me-M9, DMZ-N-Me-mPy-P13, DMZ-mPy-P5-2HCl, DMZ-mPy-P13, DMZ-mF-P5, DMZ-P5-mono, DMZ-mF-P13, DMZ-mF-P13-mono, DMZ-P29, DMZ-P29-mono, DMZ-P0-P5, DMZ-P30, DMZ-P30-mono, DMZ-mMe-P5, DMZ-mMe-P5-mono, DMZ-mMe-P13, DMZ-mMe-P13-mono, DMZ-N-Me-P5, DMZ-N-Me-P13, DMZ-P31, DMZ-P0-P32, DMZ-oMe-P5, DMZ-oMe-P5-mono, DMZ-M5, DMZ-M5-mono, DMZ-M13, Aniline-P13, DMZ-P0-P13, DMZ-M4-P5, DMZ-M4-P13, DMZ-P4, DMZ-P4-mono, DMZ-P5-P13, DMZ-P33, DMZ-P33-mono, DMZ-P2, DMZ-P2-mono, DMZ-P32, DMZ-P35, DMZ-oMe-P13, DMZ-N-Et-P13, DMZ-P34, DMZ-N-Me-M13, DMZ-N-Me-mPy-P13, DMZ-N-Et-P5, DMZ-N-Me-P36, DMZ-N-Me-P36-mono, DMZ-P37, DMZ-P37-mono, DMZ-N-nPr-P13, DMZ-N-Me-P38, DMZ-N-Me-M9, or any combination thereof, and one or more carriers and/or excipients.

In an aspect, a disclosed composition can target a disclosed RNA or a disclosed RNA structure. In an aspect, a disclosed RNA or a disclosed RNA structure can comprise a functionally important RNA.

In an aspect, a disclosed composition can target a bulge, a S-turn, an internal loop, a 3-multiloop, a hairpin loop, a pseudoknot, an apical loop, a kissing loop, a coaxial helix, a stacking helix, a two-way junction, a three-way junction, a four-way junction, or any combination thereof.

In an aspect, a disclosed composition can target a GNAA tetraloop, a GNGA loop, a T-loop, a C-loop, a E-loop, a Sarcin-ricin loop, a Kink-turn, a Reverse kink-turn, a Hook-turn, a Tandem shear, a Tetraloop-receptor motifs, an interacting tetraloop (e.g., GNRA, GANC, and GAAA tetraloops), a triple-stranded RNA, or any combination thereof.

In an aspect, a disclosed composition can modulate a disclosed secondary RNA structure, a disclosed tertiary RNA structure, a disclosed RNA triplex, a disclosed lncRNA, or any combination thereof. In an aspect, a disclosed composition can decrease the expression and/or activity level of a disclosed secondary RNA structure, a disclosed tertiary RNA structure, a disclosed RNA triplex, a disclosed lncRNA, or any combination thereof. In an aspect, a disclosed composition can increase the expression and/or activity level of a disclosed secondary RNA structure, a disclosed tertiary RNA structure, a disclosed RNA triplex, a disclosed lncRNA, or any combination thereof. In an aspect, a disclosed composition can increase the stability of a disclosed secondary RNA structure, a disclosed tertiary RNA structure, a disclosed RNA triplex, a disclosed lncRNA, or any combination thereof. In an aspect, a disclosed composition can decrease the stability of a disclosed secondary RNA structure, a disclosed tertiary RNA structure, a disclosed RNA triplex, a disclosed lncRNA, or any combination thereof.

Disclosed secondary RNA structures are discussed supra. Disclosed tertiary RNA structures are discussed supra. Disclosed RNA triplexes are discussed supra. Disclosed lncRNA are discussed supra.

In an aspect, stabilizing a disclosed secondary RNA structure, a disclosed tertiary RNA structure, a disclosed RNA triplex, a disclosed lncRNA can comprise measuring less degradation of the disclosed secondary RNA structure, a disclosed tertiary RNA structure, a disclosed RNA triplex, a disclosed lncRNA, or any combination thereof in the presence of the disclosed composition when subjected to an enzymatic assay in the presence of the composition than a control or reference level of degradation. In an aspect, destabilizing a disclosed secondary RNA structure, a disclosed tertiary RNA structure, a disclosed RNA triplex, a disclosed lncRNA can comprise measuring more degradation of the disclosed secondary RNA structure, a disclosed tertiary RNA structure, a disclosed RNA triplex, a disclosed lncRNA, or any combination thereof in the presence of the disclosed composition when subjected to an enzymatic assay in the presence of the composition than a control or reference level of degradation.

In an aspect, a disclosed secondary RNA structure, a disclosed tertiary RNA structure, a disclosed RNA triplex, a disclosed lncRNA, or any combination thereof can comprise one or more functionally important RNAs. In an aspect, a disclosed secondary RNA structure, a disclosed tertiary RNA structure, a disclosed RNA triplex, a disclosed lncRNA, or any combination thereof can comprise one or more disease-associated RNAs or disease relevant RNAs.

In an aspect, a disclosed composition can be toxic to one or more cell types. In an aspect, a disclosed composition can disrupt and/or impair the functionality of one or more cell types. In an aspect, a disclosed cell type can comprise cancer cells or tumor cells. Cancer cells and tumor cells are known to the art and are discussed supra. In an aspect, a disclosed composition can initiate, induce, promote, elicit, hasten, and/or cause the death of the cancer cell or tumor cell. In an aspect, a disclosed composition can reduce and/or minimize the likelihood of metastasis. In an aspect, cell death can be apoptotic and/or necrotic.

In an aspect, a disclosed composition can physically disturb and/or disrupt and/or interrupt the binding of one or more disclosed secondary RNA structures, one or more tertiary RNA structures, or any combination thereof to another structure or molecule. In an aspect, a disclosed composition can physically prevent the binding of one or more disclosed secondary RNA structures, one or more tertiary RNA structures, or any combination thereof to another structure or molecule.

### I. Inventive Pharmaceutical Formulations

Disclosed herein is a pharmaceutical formulation comprising a disclosed compound and one or more pharmaceutically acceptable carriers. Disclosed herein is pharmaceutical formulation comprising a disclosed composition and one or more pharmaceutically acceptable carriers. Disclosed herein is a pharmaceutical formulation comprising one or more disclosed compounds and one or more pharmaceutically acceptable carriers. Disclosed herein is pharmaceutical formulation comprising one or more disclosed compositions and one or more pharmaceutically acceptable carriers.

Disclosed herein is a pharmaceutical formulation comprising DMZ-P1, DMZ-P5, DMZ-P8, DMZ-P9, DMZ-P13, DMZ-P14, DMZ-P17, DMZ-O1, DMZ-O2, DMZ-O4, DMZ-O5, DMZ-06, DMZ-M1, DMZ-M3, DMZ-M4, DMZ-M7, DMZ-M10, DMZ-M15, DMZ-M22, DMZ-M24, DMZ-N-Me-M9, DMZ-N-Me-mPy-P13, DMZ-mPy-P5-2HCl, DMZ-mPy-P13, DMZ-mF-P5, DMZ-P5-mono, DMZ-mF-P13, DMZ-mF-P13-mono, DMZ-P29, DMZ-P29-mono, DMZ-P0-P5, DMZ-P30, DMZ-P30-mono, DMZ-mMe-P5, DMZ-mMe-P5-mono, DMZ-mMe-P13, DMZ-mMe-P13-mono, DMZ-N-Me-P5, DMZ-N-Me-P13, DMZ-P31, DMZ-P0-P32, DMZ-oMe-P5, DMZ-oMe-P5-mono, DMZ-M5, DMZ-M5-mono, DMZ-M13, Aniline-P13, DMZ-P0-P13, DMZ-M4-P5, DMZ-M4-P13, DMZ-P4, DMZ-P4-mono, DMZ-P5-P13, DMZ-P33, DMZ-P33-mono, DMZ-P2, DMZ-P2-mono, DMZ-P32, DMZ-P35, DMZ-oMe-P13, DMZ-N-Et-P13, DMZ-P34, DMZ-N-Me-M13, DMZ-N-Me-mPy-P13, DMZ-N-Et-P5, DMZ-N-Me-P36, DMZ-N-Me-P36-mono, DMZ-P37, DMZ-P37-mono, DMZ-N-nPr-P13, DMZ-N-Me-P38, DMZ-N-Me-M9, or any combination thereof, and one or more carriers and/or excipients.

In an aspect, a disclosed pharmaceutical formulation can target a disclosed RNA or a disclosed RNA structure. In an aspect, a disclosed RNA or a disclosed RNA structure can comprise a functionally important RNA.

In an aspect, a disclosed pharmaceutical formulation can target a bulge, a S-turn, an internal loop, a 3-multiloop, a hairpin loop, a pseudoknot, an apical loop, a kissing loop, a coaxial helix, a stacking helix, a two-way junction, a three-way junction, a four-way junction, or any combination thereof. In an aspect, a disclosed pharmaceutical formulation can target a GNAA tetraloop, a GNGA loop, a T-loop, a C-loop, a E-loop, a Sarcin-ricin loop, a Kink-turn, a Reverse kink-turn, a Hook-turn, a Tandem shear, a Tetraloop-receptor motifs, an interacting tetraloop (e.g., GNRA, GANC, and GAAA tetraloops), a triple-stranded RNA, or any combination thereof.

In an aspect, a disclosed pharmaceutical formulation can modulate a disclosed secondary RNA structure, a disclosed tertiary RNA structure, a disclosed RNA triplex, a disclosed lncRNA, or any combination thereof. In an aspect, a disclosed pharmaceutical formulation can decrease the expression and/or activity level of a disclosed secondary RNA structure, a disclosed tertiary RNA structure, a disclosed RNA triplex, a disclosed lncRNA, or any combination thereof. In an aspect, a disclosed pharmaceutical formulation composition can increase the expression and/or activity level of a disclosed secondary RNA structure, a disclosed tertiary RNA structure, a disclosed RNA triplex, a disclosed lncRNA, or any combination thereof. In an aspect, a disclosed pharmaceutical formulation can increase the stability of a disclosed secondary RNA structure, a disclosed tertiary RNA structure, a disclosed RNA triplex, a disclosed lncRNA, or any combination thereof. In an aspect, a disclosed pharmaceutical formulation can decrease the stability of a disclosed secondary RNA structure, a disclosed tertiary RNA structure, a disclosed RNA triplex, a disclosed lncRNA, or any combination thereof. Disclosed secondary RNA structures are discussed supra. Disclosed tertiary RNA structures are discussed supra. Disclosed RNA triplexes are discussed supra. Disclosed lncRNA are discussed supra.

In an aspect, stabilizing a disclosed secondary RNA structure, a disclosed tertiary RNA structure, a disclosed RNA triplex, a disclosed lncRNA can comprise measuring less degradation of the disclosed secondary RNA structure, a disclosed tertiary RNA structure, a disclosed RNA triplex, a disclosed lncRNA, or any combination thereof when subjected to an enzymatic assay in the presence of the disclosed pharmaceutical formulation a control or reference level of degradation. In an aspect, destabilizing a disclosed secondary RNA structure, a disclosed tertiary RNA structure, a disclosed RNA triplex, a disclosed lncRNA can comprise measuring more degradation of the disclosed secondary RNA structure, a disclosed tertiary RNA structure, a disclosed RNA triplex, a disclosed lncRNA, or any combination thereof when subjected to an enzymatic assay in the presence of the disclosed pharmaceutical formulation than a control or reference level of degradation.

In an aspect, a disclosed secondary RNA structure, a disclosed tertiary RNA structure, a disclosed RNA triplex, a disclosed lncRNA, or any combination thereof can comprise one or more functionally important RNAs. In an aspect, a disclosed secondary RNA structure, a disclosed tertiary RNA structure, a disclosed RNA triplex, a disclosed lncRNA, or any combination thereof can comprise one or more disease-associated RNAs or disease relevant RNAs.

In an aspect, a disclosed pharmaceutical formulation can disrupt and/or impair the functionality of one or more cell types.

In an aspect, a disclosed pharmaceutical formulation composition can initiate, induce, promote, elicit, hasten, and/or cause the death of the cancer cell or tumor cell.

In an aspect, a disclosed pharmaceutical formulation can reduce and/or minimize the likelihood of metastasis. In an aspect, cell death can be apoptotic and/or necrotic.

In an aspect, a disclosed pharmaceutical formulation can physically disturb and/or disrupt and/or interrupt the binding of one or more disclosed secondary RNA structures, one or more tertiary RNA structures, or any combination thereof to another structure or molecule. In an aspect, a disclosed pharmaceutical formulation can physically prevent the binding of one or more disclosed secondary RNA structures, one or more tertiary RNA structures, or any combination thereof to another structure or molecule.

In an aspect, a disclosed pharmaceutical formulation can be toxic to one or more cell types. In an aspect, a disclosed composition can disrupt and/or impair the functionality of one or more cell types. In an aspect, a disclosed cell type can comprise cancer cells or tumor cells. Cancer cells and tumor cells are known to the art and are discussed supra. In an aspect, a disclosed pharmaceutical formulation can initiate, induce, promote, elicit, hasten, and/or cause the death of the cancer cell or tumor cell. In an aspect, a disclosed pharmaceutical formulation can reduce and/or minimize the likelihood of metastasis. In an aspect, cell death can be apoptotic and/or necrotic.

In an aspect, a disclosed pharmaceutical formulation can comprise (i) one or more active agents, (ii) biologically active agents, (iii) one or more pharmaceutically active agents, (iv) one or more immune-based therapeutic agents, (v) one or more clinically approved agents, or (vi) a combination thereof.

Disclosed herein are pharmaceutical formulations one or more compounds made by a method of assessing a library of shape diverse small molecules.

### J. Inventive Library

Disclosed herein is a library of compounds made by a method of assessing a library of shape diverse small molecules.

Disclosed herein is a library comprising one or more disclosed small molecules having a diminazene scaffold. Disclosed herein is a library comprising one or more disclosed shape diverse small molecules having a diminazene scaffold. Disclosed herein is library comprising one or more disclosed small molecules having a diminazene scaffold and a diamidine moiety.

Disclosed herein is a library comprising one or more of DMZ-P1, DMZ-P5, DMZ-P8, DMZ-P9, DMZ-P13, DMZ-P14, DMZ-P17, DMZ-O1, DMZ-O2, DMZ-O4, DMZ-O5, DMZ-O6, DMZ-M1, DMZ-M3, DMZ-M4, DMZ-M7, DMZ-M10, DMZ-M15, DMZ-M22, DMZ-M24, DMZ-N-Me-M9, DMZ-N-Me-mPy-P13, DMZ-mPy-P5-2HCl, DMZ-mPy-P13, DMZ-mF-P5, DMZ-P5-mono, DMZ-mF-P13, DMZ-mF-P13-mono, DMZ-P29, DMZ-P29-mono, DMZ-P0-P5, DMZ-P30, DMZ-P30-mono, DMZ-mMe-P5, DMZ-mMe-P5-mono, DMZ-mMe-P13, DMZ-mMe-P13-mono, DMZ-N-Me-P5, DMZ-N-Me-P13, DMZ-P31, DMZ-P0-P32, DMZ-oMe-P5, DMZ-oMe-P5-mono, DMZ-M5, DMZ-M5-mono, DMZ-M13, Aniline-P13, DMZ-P0-P13, DMZ-M4-P5, DMZ-M4-P13, DMZ-P4, DMZ-P4-mono, DMZ-P5-P13, DMZ-P33, DMZ-P33-mono, DMZ-P2, DMZ-P2-mono, DMZ-P32, DMZ-P35, DMZ-oMe-P13, DMZ-N-Et-P13, DMZ-P34, DMZ-N-Me-M13, DMZ-N-Me-mPy-P13, DMZ-N-Et-P5, DMZ-N-Me-P36, DMZ-N-Me-P36-mono, DMZ-P37, DMZ-P37-mono, DMZ-N-nPr-P13, DMZ-N-Me-P38, DMZ-N-Me-M9, or any combination thereof.

### K. Methods of Treating a Subject

Disclosed herein is a method of treating a subject in need thereof, the method comprising administering to a subject one or more disclosed compounds. Disclosed herein is a method of treating a subject in need thereof, the method comprising administering to a subject one or more disclosed compositions. Disclosed herein is a method of treating a subject in need thereof, the method comprising administering to a subject one or more disclosed pharmaceutical formulations.

Disclosed herein is a method of treating a subject in need thereof, the method comprising administering to a subject one or more disclosed compounds, and treating a disease, condition, or disorder in the subject. Disclosed herein is a method of treating a subject in need thereof, the method comprising administering to a subject one or more disclosed compositions, and treating a disease, condition, or disorder in the subject. Disclosed herein is a method of treating a subject in need thereof, the method comprising administering to a subject one or more disclosed pharmaceutical formulations, and treating a disease, condition, or disorder in the subject.

Disclosed herein is a method of treating a subject in need thereof, the method comprising administering to a subject one or more disclosed compounds, wherein one or more secondary RNA structures are modulated. Disclosed herein is a method of treating a subject in need thereof, the method comprising administering to a subject one or more disclosed compositions, wherein one or more secondary RNA structures are modulated. Disclosed herein is a method of treating a subject in need thereof, the method comprising administering to a subject one or more disclosed pharmaceutical formulations, wherein one or more secondary RNA structures are modulated.

Disclosed herein is a method of treating a subject in need thereof, the method comprising administering to a subject one or more disclosed compounds, wherein one or more tertiary RNA structures are modulated. Disclosed herein is a method of treating a subject in need thereof, the method comprising administering to a subject one or more disclosed compositions, wherein one or more tertiary RNA structures are modulated. Disclosed herein is a method of treating a subject in need thereof, the method comprising administering to a subject one or more disclosed pharmaceutical formulations, wherein one or more tertiary RNA structures are modulated.

Disclosed herein is a method of treating a subject in need thereof, the method comprising administering to a subject one or more disclosed compounds, wherein one or more secondary RNA structures are stabilized. Disclosed herein is a method of treating a subject in need thereof, the method comprising administering to a subject one or more disclosed compositions, wherein one or more secondary RNA structures are stabilized. Disclosed herein is a method of treating a subject in need thereof, the method comprising administering to a subject one or more disclosed pharmaceutical formulations, wherein one or more secondary RNA structures are stabilized.

Disclosed herein is a method of treating a subject in need thereof, the method comprising administering to a subject one or more disclosed compounds, wherein one or more tertiary RNA structures are stabilized. Disclosed herein is a method of treating a subject in need thereof, the method comprising administering to a subject one or more disclosed compositions, wherein one or more tertiary RNA structures are stabilized. Disclosed herein is a method of treating a subject in need thereof, the method comprising administering to a subject one or more disclosed pharmaceutical formulations, wherein one or more tertiary RNA structures are stabilized.

Disclosed herein is a method of treating a subject in need thereof, the method comprising administering to a subject one or more disclosed compounds, wherein one or more secondary RNA structures are destabilized. Disclosed herein is a method of treating a subject in need thereof, the method comprising administering to a subject one or more disclosed compositions, wherein one or more secondary RNA structures are destabilized. Disclosed herein is a method of treating a subject in need thereof, the method comprising administering to a subject one or more disclosed pharmaceutical formulations, wherein one or more secondary RNA structures are destabilized.

Disclosed herein is a method of treating a subject in need thereof, the method comprising administering to a subject one or more disclosed compounds, wherein one or more tertiary RNA structures are destabilized. Disclosed herein is a method of treating a subject in need thereof, the method comprising administering to a subject one or more disclosed compositions, wherein one or more tertiary RNA structures are destabilized. Disclosed herein is a method of treating a subject in need thereof, the method comprising administering to a subject one or more disclosed pharmaceutical formulations, wherein one or more tertiary RNA structures are destabilized.

Disclosed herein is a method of treating a subject in need thereof, the method comprising administering to a subject one or more disclosed compounds, wherein the expression and/or activity level of one or more secondary RNA structures are decreased. Disclosed herein is a method of treating a subject in need thereof, the method comprising administering to a subject one or more disclosed compositions, wherein the expression and/or activity level of one or more secondary RNA structures are decreased. Disclosed herein is a method of treating a subject in need thereof, the method comprising administering to a subject one or more disclosed pharmaceutical formulations, wherein the expression and/or activity level of one or more secondary RNA structures are decreased.

Disclosed herein is a method of treating a subject in need thereof, the method comprising administering to a subject one or more disclosed compounds, wherein the expression and/or activity level of one or more tertiary RNA structures are decreased. Disclosed herein is a method of treating a subject in need thereof, the method comprising administering to a subject one or more disclosed compositions, wherein the expression and/or activity level of one or more tertiary RNA structures are decreased. Disclosed herein is a method of treating a subject in need thereof, the method comprising administering to a subject one or more disclosed pharmaceutical formulations, wherein the expression and/or activity level of one or more tertiary RNA structures are decreased.

Disclosed herein is a method of treating a subject in need thereof, the method comprising administering to a subject one or more disclosed compounds, wherein the expression and/or activity level of one or more secondary RNA structures are increased. Disclosed herein is a method of treating a subject in need thereof, the method comprising administering to a subject one or more disclosed compositions, wherein the expression and/or activity level of one or more secondary RNA structures are increased. Disclosed herein is a method of treating a subject in need thereof, the method comprising administering to a subject one or more disclosed pharmaceutical formulations, wherein the expression and/or activity level of one or more secondary RNA structures are increased.

Disclosed herein is a method of treating a subject in need thereof, the method comprising administering to a subject one or more disclosed compounds, wherein the expression and/or activity level of one or more tertiary RNA structures are decreased. Disclosed herein is a method of treating a subject in need thereof, the method comprising administering to a subject one or more disclosed compositions, wherein the expression and/or activity level of one or more tertiary RNA structures are decreased. Disclosed herein is a method of treating a subject in need thereof, the method comprising administering to a subject one or more disclosed pharmaceutical formulations, wherein the expression and/or activity lev

In an aspect, a disclosed lncRNA can comprise any disclosed lncRNA (e.g., for example, those listed in one or more databases such as LincSNP, LncVar, LncRNADisease, EVLncRNAs, CLC, Lnc2Cancer, LncBook, NONCODE, LncRNAWiki, or any combination thereof).

In an aspect, a disclosed lncRNA can comprise ALAL-1, ANRIL, ATXN80S, CCAT2, CCR5AS, CUPID1/2, DA125942, DBE-T, DIRC3, DISC2, FALI, Gas5, HOTAIR, HULC, H19, IPW, IFNG-AS, Lnc-NR2F1, Lnc13, LINC00237, LINC00305, Linc-HELLP, LOC285194, MIAT, MIR2052HG, NEAT1, OVAL, PCAN-R1, PCAN-R2, PCAT1, PCAT1, PRAL, PTCSC3, PVT1, RMRP, RMST, RUNXOR, SAMMSON, SNHG17, or any combination thereof.

In an aspect, a disclosed RNA triplex can comprise a disclosed MALAT1 triple helix, a disclosed NEAT1 triplex, a disclosed PAN triplex, or any combination thereof.

In an aspect, a disclosed tertiary RNA structure can a disclosed SARS-CoV-2 pseudoknot, a disclosed HIV packaging signal, a disclosed complex junction in the long noncoding RNA SChLAP1, and combinations thereof.

In an aspect of a disclosed method, (i) a disease, condition, or disorder in the subject can treated, (ii) one or more symptoms of a disease, condition, or disorder in the subject can be attenuated, ameliorated, or eliminated, (iii) the onset of one or more symptoms of a disease, condition, or disorder in the subject can be delayed, (iv) the progression of the disease, condition, or disorder in the subject can be slowed and/or diminished, or any combination thereof.

In an aspect, a disclosed subject can be a human, can be male or female, and can be of any age (e.g., adult, adolescent, child, baby, etc.). In an aspect of a disclosed method, a disclosed subject can be suspected of having or can be diagnosed with having a disease, condition, or disorder caused by, related to, and/or exacerbated by the presence of, expression of, and/or activity of one or more secondary RNA structures, one or more tertiary RNA structures, or any combination thereof. In an aspect, a disclosed subject can be symptomatic or asymptomatic. In an aspect of a disclosed method, a disclosed subject can be suspected of having or can be diagnosed with having a disease, condition, or disorder caused by, related to, and/or exacerbated by the absence of, lack of expression of, and/or lack of activity of one or more secondary RNA structures, one or more tertiary RNA structures, or any combination thereof. In an aspect, a disclosed subject can be symptomatic or asymptomatic.

In an aspect of a disclosed method, the subject can have one or more cancers and/or one or more tumors. Cancers and tumors are known to the skilled person. In an aspect, a disclosed cancer can comprise adenocarcinoma of the ileum; adenocarcinoma; AIDS associated leukemias and adult T-cell leukemia lymphoma; basal cell carcinoma; basocellular cancer; bile duct cancer; biliary tract cancer; bladder cancer; brain cancer including glioblastomas and medulloblastomas; breast cancer; carcinosarcoma; cervical cancer; choriocarcinoma; clear cell renal cell carcinoma; colon cancer; embryonal carcinoma; embryonic testicular cancer; endometrial cancer; esophageal cancer; gastric cancer; head and neck cancer; hematological neoplasms (e.g., acute lymphocytic and myelogenous leukemia); hepatic cancer; intraepithelial neoplasms (e.g., Bowen's disease and Paget's disease); Kaposi's sarcoma; kidney cancer; liver cancer; lung cancer; lymphomas including Hodgkin's disease and lymphocytic lymphomas; malignant melanoma; malignant pleomorphic adenoma; melanoma; multiple myeloma; neuroblastomas; non-small cell lung cancer (NSCLC); oral cancer (e.g., squamous cell carcinoma); ovarian adenocarcinoma; ovarian cancer (e.g., including those arising and/or affect epithelial cells; stromal cells; germ cells and mesenchymal cells); ovarian teratocarcinoma; pancreatic cancer; papillary carcinoma; papillary renal cell carcinoma; placental choriocarcinoma; prostate cancer; rectal cancer; renal cell carcinoma; renal cancer including adenocarcinoma and Wilms tumor; sarcomas (e.g., leiomyosarcoma, rhabdomyosarcoma, liposarcoma, fibrosarcoma, osteosarcoma, etc.); skin cancer; small bowel adenocarcinoma; small bowel cancer; small cell lung cancer (SCLC); squamous cell carcinoma; squamous cell lung carcinoma; synovial sarcoma; teratocarcinoma; testicular embryonal carcinoma; testicular seminoma; testicular teratoma; testicular cancer including germinal tumors (seminoma, non-seminoma[teratomas; choriocarcinomas]), stromal tumors and germ cell tumors); thyroid cancer including thyroid adenocarcinoma and medullar carcinoma; transitional cell carcinoma; uterine cancer; or any combination thereof.

In an aspect of a disclosed method, administering a disclosed compound, a disclosed composition, a disclosed pharmaceutical formulation, or any combination thereof can comprise systemic administration and/or local administration. Methods of administration of known to the art. For example, in an aspect, administration can comprise oral administration, transdermal administration, administration by inhalation, nasal administration, topical administration, in utero administration, intrahepatic administration, intravaginal administration, epidural administration (such as epidural injection), intracerebroventricular (ICV) administration, ophthalmic administration, intraaural administration, depot administration, topical (skin) administration, otic administration, intra-articular (such as joint or vertebrate injection), intracerebral administration, rectal administration, sublingual administration, buccal administration, and parenteral administration, including injectable such as intravenous administration, intra-CSF administration, intra-cistern magna (ICM) administration, intra-arterial administration, intrathecal (ITH) administration, intramuscular administration, and subcutaneous administration.

Administration of a disclosed compound, a disclosed composition, a disclosed pharmaceutical formulation, or any combination thereof can comprise administration directly into the CNS or the PNS. In an aspect, a disclosed compound, a disclosed composition, a disclosed pharmaceutical formulation, or any combination thereof can be administered via LNP administration.

In an aspect, administration can be continuous or intermittent. Administration can comprise a combination of one or more routes. In an aspect, a disclosed compound, a disclosed composition, a disclosed pharmaceutical formulation, or any combination thereof can be concurrently and/or serially administered to a subject via multiple routes of administration. In an aspect, aspect of administration can be informed on the likelihood of the disclosed compound, the disclosed composition, the disclosed pharmaceutical formulation, or any combination thereof achieving a pharmacological effect. In an aspect, concomitant administration does not require a single composition or pharmaceutical formulation, in the same dosage form, by the same route of administration, or at the same time. Rather, in an aspect, the effects of both compounds, compositions, and/or pharmaceutical formulations need not manifest themselves at the same time (e.g., effects need only be overlapping for a period and need not be coextensive). In an aspect, concomitant administration or co-administration can comprise administration in parallel or sequentially.

In an aspect, administering to the subject can comprise contacting one or more cells affected by the targeted RNA or targeted RNA structure. In an aspect, after the administering step, one or more cells affected by the tarted RNA or targeted RNA structure in one or more of the subject's body systems are contacted by a disclosed compound, a disclosed composition, a disclosed pharmaceutical formulation, or any combination thereof.

In an aspect, local administration can comprise delivery to one or more of the subject's body systems affected by the targeted RNA or targeted RNA structure. In an aspect, the one or more body systems affected by the targeted RNA or targeted RNA structure can comprise the cardiovascular system, the digestive system, the endocrine system, the lymphatic system, the muscular system, the nervous system, the reproductive system, the respiratory system, the skeletal system, the urinary system, the integumentary system, or any combination thereof.

In an aspect of a disclosed method, following the administering step, the subject's symptoms can be diminished and/or decreased.

In an aspect of a disclosed method, following the administering step, the subject's quality of life can be improved and/or enhanced. In an aspect of a disclosed method, following the administering step, one or more of the subject's body systems can experience and/or show signs of normal physiology and/or cellular homeostasis. In an aspect, a disclosed method can comprise administering to the subject one or more therapeutic agents and/or active agents. In an aspect, a disclosed therapeutic agent and/or active agent can be any disclosed agent that effects a desired clinical outcome. In an aspect, one or more disclosed therapeutic agents and/or active agents comprises an anti-cancer agent.

In an aspect, a disclosed method can comprise repeating an administering step one or more times. In an aspect, a disclosed method can comprise monitoring the subject for adverse effects. In an aspect, in the absence of adverse effects, the method can comprise continuing to treat the subject and/or continuing to monitor the subject. In an aspect, in the presence of adverse effects, the method can comprise modifying one or more steps of the method.

In an aspect, a disclosed method can comprise reducing the pathological phenotype associated with a disease, condition, or disorder caused by, related to, and/or exacerbated by the presence of, expression of, and/or activity of one or more secondary RNA structures, one or more tertiary RNA structures, or any combination thereof.

In an aspect, a disclosed method can comprise diagnosing the subject as having a disease, condition, or disorder caused by, related to, and/or exacerbated by the presence of, expression of, and/or activity of one or more secondary RNA structures, one or more tertiary RNA structures, or any combination thereof. In an aspect, a disclosed method can further treat one or more symptoms of the subject.

In an aspect, a disclosed method can restore one or more aspects of cellular homeostasis and/or cellular functionality and/or metabolic dysregulation. In an aspect, restoring one or more aspects of cellular homeostasis and/or cellular functionality and/or metabolic dysregulation can comprise reducing the expression and/or activity level of one or more secondary RNA structures, one or more tertiary RNA structures, or any combination thereof that causes, relates to, elicits, and/or exacerbated a disease, disorder, and/or condition in the subject.

In an aspect, restoring one or more aspects of cellular homeostasis and/or cellular functionality can comprise one or more of the following: (i) correcting cell starvation in one or more cell types; (ii) normalizing aspects of the autophagy pathway (such as, for example, correcting, preventing, reducing, and/or ameliorating autophagy); (iii) improving, enhancing, restoring, and/or preserving mitochondrial functionality and/or structural integrity; (iv) improving, enhancing, restoring, and/or preserving organelle functionality and/or structural integrity; (v) correcting enzyme dysregulation; (vi) reversing, inhibiting, preventing, stabilizing, and/or slowing the rate of progression of the multi-systemic manifestations of a genetic disease or disorder; (vii) reversing, inhibiting, preventing, stabilizing, and/or slowing the rate of progression of a genetic disease or disorder, or (viii) any combination thereof. In an aspect, restoring one or more aspects of cellular homeostasis can comprise improving, enhancing, restoring, and/or preserving one or more aspects of cellular structural and/or functional integrity.

In an aspect, a disclosed method can restore the functionality and/or structural integrity of a missing, affected, deficient, and/or mutant protein or enzyme. In an aspect, restoring the activity and/or functionality of a missing, affected, deficient, and/or mutant protein or enzyme can comprise a 10%, 20%, 30%, 40%, 50%, 60%, 70%, 80%, 90%, 100%, or any amount of restoration when compared to a pre-existing level such as, for example, a pre-treatment level. In an aspect, the amount of restoration can be 10-20%, 20-30%, 30-40%, 40-50%, 50-60%, 60-70%, 70-80%, 80-90%, or 90-100% more than a pre-existing level such as, for example, a pre-treatment level. In an aspect, restoration can be measured against a control level or a reference level (e.g., determined, for example, using one or more subjects not having a missing, deficient, and/or mutant protein or enzyme). In an aspect, restoration can be a partial or incomplete restoration. In an aspect, restoration can be complete or near complete restoration such that the level of expression, activity, and/or functionality is like that of a wild-type or control level.

In an aspect, restoring the activity and/or functionality of an affected gene of interest can comprise increasing or enhancing the expression and/or activity level of that affected gene. In an aspect, increasing or enhancing can comprise an elevation of at least about 5%, 10%, 15%, 20%, 25%, 35%, 50%, 75%, 80%, 85%, 90%, 95%, 97%, 98%, 99%, 100%, 200%, 300%, 400%, 500%, or more as compared to a control (such as a pre-treatment level). In an aspect, restoring the activity and/or functionality of an affected gene of interest can comprise decreasing or reducing the expression and/or activity level of that affected gene. In an aspect, decreasing or reducing can comprise a decrease of at least about 5%, 10%, 15%, 20%, 25%, 35%, 50%, 75%, 80%, 85%, 90%, 95%, 97%, 98%, 99%, 100%, 200%, 300%, 400%, 500%, or more as compared to a control (such as a pre-treatment level).

In an aspect of a disclosed method of treating a subject, techniques to monitor, measure, and/or assess the restoring one or more aspects of cellular homeostasis and/or cellular functionality can comprise qualitative (or subjective) means as well as quantitative (or objective) means. These means are known to the skilled person. For example, representative regulated variables and sensors relating to systemic homeostasis are discussed supra.

In an aspect, a disclosed method can comprise measuring and/or ascertaining the expression and/or activity level and/or stability of one or more disclosed secondary RNA structures, one or more disclosed tertiary RNA structures, or any combination thereof in the subject prior to treatment or prior to the administering of a disclosed compound, a disclosed composition, a disclosed pharmaceutical formulation, or any combination thereof. In an aspect, a disclosed method can comprise measuring and/or ascertaining the expression and/or activity level and/or stability of one or more disclosed secondary RNA structures, one or more disclosed tertiary RNA structures, or any combination thereof in the subject after treatment or after the administering of a disclosed compound, a disclosed composition, a disclosed pharmaceutical formulation, or any combination thereof. In an aspect, a disclosed method can comprise measuring and/or ascertaining the expression and/or activity level and/or stability of one or more disclosed secondary RNA structures, one or more disclosed tertiary RNA structures, or any combination thereof in the subject one or more times.

In an aspect, a disclosed subject can be symptomatic or asymptomatic.

In an aspect, the disclosed compound, a disclosed composition, a disclosed pharmaceutical formulation, or any combination thereof can be administered in a single dose, or in multiple doses (such as 2, 3, 4, 5, 6, 7, 8, 9 or 10 doses) as needed for the desired therapeutic results.

In an aspect, a disclosed method of treating a subject can further comprise administering to the subject a therapeutically effective amount of an active agent and/or therapeutic that can correct one or more aspects of a dysregulated metabolic or enzymatic pathway caused by, related to, and/or exacerbated by the presence of, expression of, and/or activity of one or more secondary RNA structures, one or more tertiary RNA structures, or any combination thereof.

In an aspect, a disclosed method can comprise repeating one or more steps of the method and/or modifying one or more steps of the method (such as, for example, an administering step). In an aspect, a disclosed method of treating a subject can comprise modifying one or more of the disclosed steps. For example, modifying one or more of steps of a disclosed method can comprise modifying or changing one or more features or aspects of one or more steps of a disclosed method. For example, in an aspect, a method can be altered by changing the amount of a disclosed compound, a disclosed composition, a disclosed pharmaceutical formulation, or any combination thereof administered to a subject, or by changing the frequency of administration of a disclosed compound, a disclosed composition, a disclosed pharmaceutical formulation, or any combination thereof to a subject, or by changing the duration of time a disclosed compound, a disclosed composition, a disclosed pharmaceutical formulation, or any combination thereof is administered to a subject.

In an aspect, a disclosed method of treating a subject can be altered by changing the amount of a disclosed therapeutic agent and/or active agent administered to a subject, or by changing the frequency of administration of a disclosed therapeutic agent and/or active agent administered to a subject, or by changing the duration of time a disclosed therapeutic agent and/or active agent is administered to the subject.

In an aspect, a disclosed method of treating a subject can comprise generating and/or validating one or more of disclosed compounds, disclosed compositions, disclosed pharmaceutical formulation, or any combination thereof. Methods of generating and/or validating disclosed compounds, disclosed compositions, disclosed pharmaceutical formulations, or any combination thereof are discussed infra. In an aspect, a disclosed method of treating a subject can further comprise administering to the subject one or more additional disclosed compounds, disclosed compositions, disclosed pharmaceutical formulations, or any combination thereof.

In an aspect, a disclosed therapeutic agent and/or active agent can comprise (i) one or more active agents, (ii) biologically active agents, (iii) one or more pharmaceutically active agents, (iv) one or more immune-based therapeutic agents, (v) one or more clinically approved agents, or (vi) a combination thereof.

In an aspect of a disclosed method, a disclosed compound, a disclosed composition, a disclosed pharmaceutical formulation, or any combination thereof can physically disturb and/or disrupt and/or interrupt the binding of one or more disclosed secondary RNA structures, one or more tertiary RNA structures, or any combination thereof to another structure or molecule.

In an aspect of a disclosed method, a disclosed compound, a disclosed composition, a disclosed pharmaceutical formulation, or any combination thereof can physically prevent the binding of one or more disclosed secondary RNA structures, one or more tertiary RNA structures, or any combination thereof to another structure or molecule.

In an aspect of a disclosed method, a disclosed compound, a disclosed composition, a disclosed pharmaceutical formulation, or any combination thereof can be toxic to one or more cell types. In an aspect, a disclosed compound, a disclosed composition, a disclosed pharmaceutical formulation, or any combination thereof can disrupt and/or impair the functionality of one or more cell types. In an aspect, a disclosed cell type can comprise cancer cells or tumor cells. Cancer cells and tumor cells are known to the art and are discussed supra. In an aspect of a disclosed method, a disclosed compound, a disclosed composition, a disclosed pharmaceutical formulation, or any combination thereof can initiate, induce, promote, elicit, hasten, and/or cause the death of the cancer cell or tumor cell. In an aspect, a disclosed pharmaceutical formulation can reduce and/or minimize the likelihood of metastasis. In an aspect, cell death can be apoptotic and/or necrotic.

In an aspect, a disclosed method can comprise administering to the subject DMZ-P1, DMZ-P5, DMZ-P8, DMZ-P9, DMZ-P13, DMZ-P14, DMZ-P17, DMZ-O1, DMZ-O2, DMZ-O4, DMZ-O5, DMZ-O6, DMZ-M1, DMZ-M3, DMZ-M4, DMZ-M7, DMZ-M10, DMZ-M15, DMZ-M22, DMZ-M24, DMZ-N-Me-M9, DMZ-N-Me-mPy-P13, DMZ-mPy-P5-2HCl, DMZ-mPy-P13, DMZ-mF-P5, DMZ-P5-mono, DMZ-mF-P13, DMZ-mF-P13-mono, DMZ-P29, DMZ-P29-mono, DMZ-P0-P5, DMZ-P30, DMZ-P30-mono, DMZ-mMe-P5, DMZ-mMe-P5-mono, DMZ-mMe-P13, DMZ-mMe-P13-mono, DMZ-N-Me-P5, DMZ-N-Me-P13, DMZ-P31, DMZ-P0-P32, DMZ-oMe-P5, DMZ-oMe-P5-mono, DMZ-M5, DMZ-M5-mono, DMZ-M13, Aniline-P13, DMZ-P0-P13, DMZ-M4-P5, DMZ-M4-P13, DMZ-P4, DMZ-P4-mono, DMZ-P5-P13, DMZ-P33, DMZ-P33-mono, DMZ-P2, DMZ-P2-mono, DMZ-P32, DMZ-P35, DMZ-oMe-P13, DMZ-N-Et-P13, DMZ-P34, DMZ-N-Me-M13, DMZ-N-Me-mPy-P13, DMZ-N-Et-P5, DMZ-N-Me-P36, DMZ-N-Me-P36-mono, DMZ-P37, DMZ-P37-mono, DMZ-N-nPr-P13, DMZ-N-Me-P38, DMZ-N-Me-M9, or a pharmaceutically acceptable salt, a hydrate, a prodrug, an ester, or a derivative thereof, or a composition thereof, or a pharmaceutical formulation thereof.

### L. Methods of Modulating Secondary and/or Tertiary RNA Structure

Disclosed herein is a method of modulating one or more secondary RNA structures and/or tertiary RNA structures, the method comprising contacting one or more RNA structures with one or more disclosed compounds, disclosed compositions, disclosed pharmaceutical formulations, or any combination thereof.

In an aspect of a disclosed method, the one or more RNA structures are in a cell or in cells. In an aspect, the cell or cells are in a subject. In an aspect, a subject has been diagnosed with or is suspected of having a disease, condition, or disorder caused by, related to, and/or exacerbated by the presence of, expression of, and/or activity of one or more secondary RNA structures, one or more tertiary RNA structures, or any combination thereof.

In an aspect, the one or more disclosed secondary RNA structures are modulated. In an aspect, the one or more disclosed tertiary RNA structures are modulated. In an aspect, the one or more disclosed secondary RNA structures are stabilized. In an aspect, the one or more disclosed secondary RNA structures are destabilized. In an aspect, the one or more tertiary RNA structures are stabilized. In an aspect, the one or more tertiary RNA structures are destabilized. In an aspect, the expression and/or activity level of one or more disclosed secondary RNA structures are decreased. In an aspect, the expression and/or activity level of one or more disclosed tertiary RNA structures are decreased. In an aspect, the expression and/or activity level of one or more disclosed secondary RNA structures are increased. In an aspect, the expression and/or activity level of one or more disclosed tertiary RNA structures are decreased.

In an aspect, the binding of one or more disclosed secondary RNA structures, one or more tertiary RNA structures, or any combination thereof to another structure or molecule can be physically disturbed and/or disrupted and/or interrupted. In an aspect, the binding of one or more disclosed secondary RNA structures, one or more tertiary RNA structures, or any combination thereof to another structure or molecule can be physically prevented.

In an aspect of a disclosed method, modulating one or more secondary RNA structures and/or tertiary RNA structures be toxic to one or more cell types. In an aspect, modulating one or more secondary RNA structures and/or tertiary RNA structures can disrupt and/or impair the functionality of one or more cell types (such as, for example, cancer cells or tumor cells). Cancer cells and tumor cells are known to the art and are discussed supra. In an aspect, modulating one or more secondary RNA structures and/or tertiary RNA structures can initiate, induce, promote, elicit, hasten, and/or cause the death of the cancer cell or tumor cell. In an aspect, modulating one or more secondary RNA structures and/or tertiary RNA structures can reduce and/or minimize the likelihood of metastasis. In an aspect, cell death can be apoptotic and/or necrotic.

In an aspect, a disclosed method of modulating one or more secondary RNA structures and/or tertiary RNA structures can comprise administering to a subject (i) one or more active agents, (ii) biologically active agents, (iii) one or more pharmaceutically active agents, (iv) one or more immune-based therapeutic agents, (v) one or more clinically approved agents, or (vi) a combination thereof.

In an aspect, a disclosed method can comprise contacting the cells and/or administering to the subject DMZ-P1, DMZ-P5, DMZ-P8, DMZ-P9, DMZ-P13, DMZ-P14, DMZ-P17, DMZ-O1, DMZ-O2, DMZ-O4, DMZ-O5, DMZ-O6, DMZ-M1, DMZ-M3, DMZ-M4, DMZ-M7, DMZ-M10, DMZ-M15, DMZ-M22, DMZ-M24, DMZ-N-Me-M9, DMZ-N-Me-mPy-P13, DMZ-mPy-P5-2HCl, DMZ-mPy-P13, DMZ-mF-P5, DMZ-P5-mono, DMZ-mF-P13, DMZ-mF-P13-mono, DMZ-P29, DMZ-P29-mono, DMZ-P0-P5, DMZ-P30, DMZ-P30-mono, DMZ-mMe-P5, DMZ-mMe-P5-mono, DMZ-mMe-P13, DMZ-mMe-P13-mono, DMZ-N-Me-P5, DMZ-N-Me-P13, DMZ-P31, DMZ-P0-P32, DMZ-oMe-P5, DMZ-oMe-P5-mono, DMZ-M5, DMZ-M5-mono, DMZ-M13, Aniline-P13, DMZ-P0-P13, DMZ-M4-P5, DMZ-M4-P13, DMZ-P4, DMZ-P4-mono, DMZ-P5-P13, DMZ-P33, DMZ-P33-mono, DMZ-P2, DMZ-P2-mono, DMZ-P32, DMZ-P35, DMZ-oMe-P13, DMZ-N-Et-P13, DMZ-P34, DMZ-N-Me-M13, DMZ-N-Me-mPy-P13, DMZ-N-Et-P5, DMZ-N-Me-P36, DMZ-N-Me-P36-mono, DMZ-P37, DMZ-P37-mono, DMZ-N-nPr-P13, DMZ-N-Me-P38, DMZ-N-Me-M9, or a pharmaceutically acceptable salt, a hydrate, a prodrug, an ester, or a derivative thereof, or a composition thereof, or a pharmaceutical formulation thereof.

### M. Methods of Assessing a Library of Small Molecules

Disclosed herein is a method of assessing a library of small molecules that target RNA, the method comprising using a multi-dimensional approach comprising designing and optimizing affinity-based and structural stability-based assays.

Disclosed herein is a method of assessing a library of shape diverse small molecule analogs that modulate a targeted RNA or a targeted RNA structure, the method comprising choosing a candidate small molecule; generating a theoretical library of shape diverse small molecule analogs based on the candidate small molecule; evaluating the three-dimensional shape of one or more shape diverse small molecule analogs of the theoretical library; selecting one or more shape diverse small molecules for synthesis; synthesizing one or more shape diverse small molecule analogs in the theoretic library; and characterizing the one or more synthesized shape diverse small molecule analogs.

In an aspect of a disclosed method, characterizing the one or more synthesized shape diverse small molecule analogs can comprise employing one or more assays. In an aspect, one or more disclosed assays can comprise an indicator displacement assay (IDA), isothermal titration calorimetry (ITC), assay differential scanning fluorimetry (DSF), enzymatic degradation assays, NMR and HPLC analysis, or any combination thereof.

Disclosed herein is a method of assessing a library of shape diverse small molecule analogs that modulate a targeted RNA or a targeted RNA structure, the method comprising choosing a candidate small molecule; generating a theoretical library of shape diverse small molecule analogs based on the candidate small molecule; evaluating the three-dimensional shape of one or more shape diverse small molecule analogs of the theoretical library; selecting one or more shape diverse small molecules for synthesis; synthesizing one or more shape diverse small molecule analogs in the theoretic library; and screening the one or more synthesized shape diverse small molecule analogs.

In an aspect of a disclosed method, generating the disclosed theoretical library of shape diverse small molecule can comprise substituting one or more subunits at the ortho, para, and meta-substituted scaffolds of the candidate small molecule. In an aspect, the subunits are amine subunits.

In an aspect of a disclosed method, evaluating the 3D shape can comprise determining the theoretical principal moments of inertia of the one or more shape diverse small molecule analogs of the theoretical library.

In an aspect, a disclosed method can further comprise correcting the protonation state of the one or more shape diverse small molecule analogs. In an aspect of a disclosed method, correcting the protonation state can comprise adjusting the SMILES strings of the one or more shape diverse small molecule analogs. In an aspect of a disclosed method, adjusting the SMILE strings can comprise using a python code. In an aspect, correcting the protonation state can comprise employing one or more computational software programs. In an aspect, the disclosed one or more computations software programs can comprise ChemAxon, Schrodinger, CREST, or any combination thereof. In an aspect, a disclosed method can further comprise correcting the tautomerization state of the one or more shape diverse small molecule analogs.

In an aspect, a disclosed method can further comprise normalizing the theoretical principal moments of inertia for the one or more shape diverse small molecule analogs of the library. In an aspect, a disclosed method can further comprise visualizing the data by plotting the normalized theoretical PMI for the one or more shape diverse small molecule analogs of the theoretical library on an envelope diagram. In an aspect of a disclosed method, selecting one or more shape diverse small molecule analogs can comprise using one or more computational software programs. In an aspect of a disclosed method, selecting one or more shape diverse small molecule analogs can comprise using R, Python, Java, MatLab, or any combination thereof to maximize shape representation. In an aspect of a disclosed method, selecting one or more shape diverse small molecule analogs can comprise using Kennard-Stone algorithm to maximize shape representation. In an aspect of a disclosed method, evaluating the three-dimensional shape of one or more shape diverse small molecule analogs of the theoretical library can comprise performing a QSAR analysis using multiple descriptors. In an aspect, a disclosed QSAR analysis can employ a commercially available computational program. In an aspect, a disclosed QSAR analysis can employ R, Python, Java, MatLab, or any combination thereof. In an aspect, a disclosed QSAR analysis can comprise using molecular operating environment (MOE), ChemAxon, Schrodinger, KNIME, Mordred, E-Dragon, or any combination thereof.

In an aspect, disclosed descriptors can comprise 2D and 3D descriptors. In an aspect, disclosed descriptors can comprise numerical descriptors, constitutional descriptors, topological descriptors, electronic descriptors, hybrid descriptors, geometric descriptors, QM descriptors, or any combination thereof. Descriptors include, but are not limited to the following: FID, rgyr, glob, AM1_dipole, AM1_E, AM1_Eele, AM1_HF, AM1_HOMO, AM1_IP, AM1_LUMO, apol, ASA, ASA+, ASA−, ASA_H, ASA_P, ast_violation, ast_violation_ext, a_acc, a_aro, a_base, a_count, a_donacc, a_heavy, a_hyd, a_IC, a_ICM, a_nC, a_nCl, a_nH, a_nN, a_nO, balabanJ, BCUT_PEOE_0, BCUT_PEOE_1, BCUT_PEOE_2, BCUT_PEOE_3, BCUT_SLOGP_0, BCUT_SLOGP_1, BCUT_SLOGP_2, BCUT_SLOGP_3, BCUT_SMR_0, BCUT_SMR_1, BCUT_SMR_2, BCUT_SMR_3, bpol, b_1rotN, b_1rotR, b_ar, b_count, b_heavy, b_maxllen, b_rotN, b_rotR, b_single, CASA+, CASA−, chi0, chi0v, chi0v_C, chi0_C, chi1, chi1v, chi1v_C, chi1_C, DASA, DCASA, dens, density, diameter, dipole, dipoleX, dipoleY, dipoleZ, E_ang, E_ele, E_nb, E_oop, E_rnb, E_rsol, E_sol, E_stb, E_str, E_strain, E_tor, E_vdw, FASA+, FASA−, FASA H, FASA_P, FCASA+, FCASA−, FCharge, GCUT_PEOE_0, GCUT_PEOE_1, GCUT_PEOE_2, GCUT_PEOE_3, GCUT_SLOGP_0, GCUT_SLOGP_1, GCUT_SLOGP_2, GCUT_SLOGP_3, GCUT_SMR_0, GCUT_SMR_1, GCUT_SMR_2, GCUT_SMR_3, h_ema, h_emd, h_emd_C, h_logD, h_logP, h_log S, h_log_dbo, h_log_pbo, h_mr, h_pavgQ, h_pKa, h_pKb, h_pstates, h_pstrain, Kier1, Kier2, Kier3, KierA1, KierA2, KierA3, KierFlex, lip_acc, lip_don, lip_druglike, lip_violation, log P(o/w), log S, MNDO_dipole, MNDO_E, MNDO_Eele, MNDO_HF, MNDO_HOMO, MNDO_IP, MNDO_LUMO, mr, mutagenic, nmol, npr1, npr2, opr_brigid, opr_leadlike, opr_nring, opr_nrot, opr_violation, PC+, PC−, PEOE_PC+, PEOE_PC−, PEOE_RPC+, PEOE_RPC−, PEOE_VSA+0, PEOE_VSA+1, PEOE_VSA+2, PEOE_VSA+3, PEOE_VSA+4, PEOE_VSA+5, PEOE_VSA+6, PEOE_VSA−0, PEOE_VSA−1, PEOE_VSA−2, PEOE_VSA−3, PEOE_VSA−4, PEOE_VSA−5, PEOE_VSA−6, PEOE_VSA_FHYD, PEOE_VSA_FNEG, PEOE_VSA_FPNEG, PEOE_VSA_FPOL, PEOE_VSA_FPOS, PEOE_VSA FPPOS, PEOE_VSA_HYD, PEOE_VSA_NEG, PEOE_VSA_PNEG, PEOE_VSA_POL, PEOE_VSA_POS, PEOE_VSA_PPOS, petitjean, petitjeanSC, PM3_dipole, PM3_E, PM3_Eele, PM3_HF, PM3_HOMO, PM3_IP, PM3_LUMO, pmi, pmi1, pmi2, pmi3, pmiX, pmiY, pmiZ, Q_PC+, Q_PC−, Q_RPC+, Q_RPC−, Q_VSA_FHYD, Q_VSA_FNEG, Q_VSA_FPNEG, Q_VSA_FPOL, Q_VSA_FPOS, Q_VSA_FPPOS, Q_VSA_HYD, Q_VSA_NEG, Q_VSA_PNEG, Q_VSA_POL, Q_VSA_POS, Q_VSA_PPOS, radius, reactive, rings, RPC+, RPC−, rsynth, SlogP, SlogP_VSA0, SlogP_VSA1, SlogP_VSA2, SlogP_VSA3, SlogP_VSA4, SlogP_VSA5, SlogP_VSA6, SlogP_VSA7, SlogP_VSA8, SlogP_VSA9, SMR, SMR_VSA0, SMR_VSA1, SMR_VSA2, SMR_VSA3, SMR_VSA4, SMR_VSA5, SMR_VSA6, SMR_VSA7, std_dim1, std_dim2, std_dim3, TPSA, VAdjEq, VAdjMa, VDistEq, VDistMa, vdw_area, vdw_vol, vol, VSA, vsa_acc, vsa_acid, vsa_base, vsa_don, vsa_hyd, vsa_other, vsa_pol, vsurf_A, vsurf_CP, vsurf_CW1, vsurf_CW2, vsurf_CW3, vsurf_CW4, vsurf_CW5, vsurf_CW6, vsurf_CW7, vsurf_CW8, vsurf_D1, vsurf_D2, vsurf_D3, vsurf_D4, vsurf_D5, vsurf_D6, vsurf_D7, vsurf_D8, vsurf_DD12, vsurf_DD13, vsurf_DD23, vsurf_DW12, vsurf_DW13, vsurf_DW23, vsurf_EDmin1, vsurf_EDmin2, vsurf_EDmin3, vsurf_EWmin1, vsurf_EWmin2, vsurf_EWmin3, vsurf_G, vsurf_HB1, vsurf_HB2, vsurf_HB3, vsurf_HB4, vsurf_HB5, vsurf_HB6, vsurf_HB7, vsurf_HB8, vsurf_HL1, vsurf_HL2, vsurf_ID1, vsurf_ID2, vsurf_ID3, vsurf_ID4, vsurf_ID5, vsurf_ID6, vsurf_ID7, vsurf_ID8, vsurf_IW1, vsurf_IW2, vsurf_IW3, vsurf_IW4, vsurf_IW5, vsurf_IW6, vsurf_IW7, vsurf_IW8, vsurf_R, vsurf_S, vsurf_V, vsurf_W1, vsurf_W2, vsurf_W3, vsurf_W4, vsurf_W5, vsurf_W6, vsurf_W7, vsurf_W8, vsurf_Wp1, vsurf_Wp2, vsurf_Wp3, vsurf_Wp4, vsurf_Wp5, vsurf_Wp6, vsurf_Wp7, vsurf_Wp8, Weight, weinerPath, weinerPol, Zagreb, or any combination thereof. In an aspect, a disclosed QSAR analysis using multiple descriptors can generate and/or can provide a predictive model. In an aspect, disclosed 2D descriptors can consider atoms and connectivity information. In an aspect, disclosed 2D descriptors can comprise Q_VSA_PNEG, Q_VSA_POL, SMR_VSA7, or any combination thereof. In an aspect, disclosed 3D descriptor can consider coordinates and individual conformations. In an aspect, disclosed 3D descriptors can comprise glob, vsurf_EDmin2, vsurf_EDmin3, vsurf_DD23, E_stb, or any combination thereof. In an aspect, a disclosed QSAR analysis can comprise about 50-100 descriptors, about 100-150 descriptors, about 150-200 descriptors, about 200-250 descriptors, about 250-300 descriptors, about 300-350 descriptors, or more than 350 descriptors.

In an aspect of a disclosed method, screening the one or more shape diverse small molecule analogs can comprise performing indicator displacement assay (IDA), isothermal titration calorimetry (ITC), differential scanning fluorimetry (DSF), or any combination thereof. In an aspect of a disclosed method, screening the one or more shape diverse small molecule analogs can comprise obtaining the targeted RNA or targeted RNA structure. In an aspect of a disclosed method, obtaining the targeted RNA or targeted RNA structure can comprise synthesizing and/or purifying the targeted RNA or targeted RNA structure. In an aspect of a disclosed method, screening the one or more shape diverse small molecule analogs can comprise measuring the affinity of the one or more shape diverse small molecule analogs against the targeted RNA or targeted RNA structure.

In an aspect of a disclosed method, measuring affinity of the one or more shape diverse small molecule analogs can comprise using an indicator displacement assay (IDA). In an aspect, a disclosed indicator displacement assay can comprise an enantioselective indicator displacement assay (eIDA), a fluorescent indicator displacement assay (FIDA), a reaction-based indicator displacement assay (RIA), a DimerDye disassembly assays (DDAs), an intramolecular indicator displacement assay (IIDA), an allosteric indicator displacement assay (AIDA), a mechanically controlled indicator displacement assay (MC-IDA), a quencher displacement assay (QDA), or any combination thereof. In an aspect of a disclosed method, measured affinity can comprise high affinity (CD50<5 μM), medium affinity (5 μM<CD50<30 μM), or low affinity (CD50 >30 μM).

In an aspect, a disclosed method can further comprise measuring one or more thermodynamic parameters of the biomolecular interaction between the one or more synthesized shape diverse small molecule analogs and the targeted RNA or targeting RNA structure using isothermal titration calorimetry. In an aspect, disclosed thermodynamic parameters of the one or more biomolecular interactions can comprise affinity (KA), enthalpy (ΔH), entropy (ΔS), stoichiometry (n), or any combination thereof.

In an aspect, a disclosed method can further comprise comparing the affinity measured by IDA to the affinity measured by ITC. In an aspect, a disclosed method can further comprise assessing thermal stability of the one or more shape diverse small molecule analogs and the targeted RNA or targeted RNA structure. In an aspect of a disclosed method, wherein assessing the thermal stability can comprise differential screen fluorimetry (DSF) of (i) the one or more synthesized shape diverse small molecule analogs and the targeted RNA structure, and (ii) the one or more synthesized shape diverse small molecule analogs and a control or vehicle.

In an aspect, a disclosed method can further comprise performing QSAR modeling to test whether the same parameters that yielded a predictive model for IDA are the same that govern thermal stabilization interactions as determined by DSF. In an aspect, a disclosed QSAR analysis using both 2D and 3D descriptors can generate and/or produce a predictive model. In an aspect, disclosed 2D descriptors can consider atoms and connectivity information. In an aspect, disclosed 2D descriptors can comprise Q_VSA_PNEG, Q_VSA_POL, SMR_VSA7, or any combination thereof. In an aspect, disclosed 3D descriptions can consider coordinates and individual conformations. In an aspect, disclosed 3D descriptors can comprise glob, vsurf_EDmin2, vsurf_EDmin3, vsurf_DD23, E_stb, or any combination thereof.

Descriptors include, but are not limited to, the following: FID, rgyr, glob, AM1_dipole, AM1_E, AM1_Eele, AM1_HF, AM1_HOMO, AM1_IP, AM1_LUMO, apol, ASA, ASA+, ASA−, ASA_H, ASA_P, ast_violation, ast_violation_ext, a_acc, a_aro, a_base, a_count, a_donacc, a_heavy, a_hyd, a_IC, a_ICM, a nC, a nCl, a nH, a nN, a_nO, balabanJ, BCUT_PEOE_0, BCUT_PEOE_1, BCUT_PEOE_2, BCUT_PEOE_3, BCUT_SLOGP_0, BCUT_SLOGP_1, BCUT_SLOGP_2, BCUT_SLOGP_3, BCUT_SMR_0, BCUT_SMR_1, BCUT_SMR_2, BCUT_SMR_3, bpol, b_1rotN, b_1rotR, b_ar, b_count, b_heavy, b_maxllen, b_rotN, b_rotR, b_single, CASA+, CASA−, chi0, chi0v, chi0v_C, chi0_C, chi1, chi1v, chi1v_C, chi1_C, DASA, DCASA, dens, density, diameter, dipole, dipoleX, dipoleY, dipoleZ, E_ang, E_ele, E_nb, E_oop, E_mnb, E_rsol, E_sol, E_stb, E_str, E_strain, E_tor, E_vdw, FASA+, FASA−, FASA_H, FASA_P, FCASA+, FCASA−, FCharge, GCUT_PEOE_0, GCUT_PEOE_1, GCUT_PEOE_2, GCUT_PEOE_3, GCUT_SLOGP_0, GCUT_SLOGP_1, GCUT_SLOGP_2, GCUT_SLOGP_3, GCUT_SMR_0, GCUT_SMR_1, GCUT_SMR_2, GCUT_SMR_3, h_ema, h_emd, h_emd_C, h_logD, h_logP, h_log S, h_log_dbo, h_log_pbo, h_mr, h_pavgQ, h_pKa, h_pKb, h_pstates, h_pstrain, Kier1, Kier2, Kier3, KierA1, KierA2, KierA3, KierFlex, lip_acc, lip_don, lip_druglike, lip_violation, log P(o/w), log S, MNDO_dipole, MNDO_E, MNDO_Eele, MNDO_HF, MNDO_HOMO, MNDO_IP, MNDO_LUMO, mr, mutagenic, nmol, npr1, npr2, opr_brigid, opr_leadlike, opr_nring, opr_nrot, opr_violation, PC+, PC−, PEOE_PC+, PEOE_PC−, PEOE_RPC+, PEOE_RPC−, PEOE_VSA+0, PEOE_VSA+1, PEOE_VSA+2, PEOE_VSA+3, PEOE_VSA+4, PEOE_VSA+5, PEOE_VSA+6, PEOE_VSA−0, PEOE_VSA−1, PEOE_VSA−2, PEOE_VSA−3, PEOE_VSA−4, PEOE_VSA−5, PEOE_VSA−6, PEOE_VSA_FHYD, PEOE_VSA_FNEG, PEOE_VSA_FPNEG, PEOE_VSA_FPOL, PEOE_VSA_FPOS, PEOE_VSA_FPPOS, PEOE_VSA_HYD, PEOE_VSA_NEG, PEOE_VSA_PNEG, PEOE_VSA_POL, PEOE_VSA_POS, PEOE_VSA_PPOS, petitjean, petitjeanSC, PM3_dipole, PM3_E, PM3_Eele, PM3_HF, PM3_HOMO, PM3_IP, PM3_LUMO, pmi, pmi1, pmi2, pmi3, pmiX, pmiY, pmiZ, Q_PC+, Q_PC−, Q_RPC+, Q_RPC−, Q_VSA_FHYD, Q_VSA_FNEG, Q_VSA_FPNEG, Q_VSA_FPOL, Q_VSA_FPOS, Q_VSA_FPPOS, Q_VSA_HYD, Q_VSA_NEG, Q_VSA_PNEG, Q_VSA_POL, Q_VSA_POS, Q_VSA_PPOS, radius, reactive, rings, RPC+, RPC−, rsynth, SlogP, SlogP_VSA0, SlogP_VSA1, SlogP_VSA2, SlogP_VSA3, SlogP_VSA4, SlogP_VSA5, SlogP_VSA6, SlogP_VSA7, SlogP_VSA8, SlogP_VSA9, SMR, SMR_VSA0, SMR_VSA1, SMR_VSA2, SMR_VSA3, SMR_VSA4, SMR_VSA5, SMR_VSA6, SMR_VSA7, std_dim1, std_dim2, std_dim3, TPSA, VAdjEq, VAdjMa, VDistEq, VDistMa, vdw_area, vdw_vol, vol, VSA, vsa_acc, vsa_acid, vsa_base, vsa_don, vsa_hyd, vsa_other, vsa_pol, vsurf_A, vsurf_CP, vsurf_CW1, vsurf_CW2, vsurf_CW3, vsurf_CW4, vsurf_CW5, vsurf_CW6, vsurf_CW7, vsurf_CW8, vsurf_D1, vsurf_D2, vsurf_D3, vsurf_D4, vsurf_D5, vsurf_D6, vsurf_D7, vsurf_D8, vsurf_DD12, vsurf_DD13, vsurf_DD23, vsurf_DW12, vsurf_DW13, vsurf_DW23, vsurf_EDmin1, vsurf_EDmin2, vsurf_EDmin3, vsurf_EWmin1, vsurf_EWmin2, vsurf_EWmin3, vsurf_G, vsurf_HB1, vsurf_HB2, vsurf_HB3, vsurf_HB4, vsurf_HB5, vsurf_HB6, vsurf_HB7, vsurf_HB8, vsurf_HL1, vsurf_HL2, vsurf_ID1, vsurf_ID2, vsurf_ID3, vsurf_ID4, vsurf_ID5, vsurf_ID6, vsurf_ID7, vsurf_ID8, vsurf_IW1, vsurf_IW2, vsurf_IW3, vsurf_IW4, vsurf_IW5, vsurf_IW6, vsurf_IW7, vsurf_IW8, vsurf_R, vsurf_S, vsurf_V, vsurf_W1, vsurf_W2, vsurf_W3, vsurf_W4, vsurf_W5, vsurf_W6, vsurf_W7, vsurf_W8, vsurf_Wp1, vsurf_Wp2, vsurf_Wp3, vsurf_Wp4, vsurf_Wp5, vsurf_Wp6, vsurf_Wp7, vsurf_Wp8, Weight, weinerPath, weinerPol, Zagreb, or any combination thereof.

In an aspect, a disclosed QSAR analysis can comprise about 50-100 descriptors, about 100-150 descriptors, about 150-200 descriptors, about 200-250 descriptors, about 250-300 descriptors, about 300-350 descriptors, or more than 350 descriptors. In an aspect of a disclosed method, wherein performing QSAR modeling can comprise using the change in RNA degradation induced by the one or more 3D shape diverse small molecules analogs and identifying one or more 3D descriptors as contributing factors.

In an aspect of a disclosed method, screening the one or more shape diverse small molecule analogs can comprise subjecting the one or more molecule analogs to an enzymatic degradation assay. In an aspect, a disclosed enzymatic degradation assay can comprise a RNase. In an aspect, a disclosed RNase can comprise an endoribonuclease or an exoribonuclease. In an aspect, a disclosed endoribonuclease can comprise RNase A, RNase P, RNase H, RNase I, RNase III, RNase T1, RNase T2, RNase U2, RNase V1, RNase PhyM, RNase V, or any combination thereof.

In an aspect, a disclosed RNase can comprise RNase A. In an aspect, a disclosed exoribonuclease can comprise RNase PH, RNase II, RNase R, RNase D, RNA T, or any combination thereof. In an aspect, a disclosed targeted RNA or disclosed targeted RNA structure can comprise a tertiary RNA structure.

In an aspect, a disclosed enzymatic degradation assay can measure the stabilizing or destabilizing effect on the tertiary RNA structure asserted by the one or more shape diverse small molecule analogs. In an aspect of a disclosed method, wherein stabilizing the tertiary RNA structure can comprise measuring less degradation of the tertiary RNA structure when subjected to an enzymatic assay in the presence of the compound than a control or reference level of degradation. In an aspect of a disclosed method, wherein stabilizing tertiary RNA structure ex can comprise measuring less degradation of the tertiary RNA structure when subjected to an enzymatic assay in the presence of the compound than the level of degradation in a control or reference sample. In an aspect of a disclosed method, wherein destabilizing tertiary RNA structure can comprise measuring more degradation of the tertiary RNA structure when subjected to an enzymatic assay in the presence of the compound than a control or reference level of degradation. In an aspect of a disclosed method, wherein destabilizing tertiary RNA structure can comprise measuring more degradation of the tertiary RNA structure when subjected to an enzymatic assay in the presence of the compound than the level of degradation in a control or reference sample.

In an aspect, a disclosed method can further comprise obtaining the control or reference level of degradation. In an aspect of a disclosed method, wherein obtaining the control or reference level of degradation can comprise subjecting the tertiary RNA structure to a RNase in the absence of the compound. In an aspect, a disclosed method can further comprise obtaining the level of degradation in a control or reference sample. In an aspect of a disclosed method, wherein obtaining the level of degradation in a control or reference sample can comprise subjecting the tertiary RNA structure to a RNase in the absence of the compound. In an aspect, a disclosed targeted RNA or a disclosed targeted RNA structure can comprise a RNA triplex. In an aspect of a disclosed method, wherein stabilizing the RNA triplex can comprise measuring less degradation of the RNA triplex when subjected to an enzymatic assay than a control or reference level of degradation. In an aspect of a disclosed method, wherein stabilizing the RNA triplex can comprise measuring less degradation of the RNA triplex when subjected to an enzymatic assay than the level of degradation in a control or reference sample.

In an aspect of a disclosed method, wherein destabilizing the RNA triplex can comprise measuring more degradation of the RNA triplex when subjected to an enzymatic assay than a control or reference level of degradation. In an aspect of a disclosed method, wherein destabilizing the RNA triplex can comprise measuring more degradation of the RNA triplex when subjected to an enzymatic assay than the level of degradation in a control or reference sample. In an aspect, a disclosed method can further comprise obtaining the control or reference level of degradation. In an aspect of a disclosed method, wherein obtaining the control or reference level of degradation can comprise subjecting the RNA triplex to a RNase in the absence of the compound. In an aspect, a disclosed method can further comprise obtaining the level of degradation in a control or reference sample. In an aspect of a disclosed method, wherein obtaining the level of degradation in a control or reference sample can comprise subjecting the RNA triplex to a RNase in the absence of the compound. In an aspect, disclosed shape diverse small molecules that stabilize the targeted RNA or targeted RNA structure can show less endonuclease driven degradation. In an aspect, disclosed shape diverse small molecules that stabilize non-targeted targeted RNA or targeted RNA can show more endonuclease driven degradation.

In an aspect, a disclosed method can further comprise performing a QSAR analysis using the level of degradation generated by the one or more shape diverse small molecule analogs.

In an aspect of a disclosed method, wherein screening the one or more shape diverse small molecule analogs further can comprise measuring the ¹H and ¹³C NMR spectra of the one or more shape diverse small molecule analogs.

In an aspect, a disclosed method can further comprise characterizing the one or more shape diverse small molecule analogs as a stabilizer or destabilizer of the targeted RNA or targeted RNA structure. In an aspect, a disclosed method can further comprise using one or more shape diverse small molecule analogs in a method of stabilizing the targeted RNA or targeted RNA structure. In an aspect, a disclosed method can further comprise using one or more shape diverse small molecule analogs in a method of destabilizing the targeted RNA or targeted RNA structure.

In an aspect, a disclosed targeted RNA or disclosed targeted RNA structure can comprise a human, viral, bacterial, fungal, or yeast targeted RNA or targeted RNA structure. In an aspect, a disclosed targeted RNA or disclosed targeted RNA structure can comprise a targeted RNA or targeted RNA structure in a human, a non-human mammal, a non-mammal, a virus, a bacterium, a fungus, or some other organism. In an aspect, a disclosed targeted RNA or disclosed targeted RNA structure can comprise a synthetically designed RNA, an engineered RNA, or a recombinant RNA. In an aspect, a disclosed targeted RNA or disclosed targeted RNA structure can comprise a secondary or tertiary RNA structure in humans. In an aspect, a disclosed compound can target one or more RNA structural motifs. In an aspect, one or more disclosed RNA structural motifs can comprise a secondary RNA structural motif, a tertiary RNA structural motif, or any combination thereof. In an aspect, disclosed secondary RNA structural motif can comprise the scaffold of the tertiary RNA structural motif. In an aspect, disclosed secondary RNA structural motif can comprise collection of RNA base pairs in a tertiary RNA structural motif. In an aspect, disclosed RNA base pairs can comprise AU, GU, GC, UA, UG, or CG.

In an aspect, a disclosed tertiary RNA structural motif can comprise a bulge, a S-turn, an internal loop, a 3-multiloop, a hairpin loop, a pseudoknot, an apical loop, a kissing loop, a coaxial helix, a stacking helix, a two-way junction, a three-way junction, a four-way junction, or any combination thereof. In an aspect, a disclosed tertiary RNA structural motif can comprise a GNAA tetraloop, a GNGA loop, a T-loop, a C-loop, a E-loop, a Sarcin-ricin loop, a Kink-turn, a Reverse kink-turn, a Hook-turn, a Tandem shear, a Tetraloop-receptor motifs, an interacting tetraloop (e.g., GNRA, GANC, and GAAA tetraloops), a triple-stranded RNA, or any combination thereof. In an aspect, a disclosed RNA structural motif can comprise a RNA triplex structure.

In an aspect, a disclosed RNA triplex structure can concern one or more functionally important RNAs. In an aspect, disclosed functionally important RNAs can comprise human RNAs, viral RNAs, bacterial RNAs, fungal RNAs, yeast RNAs, RNAs of an organism, or any combination thereof. In an aspect, a disclosed targeted RNA or targeted RNA structure can comprise a targeted RNA or targeted RNA structure in a human, a non-human mammal, a non-mammal, a virus, a bacterium, a fungus, or some other organism. In an aspect, disclosed functionally important RNAs can comprise telomerase RNAs, group I and II introns, long noncoding RNAs (lncRNAs), ribosomal RNAs, or any combination thereof. In an aspect, disclosed lncRNAs can comprise TTF-I, FENDRR, MEG3, HIF1α-AS1PARTICLE, KHPS1, HOTAIR, MIR100HG, CHD4/NuRD, or any combination thereof.

In an aspect, functionally important human RNA can comprise NEAT1 ncRNA 3′-end triple helix, G-quadraplexes, other functional secondary and tertiary structures, or any combination thereof. In an aspect, disclosed functionally important human RNA can comprise MALAT1 ncRNA 3′-end triple helix, G-quadraplexes, other functional secondary and tertiary structures, or any combination thereof. In an aspect, disclosed functionally important human RNA can comprise SChLAP1 ncRNA functional secondary and tertiary structures. In an aspect, disclosed SChLAP1 ncRNA functional secondary structures and tertiary structures can comprise stem loops and/or complex junctions near the 3′-end. In an aspect, disclosed functionally important human RNA can comprise AR mRNA functional secondary structures and tertiary structures including splice sites. In an aspect, disclosed AR mRNA functional secondary structures and tertiary structures can comprise a cryptic exon 3. In an aspect, disclosed functionally important human RNA can comprise LMO7 mRNA functional secondary structures and tertiary structures. In an aspect, disclosed LMO7 mRNA functional secondary and tertiary structures can comprise splice sites associated with prostate cancer.

In an aspect, disclosed functionally important viral RNAs can comprise Kaposi's sarcoma-associated herpesvirus (KSHV) polyadenylated nuclear (PAN) 3′-end triple helix. In an aspect, disclosed functionally important viral RNAs can comprise Kaposi's sarcoma-associated herpesvirus (KSHV) polyadenylated nuclear (PAN) functional secondary structures and/or tertiary structures. In an aspect, disclosed functionally important viral RNAs can comprise SARS-CoV-2 5′-untranslated region structures and/or 3′-untranslated regions. In an aspect, a disclosed SARS-CoV-2 5′-untranslated region structure can comprise a frameshifting pseudoknot. In an aspect, disclosed functionally important viral RNAs can comprise one or more HIV elements. In an aspect, the HIV elements can comprise a trans-activation response (TAR) element, a Rev response element (RRE), EESV, a frameshift site, a packing signal (interaction between (w) stem-loop 3 (SL3) RNA and Gag), or any combination thereof. In an aspect, disclosed HIV elements can comprise other functional secondary structures and/or functional tertiary structures.

In an aspect, disclosed functionally important viral RNAs can comprise one or more enterovirus elements. In an aspect, disclosed enterovirus elements can comprise the 5′-untranslated region structures, internal ribosomal entry sites (IRES), functional coding region structures, a frameshift pseudoknot, a 3′-untranslated region structures, or any combination thereof.

In an aspect, disclosed functionally important viral RNAs can comprise one or more viral secondary structures or tertiary structures. In an aspect, disclosed functionally important viral RNAs can comprise one or more viral secondary structures or tertiary structures in a poliovirus, a rhinovirus, an encephalomyocarditis virus, a foot-and-mouth disease virus, a Kaposi's sarcoma-associated herpesvirus, a hepatitis A virus, or a hepatitis C virus. In an aspect, disclosed functionally important viral RNAs can comprise a secondary structure or a tertiary structure in the 5′-untranslated region or the internal ribosome entry site of a poliovirus, a rhinovirus, an encephalomyocarditis virus, a foot-and-mouth disease virus, a Kaposi's sarcoma-associated herpesvirus, a hepatitis A virus, or a hepatitis C virus. In an aspect, disclosed functionally important viral RNAs can comprise one or more viral secondary structures or tertiary structures in a coronavirus, a flavivirus, alphaviruses, a picornavirus, or a positive sense RNA virus. In an aspect, disclosed functionally important viral RNAs can comprise a secondary structure or a tertiary structure in the 5′-untranslated region, coding region, or the 3′-untranslated region in a coronavirus, a flavivirus, alphaviruses, a picornavirus, or a positive sense RNA virus.

In an aspect, disclosed RNA structural motifs can comprise a RNA quadruplex structure. In an aspect, disclosed RNA quadruplex structures can comprise telomeric-repeat containing RNA (TERRA RNA).

In an aspect, one or more disclosed functionally important RNAs can comprise telomerase RNAs, group I and II introns, long noncoding RNAs (lncRNAs), ribosomal RNAs, or any combination thereof.

In an aspect of a disclosed method, a disclosed destabilizing compound can comprise DMZ-P0, DMZ-P1, DMZ-P5, DMZ-P14, DMZ-O2, DMZ-O5, DMZ-M4, DMZ-M15, DMZ-M22, DMZ-NMe-P38, DMZ-NMe-mPy-P1, or any combination thereof. In an aspect of a disclosed method, a disclosed stabilizing compound can comprise DMZ-P8, DMZ-P9, DMZ-P13, DMZ-P17, DMZ-O1, DMZ-O4, DMZ-O6, DMZ-M1, DMZ-M3, DMZ-M7, DMZ-M10, or any combination thereof.

In an aspect, disclosed method can further comprise treating a subject in need thereof by administering to the subject a disclosed compound, a disclosed composition, a disclosed pharmaceutical formulation, or any other combination thereof.

In an aspect, a disclosed method can further comprise generating and/or validating one or more of disclosed compounds, disclosed compositions, disclosed pharmaceutical formulation, or any combination thereof. Methods of generating and/or validating disclosed compounds, disclosed compositions, disclosed pharmaceutical formulations, or any combination thereof are discussed infra.

### N. Kits

Disclosed herein is a kit comprising one or more disclosed compounds, disclosed compositions, disclosed pharmaceutical formulation, or any combination thereof. Disclosed herein is a kit comprising one or more disclosed compounds, disclosed compositions, disclosed pharmaceutical formulation, or any combination thereof with or without additional therapeutic agents to effect a disclosed method of treating a subject. Disclosed herein is a kit comprising one or more disclosed compounds, disclosed compositions, disclosed pharmaceutical formulation, or any combination thereof with or without additional therapeutic agents to effect a disclosed method of modulating secondary RNA structures and/or tertiary RNA structures. Disclosed herein is a kit comprising one or more disclosed compounds, disclosed compositions, disclosed pharmaceutical formulation, or any combination thereof with or without additional therapeutic agents to effect a disclosed method of assessing a library of small molecules.

In an aspect, a disclosed kit can comprise at least two components constituting the kit. Together, the components constitute a functional unit for a given purpose (such as, for example, treating a subject diagnosed with or suspected of having or can be diagnosed with having a disease, condition, or disorder caused by, related to, and/or exacerbated by the absence of, lack of expression of, and/or lack of activity of one or more secondary RNA structures, one or more tertiary RNA structures, or any combination thereof.

Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation. In an aspect, a kit for use in a disclosed method can comprise one or more containers holding one or more disclosed compounds, disclosed compositions, disclosed pharmaceutical formulation, or any combination thereof, and a label or package insert with instructions for use. In an aspect, suitable containers include, for example, bottles, vials, syringes, blister pack, etc. The containers can be formed from a variety of materials such as glass or plastic. The container can hold, for example, one or more disclosed compounds, disclosed compositions, disclosed pharmaceutical formulation, or any combination thereof, and can have a sterile access port (for example the container may be an intravenous solution bag or a vial having a stopper pierceable by a hypodermic injection needle). The label or package insert can indicate that one or more disclosed compounds, disclosed compositions, disclosed pharmaceutical formulation, or any combination thereof can be used for treating a subject or modulating secondary RNA structures and/or tertiary RNA structures. In an aspect, a disclosed kit can comprise additional components necessary for administration such as, for example, other buffers, diluents, filters, needles, and syringes. In an aspect, a disclosed kit can comprise those components (e.g., primers) necessary to measure one or more times the level of expression and/or the level of activity of one or more disclosed secondary RNA structures and/or disclosed tertiary RNA structures.

TABLE 1
Sequence Information.

| SEQ ID | |
|---|---|
| NO | Description |
| 1 | MALAT1 TH - DNA Template |
| 2 | MALAT1 TH - Fwd primer |
| 3 | MALAT1 TH - Rev primer |
| 4 | Stem Loop DNA Template |
| 5 | Stem Loop Fwd primer |
| 6 | Stem Loop Rev primer |
| 7 | Upper Stem RNA Sequence |
| 8 | MALAT1 TH RNA Cy5 Labelled |
| 9 | MALAT1 (Metastasis Associated Lung Adenocarcinoma |
| | Transcript 1) (NR_002819.4) |
| 10 | MALAT1 (Metastasis Associated Lung Adenocarcinoma |
| | Transcript 1) (NR_144567.1) |
| 11 | MALAT1 (Metastasis Associated Lung Adenocarcinoma |
| | Transcript 1) (NR_144568.1) |
| 12 | NEAT1 (nuclear paraspeckle assembly transcript 1) |
| | (NR_028272.1) |
| 13 | NEAT1 (nuclear paraspeckle assembly transcript 1 |
| | (NR_131012.1) |
| 14 | LIM domain 7 (isoform 1) (NM_005358.5) |
| 15 | LIM domain 7 (isoform 2) (NM_001366632.2) |
| 16 | LIM domain 7 (isoform 3) (NM_001306080.2) |
| 17 | LIM domain 7 (isoform 1) (NP_005349.3) |
| 18 | LIM domain 7 (isoform 2) (NP_001353561.1) |
| 19 | LIM domain 7 (isoform 3) (NP_001293009.1) |
| 20 | SWI/SNF complex antagonist associated with prostate |
| | cancer 1 (SCHLAP1), transcript variant 1 (NR_104319.1) |
| 21 | SWI/SNF complex antagonist associated with prostate |
| | cancer 1 (SCHLAP1), transcript variant 2 (NR_104320.1) |
| 22 | SWI/SNF complex antagonist associated with prostate |
| | cancer 1 (SCHLAP1), transcript variant 3 (NR_104321.1) |
| 23 | SWI/SNF complex antagonist associated with prostate |
| | cancer 1 (SCHLAP1), transcript variant 4 (NR_104323.1) |
| 24 | Androgen Receptor (AR), transcript variant 3 |
| | (NM_001348061.1) |
| 25 | Androgen Receptor (AR), transcript variant 3 |
| | (NP_001334990.1) |

### VII. EXAMPLES

The exponential increase in identification of both coding and non-coding RNAs actively involved in human diseases has opened the door to novel therapeutic targets of all shapes, sizes, and levels of structural complexity. (Zafferani M, et al. (2021) Cell Chem Biol. 28(5):594-609; Fedorova O, et al. (2018) Nat Chem Biol. 14(12):1073-1078). RNA targeting efforts to date have revealed that traditional drug discovery approaches built for protein targets may need to be optimized or re-designed to accommodate the chemical and conformational differences of RNA. (Warner K D, et al. (2018) Nat Rev Drug Discov. 17(8):547-558). Recent surveys of known RNA-binding small molecules indicate that these probes may indeed exist in a privileged chemical space. (Padroni G, et al. (2020) RSC Med Chemy. 11(7):802-813; Rizvi N F, et al. (2020) SLAS Discov. 25(4):384-396). One specific feature that emerged from these studies is that RNA-binding small molecules more often form rod-like 3D shapes when compared to FDA-approved protein binders. (Morgan B S, et al. (2017) Angew Chem Int Ed Eng. 56(43):13498-13502). However, the relatively small size of the surveyed library and the vastly different approaches that led to ligand discovery make the generalizability of this trend unclear. Furthermore, the majority of successfully targeted RNAs are secondary structures, which may be more challenging to selectively engage with small molecules due to their structural simplicity. (Warner K D, et al. (2018) Nat Rev Drug Discov. 17(8):547-558; Carothers J M, et al. (2004) J Am Chem Soc. 126(16):5130-5137).

Consequently, experts in the field have proposed that ligand development should focus on disease-relevant targets with higher ‘bits’ of information, a unit-measure of the uniqueness of a given RNA target found to correspond with a higher probability of possessing classically-defined druggable sites and increased structural complexity. (Warner K D, et al. (2018) Nat Rev Drug Discov. 17(8):547-558).

There is thus a clear need to test the applicability of identified RNA-targeted chemical space to more complex RNAs and to develop new methods to better understand these molecular recognition events.

In this study, a multi-dimensional assessment of a synthetic library targeting a single RNA tertiary structure motif, namely the RNA triple helix present within the long non-coding RNA (lncRNA) metastasis-associated lung adenocarcinoma transcript 1 (MALAT1), was employed.

Recognition of the pervasiveness of RNA triple helices in the transcriptome of multiple species led to coining of the word ‘triplexome’ and underlined the need to expand the currently limited set of chemical probes aimed at targeting and modulating RNA triple helices. (Brown J A, (2020) WIREs RNA. 11(6):e1598; Conrad N K. (2014) WIREs RNA. 5(1):15-29).

The MALAT1 triplex, present at the 3′-end of a 6.7 kb mature lncRNA, was chosen as the case study for this report due to its established biological relevance and the precedented, though limited, small molecule ligands that can be used for comparison. (Donlic A, et al. (2020) Nucleic Acids Res. 48(14):7653-7664; Donlic A, et al. (2018) Angew Chem Int Ed Eng. 57(40):13242-13247; Abulwerdi F A, et al. (2019) ACS Chem Biol. 14(2):223-235). MALAT1 has been found to be overexpressed in a plethora of diseases, including cancer and diabetes. (Abdulle L E, et al. (2019) Int J Med Sci. 16(4):548-555; Sun Y, et al. (2019) Cancers (Basel). 11(2):216; Yan Y, et al. (2020) IUBMB Life. 72(3):334-342; Amodio N, et al. (2018) J Hematol Oncol. 11(1):63).

While all its functions are still being elucidated and the structure of the entire transcript remains elusive, the 3′-end blunt formation of an A-U rich RNA triple helix has been found to play an active role in the cellular accumulation of the transcript. (Wilusz J E, et al. (2012) Genes Dev. 26(21):2392-2407). One of the first identified and best characterized RNA triple helices, the MALAT1 triplex forms through the sequestration of its genomically encoded poly(A) tail in the major groove of a uridine-rich stem loop, ultimately protecting the transcript from RNase-mediated digestion and increasing its half-life. (Wilusz J E, et al. (2012) Genes Dev. 26(21):2392-2407; Brown J A, et al. (2014) Nat Struct Mol Biol. 21(7):633-640). Consequently, the MALAT1 triple helix has been the subject of small molecule targeting aimed at modulating the stability of the triplex. (Donlic A, et al. (2020) Nucleic Acids Res. 48(14):7653-7664; Donlic A, et al. (2018) Angew Chem Int Ed Eng. 57(40):13242-13247; Abulwerdi F A, et al. (2019) ACS Chem Biol. 14(2):223-235).

To elucidate fundamental recognition properties of triplex modulators, a focused library was built around the diminazene scaffold due to its synthetic accessibility and known nucleic acid binding properties. The scaffold architecture and subunit chemical identity were varied to maximize diversity in 3D shape of the analogues. For a holistic approach, a 21-member library was evaluated using four in vitro assays designed and/or optimized within this work to specifically evaluate small molecule:RNA triplex interactions.

Rod-like 3D shape trends held true for the newly synthesized focused library with regards to affinity for the MALAT1 triplex. While affinity also correlated with the effect of small molecules on triplex thermal stability, vastly different trends were found in enzymatic degradation assays, showcasing the importance of assessing RNA-targeted small molecules using multiple complementary methods.

Furthermore, employment of quantitative structure activity relationships (QSAR) afforded predictive models that revealed the importance of 3D shape small molecule descriptors in relationship to enzymatic degradation, yielding a single computational model that can predict both stabilizing and destabilizing effects. The diminazene scaffold utilized in this work established the feasability of bidirectional modulation of RNA tertiary structure degradation with rational synthetic tuning.

### Materials and Methods

### Theoretical Library and Principal Moments of Inertia

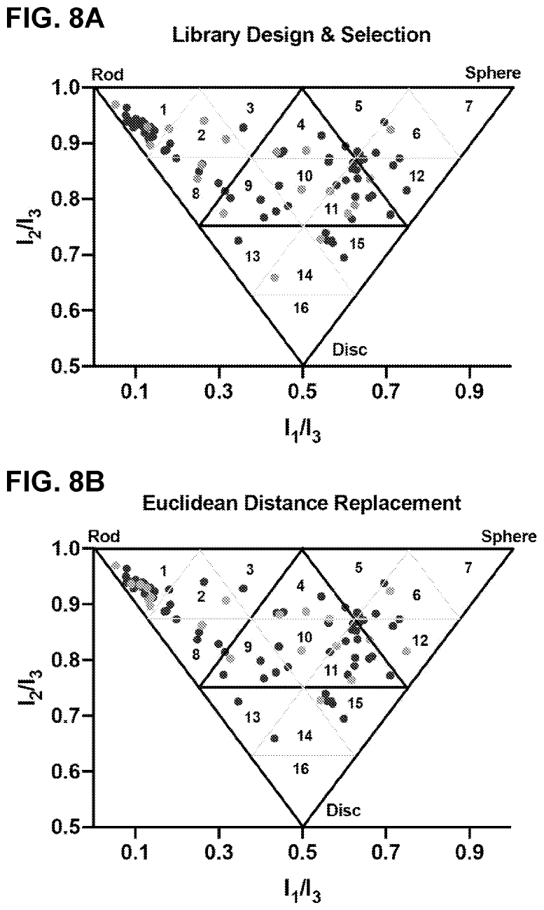

A theoretical 90-member library was built by coupling commercially available amine subunits to the ortho, para, and meta-substituted scaffolds. The SMILES strings of each small molecule were then adjusted for protonation at pH=7.4 using a python code (described supra). The corrected SMILES were then loaded on Molecular Operating Environment (MOE, 2018.01) to perform a conformational search. (Donlic A, et al. (2018) Angew Chem Int Ed Eng. 57(40):13242-13247). Small molecule descriptors were calculated on the lowest energy conformer of each small molecule for a total of 339 parameters. Small molecule shape was evaluated using normalized principal moments of inertia (npr1, npr2) and plotted on an envelope diagram. A 20 small molecule sub-set was chosen for synthesis using the Kennard Stone algorithm (presented above) to ensure maximum representation of small molecule 3D shape diversity (FIG. 8A). Extensively conjugated anilines resulted in poor reactivity under a plethora of conditions and with various Lewis acids. Due to this, small molecules closest in Euclidean distance were chosen for synthetic investigation. To quantify possible alteration in 3D shape representation of the small molecule sub-set, the envelope diagram was further divided in 16 sub-triangles. Except for sub-triangle #14 which contained a single molecule, the remaining sub-triangles retained at least one representative small molecule (FIG. 8B).

### Library Synthesis and Purification

Reagents were purchased from commercial suppliers and were used without further purification. All solvents were ACS grade or better and used without further purification. Anhydrous toluene was obtained by storing ACS-grade toluene over 4 A molecular sieves while anhydrous THF was dispensed from VWR SureSeal bottles and kept under argon. All microwave reactions were run on a Biotage Initiator+ reactor from Biotage Inc. and under argon inert atmosphere. All chromatographic purifications were conducted via flash chromatography using ultra-pure silica gel (230-400 mesh, 60 Å) purchased from Silicycle as the stationary phase. Thin Layer Chromatography was performed with glass-backed silica gel plates purchased from VWR and visualized with 254 nm UV light. All deuterated solvents for NMR experiments were purchased from Cambridge Isotope Laboratories. All ¹H and ¹³C NMR spectra were recorded using a 500 MHz Bruker spectrometer. The corresponding ¹³C resonance frequencies were 100 MHz and 125 MHz, respectively. Chemical shifts are expressed as parts per million from tetramethylsilane. ¹H chemical shifts were referenced with that of the solvent (7.26 for CDCl₃, 3.31 for CD₃OD and 4.87 for D₂O) and coupling constants (J values) are reported in units of Hertz (Hz). Splitting patterns have been designated as follows: s (singlet), d (doublet), t (triplet) and m (multiplet), br (broad). Low and high-resolution electrospray ionization (ESI) and mass spectra were recorded on an Agilent MSD-trap Spectrometer. HPLC spectra were recorded using a Shimadzu SIL-20AHT Prominence instrument. All HPLC experiments S19 were run at room temperature using gradients or isocratic mixtures of 0.1% TFA in water and acetonitrile as solvents A and B, respectively. Yields refer to ≥95% spectroscopically and chromatographically pure compounds.

### Synthesis of Bis-Cyano Scaffold

The synthesis of the bis-cyano scaffold is described below.

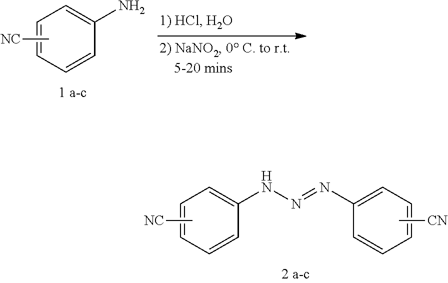

4-(1a), 3-(1b), or 2-aminobenzonitrile (1c) (16.9 mmol) was dissolved in 0.5 M HCl (80 mL) in a 200 mL round bottom flask and cooled to 0° C. on an ice bath. Upon complete dissolution of the aniline, 2 mL of a 4.5 M solution of sodium nitrite was added dropwise. A precipitate formed immediately, and each coupling was allowed to react at room temperature until a dense solution formed. The reaction was then filtered through a fritz funnel and solid washed with ice-cold deuterated water. Precipitate was left drying overnight under vacuum and used in the scaffold decoration (the procedure was adapted from previous methodology). (Cappoen D, et al. (2014) Eur J Med. Chem. 77:193-203).

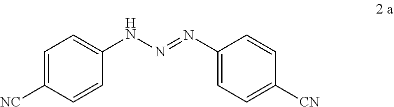 [SMILES: N#Cc1ccc(/N=N/Nc2ccc(C#N)cc2)cc1]

Bright yellow solid; Yield=88%; ¹H NMR (500 MHZ, D₂O) δ 13.28 (s, 1H), 7.97-7.54 (m, 9H). ¹³C NMR (126 MHz, D2O) δ 134.90, 133.58, 119.47. Calcd for C14H9N5 ([M+H]+): 248.0; found: 247.1(±2.1 ppm).

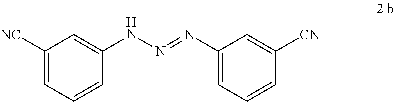 [SMILES: N#Cc1cccc(/N=N/Nc2cccc(C#N)c2)c1]

Pale beige solid; Yield=53%; ¹H NMR (500 MHZ, DMSO-d6) δ 12.98 (s, 1H), 7.96 (s, 1H), 7.79 (d, J=8.3 Hz, 2H), 7.61 (q, J=8.7, 7.5 Hz, 4H), 6.88-6.82 (m, 1H). ¹³C NMR (126 MHz, DMSO) δ 132.03, 130.50, 119.09, 112.76. Calcd for C₁₄H₉N₅ ([M+H]+): 248.0; found: 249.1(±1.8 ppm).

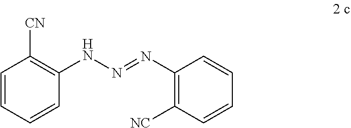 [SMILES: N#Cc1ccccc1/N=N/Nc1ccccc1C#N]

Bright yellow solid; Yield=74%; ¹H NMR (500 MHZ, DMSO-d6) δ 13.41 (s, 1H), 7.96 (d, J=8.2 Hz, 1H), 7.82 (dd, J=37.5, 8.0 Hz, 2H), 7.69 (dt, J=21.9, 8.5 Hz, 2H), 7.48-7.40 (m, 2H), 7.27-7.19 (m, 1H). ¹³C NMR (126 MHz, DMSO) δ 152.05-151.93, 142.93, 135.56-132.83, 128.36, 124.26, 119.00-108.57, 98.40, 93.96, 55.32, 40.50-39.50, 29.99. Calcd for C₁₄H₉N₅ ([M+H]+): 248.0; found: 248.1(±1.6 ppm).

### General Procedure for Amidine Formation

2a-c (0.41 mmol) and DABAL-Me3 (1.2 mmol) was added to a 5 mL over-dried pressure vial under argon. The solids were dissolved in anhydrous THF or toluene (2.5 mL) and a primary amine (1 mmol) was added drop-wise in a 5 mL over-dried pressure vial under argon and heated to 105° C. for 4.5 hours. After running, the reaction was diluted in dichloromethane and quenched with acetonitrile dropwise while stirring. The solution was then evaporated under vacuum. The solid was re-dissolved in methanol and a 5:1 ratio of celite:starting material was added. Compounds were purified using silica column chromatography in a gradient 95:4:1 dichloromethane:methanol:ammonium hydroxide to 85:14:1 dichloromethane:methanol:ammonium hydroxide to yield the final compounds. Procedure was adapted from previously published synthesis. (Donlic A, et al. (2018) Angew Chem Int Ed Eng. 57(40):13242-13247).

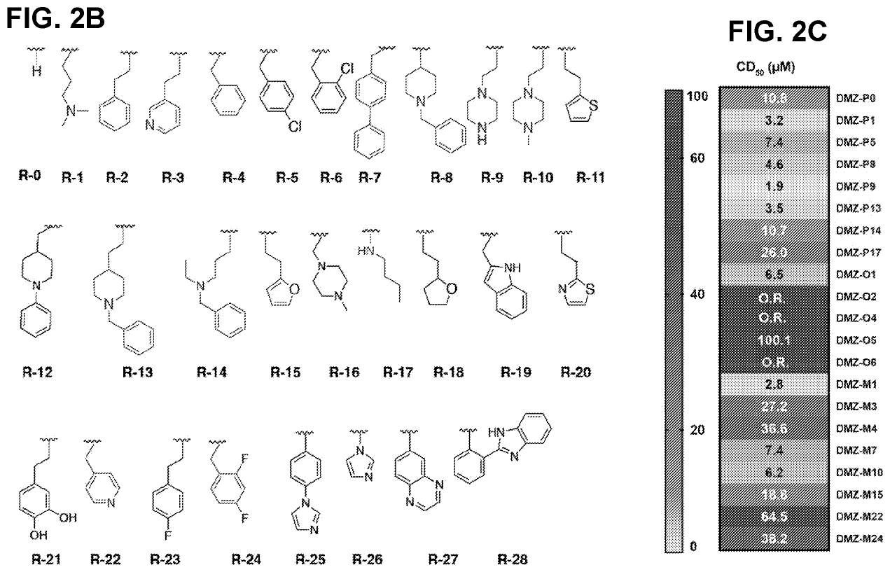

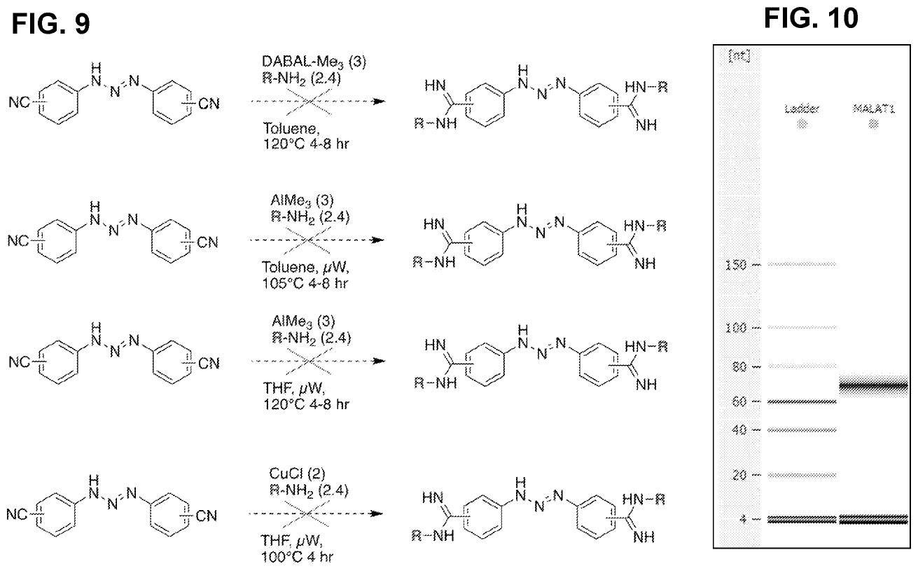

Extensively conjugated anilines have been found to be unreactive with various Lewis acids and under various conditions as listed below (FIG. 9). Specifically, no diamidine formation was observed in the presence of R-19, R-25, R-27, and R-28 (FIG. 2).

### Indicator Displacement Assay (DIA)

The protocol was adapted from a previously reported procedure. (Donlic A, et al. (2020) Nucleic Acids Res. 48(14):7653-7664). A serial dilution of the MALAT1 triple helix RNA was performed in HEPES buffer (20 mM HEPES-KOH, 52.6 mM KCl, 0.1 mM MgCl2, pH=7.4) in a 96 well plate in triplicate. 8 mL of each dilution were transferred to a 384 well plate followed by 8 mL of a 500 nM solution of RiboGreen dye in the same buffer (Invitrogen™). The plates were excited at 487 nm (8 nm slit) and emission was recorded at 530 nm (8 nm slit, focal height 11.3 mm) using a CLARIOstar plate reader (BMG labtech). The affinity of the dye for the RNA construct was determined by fitting the raw fluorescence in in GraphPad Prism version 8.3.1 for Macintosh (GraphPad Software, La Jolla California USA, (www.graphpad.com)) by fitting to the Log[Agonist] vs. response—variable slope model that using Equation (1) below.

$$Y = Bottom + ( {X}^{⋀} HillSlope ) * ( Top - Bottom ) / ( {X}^{⋀} HillSlope + LogCD {50}^{⋀} HillSlope ) \qquad ( Equation 1 )$$

where Y is normalized % change in fluorescence intensity, X is RNA concentration, Bottom is lowest fluorescence % change and Top is highest fluorescence % change. Affinity of the dye for the RNA construct was used as the ideal RNA concentration (174 nM) for small molecule titrations.

A serial dilution of small molecule (0, 0.5 μM, 1.0 μM, 2.5 μM, 5 μM, 7.5 μM, 10.0 μM, 12.5 μM, 25 μM, 50 UM, 75 μM, 100 μM, 125 μM, 150 μM, 250 μM, 500 M) was performed in HEPES buffer (20 mM HEPES-KOH, 52.6 mM KCl, 0.1 mM MgCl₂, pH=7.4) in a 96 well plate in triplicate. Then, 8 μL of each dilution were transferred to a 384 well plate, followed by 8 μL of a 174 nM solution of MALAT1 RNA and 0.5 mM of RiboGreen™ (Invitrogen™). The RNA was previously annealed by heating to 95° C. for 5 minutes and cooling to 0° C. on ice for 30 minutes. The 384 well plates were shaken for 5 minutes, centrifuged at 4000 rpm for 1 minute, and incubated in the dark for 20 minutes. The plates were excited at 487 nm (8 nm slit) and emission was read at 525 nm (8 nm slit, focal height 11.3 nm) using a CLARIOstar plate reader (BMG Labtech). Percent fluorescence indicator displacement (% FID) was calculated by subtracting and, subsequently, dividing by the blank wells with RNA-dye complex and no small molecule as shown in Equation (2).

$$\% F I D = ( \frac{{F}_{0} - F}{{F}_{0}} ) * 100 \qquad ( Equation 2 )$$

Where Fₒ is the fluorescence of the blank well with RNA+dye and no small molecule and F is the fluorescence of the well with all three components (RNA+dye+small molecule).

Each technical triplicate was averaged, and the resulting FID values were averaged between three independent experiments. The binding curve and Log (CD₅₀) value was obtained by using a non-linear fit curve (Log[Agonist] vs. response) with variable slope at four parameters (GraphPad Prism Software version for Macintosh 8.3.1, La Jolla, California, USA (www.graphpadprism.com)) as shown in Equation 1. Reported values are averages of three independent experiments=standard deviation.

### Differential Scanning Fluorimetry

Differential scanning fluorimetry experiments were performed as previously reported. (Donlic A, et al. (2020) Nucleic Acids Res. 48(14):7653-7664). All experiments were performed in white 96 well plates in a LightCycler® 96 (Roche). In a typical experiment, 4 μM of RNA was annealed in the same buffer used for indicator displacement assays (20 mM HEPES-KOH, 52.6 mM KCl, 0.1 mM MgCl₂, pH=7.4) by heating at 95° C. for 3 minutes, snap-cooling on ice for 10 minutes, and leaving the solution at room temperature for 1 hr. Then, 42 μL of this solution was aliquoted to wells and 5 μM of ligand was added (2.5 μL of water, 2.0 μL of DMSO, 0.5 μL of 500 μM small molecule stock). The mixture was left to incubate at room temperature for 5 minutes, after which 3 μL of a 20 μM Quant-iT™ RiboGreen™ (ThermoFisher Scientific) dye stock solution in appropriate buffer was added to each well. The 96 well plate was then sealed with an optically clear foil and centrifuged for 1 minute at 4000 rpm prior to being placed in the instrument. The light cycler program was created by selecting the melting curve method; fluorescence intensity was monitored using the SYBR Green I/HRM dye filter combination (465-510 nm) from 37-98° C. at a ramp rate of 0.01° C./second with 150 acquisitions per° C. Melting profiles were obtained by Tₘ analysis in the LightCycler® 96 software (SW 1.1).

### RNA Synthesis and Purification

DNA template sequence was purchased from Dharmacon and forward and reverse primers were purchased from Integrated DNA Technologies (IDT). For PCR amplification the following reagents were added to for a given 50 mL final reaction volume. First, the entire working space was sprayed with RNase Zap to prevent any contamination. Next, in the desired amount of sterile PCR tubes (ThermoFisher), 12.5 mL of RNase free water and 12.5 mL of Q5 reaction buffer were added, followed by 1 mL of dNTPs (10 mM), 2.5 mL of forward and reverse primer (10 mM), 1 mL of DNA template (50 ng/mL) and lastly 0.5 mL of Q5 polymerase (New England Biolabs). The DNA template was then amplified for 30 cycles in an Eppendorf Echo thermocycler. A Zymo DNA-clean-up kit was then utilized to clean-up the desired DNA sequence. A solution of amplified DNA in water was made to reach 28-35 ng/mL. The sequence was then in vitro transcribed (IVT) using the following protocol. For a given 50 mL IVT reaction the following were added in respective order: 31.75 mL of RNase free water, 1.25 mL MgCl₂ (1 M), 2 mL Tris-HCl (1 M at pH 8.0), 1.25 mL of spermidine (0.1 M), 0.5 mL of Triton-X (0.1%), 0.5 mL of DTT (1 M), 0.2 mL of pyrophosphatase enzyme (100 U/mL), 5 mL of DNA template and 2.5 mL of T7 polymerase enzyme kindly provided by the Tolbert lab at Case Western University.

The reaction was then incubated at 37° C. for 12 hours. Following incubation, the reaction was treated with DNase I buffer and DNase I (New England Biolabs) twice in intervals of 30 minutes, followed by addition of 10% of the reaction volume of EDTA. The desired RNA was then extracted using phenol chloroform extraction and further purified via ethanol precipitation. Purity and size of the RNA construct was confirmed by 15% TBE denaturing gel (see RNase A section) and by Small RNA chip on an Agilent Bioanalyzer. Upper stem RNA was purchased from Dharmacon (Horizon Discovery), Cy5-labelled MALAT1 Triple Helix was ordered from Dharmacon (Horizon Discovery), and upper stem loop DNA sequence was purchased from IDT.

TABLE 2
Sequences of the DNa Template and Primers used in In Vitro Synthesis
of MALAT1 Triple Helix and Stem Loop

| Description | Sequence |
|---|---|
| MALAT1 TH | GGAAGGTTTTTCTTTTCCTGAGAAAACAACACGTATTGTTT |
| DNA Template | TCTCAGGTTTTGCTTTTTGGCCTTTTTCTAGCTTAAAAAAAA |
| | AAAAAGCAAAA (SEQ ID NO: 01) |
| MALAT1 TH | GAAATTAATACGACTCACTATAGGAAGGTTTTTCTTTTCCT |
| Fwd primer | GAGAAAACAACACGTATT (SEQ ID NO: 02) |
| MALAT1 TH | TmU*TTGCTTTTTTTTTTTTTAAGC (SEQ ID NO: 03) |
| Rev primer | |
| Stem Loop | GGAAGGTTTTTCTTTTCCTGAGAAAACAACACGTATTGTT |
| DNA Template | TTCTCAGGTTTTGCTTTTTGGCCTTTT (SEQ ID NO: 04) |
| Stem Loop | GAAATTAATACGACTCACTATAGGAAGATTTTTCTTTTCCT |
| Fwd primer | GAGAAAACAACACGTATT (SEQ ID NO: 05) |
| Stem Loop | A[mA]AAGGCCAAAAAGCAA (SEQ ID NO: 06) |
| Rev primer | |
| Upper Stem RNA | CCUGAGAAAACAACACGUAUUGUUUUCUCAGG (SEQ ID |
| Sequence | NO: 07) |
| MALAT1 TH RNA | Cy5GGAAGGUUUUUCUUUUCCUGAGAAAACAACACGUAU |
| Cy5 Labelled | UGUUUUCUCAGGUUUUGCUUUUUGGCCUUUUUCUAGCUU |
| | AAAAAAAAAAAAAGCAAAA (SEQ ID NO: 08) |

### RNase A Enzymatic Degradation Assay

The procedure was adapted from a published protocol for exonucleolytic enzymatic degradation assay. (Donlic A, et al. (2020) Nucleic Acids Res. 48(14):7653-7664). The MALAT1 destabilizing small molecules published by Le Grice (Abulwerdi F A, et al. (2019) ACS Chem Biol. 14(2):223-235) and co-workers SM5 and SM16 were purchased from ChemBridge. For a typical 70 μL reaction, 0.2 mM of MALAT1 triple helix RNA was annealed in the same buffer used for IDA and DSF experiments (20 mM HEPES-KOH, 52 mM KCl, 0.1 mM MgCl₂, pH=7.4) by heating at 95° C. for 3 minutes, snap-cooling on ice for 10 minutes, and leaving the solution at room temperature for 1 hr.

RNase A enzyme (ThermoFisher) was diluted to 1.46 nM and incubated with 1 M NaCl for 30-35 minutes to achieve ssRNA selectivity as reported. (Struhl K, et al. (1989) Current Protocols in Molecular Biology. 8(1):3.13.1-3.13.3). Next, the RNA was mixed with 0.2 μM of a GC-rich DNA duplex, 0.2 mM of small molecule/DMSO and incubated at room temperature for 20 minutes. Following the incubation, 1 mL of RNase A was added, and the reaction was incubated at 37° C. for 1 hour in a thermal cycler with a heated lid (Eppendorf). 4 μL timepoint aliquots were taken at 0, 5, 10, 30, and 60 minutes, 4 μL of 2×RNA loading dye was added to each aliquot (ThermoFischer Scientific), and samples were stored at −80° C. prior to gel analysis. Reactions were analyzed on pre-cast 15% PAGE-Urea gels (Invitrogen) in 1×TBE buffer. Aliquots were thawed from −80° C. storage and heated to 95° C. for 15 minutes prior to gel loading and 6 mL were loaded in each well. Gels were run for 45 minutes at 180 V, stained with Diamond™ Nucleic Acid dye (Promega), and visualized on an iBright 500 (ThermoFisher). DMSO controls were ran with every set of 4 small molecules to ensure consistency of enzyme activity and RNA integrity. Gel band intensity of the MALAT1 triple helix and DNA loading control were quantified using ImageJ software. (Rueden C T, et al. (2017) BMC Bioinformatics. 18(1):529).

To quantify time-dependent endonucleolytic degradation for each sample, the intensity was normalized using Equation (3), and fold-difference was calculated as listed in Equation (4) as previously reported. (Donlic A, et al. (2020) Nucleic Acids Res. 48(14):7653-7664).

$$y = ( T H Area ( tx ) ) \frac{L C Area ( t 0 )}{L C Area ( tx )} \qquad ( Equation 3 )$$

where y is normalized RNA area for a specific time point and the sample (tx), TH (triple helix). Area is the raw band intensity for the RNA in the lane corresponding to time point x for that sample, LC (loading control). Area (t0) is the raw band intensity for the DNA in the lane corresponding to the 0 minute time point for the sample, and LC Area (tx) is the raw band intensity for the DNA in the lane corresponding to time point x for that sample.

$$y = \frac{Normalized Area ( tx )}{Normalized Area ( t 0 )} \qquad ( Equation 4 )$$

where y is fold-difference of the exonucleolytic degradation for the specific sample, with normalized areas for its specific time point x and time point 0 calculated as described in Equation (3). Percent RNA degraded was calculated for every time point using Equation (5).

% RNA degraded=(Normalized area (t0)−Normalized area (tx))*100 (Equation 5)

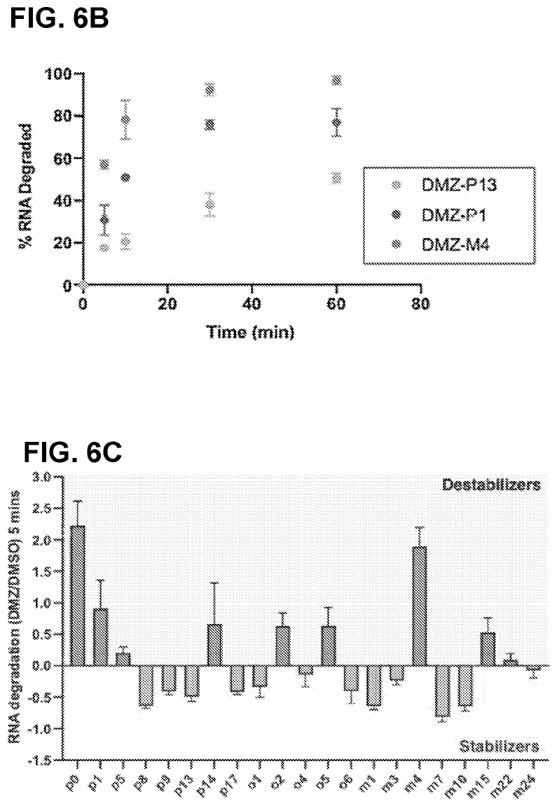

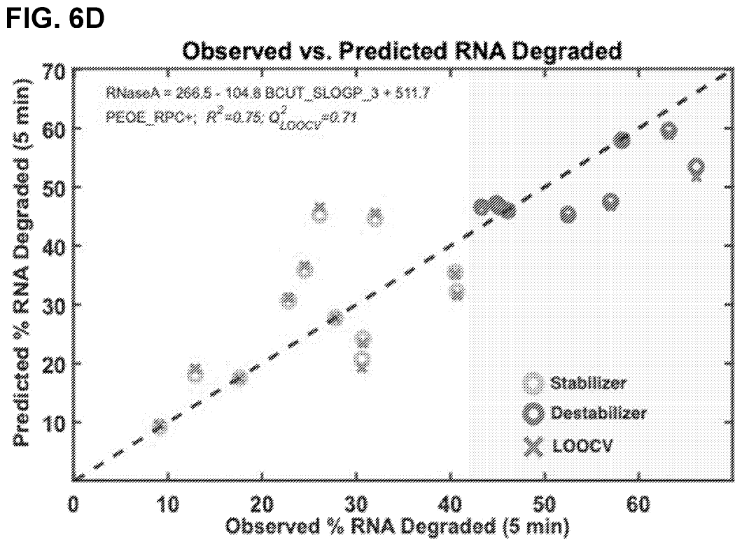

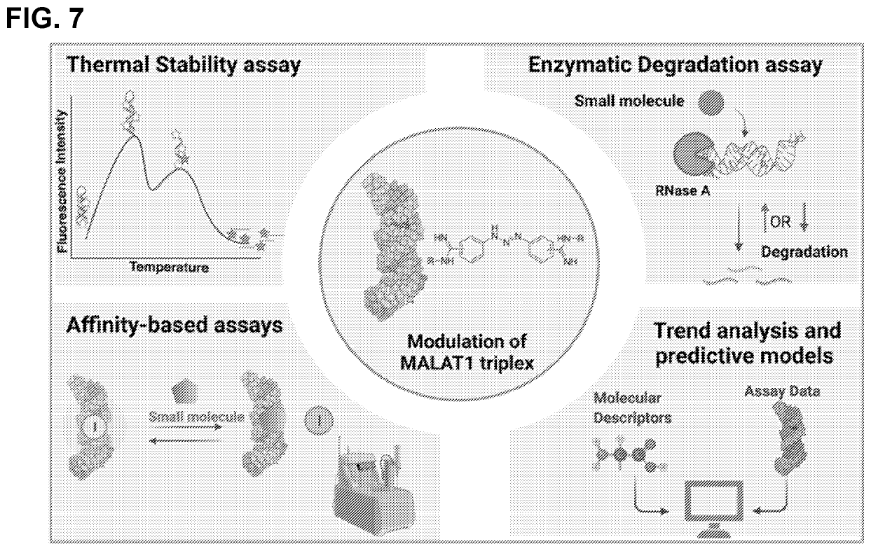

Experimental results shown in FIG. 6C are plotted as three independent replicates and standard deviation between replicates as error bars. Relative fold change between treated condition and DMSO was calculated from Equation (6).

$$Fold change at 5 min = \frac{\% R N A {Degraded}_{Treated} - \% R N A {Degraded}_{DMSO}}{\% R N A {Degraded}_{DMSO}} \qquad ( Equation 6 )$$

### Protonation Correction

A python code was used to adjust the SMILES strings of each small molecule for protonation at pH=7.4. The python code is presented below.

```
#!/bin/bash
  evaluate | grep “version “
  cxcalc -o 01-CanRes.sdf Input.txt -N ih canonicalResonant -f sdf
  cxcalc -o 02-MMcorrected.txt 01-CanRes.sdf -N ih -g majormicrospecies -H 7.4 ### -M for
notaut comment-out -M
  cxcalc | grep “version “
  evaluate -g 02-MMcorrected.txt -e
“molString(′smiles′);mass( );acceptorCount( );donorCount( );logPKLOP( );rotatableBondCount(
);PSA( );logD(′7.4′);atomCount(′7′);atomCount(′8′);ringCount( );aromaticRingCount( );heteroRi
ngCount( );ringSystemCount( );ringCount( )/ringSystemCount( );count(filter(′atno( )==6&&conn
ections( )==4′))/atomCount(′6′);chiralCenterCount( );ASA( );PSA( )/vanDerWaalsSurfaceArea( )
;totalCharge( );vanDerWaalsSurfaceArea( )” -o 03-OutputCalcs.txt 2> 03-output-err.txt
  cat 03-OutputCalcs.txt | tr -d, | tr ′;′′,′> 03-OutputCalcs.csv ## translate semi-colon in
output.txt to comma for output.csv
  cat 03-OutputCalcs.csv | awk -F\, ′{print $ 1}′ > 03-OutputCalcs.smi ## create file with just
first column of smiles
  [ -s 03-output-err.txt ] || rm 03-output-err.txt ## remove err if empty
  evaluate | grep “version “
  wc *
  echo -n “Finished” && date
```

### Small Molecule Selection

The Kennard Stone algorithm was used during selection of the small molecules for synthesis to ensure maximum representation of small molecule 3D shape diversity (FIG. 8A).

```
function [model,test]=kenstone(X,k)
[m,n]=size(X);
if k>=m | k<=0
  h=errordlg(‘Wrongly specified number of objects to be selected to model set.’,‘Error’);
  model=[ ];
  if nargout==2
    test=[ ];
  end
  waitfor(h)
  return
end
x=[[1:size(X,1)]’X];
n=size(x,2);
[i1,ind1]=min(fastdist(mean(x(:,2:n)),x(:,2:n))
model(1)=x(ind1,1);
x(ind1,:)=[ ];
[i2,ind2]=max(fastdist(X(model(1),:),x(:,2:n)));
model(2)=x(ind2,1);
x(ind2,:)=[ ];
for d=3:k
  [ii, ww]=max(min(fastdist(x(:,2:n),X(model,:))));
      model(d)=x(ww,1);
      x(ww,:)=[ ];
end
if nargout==2
  test=1:size(X,1);
  test(model)=[ ];
end
```

### QSAR Modeling

Before calculation, all the ligands were tuned to the correct protonation and tautomerization states using above protonation correction steps. The optimized protonation and tautomerization states were sent to conformational search in Molecular Operating Environment (MOE, Chemical Computing Group, 2018.01) individually to account for the flexibility of the ligand. Low energy conformations of each molecule were calculated using the Conformation Search algorithm in MOE. The Conformation Search function was performed using the stochastic method with the MMFF94 force field and generalized Born solvation model. The input for each parameter is listed in Table 3, and the following options were checked: hydrogens.

TABLE 3
Parameters Used for Conformation Search.

| Parameter | Input |
|---|---|
| Rejection limit | 100 |
| Iteration limit | 10000 |
| RMS gradient | 0.005 |
| MM iteration limit | 500 |
| RMSD limit | 0.15 |
| Energy window | 3 |
| Conformation limit | 10000 |

The 3 kcal/mol energy window was selected to survey biologically relevant conformation space and to obtain a representative population of conformers at equilibrium (>99%) as described by Equation S1.

$$\frac{{N}_{1}}{{N}_{o}} = {e}^{- Δ E / RT} \qquad ( Equation S1 )$$

where N₁/N₀ is the ratio of the number of molecules in the relative energy states, ΔE is the energy difference between N₀ and N₁ (3 kcal/mol), R is the ideal gas constant (0.00198588 kcal/K mol), and T is the temperature (298 K). After the conformation search was complete, the 339 descriptors (2D and 3D descriptors), ranging from electrostatic properties to topological terms, were calculated for each conformation and averaged using the Boltzmann weighted equation (Equation S2).

$$A = \frac{{\sum }_{i} {A}_{i} {e}^{- \frac{{E}_{i}}{{k}_{B} T}}}{{\sum }_{i} {e}^{- \frac{{E}_{i}}{{k}_{B} T}}} \qquad ( Equation S2 )$$

### MatLab Modeling and Scripts

Modeling was performed on MATLAB (R2020a). Model search, selection, LOOCV, and weights calculation.

```
load(′FIDdata.mat′); % this matrix contains the 1st row as the index of the variable names, 0 for
response variable
% find features with constant entry>=80%, delete such features resulting new
% dataset called:data_nonconst
data=FIDdata;
for i=2:size(data,2)
    Y(i)=max(sum(data(:,i)==data(:,i)′));
end
idx_const=find(Y(:)>=0.8*(size(data,1)−1));
data_nonconst=data;
data_nonconst(:,idx_const)=[ ];
% find multicolinerity (abs(rho)>0.95) between features, delete ones with more than 1
% multicolinerity, based on the max number of multicolinerity, saved the
% refined data in the data_refine.
data_refine=data_nonconst;
cor=corrcoef(data_refine(2:size(data_refine,1),2:size(data_refine,2)));
cor=abs(cor);
[a,b]=find(cor>0.95);
A=[b,a];
id=find(b>=a);
A(id,:) = [ ];
uni=unique(A(:,1));
num=zeros(size(uni,1),1);
for i=1:size(uni,1)
    idx=find(uni(i)==A(:,1));
    num(i)=size(idx,1);
end
n=0;
while max(num)>1 % repeat until only one more feature is correlated
id_max=find(num==max(num));
if size(id_max,1)>1
    id_max = id_max(1,1);
else
    id_max=id_max;
end
del_col=find(A(:,1)==uni(id_max));
id_del=A(del_col,2);
data_refine(:,id_del+1)=[ ];
cor=corrcoef(data_refine(2:size(data_refine,1),2:size(data_refine,2)));
cor=abs(cor);
[a,b]=find(cor>0.95);
A=[b,a];
id=find(b>=a);
A(id,:) = [ ];
uni=unique(A(:,1));
num=zeros(size(uni,1),1);
for i=1:size(uni,1)
    idx=find(uni(i)==A(:,1));
    num(i)=size(idx,1);
end
n=n+1; % record how many steps take to complete this task
end
% in a pair of multicorrelation, delete the one with lower cor to the y
% variable
m=0;
while size(A,1)>0
    cor1=abs(corrcoef(data_refine(:,1),data_refine(:,A(1,1)+1)));
    cor1=cor1(1,end);
    cor2=abs(corrcoef(data_refine(:,1),data_refine(:,A(1,2)+1)));
    cor2=cor2(1,end);
    if cor1>=cor2
      id_del=A(1,2);
      data_refine(:,id_del+1)=[ ];
    else
      id_del=A(1,1);
      data_refine(:,id_del+1)=[ ];
    end
    cor=corrcoef(data_refine(2:size(data_refine,1),2:size(data_refine,2)));
    cor=abs(cor);
    [a,b]=find(cor>0.95);
    A=[b,a];
    id=find(b>=a);
    A(id,:) = [ ];
    m=m+1;
end
save(′FID_data_refine.mat′,′data_refine′);
%% two-parameter linear model
% T_FID.mat is a table contains variable names from data_refine.
idx_keep=data_refine(1,:)+1;
T_FID=readtable(′FIDdata.csv′,′PreserveVariableNames′,true);
T_FID=T_FID(:,idx_keep);
save(′T_FID.mat′,′T_FID′);
x=T_FID(:,2:size(T_FID,2));
y=T_FID(:,1);
Results=[ ];
for i=1:size(x,2)−1
  for j=(i+1):size(x,2)
      X=[x(:,i),x(:,j)];
      mdl=fitlm([X,y]);
      r_A=mdl.Rsquared.Adjusted;
      r_O=mdl.Rsquared.Ordinary;
      Results=[Results;r_Ar_O i j];
      X=sprintf(′This is the run of %d, %d′, i,j);
      disp(X)
  end
end
%sort the R2 from high to low. Chose the best model based on the R2 and Q2, here pick the first
one as the only qualifiable one (with feature index 11 and 91) for loocv.
Results=sortrows(Results,1,′descend′);
% summarzie overall R2 and model statistics
data_mdl=[T_FID(:,11),T_FID(:,91),T_FID(:,1)];
mdl_overall=fitlm(data_mdl)
% LOOCV for the top model
yhat=zeros(size(data_mdl,1),1);
for i=1:size(data_mdl,1)
    test=data_mdl(i,:);
    train=data_mdl;
    train(i,:)=[ ];
    mdl=fitlm(train);
    yhat(i)=predict(mdl,test);
end
%calculate Q2_loocv
Rsquare(table2array(data_mdl(:,3)),yhat)
% calculate the weight of top features, threshold as >=0.6 R2_ordinary
idx_top=find(Results(:,2)>=0.6);
feature_top=Results(idx_top,3:4);
%% Occur is an*1 vector containing all the descriptors appeared in the well fitted models
%% Y =n*2 matrix, first column is the descriptor ID, the second column is their occurence.
Occur=[feature_top(:,1);feature_top(:,2)];
Y=(sum(Occur==Occur′))′;
Y=[Occur, Y];
[~,idu]=unique(Occur(:,1));
Y=Y(idu,:);
histogram(Occur,′BinWidth′,1);
title(′Occurrence − FID′,′FontSize′,18);
xlabel(′Descriptor ID′,′FontSize′,18);
ylabel(′Counts′,′FontSize′,18);
% plot the figure
scatter(table2array(T_FID(:,1)),mdl_overall.Fitted,100,′MarkerEdgeColor′,[0.00 0.45 0.74], ...
    ′MarkerFaceColor′,′none′,′LineWidth′,2)
ax=gca;
ax.FontSize=15;
hold on
scatter(table2array(T_FID(:,1)),yhat,100,′x′,′MarkerEdgeColor′,[1 0 0], ...
    ′MarkerFaceColor′,′none′,′LineWidth′,2)
x=linspace(0,2.5);
y=x;
plot(x,y,′LineWidth′,2,′LineStyle′,”--”,”Color”,′k′)
legend(′Fitted′,′LOOCV′,′FontSize′,15,′TextColor′,′k′,′Location′,′southeast′,′FontWeight′,′bold′)
title(′Pred. vs Observed log(FID)′,′FontSize′,20,′FontWeight′,”bold”)
xlabel(′Observed′,′FontSize′,20,′FontWeight′,”bold”)
ylabel(′Predicted′,′FontSize′,20,′FontWeight′,”bold”)
annotation(′textbox′,[0.15,0.8,0.45,0.1],′FontSize′,12,′String′, ...
    strcat(′log(FID) = 1.7 − 0.007 ASA\_P + 0.036 PEOE\_VSA-5′,′;′,{32},′\it R\hat{ }2=0.69′,′;′,{32},
...
    ′Q\hat{ }2_{LOOCV}=0.60′),′EdgeColor′,′none′);
```

For the descriptor weight calculation, the threshold for the top models were set for R2_ordinary. IDA Data was 0.69, RNase was 0.75, and Tₘ was 0.90. All the models (31375), then R²≥0.6, then good models, then R²_LOOCV≥0.40 and Q²_{F2}≥0.5, then top models.

### QSAR Modeling

The MTT reagent (3-(4,5-dimethylthiazol-2-yl)-2,5-diphenyl-2H-tetrazolium bromide) is a mono-tetrazolium salt that consists of a positively charged quaternary tetrazole ring core containing four nitrogen atoms surrounded by three aromatic rings including two phenyl moieties and one thiazolyl ring. Reduction of MTT results in disruption of the core tetrazole ring and the formation of a violet-blue water-insoluble molecule called formazan. The MTT reagent can pass through the cell membrane as well as the mitochondrial inner membrane of viable cells presumably due to its positive charge as well as its lipophilic structure and is reduced to formazan by metabolically active cells. The chromogenic nature of this redox chemical reaction provides a colorimetric-based measurement of intracellular formazan production based on which the MTT assay was developed. Consequently, the assay has extensive utility as a cell metabolic activity assay.

### Example 1

### Design and Synthesis of Diminazene Centric Library

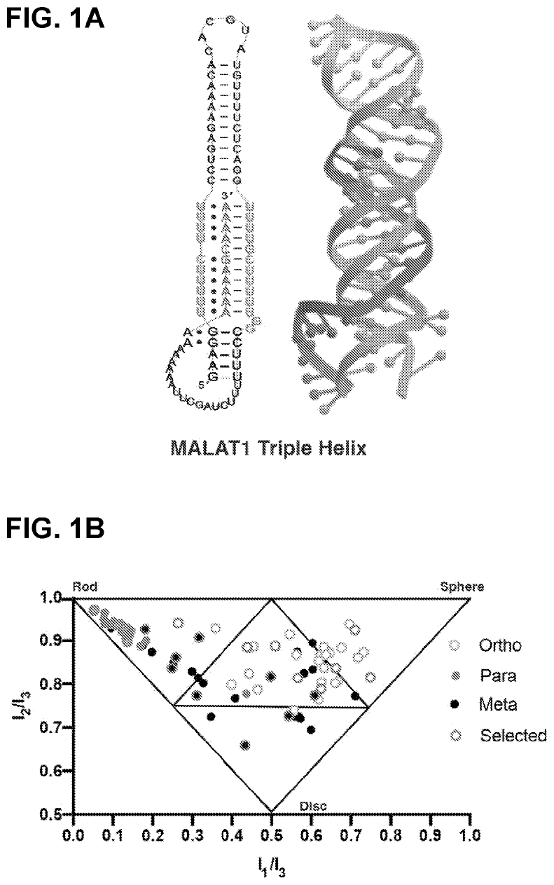

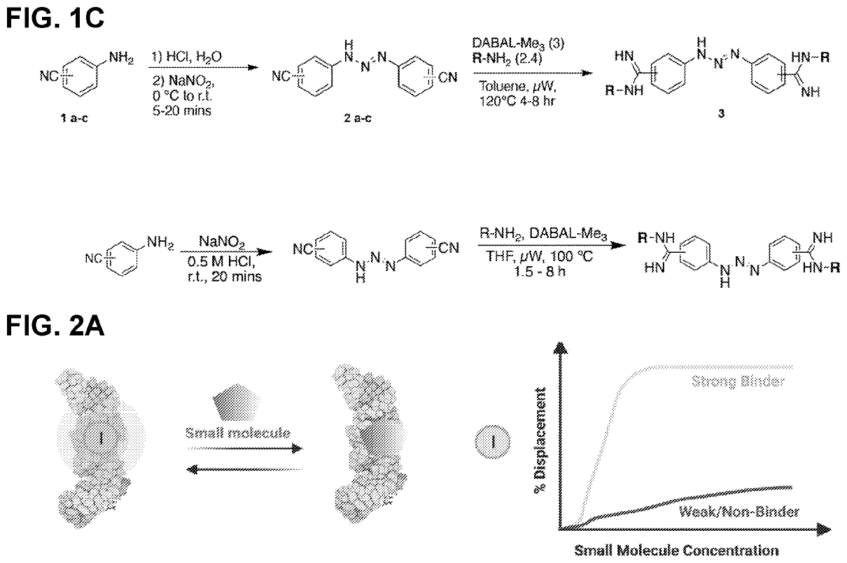

The diminazene scaffold was selected due to a number of reported properties, including bioavailability, solubility, ease of synthetic tuning, and reported nucleic acid binding. Also known as Berenil®, diminazene is an FDA approved (Rajapaksha I G, et al. (2018) Sci Re. 8(1):10175) antiparasitic agent and a known nucleic acid binder. (Nguyen B, et al. (2009) Acc Chem Res. 42(1):11-21; Pilch D S, et al. (1995) Biochemistry. 34(49):16107-16124; Nguyen B, et al. (2004) Biophys J. 86(2):1028-1041). It has also been shown to preferentially bind A-U rich RNA triple helices, (Pilch D S, et al. (1995) Biochemistry. 34(49):16107-16124) making it an attractive case study for targeting the A-U rich MALAT1 triple helix (FIG. 1A). In addition, the synthetic versatility of this scaffold allowed maximization of 3D shape diversity while minimizing confounding variables that would arise from using different chemotypes and might bias shape-based analyses. This scaffold, and the scaffold-based library approach in general, allows us to identify the key moieties needed for engagement of the intended RNA target while revealing cheminformatic trends that can guide future synthetic efforts.

First, to ensure the suitability of the diminazene scaffold to yield 3D shape diverse analogues, a 90-member theoretical library was created by coupling commercially available amines with the three possible regioisomers of the scaffold precursor (FIG. 1B, white, grey, black). All analogues were equipped with the diamidine moiety as it is appears to be necessary for small molecule interactions in the minor groove of A-U rich sequences. (Pilch D S, et al. (1994) Proc Natl Acad Sci USA. 91(20):9332-9336).

To afford a direct comparison with known RNA-binding small molecules, the 3D shape of the analogues was evaluated by calculating the principal moment of inertia (PMI) on the pH-adjusted lowest energy conformers of each theoretical ligand. (Morgan B S, et al. (2017) Angew Chem Int Ed Eng. 56(43):13498-13502).

Normalized principal moments of inertia (npr1: I₁/I₃, npr2: I₂/I₃) were calculated and plotted on the envelope diagram commonly used for 2D visualization of the data. Notably, most of the para-substituted analogues clustered in the rod-like sub-triangle while the ortho- and meta-substituted subsets were scattered across the hybrid, sphere, and disc sub-triangles, affording a suitable coverage of 3D shape diversity.

To maximize shape representation within the synthesized subset, the Kennard-Stone algorithm (Morais C L M, et al. (2019) Bioinformatics. 35(24):5257-5263) was used to select 21 small molecules for synthetic evaluation (FIG. 1B, red).

The synthetic route designed for the focused library capitalized on the oxidation of a 2-, 3-, or 4-aminobenzonitrile (1 a-c) to form a diazonium ion intermediate that is subsequently coupled with another equivalent of the same aniline (FIG. 1C). (Hill D T, et al. (1983) J Med Chem. 26(6):865-869; Rastogi S K, et al. (2018) Eur J Med Chem. 143:1-7). Purified intermediate (2 a-c) of each biscyano regioisomer was then submitted to diamidine formation through activation of the nitrile functional group by the Lewis acid DABAL-Me₃, optimizing a previously reported procedure (3). (Donlic A, et al. (2018) Angew Chem Int Ed Eng. 57(40):13242-13247).

Three of the initially selected subunits contained extensively conjugated anilines that were found to be unreactive in the presence of several Lewis acids and under various reaction conditions (FIG. 9).

To choose replacement library members, Euclidean distance was used to identify the analogues in the theoretical library closest in distance on the PMI envelope diagram to each initially selected untractable ligand, thus preserving diversity in the final 21-member library (FIG. 2B, FIG. 8A-FIG. 8B). In total, 21 symmetrical analogues were afforded in good yield and purity.

A series of affinity-based methods were used as screening and selection benchmarks to identify small molecule leads that were further assessed via thermal stability and enzymatic degradation assays. (Wicks S L, et al. (2019) Methods. 167:3-14). This range of in vitro methods was chosen to assess 3D shape trends as a function of a variety of outputs, allowing assessment of discrepancies, if any, between measured affinity, thermal stability, and profiles of enzymatic degradation.

### Example 2

### Indicator Displacement Assay (IDA)

IDAs are valuable high throughput screening (HTS) tools in RNA ligand discovery, yet the vast majority of currently reported IDAs have been optimized for RNA secondary structures utilizing peptides and fluorescent indicators reported to bind bulges and/or internal loops. (Wicks S L, et al. (2019) Methods. 167:3-14).

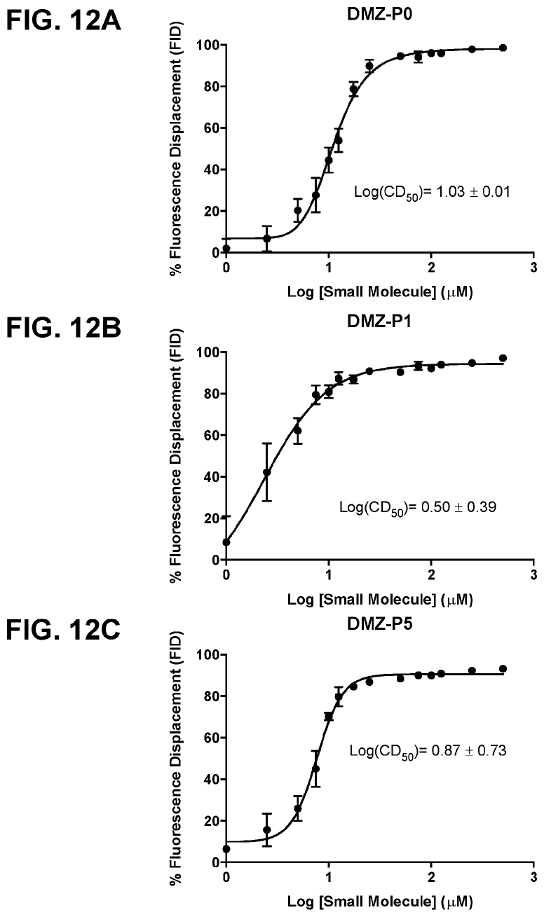

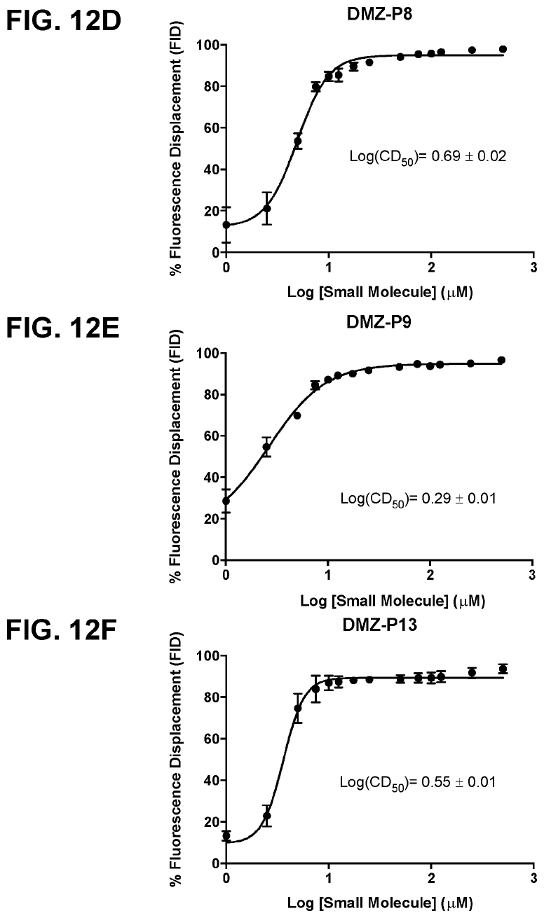

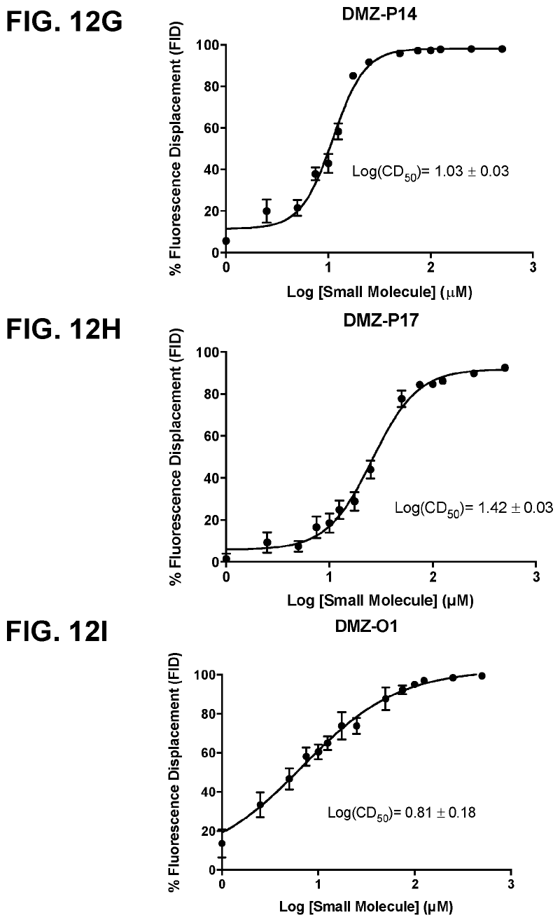

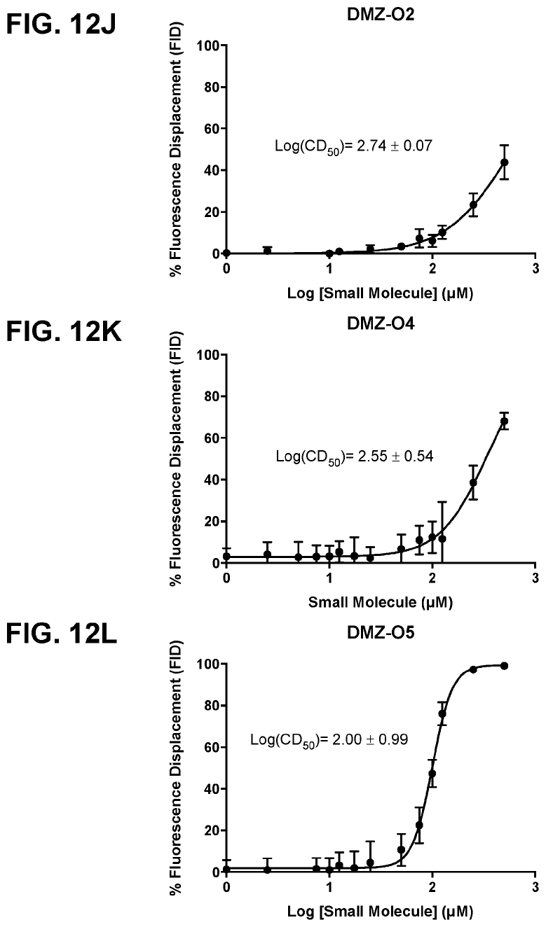

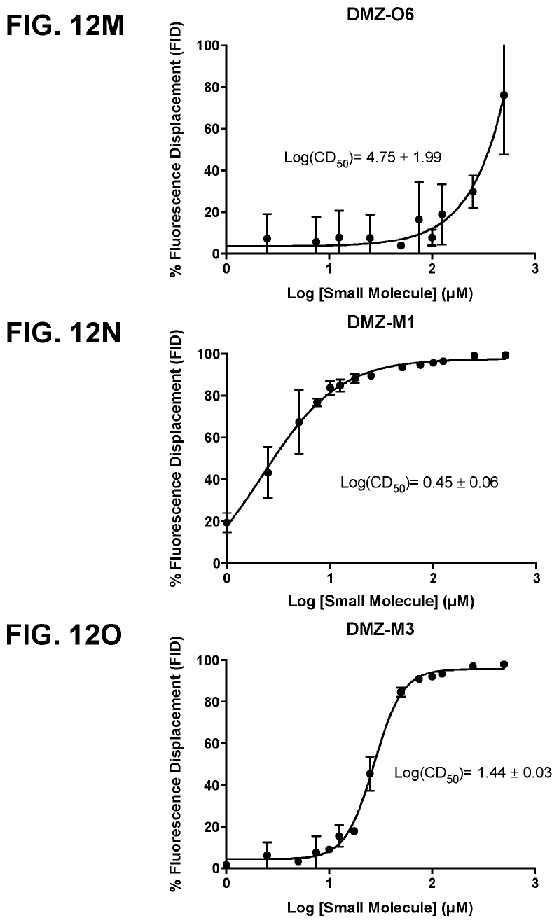

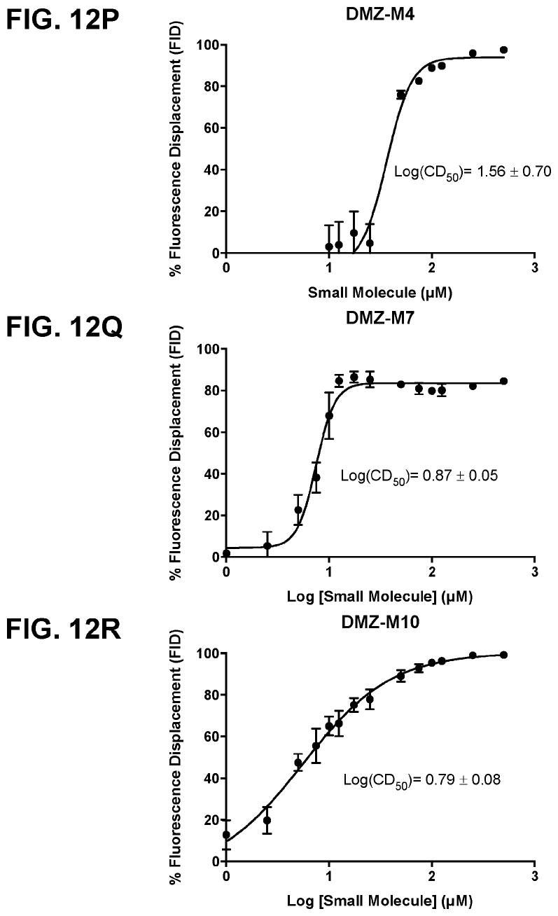

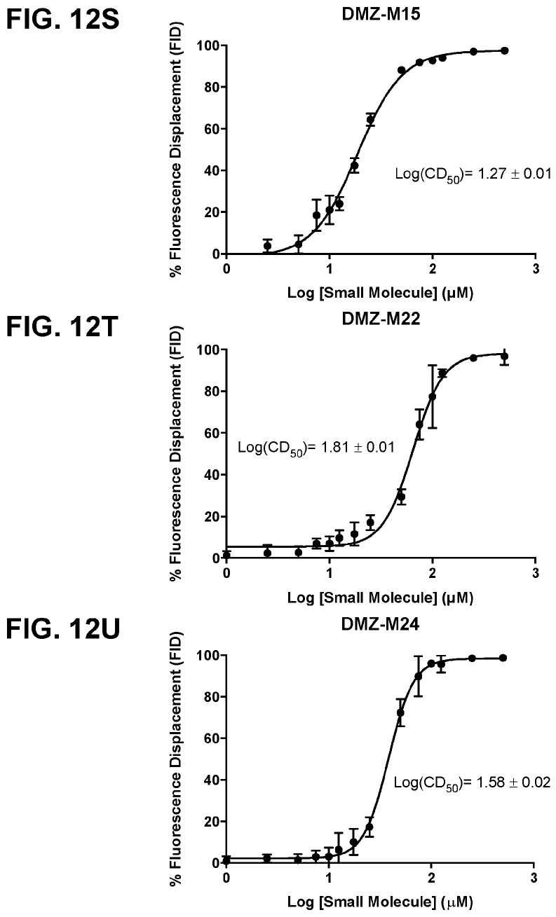

Given the structural differences between secondary and tertiary structures, where more continuous and complex base-pairing occurs, the design and optimization of an RNA triple helix IDA amenable to HTS was the first focused on. The utility of RiboGreen™ dye was confirmed as an indicator. The RiboGreen™ structure is proprietary, and stoichiometry is thus unconfirmed. Upon assay optimization, the 21-member library was screened using a 16-point titration in three independent replicates (FIG. 2A, FIG. 12A-FIG. 12U).

The data showed a range of affinities from low to high micromolar, indicating the potential tunability of the diminazene scaffold for targeting the MALAT1 triple helix (FIG. 2C). Aliphatic subunits with protonated nitrogens (FIG. 2B; see R-1, R-9, R-10) were among the best binders regardless of the regioisomer, indicating a charge-driven binding event.

In contrast, regioisomer discrimination was observed for aromatic subunit R-4, which showed no affinity in the ortho analogue DMZ-O4 but displayed a CD₅₀ (50% competitive displacement dose) of 36.6 mM when coupled to the meta scaffold as DMZ-M4.

A similar pattern was observed for R-5, where a 13-fold higher affinity was observed with the para scaffold (DMZ-P5) versus the ortho scaffold (DMZ-O5).

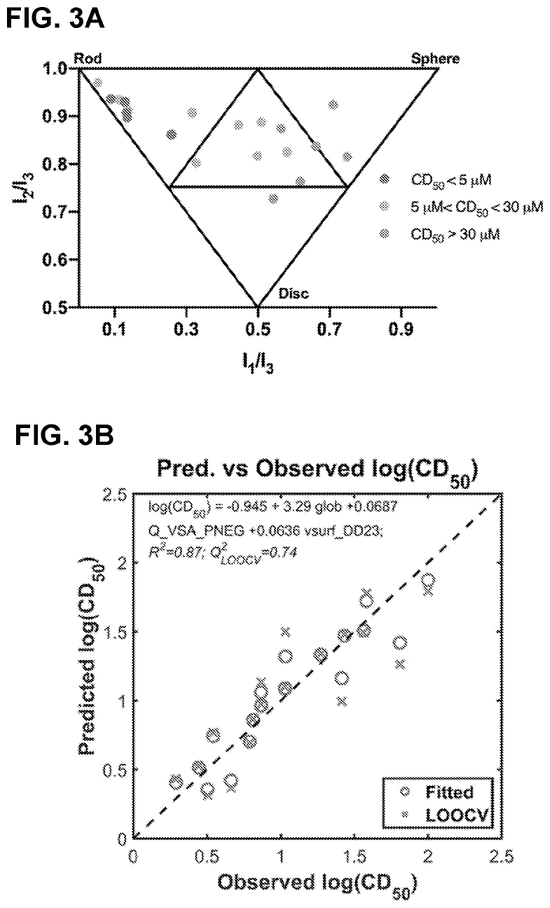

To qualitatively analyze possible 3D shape trends, the principal moment of inertia diagram for the 21 synthesized small molecules was color-coded based on high affinity (CD₅₀<5 mM, red), medium affinity (5 mM<CD₅₀<30 mM, orange), and weak affinity ligands (CD₅₀>30 mM, grey, FIG. 3A).

Notably, all the high affinity ligands clustered in the rod-like sub-triangle, while the medium affinity binders are spread between the rod-like and hybrid sub-triangle. The most hybrid, disc-like, and sphere-like ligands had weak or no affinity.

The diminazene scaffold has a flexible three nitrogen core, which might allow analogues with subunits that have a high propensity for binding to adopt conformations distinct from the lowest energy conformer in order to allow RNA:small molecule interactions. (Nguyen B, et al. (2004) Biophys J. 86(2):1028-1041; Spychala J, et al. (1994) Eur J Med Chem. 29(5):363-367). Furthermore, comparable affinity of the medium diminazene binders from different PMI sub-triangles might be indicative of binding to a different triplex conformer. (Pilch D S, et al. (1995) Biochemistry. 34(49):16107-16124).

Despite being some of the most commonly used descriptors, PMIs are not the only way to define small molecule 3D properties (Kumar A, et al. (2018) Front Chem. 6(315)) and 2D parameters might also correlate to binding trends. To further expand the set of possible predictive parameters for small molecule affinity, a quantitative structure activity relationship (QSAR) method to gain insight into small molecule descriptors that might be driving molecular recognition events was employed. (Cai Z, et al. (2022) J Med Chem. 65(10) 7262-7277).

Multiple linear regression models were iteratively generated by fitting combinations of small molecule descriptors to the small molecule's response to the RNA target which, in this case, is affinity. (Nantasenamat C, et al. (2010) Expert Opin Drug Discov. 5(7):633-654; Gramatica P. (2007) QSAR & Combinatorial Science. 26(5):694-701; Maciagiewicz I, et al. (2011) Bioorg Med Chem Lett. 21(15):4524-4527).

In this QSAR study, both 2D and 3D parameters were calculated for a total of 339 descriptors for each ligand. 2D descriptors only use the atoms and connectivity information of the molecule for calculations, while 3D descriptors consider coordinates and individual conformations. Model fitness was calculated based on the accuracy of predicted affinity of the molecules (R²) and the model robustness was evaluated by leave-one-out cross validation (LOOCV, Q²). A final predictive model with R²=0.87 and Q²_{LOOCV}=0.74 was obtained, supporting the affinity predictions for the given small molecule set (FIG. 3B). (Cai Z, et al. (2021) J Med Chem. 65(10):7262-7277).

One of the advantages of the chosen QSAR method over other machine-learning based approaches is the open framework of model construction that allows identification of parameters that directly contribute to the binding event. (Cai Z, et al. (2021) J Med Chem. 65(10):7262-7277).

For example, in the above IDA model, 3D parameters (molecular globularity, glob and contact distances of vsurf_EDmin2 and vsurf_EDmin3, vsurf_DD23) (Cruciani G, et al. (2000) J Mol Structure: THEOCHEM. 503(1):17-30), were identified to contribute positively to the binding affinity, along with a 2D descriptor (total negative polar van der Waals surface area, Q_VSA_PNEG). (Cruciani G, et al. (2000) J Mol Structure: THEOCHEM. 503(1):17-30). The QSAR analysis for the IDA assay indicates that even in an unbiased set of molecular descriptors, 3D properties are critical to predictive models of small molecule affinity to the MALAT1 triple helix.

### Example 3

### Isothermal Titration Calorimetry of Library of Small Molecule

Prior to isothermal titration calorimetry (ITC), RNA was heated at 95° C. and snap cooled on ice for 10 minutes. Then, 0.5 mL of MALAT1 triple helix RNA was loaded in a 3,000 MWKO dialysis cassette and dialyzed for 12 hours at 4° C. in a Tris-based buffer chosen to maximize the small molecule concentration range of solubility (10 mM Tris-HCl, 25 mM NaCl, 3 mM MgCl₂ pH=7.4). The dialysis cassette was suspended in 400 mL of buffer and 2 total buffer changes were performed over the course of the 12 hours of dialysis. The dialysate and final buffer were stored for ITC experiments. For each experiment 5 mM or 10 mM of RNA were used in the sample cell and 1-3 mM of small molecule were used in the syringe of a PEAQ-ITC (MalVern).

After thermal equilibration at 25° C., and initial 150 seconds delay and one initial 0.4 mL injection, 18 serial injections of 2.0 mL were performed at intervals of 150 seconds at a stirring speed of 750 rpm on high feedback mode and 10 kcal reference power. Raw data outlined below was recorded at mcal/s over time (minutes). Small molecules tested were dissolved in dialyzed buffer from powder for every experiment. Each small molecule was screened in three independent experiments and every batch of small molecule and RNA was tested by running a control experiment with buffer. For each RNA control in buffer alone a baseline DP of no more than 0.1 mcal/sec was recorded. A maximum of 3% DMSO was used with small molecules not soluble in buffer alone. In these cases, the % DMSO was matched in RNase free water in the reference cell and control experiments never exceeded 0.2 mcal/s baseline. The dissociation constant K_{d} was extracted from a curve fit calculated with the PEAQ-ITC (MalVern) software. K_{d} values reported below are the average of the three independent experiments±the error derived from the standard deviation of the three trials.

TABLE 4
Thermodynamic Parameters Derived from ITC Curves of the
Diminazene Analogues against the MALAT1 Triple Helix.

| | K_{D} (mM) | DG (kJ/mol) | DH (kJ/mol) | -TDS (kJ/mol) |
|---|---|---|---|---|
| DMZ-P0 | 34.1 | −6.10 | −7.81 | 1.71 |
| DMZ-P1 | 10.9 | −6.10 | −6.77 | −0.67 |
| DMZ-P5 | 36.2 | −6.06 | −6.76 | 0.70 |
| DMZ-P8 | 9.87 | −6.83 | −3.64 | −3.19 |
| DMZ-P9 | 25.9 | −6.26 | −1.53 | −4.74 |
| DMZ-P13 | 16.6 | −6.53 | −2.72 | −3.80 |
| DMZ-M1 | 5.01 | −7.23 | −6.75 | −0.48 |
| DMZ-M10 | 31.2 | −6.15 | −1.33 | −4.82 |
| Furamidine | 8.46 | −6.92 | −10.50 | 3.55 |

### Example 4

### Isothermal Titration Calorimetry of a Small Molecule Sub-Set

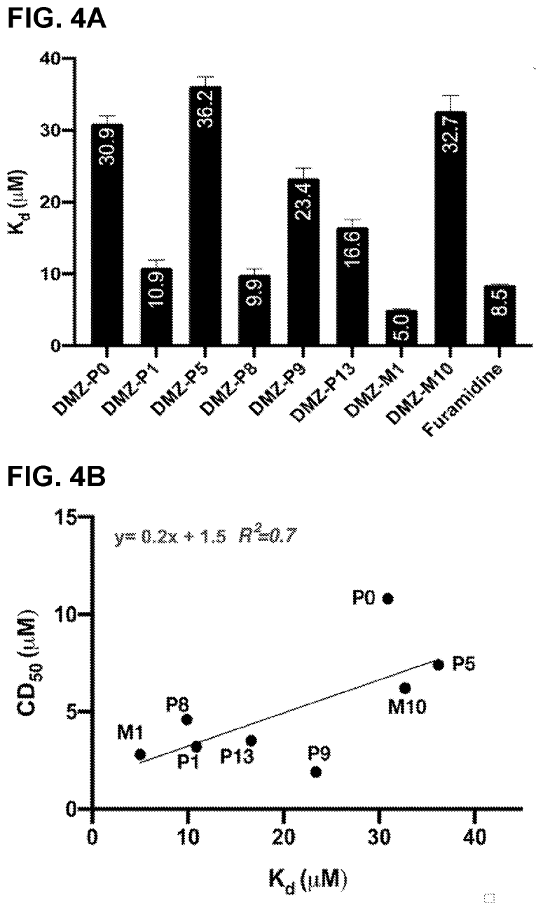

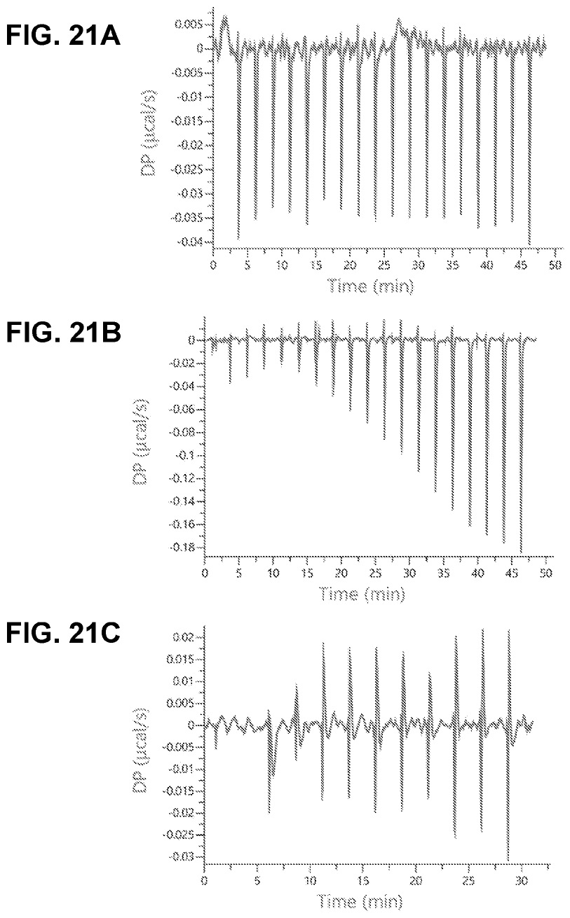

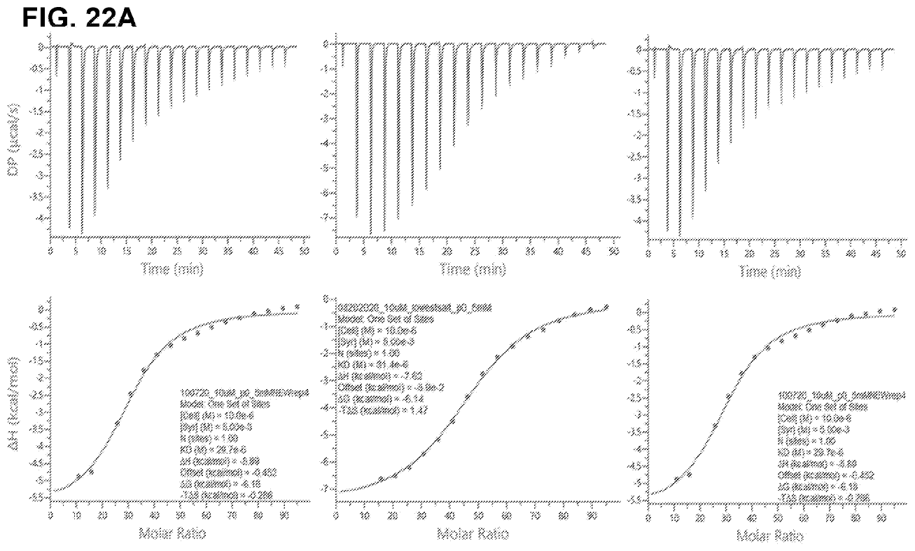

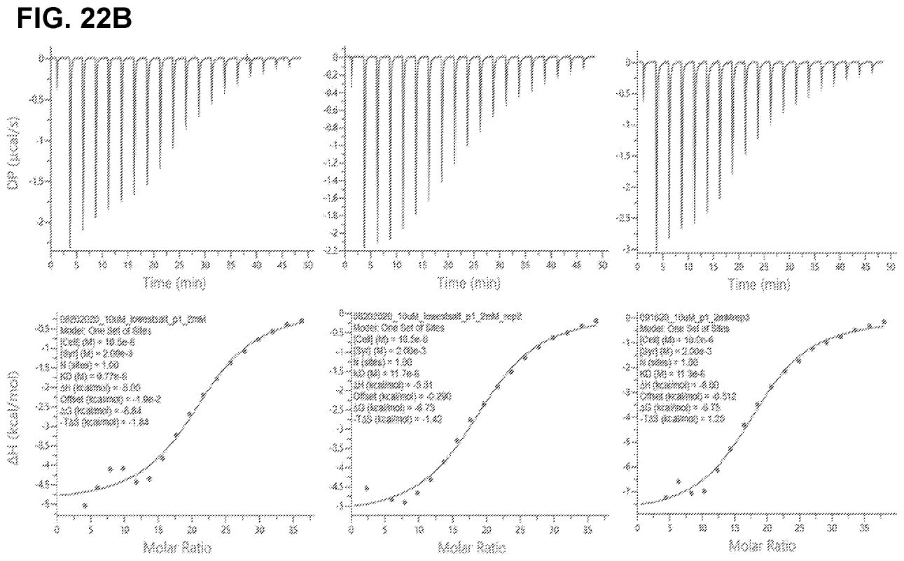

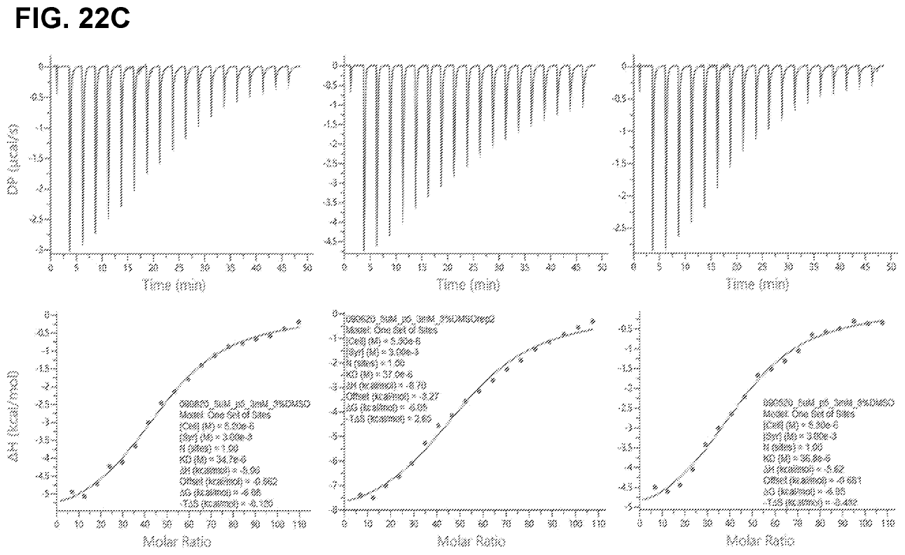

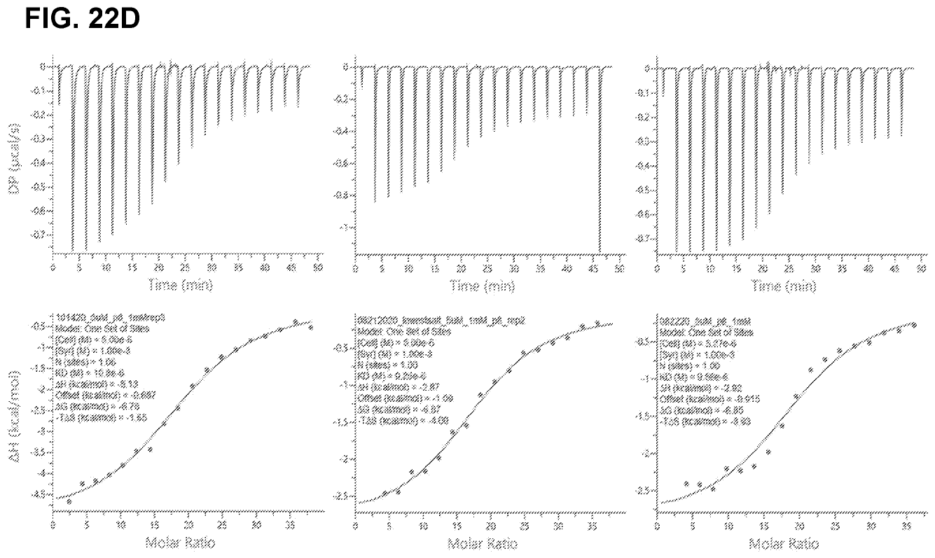

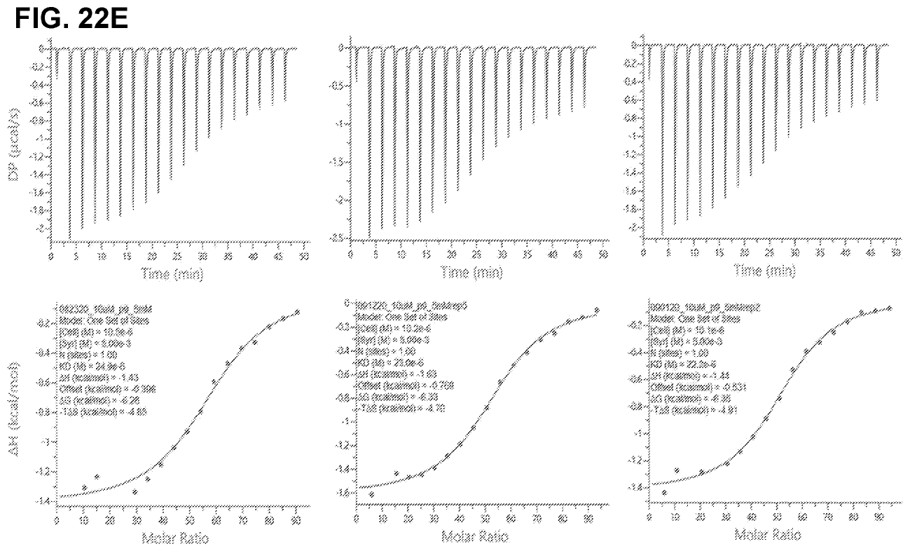

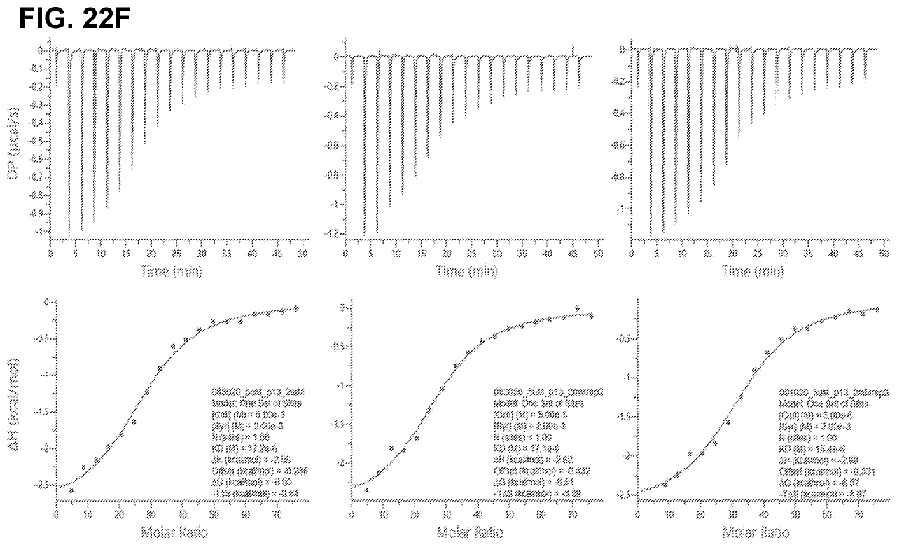

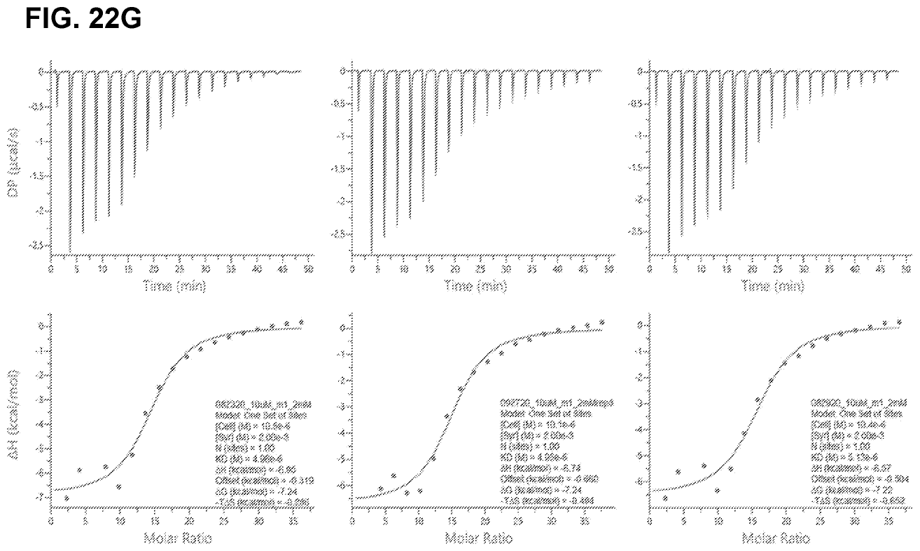

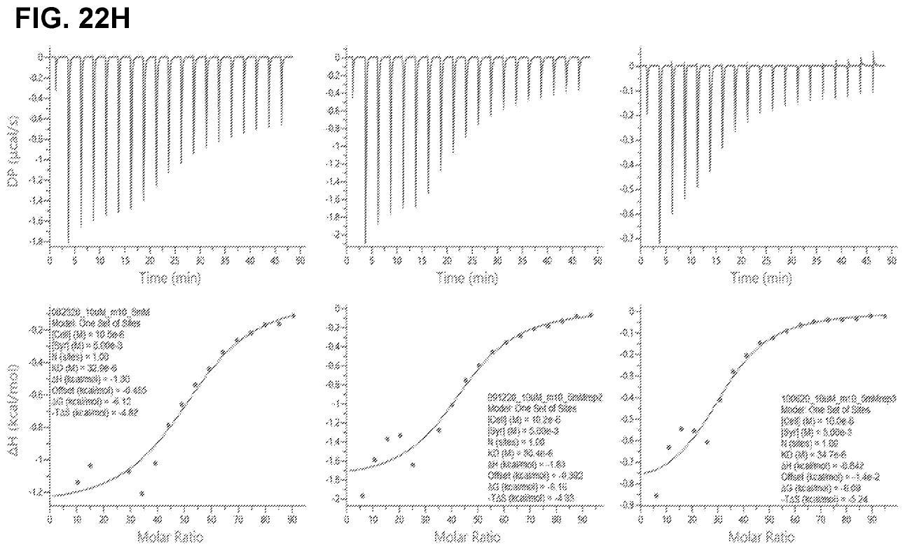

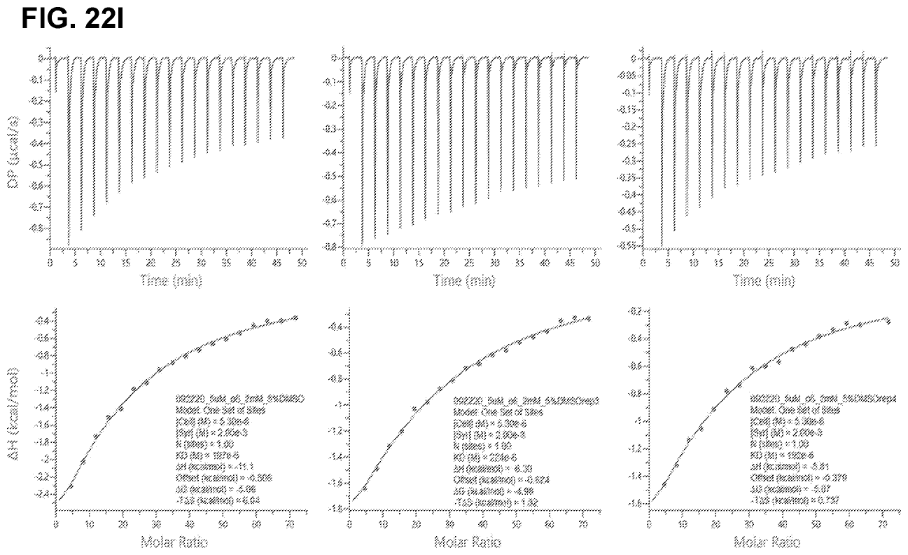

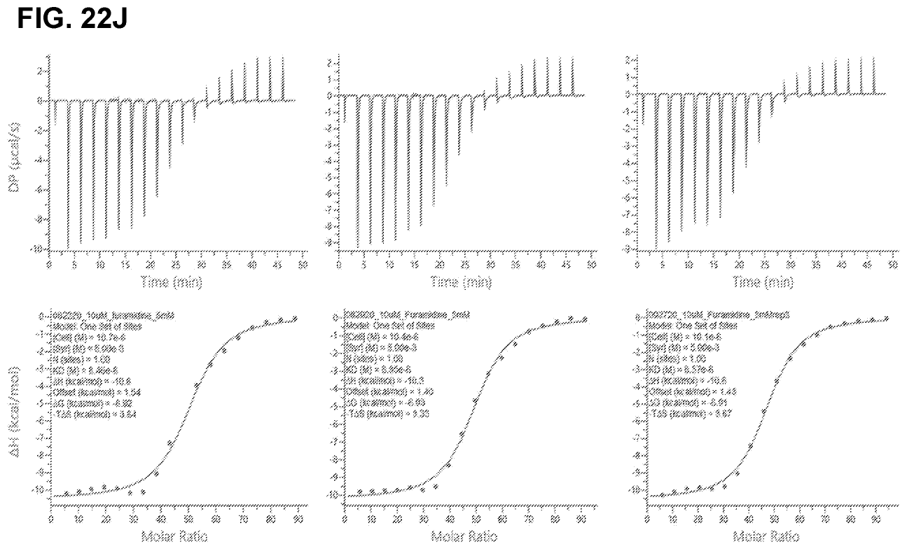

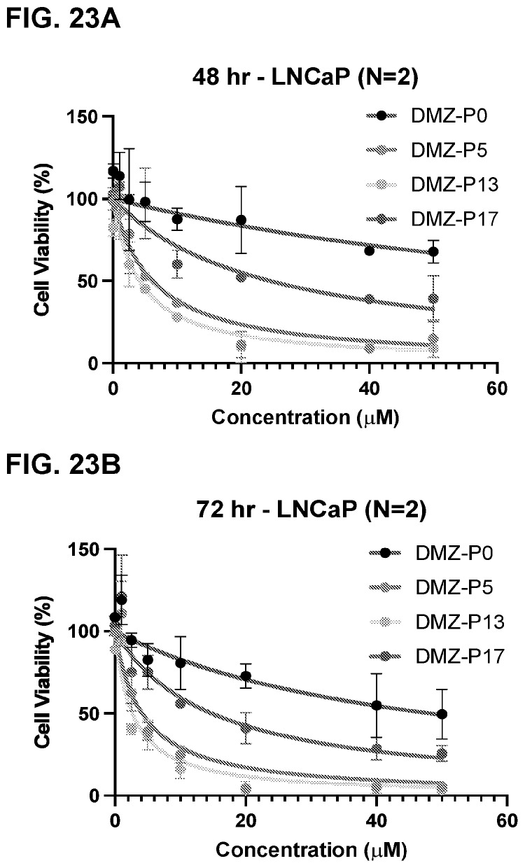

To directly assess the correlation of the IDA assay with affinity, isothermal titration calorimetry (ITC) was performed in three independent replicates for the eight diminazene small molecules that showed high enough solubility to be compatible with ITC, along with furamidine as a reference for DPFs (FIG. 4A, FIG. 21A-FIG. 21C, and FIG. 22A-FIG. 22J). The CD₅₀ values obtained via Ribogreen™ IDA and the K_{d} values obtained through ITC of the eight diminazene analogues are in good agreement, with minor discrepancies (DMZ-P9, DMZ-P0, DMZ-M10) that could be due to the differences in buffers required for the two methods or differential indicator displacement modes by the small molecules (FIG. 4B). All the DMZs screened via ITC reported a stoichiometry of n=1 by a simplex fit where all variables were allowed to float.

The necessary number of base pairs for binding was found to be proportional to the length of the ligands, with 8 base-pairs needed for the longest diminazene analogue in that study. (Nguyen B, et al. (2004) Biophys J. 86(2):1028-1041).

Given that the DMZs studied here are substantially longer than those in the previous study, a stoichiometry of n=1 for the 10-base triples of MALAT1 is consistent. (Pilch D S, et al. (1995) Biochemistry. 34(49):16107-16124; Nguyen B, et al. (2004) Biophys J. 86(2):1028-1041).

Interestingly, direct fluorescence titrations of furamidine with the MALAT1 triple helix yielded a CD₅₀ of 560 nM while ITC reported a K_{d} of 8.35 mM, indicating a possible higher stoichiometry in the direct fluorescence titration conditions.

Importantly, the reasonable correlation between the IDA assay optimized herein and the non-competitive ITC assay supports the utility of this rapid, cost-effective and general IDA method in high-throughput screens against RNA triple helices.

### Example 5

### Effects of Small Molecules on MALAT1 Triplex Thermal Stability

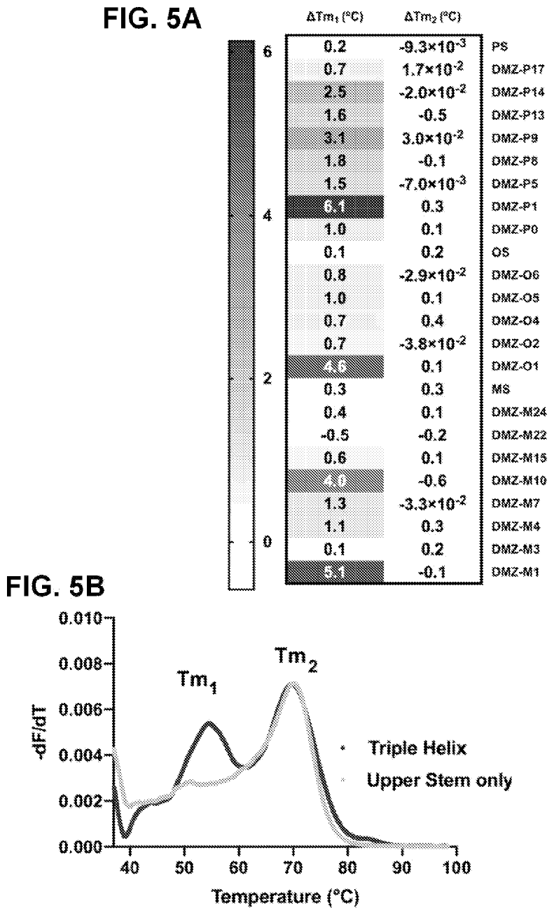

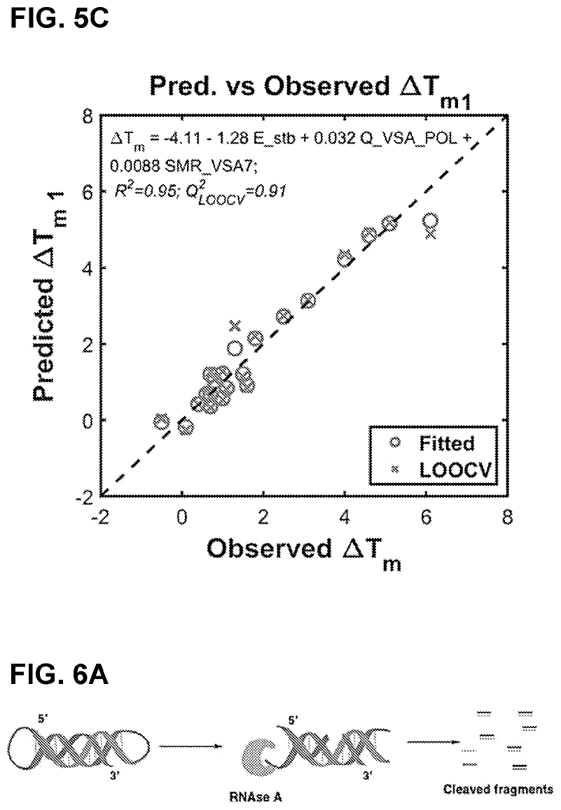

The MALAT1 triple helix is known to confer stability to the full-length transcript and protect it from RNase-mediated degradation, ultimately resulting in transcript accumulation and aggregation in nuclear speckles. (Brown J A, (2020) WIREs RNA. 11(6):e1598; Wilusz J E, et al. (2012) Genes Dev. 26(21):2392-2407; Brown J A, et al. (2014) Nat Struct Mol Biol. 21(7):633-640; Tycowski K T, et al. (2016) Cell Rep. 15(6):1266-1276). To better understand how the diminazene focused library affects the MALAT1 triplex stability, the entire library was evaluated via differential scanning fluorimetry (DSF) (FIG. 5A). This method allows for rapid assessment of small molecule effects on nucleic acid thermal stability (Silvers R, et al. (2015) Chembiochem. 16(7):1109-1114) in a 96-well plate format, substantially reducing the time and cost associated with traditional UV-melting experiments.

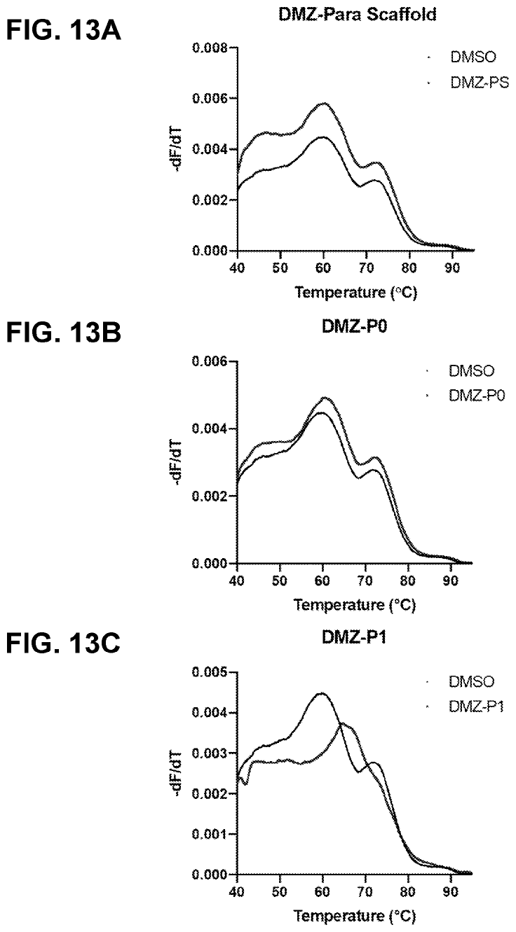

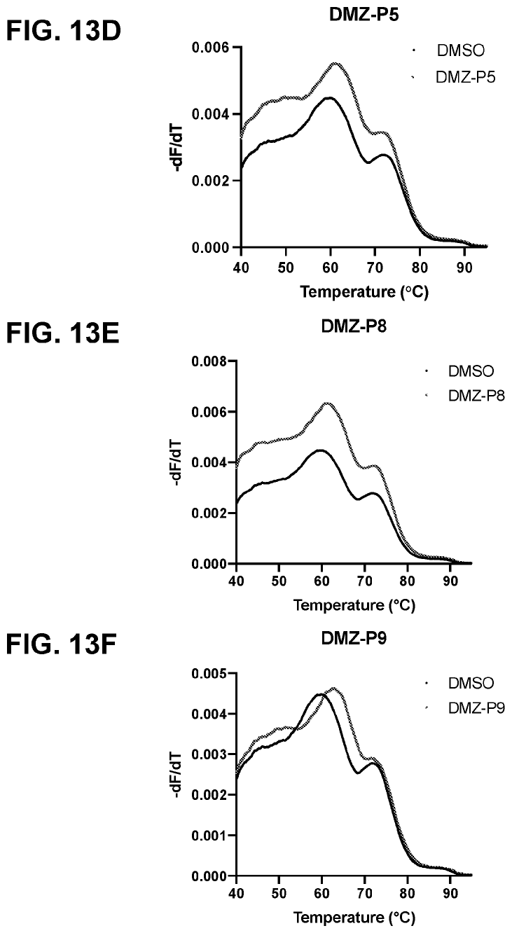

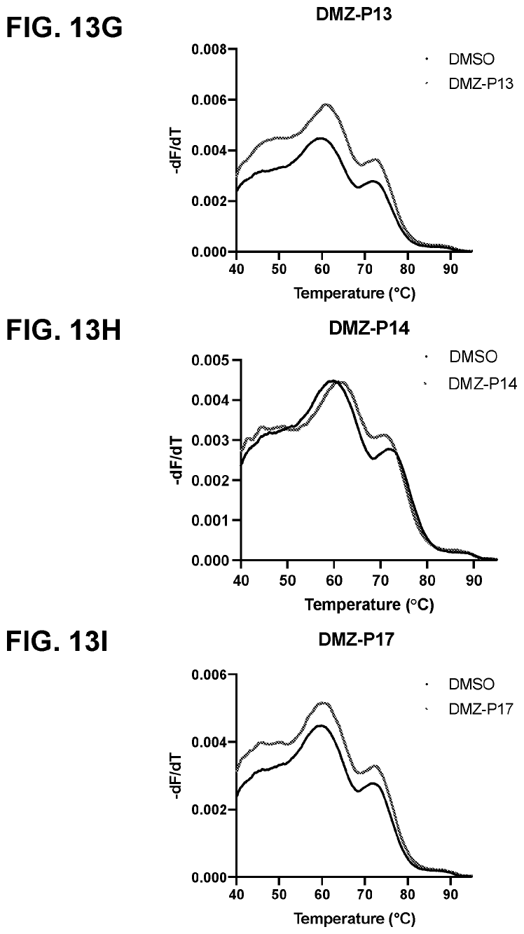

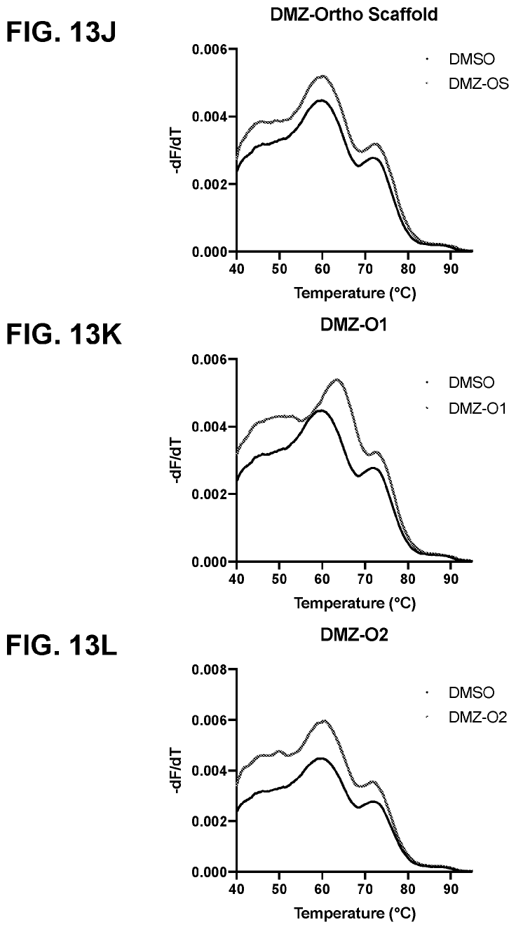

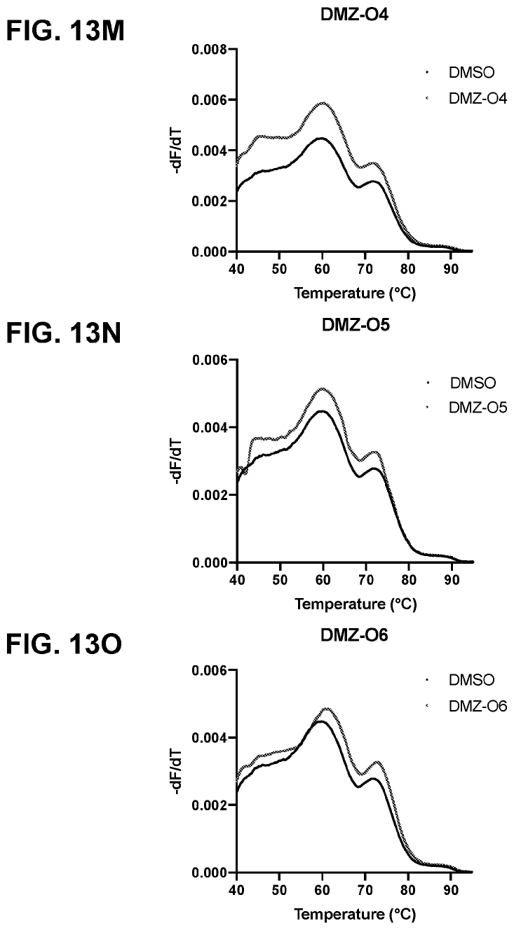

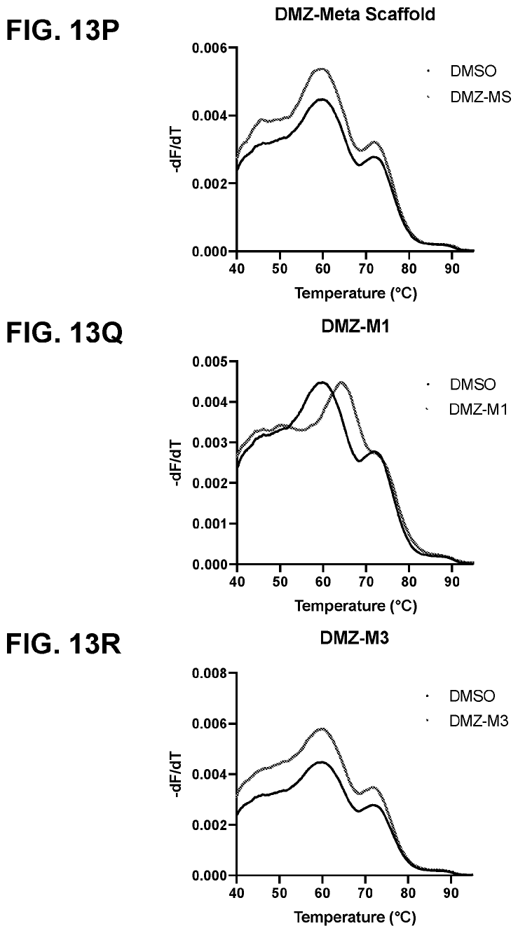

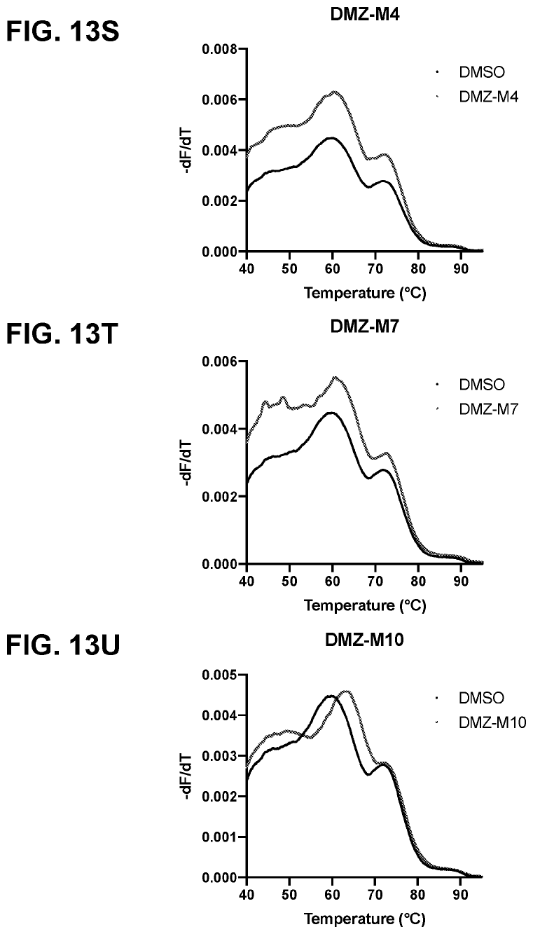

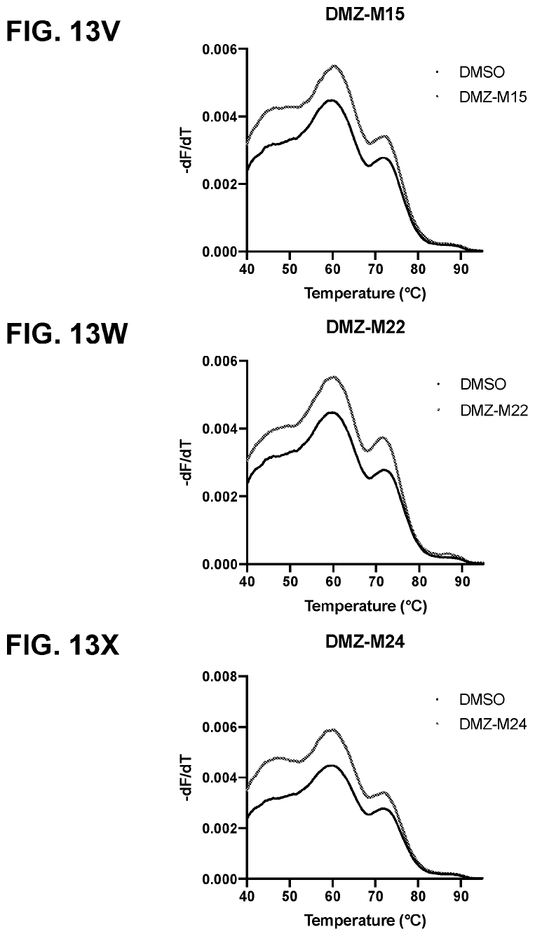

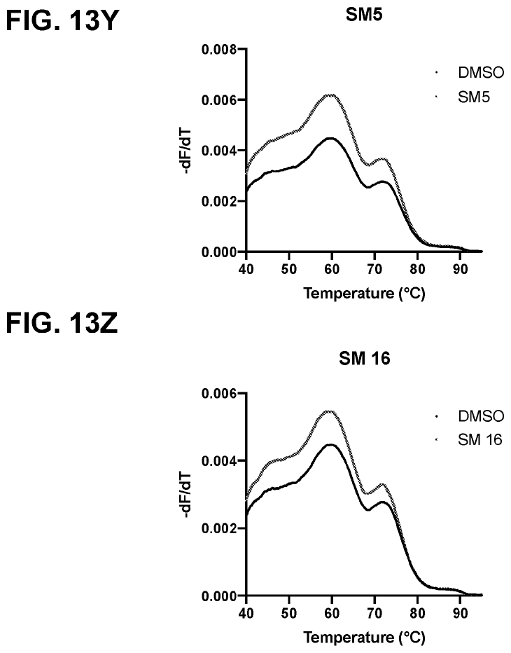

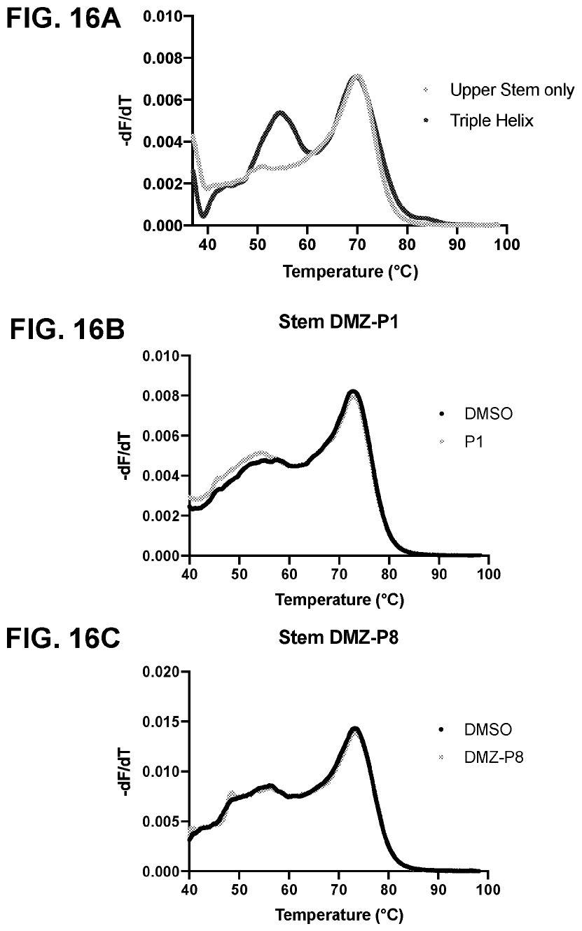

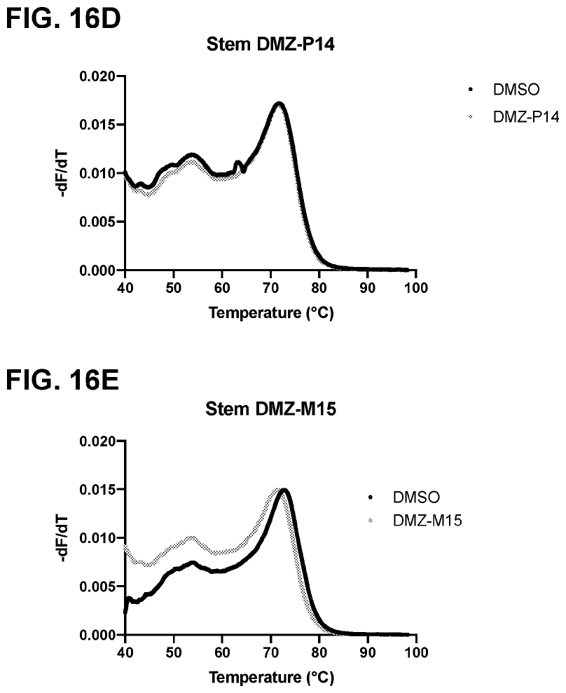

The bi-phasic melting profile distinctive of triple helices is attributed to an initial melting of the Hoogsteen triple base pairs followed by dissociation of Watson-Crick-Franklin base pairs (FIG. 16A-FIG. 16E). (Pilch D S, et al. (1995) Biochemistry. 34(49):16107-16124). At an equimolar ratio of RNA:small molecule, a range of thermal stabilization of the first (Tm₁) was observed, but not the second (Tm₂) melting peak (FIG. 5A, FIG. 13A-FIG. 13Z). At increasing small molecule concentration, a dose-dependent increase in ΔTm₁ but not in ΔTm₂ was observed, indicating small molecule interactions in the triplex region and not in the upper stem region (FIG. 15).

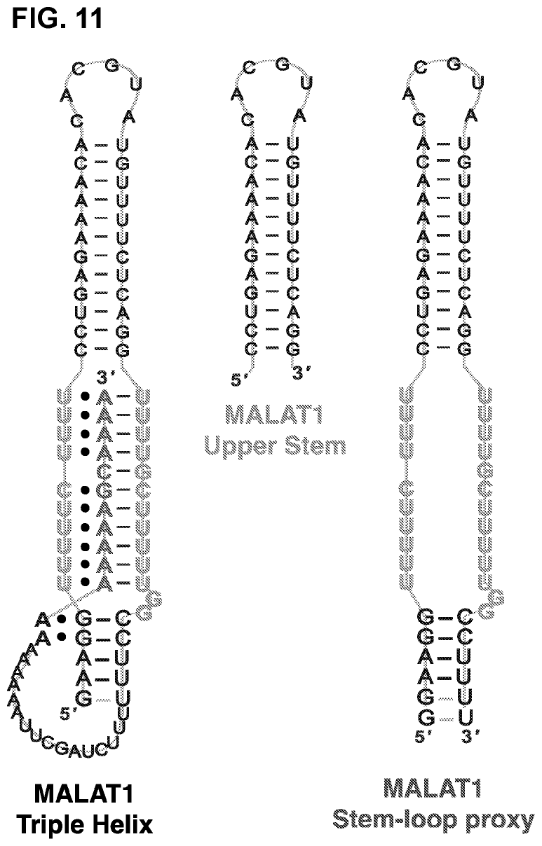

To further explore this hypothesis, a sub-set of small molecules that showed a range of changes in Tm₁ was tested against two truncated MALAT1 constructs: the upper stem of MALAT1 and the stem loop proxy obtained from deletion of the poly(A) tail (FIG. 11, FIG. 16A

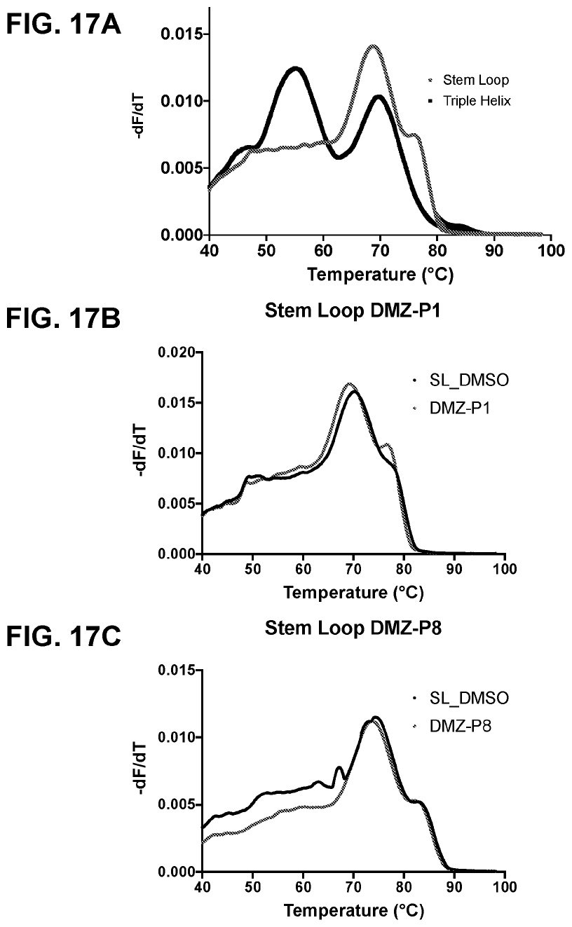

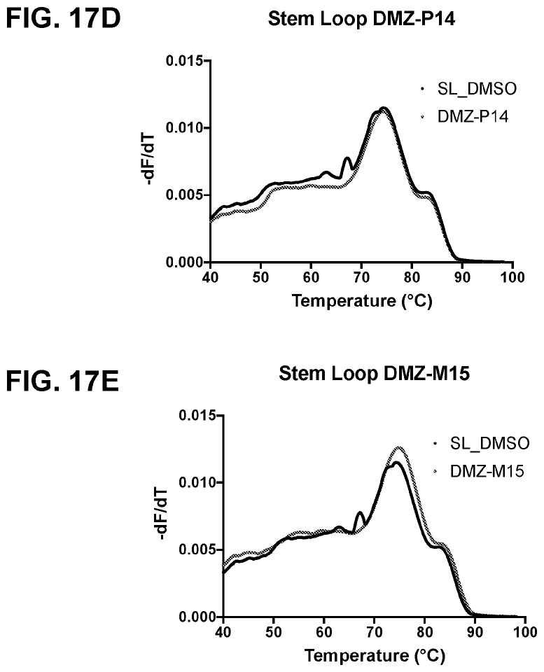

FIG. 16E, FIG. 17A-FIG. 17E). Interestingly, the melting profile of the upper stem of MALAT1 directly overlapped with the second peak of the triplex bi-phasic melting curve, consistent with correspondence of Tm₁ to the melting of the triplex tract and Tm₂ to the melting of the upper stem (FIG. 5B). None of the small molecules caused a substantial change in the melting profiles of either the upper stem or the stem loop proxy, confirming a triplex-selective interaction.

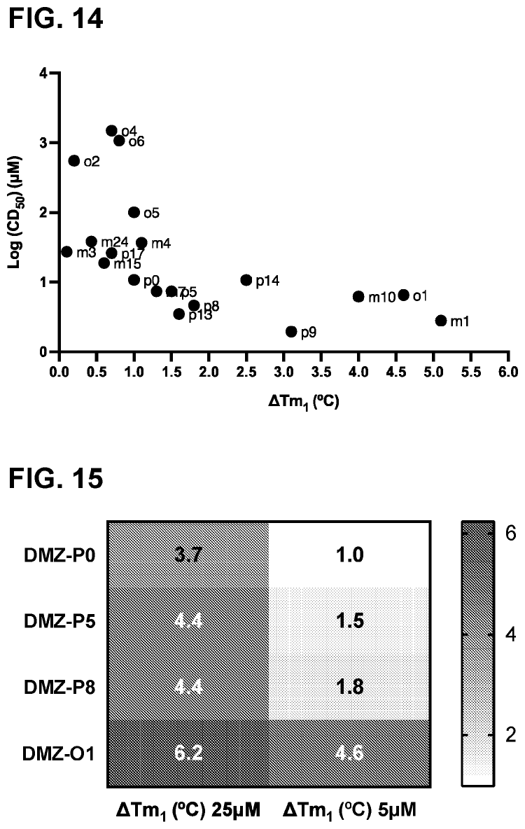

DMZ effects on Tm₁ ranged from negligible (<1° C.) for the biscyano-scaffolds (DMZ-PS, DMZ-MS, DMZ-OS) to approximately 6.1° C. for DMZ-P1. The relative affinity values obtained via the IDA assay was plotted versus the ΔTm₁ values (FIG. 14) and found reasonable correlations at both high and low affinity values (Spearman's correlation R=−0.89, p value <0.0001). Conformity between these two assays comes as a surprise as there was little to no correlation between small molecule affinity and changes in MALAT1 thermal stability. Despite the overall agreement between the two methods, a few discrepancies emerged. Specifically, DMZ-M3, DMZ-P17 and DMZ-M15 showed moderate affinity but negligible thermal stabilization. This discrepancy could be due to different binding modes and/or engagement of a less populated triplex conformer. To gain further insight, two commercially available small molecule leads published by Le Grice (Abulwerdi F A, et al. (2019) ACS Chem Biol. 14(2):223-235) and co-workers were examined using DSF. In that study, the small molecules were screened using a two-body differential scanning FRET (DS-FRET) thermal melting assay and revealed a destabilization of the triple helix complex. (Abulwerdi F A, et al. (2019) ACS Chem Biol. 14(2):223-235). Neither small molecule caused a change in thermal stability using the single body triplex in DSF, indicating that this system might be limited to primarily identifying stabilizing small molecules rather than destabilizing small molecules (FIG. 13A-FIG. 13Z).

The overall agreement between IDA and DSF data prompted us to employ QSAR to test whether the same parameters that yielded a predictive model for IDA are the same that govern thermal stabilization interactions. The model obtained for DSF has higher R² than the one obtained for IDA (R²=0.95, Q²=0.91) indicating an increased robustness of the model (FIG. 5C). The contributive factors identified by the best model are E_stb (bond stretch-bend cross-term potential energy, 3D descriptor), Q_VSA_POL (total polar van der Waals surface area, 2D descriptor) and SMR_VSA7 (sum of van der Waals surface area calculated based on the molar refractivity, 2D descriptor). Polarity, albeit in the form of a different descriptor, is a common factor contributing to IDA and DSF. Additionally, both assays have a combination of 2D and 3D descriptors present in the best models. The combination of E_stb, Q_VSA_POL and SMR_VSA7 indicate that molecular flexibility and the polarizability of the small molecules can lead to stronger complexations that are not disrupted by the addition of heat in DSF experiments and, ultimately, result in stabilization of the triple helix melting event.

### Example 6

### Small Molecule Screening Using an Enzymatic Assay

The library was screened using a more biologically relevant in vitro enzymatic degradation assay. Previous work evaluated the effect on triplex degradation of various ionic buffer conditions (Ageeli A A, et al. (2018) Nucleic Acids Res. 47(3):1468-1481) and a sub-set of DPF small molecules (Donlic A, et al. (2020) Nucleic Acids Res. 48(14):7653-7664) using E. coli RNase R exonuclease as it degrades from 3′ to 5′, reporting on the availability of the poly(A) tail normally sequestered in the triplex construct. While helpful, RNase R does not have a human paralogue and the data collection is time intensive (5 hours/experiment). To overcome these limitations, a fast one-hour enzymatic degradation assay utilizing endonuclease RNase A designed and optimized (FIG. 6A), which is one of the most used, readily available, and best studied members of the RNase superfamilies. (Aryani A, et al. (2015) BMC Res Notes. 8:164; Cuchillo C M, et al. (2011) Biochemistry. 50(37):7835-7841; Kennell D. (2002) J Bacteriol. 184(17):4645-4665). Specificity for ssRNA endonuclease activity was achieved by pre-incubating the enzyme at high salt conditions as previously reported. (Yakovlev G I, et al. (1995) Nucleic Acids Symp Ser. (33):106-108; Struhl K, et al. (1989) Current Protocols in Molecular Biology. 8(1):3.13.1-3.13.3). Aliquot time-points were collected at 0 minutes, 5 minutes, 10 minutes, 30 minutes, and 60 minutes, ultimately yielding a degradation curve for each small molecule while avoiding the non-specific RNA degradation that can occur over longer experiments (FIG. 6B). Small molecules that stabilize the triplex conformer are expected to lead to a decrease in degradation over time, as ssRNA regions are less available to the enzyme. Small molecules that stabilize non-triplex conformations are expected to result in more degradation over time as more ssRNA regions become accessible to RNase A.

The assay was first validated by screening two previously reported stabilizing and destabilizing small molecules, respectively, evaluated with RNase R. Both small molecules displayed the same effect on triplex degradation irrespective of enzyme, confirming the ability of the RNase A assay to similarly reflect small molecule impacts on MALAT1 triplex enzymatic stability.

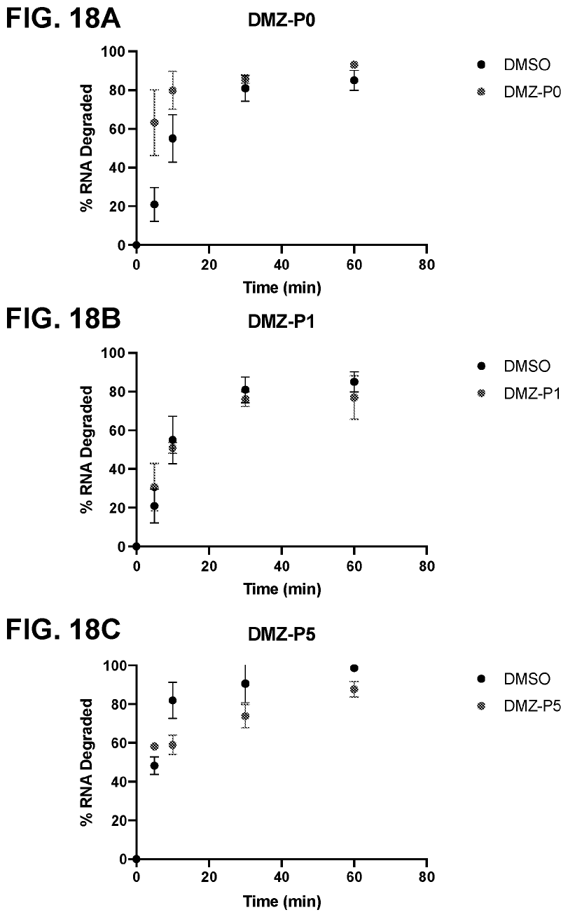

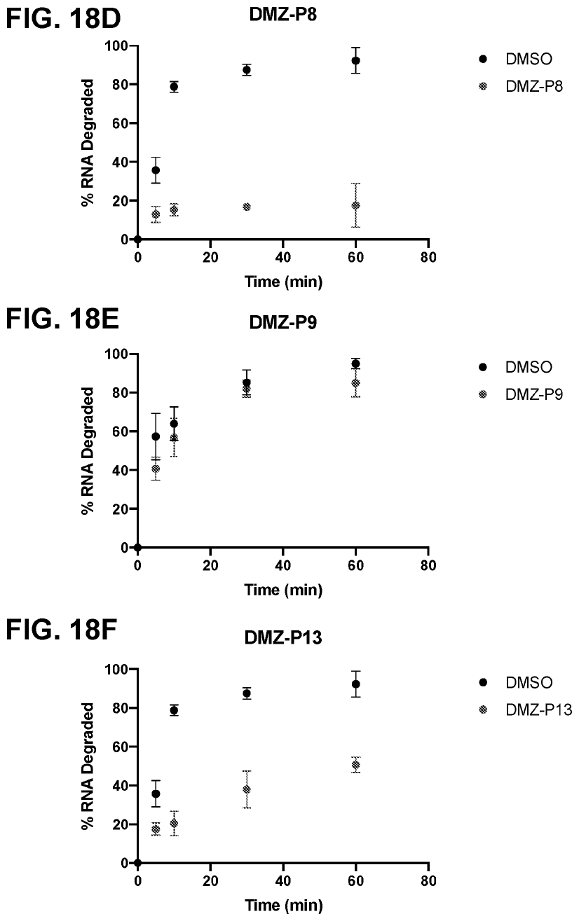

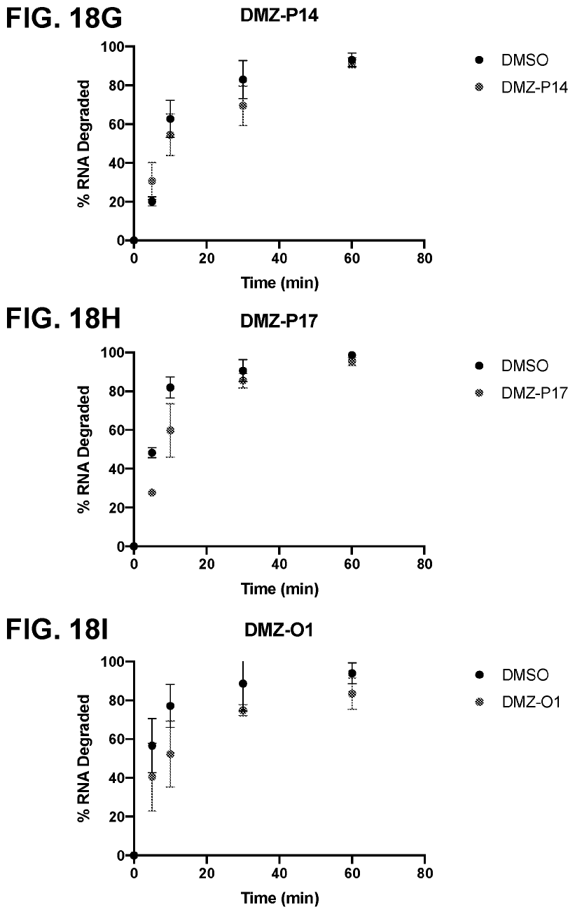

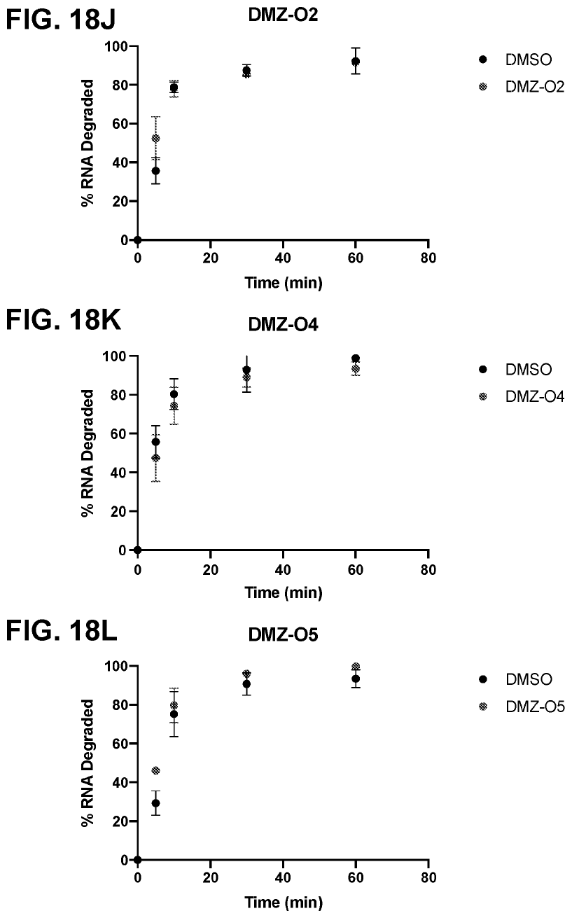

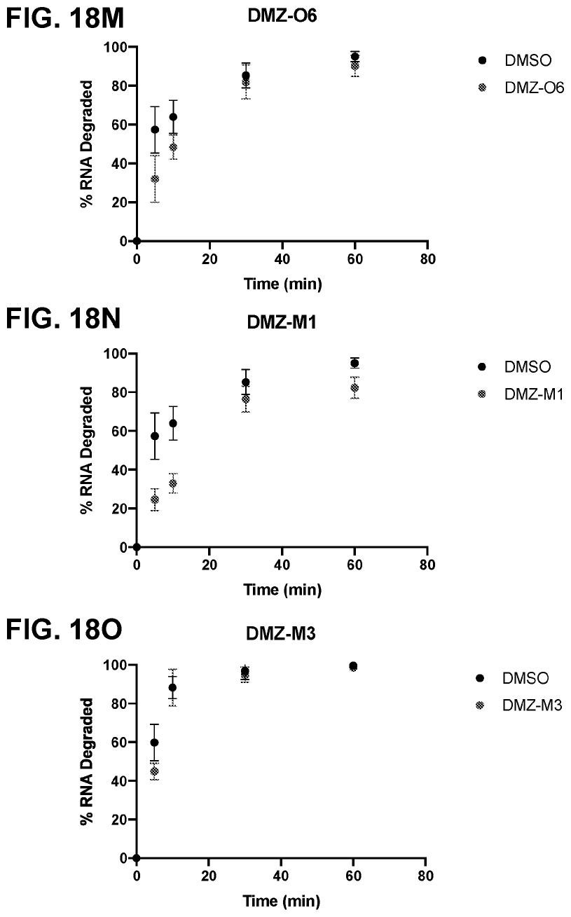

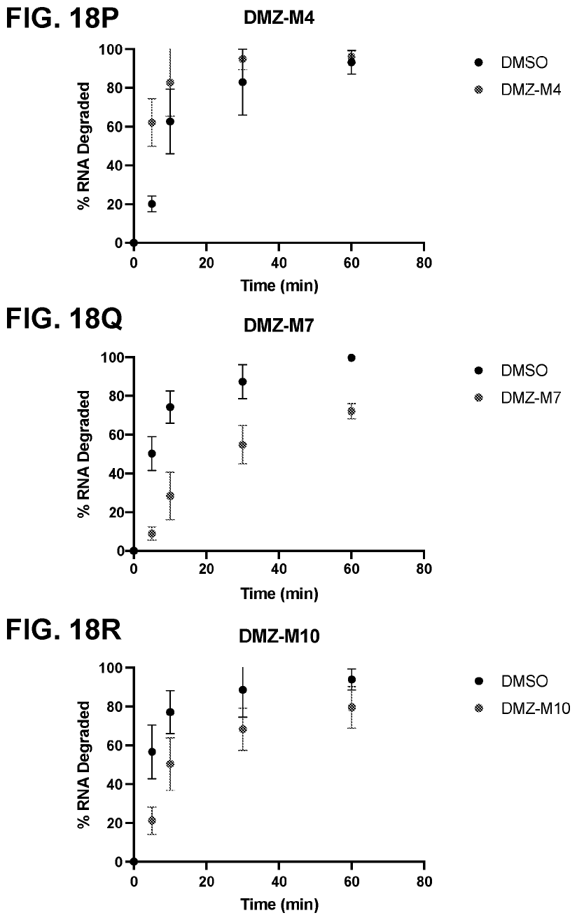

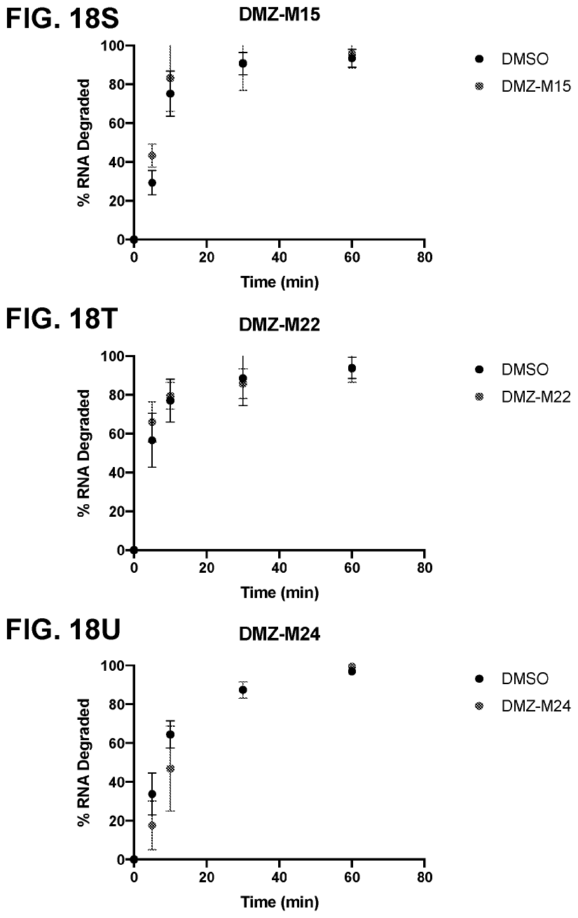

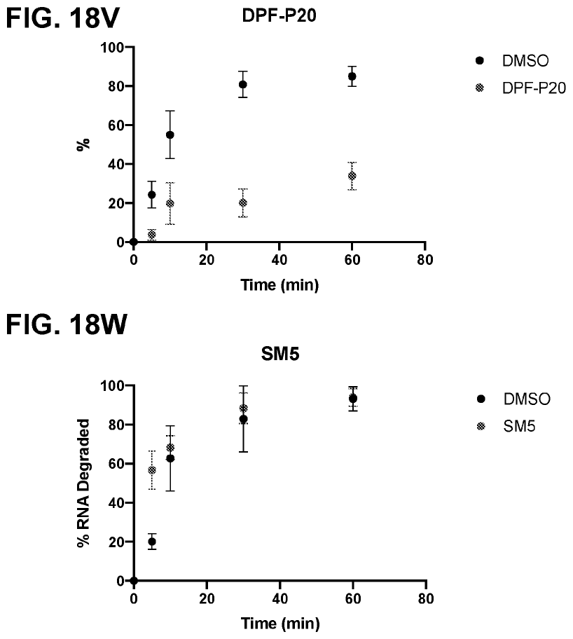

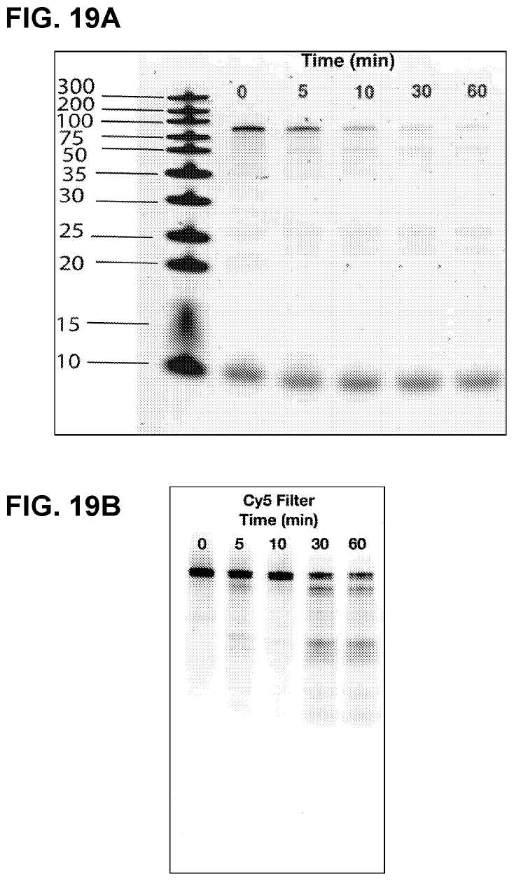

The 21 synthesized DMZ small molecules were screened in three independent replicates (FIG. 18A-FIG. 18W). The ratio of RNA remaining at 5 minutes relative to time 0 was used to classify stabilizing and destabilizing small molecules based on the assumption that the 5-minute time-point falls in the linear phase of decay (FIG. 6C).

Strikingly, 8 out of 21 total small molecules were found to increase the amount of MALAT1 triple helix degraded and were therefore classified as destabilizers. Interestingly, three of the small molecules that showed increases in thermal stability via DSF were found to be destabilizers via RNase A assay (DMZ-P0, DMZ-P1, DMZ-M4), with DMZ-M4 resulting in an increase of the amount of MALAT1 degraded at 5 minutes by 2-fold. Aliphatic subunits with positively charged nitrogens (DMZ-P1, DMZ-O1, DMZ-M1, DMZ-P9, and DMZ-M10), which have consistently shown similar affinity and thermal stability profiles, have vastly different effects on enzymatic degradation of the triplex (Table 5). Notably, DMZ-O1 and DMZ-M1 are strong stabilizers (≥50% decrease in degradation) while DMZ-P1 is a weak destabilizer (<50% increase in degradation). RNase A degradation is the only assay in this study that discriminates between the R-1 regioisomers, indicating that the regioisomers can be engaging different MALAT1 conformers or binding sites. Another example of differential modulation is DMZ-O5, which showed a much stronger destabilizing effect than DMZ-P5. DMZ-O6, on the other hand, is a strong stabilizer and differs from DMZ-O5 by just the position of the chlorine atom on the amine subunit, which is at the ortho-position in DMZ-O6 and at the para-position in DMZ-O5. In addition, subunits that have small architectural differences, like DMZ-O2 and DMZ-O4, which differ by a single carbon atom and have very similar 3D shape as calculated by PMIs, show opposite effects on triplex degradation.

TABLE 5
One-way ANOVA Test for Statistical Significance of RNase
A Normalized Values for the 5-Minute Time Point

| Zidek's Multiple | Mean | 95.00% CI | Below | | Adjusted |
|---|---|---|---|---|---|
| Comparisons Test | Diff. | of diff. | threshold? | Summary | P Value |
| p0 vs. p5 | 2.019 | 0.6813 to 3.356 | Yes | **** | <0.0001 |
| p0 vs. p8 | 2.878 | 1.540 to 4.215 | Yes | **** | <0.0001 |
| p0 vs. p9 | 2.649 | 1.311 to 3.986 | Yes | **** | <0.0001 |
| p0 vs. p13 | 2.735 | 1.397 to 4.072 | Yes | **** | <0.0001 |
| p0 vs. p14 | 1.564 | 0.2261 to 2.901 | Yes | ** | 0.0061 |
| p0 vs. p17 | 2.656 | 1.318 to 3.993 | Yes | **** | <0.0001 |
| p0 vs. o1 | 2.572 | 1.235 to 3.910 | Yes | **** | <0.0001 |
| p0 vs. o2 | 1.594 | 0.2568 to 2.932 | Yes | ** | 0.0045 |
| p0 vs. o4 | 2.38 | 1.043 to 3.718 | Yes | **** | <0.0001 |
| p0 vs. o5 | 1.591 | 0.2537 to 2.929 | Yes | ** | 0.0047 |
| p0 vs. o6 | 2.639 | 1.302 to 3.976 | Yes | **** | <0.0001 |
| p0 vs. m1 | 2.881 | 1.544 to 4.219 | Yes | **** | <0.0001 |
| p0 vs. m3 | 2.474 | 1.137 to 3.812 | Yes | **** | <0.0001 |
| p0 vs. m7 | 3.054 | 1.716 to 4.391 | Yes | **** | <0.0001 |
| p0 vs. m10 | 2.886 | 1.549 to 4.224 | Yes | **** | <0.0001 |
| p0 vs. m15 | 1.702 | 0.3642 to 3.039 | Yes | ** | 0.0016 |
| p0 vs. m22 | 2.13 | 0.7921 to 3.467 | Yes | **** | <0.0001 |
| p0 vs. m24 | 2.314 | 0.9762 to 3.651 | Yes | **** | <0.0001 |
| p1 vs. p8 | 1.559 | 0.2211 to 2.896 | Yes | ** | 0.0064 |
| p1 vs. p13 | 1.415 | 0.07774 to 2.753 | Yes | * | 0.0247 |
| p1 vs. m1 | 1.562 | 0.2244 to 2.899 | Yes | ** | 0.0062 |
| p1 vs. m7 | 1.734 | 0.3968 to 3.072 | Yes | ** | 0.0012 |
| p1 vs. m10 | 1.567 | 0.2295 to 2.904 | Yes | ** | 0.0059 |
| p5 vs. m4 | −1.685 | −3.023 to −0.3479 | Yes | ** | 0.0019 |
| p8 vs. m4 | −2.544 | −3.882 to −1.207 | Yes | **** | <0.0001 |
| p9 vs. m4 | −2.315 | −3.653 to −0.9777 | Yes | **** | <0.0001 |
| p13 vs. m4 | −2.401 | −3.739 to −1.064 | Yes | **** | <0.0001 |
| p14 vs. m7 | 1.49 | 0.1526 to 2.828 | Yes | * | 0.0123 |
| p17 vs. m4 | −2.322 | −3.660 to −0.9848 | Yes | **** | <0.0001 |
| o1 vs. m4 | −2.239 | −3.576 to −0.9015 | Yes | **** | <0.0001 |
| o2 vs. m7 | 1.459 | 0.1219 to 2.797 | Yes | * | 0.0164 |
| o4 vs. m4 | −2.047 | −3.384 to −0.7096 | Yes | **** | <0.0001 |
| o5 vs. m7 | 1.462 | 0.1250 to 2.800 | Yes | * | 0.0159 |
| o6 vs. m4 | −2.306 | −3.643 to −0.9681 | Yes | **** | <0.0001 |
| m1 vs. m4 | −2.548 | −3.885 to −1.210 | Yes | **** | <0.0001 |
| m3 vs. m4 | −2.141 | −3.478 to −0.8035 | Yes | **** | <0.0001 |
| m4 vs. m7 | 2.72 | 1.383 to 4.058 | Yes | **** | <0.0001 |
| m4 vs. m10 | 2.553 | 1.215 to 3.890 | Yes | **** | <0.0001 |
| m4 vs. m15 | 1.368 | 0.03079 to 2.706 | Yes | * | 0.0379 |
| m4 vs. m22 | 1.796 | 0.4586 to 3.134 | Yes | *** | 0.0006 |
| m4 vs. m24 | 1.98 | 0.6428 to 3.318 | Yes | *** | 0.0001 |
| m7 vs. m15 | −1.352 | −2.689 to −0.01453 | Yes | * | 0.0439 |

As a control, protein DSF was conducted on mixtures of the RNase A enzyme and small molecules to assess whether small molecules that resulted in decreased degradation of the RNA triplex over time were binding to the enzyme, which would provide enzyme inhibition as an alternative mechanism. DSF has been used as a tool to identify RNase A inhibitors and have shown that small molecule binders cause a shift in thermal melting of the enzyme, which is tracked by association of the dye SYPRO Orange® to non-solvent exposed hydrophobic regions. (Lang B E, et al. (2017) Biotechnol Prog. 33(3):677-686; Ortiz-Alcantara J, et al. (2010) Virus Adapt Treat 2(1):125-133).

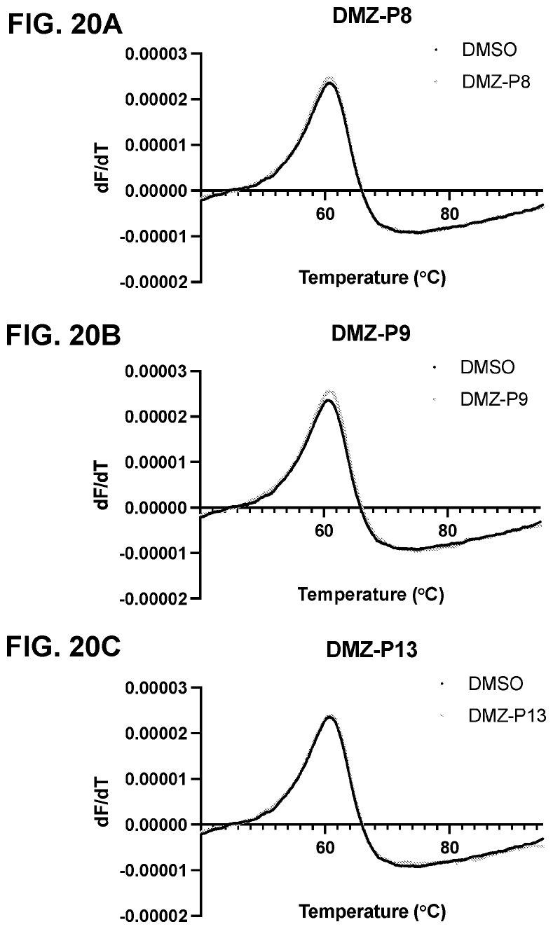

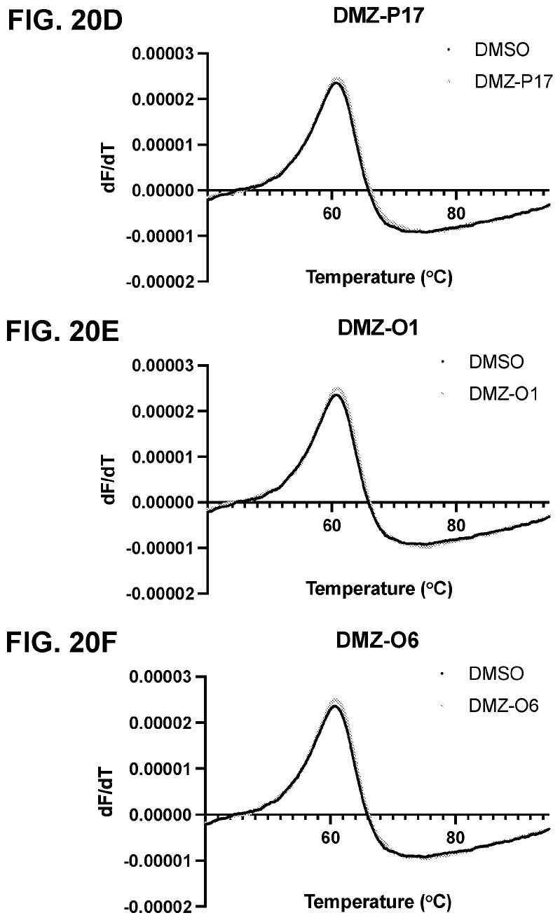

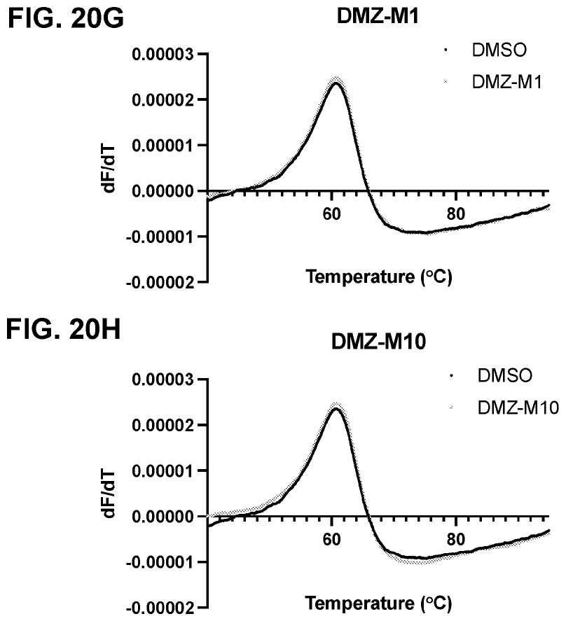

In these studies, the enzyme's melting profile agreed with previous studies and none of the small molecules tested caused an appreciable (>0.5° C.) change in thermal melting, indicating that it is unlikely that the DMZ small molecules act via enzyme inhibition (FIG. 20A-FIG. 20H).

Then, QSAR was performed utilizing the fold-change in small molecule-induced changes in RNA degradation at the 5-minute timepoint of the RNase A assay. QSAR yielded a predictive model with R²=0.74, Q²=0.61 and revealed multiple 3D descriptors as contributing factors (FIG. 6D). For instance, CASA-represents negative charge weighted surface area. Since it has a positive coefficient, the model indicates that increasing this descriptor value will increase the triplex's susceptibility to enzymatic degradation, indicating that small molecules with more negatively charged surface area will destabilize the RNA fold, which is consistent with the nature of negatively charged RNA backbone. Overall, these data underline the RNase A assay's feasibility for efficient assessment of small molecule impacts on the susceptibility of RNA structures to degradation.

This is the first report of dual modulation of an RNA triple helix within the same scaffold class, supporting diminazene as a valuable probe for future studies of MALAT1 conformational dynamics and modulation.

### Example 7

### Small Molecule Characterization

Spectral characterization for the library of small molecules was performed.

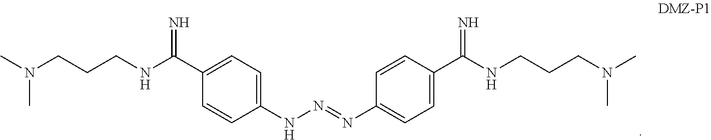

Bright red solid; Yield=36%; ¹H NMR (500 MHZ, Methanol-d4) δ 7.62-7.59 (m, 4H), 7.48-7.44 (m, 4H), 3.31 (t, J=6.8 Hz, 4H), 2.35 (t, J=7.2 Hz, 4H), 2.18 (s, 12H), 1.79 (p, J=7.0 Hz, 4H). ¹³C NMR (126 MHz, MeOD) δ 163.49, 150.80, 128.24, 127.72, 117.81, 56.34, 44.49, 44.02, 43.95, 41.15, 25.89. HRMS-ESI (m/z) Calcd for C₂₄H₃₇N₉ ([M+H]+): 452.3; found: 452.3(±0.4 ppm).

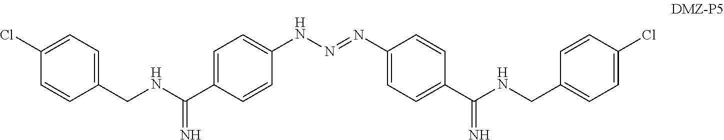 [SMILES: N=C(NCc1ccc(Cl)cc1)c1ccc(/N=N/Nc2ccc(C(=N)NCc3ccc(Cl)cc3)cc2)cc1]

Bright yellow solid; Yield=51%; ¹H NMR (500 MHZ, Methanol-d4) δ 7.65-7.61 (m, 4H), 7.43 (dd, J=8.6, 2.1 Hz, 4H), 7.29-7.23 (m, 8H), 4.39 (s, 4H). ¹³C NMR (126 MHZ, MeOD) δ 162.78, 148.62, 137.23, 132.58, 130.72, 129.06, 128.85, 128.25, 128.08, 117.45, 46.39. HRMS-ESI (m/z) Calcd for C₂₈H₂₅C₁₂N₇ ([M+H]+): 530.2; found: 530.2(±0.7 ppm).

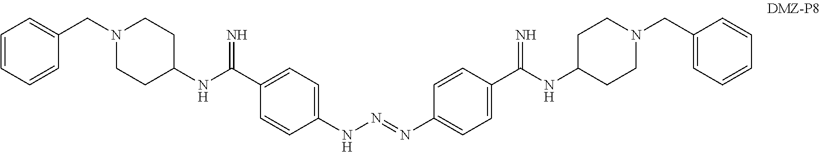 [SMILES: N=C(NC1CCN(Cc2ccccc2)CC1)c1ccc(/N=N/Nc2ccc(C(=N)NC3CCN(Cc4ccccc4)CC3)cc2)cc1]

Bright orange solid; Yield=36%; ¹H NMR (500 MHZ, Methanol-d4) δ 7.79 (d, J=8.4 Hz, 4H), 7.68 (d, J=8.4 Hz, 4H), 7.37-7.34 (m, 8H), 7.30 (dd, J=5.9, 2.8 Hz, 2H), 3.74 (ddd, J=11.2, 6.8, 4.1 Hz, 2H), 3.60 (s, 4H), 3.02 (dt, J=12.6, 3.6 Hz, 4H), 2.27-2.21 (m, 4H), 2.10-2.06 (m, 4H), 1.80 (dt, J=12.1, 6.0 Hz, 4H). ¹³C NMR (126 MHz, MeOD) δ 129.23, 127.98, 127.11, 62.39, 51.48, 48.12, 47.94, 47.77, 47.09, 30.01. HRMS-ESI (m/z) Calcd for C₃₈H₄₅N₉ ([M+H]+): 628.4; found: 628.4(±1.0 ppm).

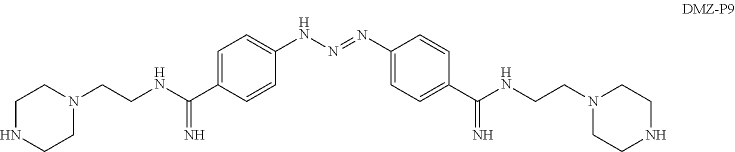 [SMILES: N=C(NCCN1CCNCC1)c1ccc(/N=N/Nc2ccc(C(=N)NCCN3CCNCC3)cc2)cc1]

Bright yellow solid; Yield=21%; ¹H NMR (500 MHZ, Deuterium Oxide) δ 7.69 (d, J=8.2 Hz, 4H), 7.54 (d, J=8.3 Hz, 4H), 3.55 (t, J=6.3 Hz, 4H), 3.25 (t, J=5.2 Hz, 8H), 2.81 (d, J=6.2 Hz, 12H). ¹³C NMR (126 MHz, D2O) δ 163.85, 129.29, 54.44, 49.15, 43.19, 39.97. HRMS-ESI (m/z) Calcd for C₂₆H₃₉N₁₁ ([M+H]+): 506.3; found: 506.3(±1.2 ppm).

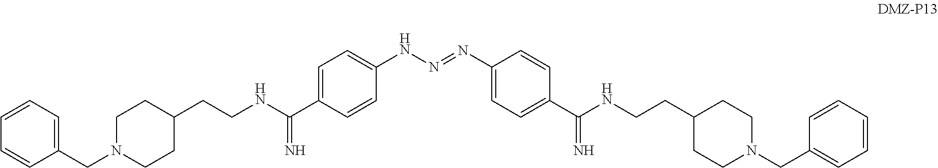 [SMILES: N=C(NCCC1CCN(Cc2ccccc2)CC1)c1ccc(/N=N/Nc2ccc(C(=N)NCCC3CCN(Cc4ccccc4)CC3)cc2)cc1]

Bright orange solid; Yield=27%; ¹H NMR (500 MHz, Methanol-d4) δ 7.82 (d, J=8.3 Hz, 4H), 7.68 (d, J=8.3 Hz, 4H), 7.55-7.45 (m, 10H), 4.28 (s, 4H), 3.56-3.44 (m, 8H), 3.00 (t, J=12.6 Hz, 4H), 2.04 (d, J=14.1 Hz, 4H), 1.78 (q, J=6.5 Hz, 6H), 1.59 (q, J=11.6, 10.9 Hz, 4H). ¹³C NMR (126 MHz, MeOD) δ 163.89, 161.77, 161.49, 130.85, 129.61, 129.13, 128.87, 118.03, 115.70, 51.89, 47.45, 47.28, 47.11, 40.34, 32.75, 31.15, 28.66. HRMS-ESI (m/z) Calcd for C₄₂H₅₃N₉ ([M+H]+): 684.45; found: 684.45(±3.3 ppm).

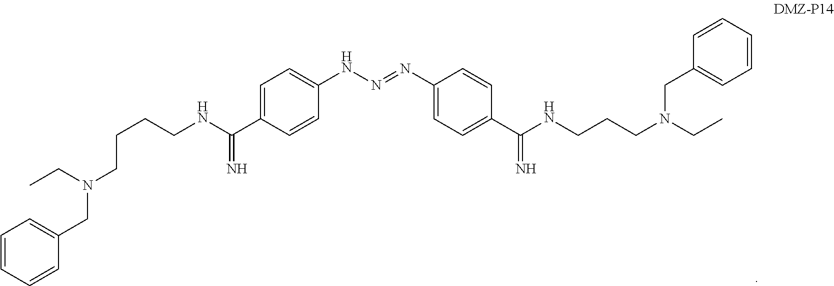 [SMILES: CCN(CCCCNC(=N)c1ccc(N/N=N/c2ccc(C(=N)NCCCN(CC)Cc3ccccc3)cc2)cc1)Cc1ccccc1]

Bright orange solid; Yield=32%; ¹H NMR (500 MHZ, Methanol-d4) δ 7.62-7.58 (m, 4H), 7.55 (d, J=8.7 Hz, 4H), 7.28-7.25 (m, 4H), 7.24-7.20 (m, 4H), 7.19-7.16 (m, 2H), 3.73 (s, 4H), 2.70 (d, J=7.7 Hz, 6H), 1.92 (p, J=6.9 Hz, 4H), 1.09 (t, J=7.1 Hz, 6H). ¹³C NMR (126 MHz, MeOD) δ 163.87, 161.78, 129.50, 129.08, 128.30, 118.03, 115.70, 57.17, 49.69, 48.12, 46.97, 44.39, 41.02, 23.78, 9.35. HRMS-ESI (m/z) Calcd for C₃₉H₅₁N₉ ([M+H]+): 645.4; found: 645.8(±2.0 ppm).

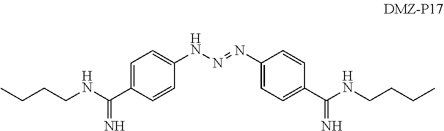 [SMILES: CCCCNC(=N)c1ccc(/N=N/Nc2ccc(C(=N)NCCCC)cc2)cc1]

Bright red solid; Yield=82%; ¹H NMR (500 MHZ, Methanol-d4) δ 7.68-7.64 (m, 4H), 7.52 (d, J=8.4 Hz, 4H), 3.31 (d, J=7.3 Hz, 4H), 1.70 (tt, J=7.8, 6.6 Hz, 4H), 1.49 (h, J=7.4 Hz, 4H), 1.02 (t, J=7.4 Hz, 6H). ¹³C NMR (126 MHz, MeOD) δ 163.48, 150.10, 130.22, 127.75, 117.56, 47.11, 45.79, 42.49, 30.68, 20.05, 12.86. HRMS-ESI (m/z) Calcd for C₂₂H₃₉N₁₁ ([M+H]+): 645.4; found: 645.8(±2.0 ppm).

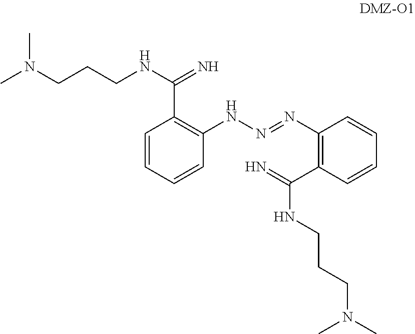

Bright red solid; Yield=45%; ¹H NMR (500 MHZ, Methanol-d4) δ 7.77 (d, J=8.3 Hz, 2H), 7.61 (d, J=7.9 Hz, 2H), 7.51 (ddd, J=8.5, 7.2, 1.4 Hz, 2H), 7.17-7.13 (m, 2H), 3.43 (t, J=6.6 Hz, 4H), 2.43 (t, J=7.2 Hz, 4H), 2.26 (s, 12H), 1.91-1.86 (m, 4H). ¹³C NMR (126 MHZ, MeOD) δ 150.85, 132.16, 127.98, 122.38, 118.18, 56.13, 43.90, 40.84, 25.71. HRMS-ESI (m/z) Calcd for C₂₄H₃₇N₉ ([M+H]+): 452.3; found: 452.3(±0.4 ppm).

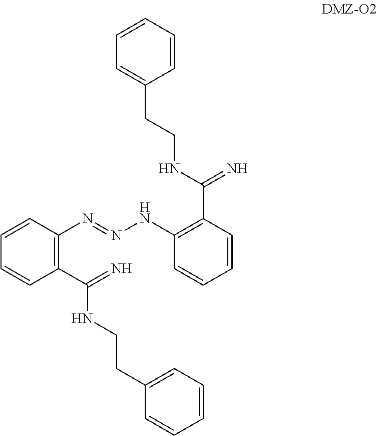 [SMILES: N=C(NCCc1ccccc1)c1ccccc1/N=N/Nc1ccccc1C(=N)NCCc1ccccc1]

Pale yellow solid; Yield=42%; ¹H NMR (500 MHZ, Methanol-d4) δ 7.82 (d, J=8.3 Hz, 2H), 7.53 (ddd, J=9.5, 6.9, 2.6 Hz, 4H), 7.30-7.25 (m, 10H), 7.17-7.14 (m, 2H), 2.89 (t, J=7.1 Hz, 4H), 2.77 (t, J=7.3 Hz, 4H). ¹³C NMR (126 MHZ, MeOD) δ 138.26, 132.32, 128.60, 128.33, 126.41, 123.36, 119.60, 117.57, 115.77, 48.15, 47.13, 45.80, 34.05. HRMS-ESI (m/z) Calcd for C₃₀H₃₁N₇ ([M+H]+): 490.3; found: 490.3(±0.9 ppm).

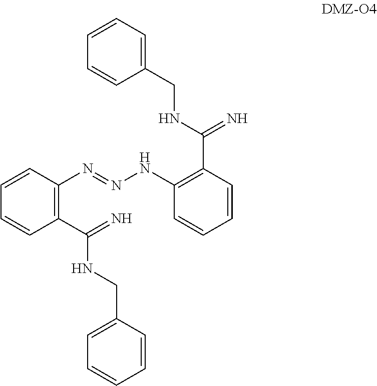 [SMILES: N=C(NCc1ccccc1)c1ccccc1/N=N/Nc1ccccc1C(=N)NCc1ccccc1]

Pale yellow solid; Yield=46%; ¹H NMR (500 MHz, Methanol-d4) δ 7.78 (d, J=8.2 Hz, 2H), 7.63 (d, J=7.8 Hz, 2H), 7.48-7.44 (m, 2H), 7.39 (d, J=7.7 Hz, 4H), 7.31 (t, J=7.6 Hz, 4H), 7.23-7.19 (m, 4H), 4.44 (s, 4H). ¹³C NMR (126 MHZ, MeOD) δ 145.61, 138.77, 130.58, 128.17, 128.15, 127.23, 126.61, 123.76, 116.80, 45.79, 44.46. HRMS-ESI (m/z) Calcd for C₂₈H₂₇N₇ ([M+H]+): 462.2; found: 462.2(±2.6 ppm).

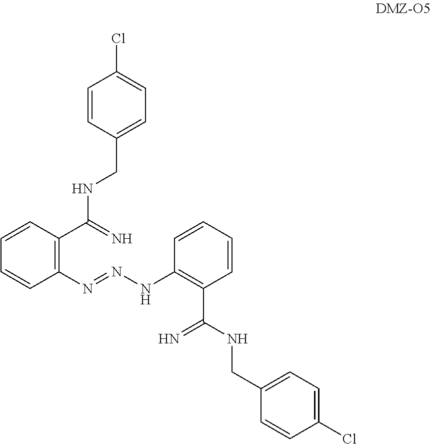 [SMILES: N=C(NCc1ccc(Cl)cc1)c1ccccc1/N=N/Nc1ccccc1C(=N)NCc1ccc(Cl)cc1]

Bright yellow solid; Yield=31%; ¹H NMR (500 MHZ, Methanol-d4) δ 7.79-7.76 (m, 2H), 7.63 (d, J=7.8 Hz, 2H), 7.48 (ddd, J=8.5, 7.3, 1.5 Hz, 2H), 7.37-7.34 (m, 4H), 7.28 (d, J=8.4 Hz, 4H), 7.22 (td, J=7.6, 1.2 Hz, 2H), 4.43 (s, 4H). ¹³C NMR (126 MHz, MeOD) δ 161.66, 145.60, 137.54, 132.25, 130.76, 128.88, 128.80, 128.18, 128.13, 123.87, 116.87, 48.48, 45.79. HRMS-ESI (m/z) Calcd for C₂₈H₂₅Cl₂N₇ ([M+H]+): 530.2; found: 530.2(±0.6 ppm).

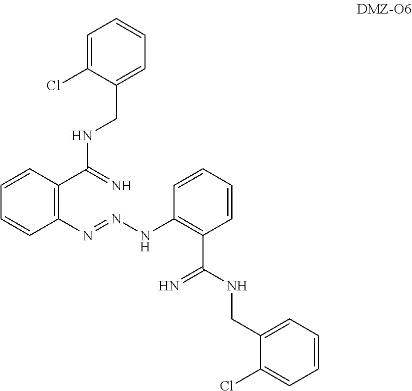 [SMILES: N=C(NCc1ccccc1Cl)c1ccccc1/N=N/Nc1ccccc1C(=N)NCc1ccccc1Cl]

Bright yellow solid: Yield=11%; ¹H NMR (500 MHZ, Methanol-d4) δ 7.78 (d, J=8.2 Hz, 2H), 7.64 (d, J=7.7 Hz, 2H), 7.53 (d, J=7.5 Hz, 2H), 7.47 (ddd, J=8.6, 7.3, 1.5 Hz, 2H), 7.39-7.34 (m, 2H), 7.26-7.19 (m, 6H), 4.49 (s, 4H). ¹³C NMR (126 MHZ, MeOD) δ 130.43, 128.93, 128.87, 128.24, 127.95, 126.76, 53.46, 47.58, 47.44, 47.42, 47.27, 47.10. HRMS-ESI (m/z) Calcd for C₂₈H₂₅C₁₂N₇ ([M+H]+): 530.1621; found: 530.1624 (=0.5 ppm).

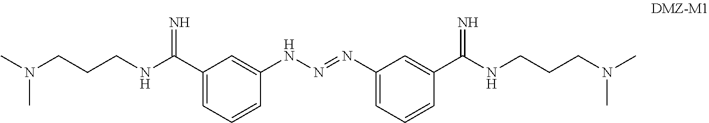

¹H NMR (500 MHZ, Methanol-d4) δ 7.77 (t, J=2.0 Hz, 2H), 7.73 (dd, J=8.4, 2.0 Hz, 2H), 7.53 (t, J=7.9 Hz, 2H), 7.46-7.43 (m, 2H), 3.46 (t, J=6.8 Hz, 4H), 2.47 (t, J=7.0 Hz, 4H), 2.27 (s, 12H), 1.88 (p, J=7.0 Hz, 4H). ¹³C NMR (126 MHz, MeOD) δ 164.55, 130.40, 130.18, 123.77, 122.05, 116.84, 55.61, 43.68, 41.04, 24.91. HRMS-ESI (m/z) Calcd for C₂₄H₃₇N₉ ([M+H]+): 452.3; found: 452.3(±1.9 ppm).

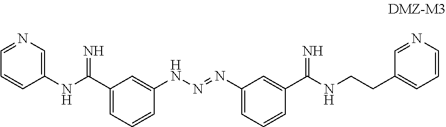 [SMILES: N=C(NCCc1cccnc1)c1cccc(/N=N/Nc2cccc(C(=N)Nc3cccnc3)c2)c1]

Beige solid; Yield=32% ¹H NMR (500 MHZ, Methanol-d4) δ 8.39 (s, 2H), 8.29 (d, J=4.7 Hz, 2H), 7.73 (d, J=7.8 Hz, 2H), 7.61 (s, 2H), 7.53 (d, J=8.1 Hz, 2H), 7.39 (t, J=7.9 Hz, 2H), 7.28 (dd, J=7.9, 4.2 Hz, 4H), 3.54 (t, J=7.3 Hz, 4H), 2.97 (t, J=7.2 Hz, 4H). ¹³C NMR (126 MHZ, MeOD) δ 163.53, 149.17, 146.83, 137.45, 135.55, 134.73, 129.57, 123.89, 123.11, 120.60, 116.07, 47.62, 47.45, 47.28, 47.11, 43.96, 31.37. HRMS-ESI (m/z) Calcd for C₂₈H₂₉N₉ ([M+H]+): 492.6; found: 246.3(±1.1 ppm) for 1/2[M+H]+.

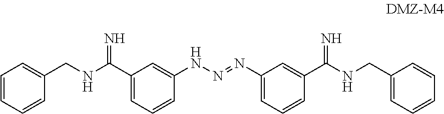 [SMILES: N=C(NCc1ccccc1)c1cccc(/N=N/Nc2cccc(C(=N)NCc3ccccc3)c2)c1]

Beige solid; Yield=21%; ¹H NMR (500 MHZ, Methanol-d4) δ 7.66 (t, J=1.9 Hz, 2H), 7.44 (dt, J=7.4, 1.9 Hz, 2H), 7.34-7.30 (m, 4H), 7.29-7.25 (m, 6H), 7.21 (dd, J=8.5, 6.9 Hz, 5H), 7.13-7.10 (m, 2H), 4.36 (s, 4H). ¹³C NMR (126 MHZ, MeOD) δ 163.02, 146.29, 139.18, 137.88, 129.15, 128.14, 127.17, 126.61, 122.84, 119.26, 115.97, 45.79. HRMS-ESI (m/z) Calcd for C₂₈H₂₉N₉ ([M+H]+): 492.6; found: 246.3(±1.1 ppm) for [M+H]+.

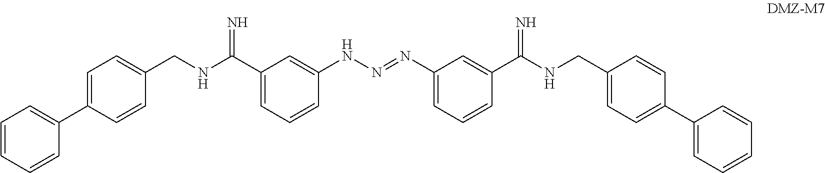 [SMILES: N=C(NCc1ccc(-c2ccccc2)cc1)c1cccc(/N=N/Nc2cccc(C(=N)NCc3ccc(-c4ccccc4)cc3)c2)c1]

Yellow solid; Yield=12%; ¹H NMR (500 MHZ, Methanol-d4) δ 7.91 (s, 2H), 7.81 (d, J=8.0 Hz, 2H), 7.71-7.68 (m, 4H), 7.63 (d, J=4.8 Hz, 6H), 7.53 (d, J=7.9 Hz, 4H), 7.45 (d, J=7.7 Hz, 4H), 7.37 (t, J=7.5 Hz, 2H), 3.33 (d, J=2.9 Hz, 4H). ¹³C NMR (126 MHZ, MeOD) δ 164.24, 141.03, 140.34, 134.22, 131.52, 130.02, 128.56, 127.88, 127.23, 127.16, 126.56, 123.71, 121.74, 116.75, 46.11, 45.77. HRMS-ESI (m/z) Calcd for C₄₀H₃₅N₇ ([M+H]+): 614.3; found: 614.3(±1.9 ppm) for [M+H]+.

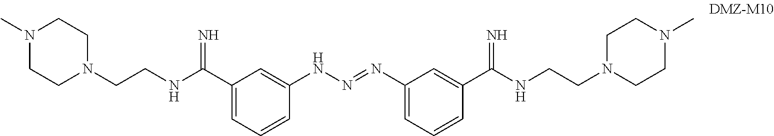

Bright yellow solid; Yield=44%; ¹H NMR (500 MHZ, Methanol-d4) δ 7.96 (t, J=2.0 Hz, 1H), 7.79-7.75 (m, 1H), 7.63-7.61 (m, 2H), 7.47 (dd, J=17.0, 7.6 Hz, 4H), 3.58 (t, J=6.8 Hz, 2H), 3.48 (t, J=6.5 Hz, 2H), 2.86 (t, J=5.1 Hz, 12H), 2.76 (t, J=6.6 Hz, 6H), 2.45 (t, J=6.7 Hz, 8H). ¹³C NMR (126 MHZ, MeOD) δ 168.42, 163.41, 146.46, 135.49, 129.32, 123.07, 120.52, 116.38, 60.73, 57.42, 53.66, 44.70, 37.43, 36.45. HRMS-ESI (m/z) Calcd for C₂₈H₂₃N₇O₂ ([M+H]+): 534.4; found: 534.4 (=1.3 ppm).

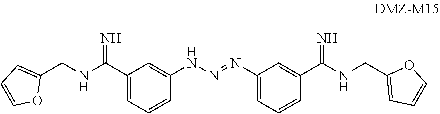 [SMILES: N=C(NCc1ccco1)c1cccc(/N=N/Nc2cccc(C(=N)NCc3ccco3)c2)c1]

Bright yellow solid; Yield=23%; ¹H NMR (500 MHZ, Methanol-d4) δ 7.65 (s, 2H), 7.50-7.44 (m, 2H), 7.35 (s, 2H), 7.33 (d, J=6.8 Hz, 4H), 6.26 (dd, J=11.6, 2.7 Hz, 4H), 4.37 (s, 4H). ¹³C NMR (126 MHz, MeOD) δ 162.67, 151.94, 146.27, 141.95, 136.32, 129.21, 123.09, 119.79, 116.15, 110.00, 106.92, 40.93. HRMS-ESI (m/z) Calcd for C24H23N11 ([M+H]+): 442.2; found: 442.2(±2.9 ppm).

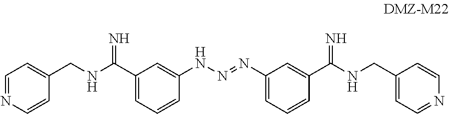 [SMILES: N=C(NCc1ccncc1)c1cccc(/N=N/Nc2cccc(C(=N)NCc3ccncc3)c2)c1]

Pale rose solid; Yield=22%; ¹H NMR (500 MHZ, Methanol-d4) δ 8.96 (s, 4H), 8.29-8.15 (m, 4H), 8.01-7.67 (m, 8H), 5.18 (d, J=11.1 Hz, 4H). ¹³C NMR (126 MHz, MeOD) δ 156.90, 150.88, 148.39, 133.73, 122.95, 122.35, 119.92, 118.36, 117.48, 117.38, 37.30. HRMS-ESI (m/z) Calcd for C₂₆H₂₅N₉ ([M+H]+): 464.2; found: 464.2(±0.6 ppm).

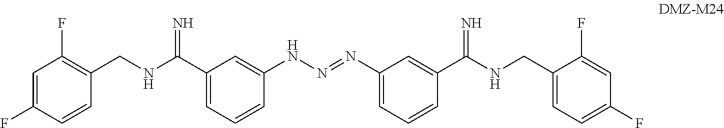 [SMILES: N=C(NCc1ccc(F)cc1F)c1cccc(/N=N/Nc2cccc(C(=N)NCc3ccc(F)cc3F)c2)c1]

Pale brown solid; Yield=11%; ¹H NMR (500 MHZ, Methanol-d4) δ 7.67 (s, 2H), 7.45 (dd, J=7.3, 2.2 Hz, 2H), 7.34 (dt, J=11.7, 7.1 Hz, 6H), 6.85-6.80 (m, 4H), 4.36 (s, 4H). ¹³C NMR (126 MHz, MeOD) δ 163.05, 162.24, 161.79, 161.08, 159.73, 146.06, 137.60, 130.30, 128.94, 122.76, 122.39, 119.07, 115.87, 110.63, 102.64, 41.01. HRMS-ESI (m/z) Calcd for C₂₈H₂₃F₄N₇ ([M+H]+): 534.2; found: 534.2(±1.5 ppm).

### Example 8

### RNase A Differential Scanning Fluorimetry (DSF)

All experiments were performed in Roche 96 well plates in a LightCycler® (Roche). In a typical experiment, 10 μM RNase A was diluted in the same buffer used for RNase A degradation assays (20 mM HEPES-KOH, 52.6 mM KCl, 0.1 mM MgCl₂, pH=7.4). 5000×SYPRO™ Orange Protein Gel Stain (ThermoFisher Scientific) dye stock solution was diluted to 10× in the same buffer with the RNase A enzyme. The mixture was then aliquoted in the light cycler plates by adding 49 μL per well. A 2.5 mM final concentration of ligand was made by adding 1 mL of DMSO or small molecule to each well. The mixture was left to incubate at room temperature for 10 minutes with a black lid, the 96 well plate was then sealed with an optically clear foil and centrifuged for 1 minute at 4000 rpm prior to being placed in the instrument. A high-resolution melting profile was obtained by monitoring fluorescence intensity using the VIC dye filter combination (577-620 nm) from 37-98° C. at a ramp rate of 0.01° C./second with 120 acquisitions per° C. Melting temperatures were obtained by Tₘ analysis in the LightCycler® software (W 1.1) of the derivative of fluorescence over time.

### Example 9

### In Vivo Toxicity in LNCaP Cells

LNCaP is a cell line derived from a metastatic lymph node lesion of human prostate cancer which is androgen receptor (AR) positive, exhibits androgen-sensitive growth. In the first set of in vivo experiments, four (4) members of the small molecule library were examined in a MTT assay. The IC₅₀ at 48 hours (FIG. 23A) and 72 hours (FIG. 23B) was determined for DMZ-P0, DMZ-P5, DMZ-P13, and DMZ-P17. At 48 hours, DMZ-P13 had the lowest IC₅₀ value of 4.1 μM. At 72 hours, DMZ-P13 had the lowest IC₅₀ value of 2.7 μM. The IC₅₀ values and trends were similar to the apparent small molecule affinities for MALAT-1, supporting a MALAT-1 related mechanism for the toxicity. DMZ-P0 was an exception, with a much higher IC₅₀, possibly indicating a lack of cell uptake or off target engagement. Ongoing cytotoxicity studies using other DMZ small molecule analogs (as well as DMZ-5, DMZ-P13, and DMZ-P17) continue to illuminate mechanism of action regarding toxicity to cancer cells.

In a second set of in vivo experiments, four (4) additional members (DMZ-M1, DMZ-M4, DMZ-M7, and DMZ-M10) of the small molecular library were examined in a MTT assay. At 48 hours, DMZ-M7 had the lowest IC₅₀ of 2.9 μM. The IC₅₀ values and trends are similar to the apparent small molecule affinities for MALAT-1, supporting a MALAT-1 related mechanism for the toxicity. Here, DMZ-M10 was an exception, with a higher IC₅₀, possibly indicating a lack of cell uptake or off target engagement. Ongoing cytotoxicity studies using other DMZ small molecule analogs (as well as DMZ-M7, DMZ-M1, and DMZ-M4) continue to illuminate mechanism of action regarding toxicity to cancer cells.

### Summary of Experiments Example 1-Example 9

In this study, a 3D-shape diverse focused library was synthesized from an under-explored scaffold to elucidate molecular recognition properties of modulators of MALAT1 triplex stability. Following the design of a focused theoretical library, 21 representative members were synthesized based on Berenil®, an FDA-approved antiparasitic scaffold with limited synthetic exploration but reported nucleic acid binding properties. To gain a holistic perspective on small molecule trends as a function of the assay, the library was assessed against the MALAT1 triple helix using a multi-dimensional approach that involved designing and optimizing both affinity-based and structural-stability based assays.

Evaluation of the library using an IDA assay reported a range of affinities from low to high micromolar that were in good agreement with ITC affinity values. Analysis of ligand 3D shape confirmed rod-likeness as a predictor of small molecule affinity for the triple helix. The diminazene molecules that showed the highest affinity measured via IDA also caused the greatest temperature increase in thermal melting via DSF. RNase A trends were vastly different, and in some cases opposing, relative to trends observed in the IDA and DSF assays. The discrepancies observed between the affinity, thermal stability, and enzymatic degradation might be due to differences in the triplex conformation reported on by each assay. Indeed, affinity and thermal stability assays are equilibrium-based and thus may report on the more populated triplex conformers. On the other hand, binding to a less populated conformer might shift the equilibrium enough to allow irreversible degradation of the RNA construct. This hypothesis could explain why, for example, DMZ-M4 showed mediocre affinity and thermal stabilization but resulted in >2-fold increase in MALAT1 triplex degraded after 5 minutes. Structural studies are underway to better understand the RNA:small molecule conformational landscape in order to gain insight into the conformers engaged by both stabilizing and destabilizing small molecules.

To gain deeper understanding of these trends, QSAR was employed. For affinity and effect on thermal stability, the best predicting models included a combination of 3D and 2D parameters. Application of QSAR on RNase A data, on the other hand, resulted in a model composed of 3D parameters only. It is noteworthy that despite the limited size of the focused library, a model with R²=0.74 was generated and was informative of the classification of stabilizers and destabilizers, with predictive classification accuracy of 0.62 (8 out of 13) and 1 (8 out of 8), respectively.

The data presented herein provide essential insight into the development of small molecule modulators of the MALAT1 triple helix. The diminazene scaffold has revealed that both stabilization and destabilization of the triple helix can be achieved through synthetic tuning of a single scaffold, indicating that DMZs may make an excellent chemical probe to study the effects of small molecules on transcript half-life and phenotypic traits in MALAT1-overexpressing cell lines.

The interrogation of the relationship between small molecule engagement of RNA triple helices and the effect on triplex conformational dynamics lays the foundation for small molecule-mediated spatiotemporal control of association of triple helix motifs with their cellular interactome.

The RNase A assay as a preliminary stepping stone toward addressing a largely unmet need in the field of RNA:small molecule studies, namely the design of functional high throughput and cost efficient in vitro assays that report on the effect of small molecules on RNA conformational dynamics. Such in vitro functional assays would be the missing bridge between affinity- and activity-based screenings to ultimately allow function-based ligand design and a better understanding of the relationship between RNA conformation and cellular function. This workflow illustrates the potential of QSAR-mediated trend analysis in search of guiding principles. Library sizes can be increased, which in turns, increase chemical diversity—all ensuring that more accurate models are obtained.

From these studies, a few points emerged: (a) small molecule targeting of the MALAT1 triple helix in vitro and in cellulo was feasible; (b) a focused library approach led to a higher hit rate than high-throughput-based approaches; (c) a possible rod-like trend was identified amongst the best small molecule binders; (d) small molecules both increased or decreased MALAT1 susceptibility to degradation; and (e) binding strength and impact on degradation may not be correlated.

Collectively, the findings and novel tools presented herein raise important considerations for ligand and assay design for RNA triple helices. The evaluation of the complete focused library using a multi-assay approach and subsequent employment of computational tools provided a better understanding of the parameters that impact different assessments and paves the way for future work to evaluate both selectivity and correlation with biological activity. The discovery of a predictive model for small molecule impacts on stabilization and destabilization of an RNA triple helix opens the door for function-based rational design of small molecule triple helix modulators, which are uniquely poised to provide essential insight into the relationship between biological outcome and modulation of a triple helix dynamic landscape.

### SUMMARY OF EXPERIMENTS

The methodical approach described herein, which uses computational parameters (e.g., shape) and are derived from analysis of bioactive small molecules that target RNA, is new and exciting. The approach is both unique and non-obvious because currently the standard in industry (as detailed in numerous publication) relies on simple binding rather than bioactivity. By considering bioactivity, this methodological approach considers uptake, selectivity, and modulation of function. Thus, this approach is significantly more effective for identifying small molecule and library design than what is currently available in the industry.

Moreover, the uses described herein are also new and exciting. The approach again differs from the industry standard in that the industry relies on the process of optimizing binding and binding-based selectivity prior to examining function in the cell. At this stage, many RNA targeting efforts in the industry fail because binding in and of itself does not provide information about the modulation of conformation. The lack of the industry's consideration of modulation of conformation is significant. The methodology described here, however, does consideration modulation of conformation and it can be examined using the disclosed function-proximal assays.

Finally, as only described herein, the use of quantitative structure activity relationship to predict small molecule binding and functional modulation of RNA has not been done. Nor has there been any use or attempt to use that relationship to target RNA.

The studies discussed herein represent the first methodical approach employing rational design and optimization of RNA targeted ligands.

1.-2. (canceled)

3. A compound of formula 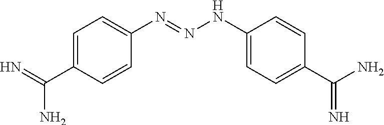 [SMILES: N=C(N)c1ccc(/N=N/Nc2ccc(C(=N)N)cc2)cc1]
having one or more of ortho, para, and/or meta substitutions, wherein the one or more substitutions comprise 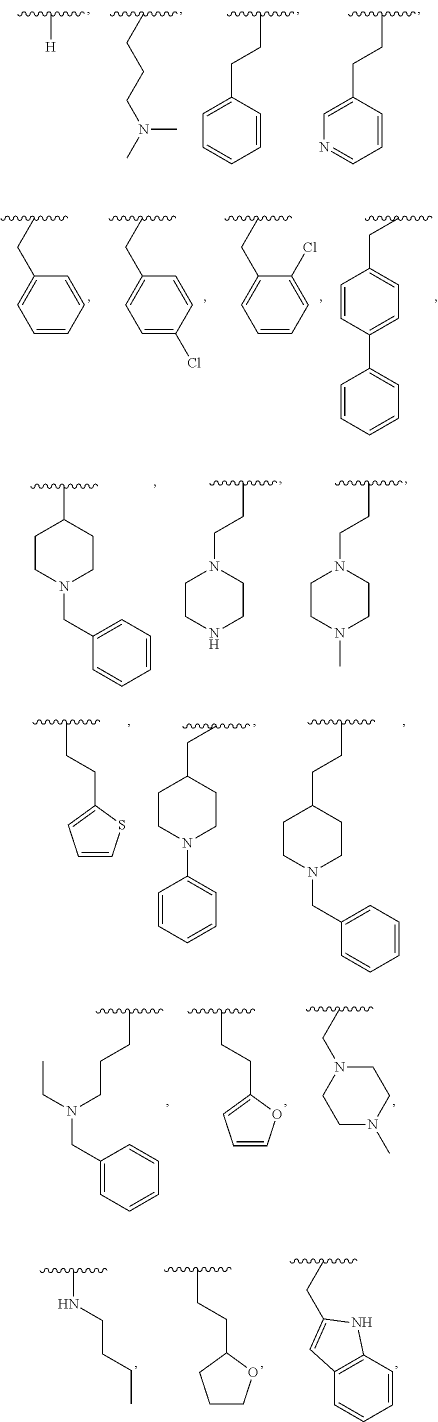 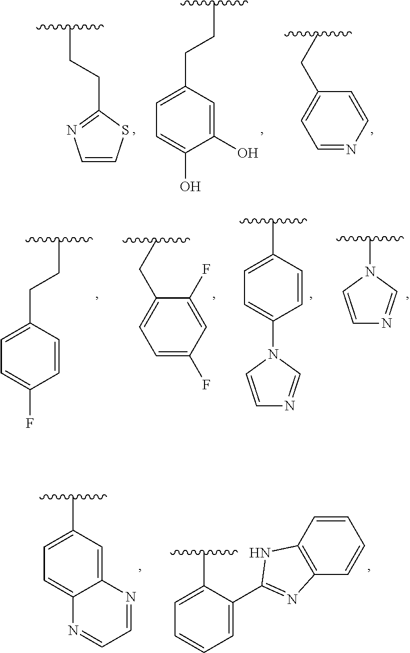
or any combination thereof.

4. (canceled)

5. The compound of claim 3, wherein the compound is

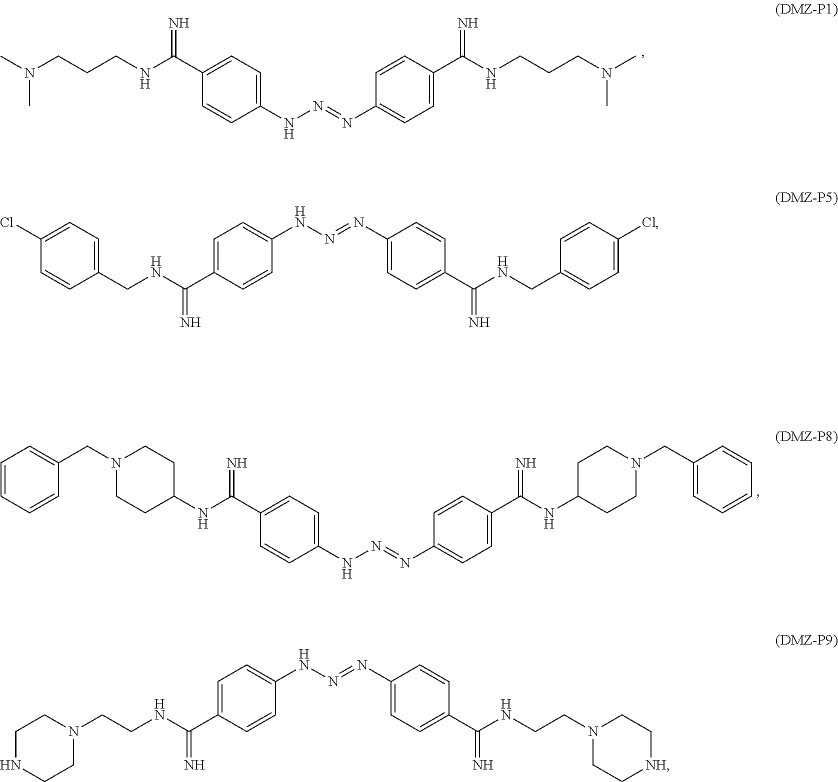

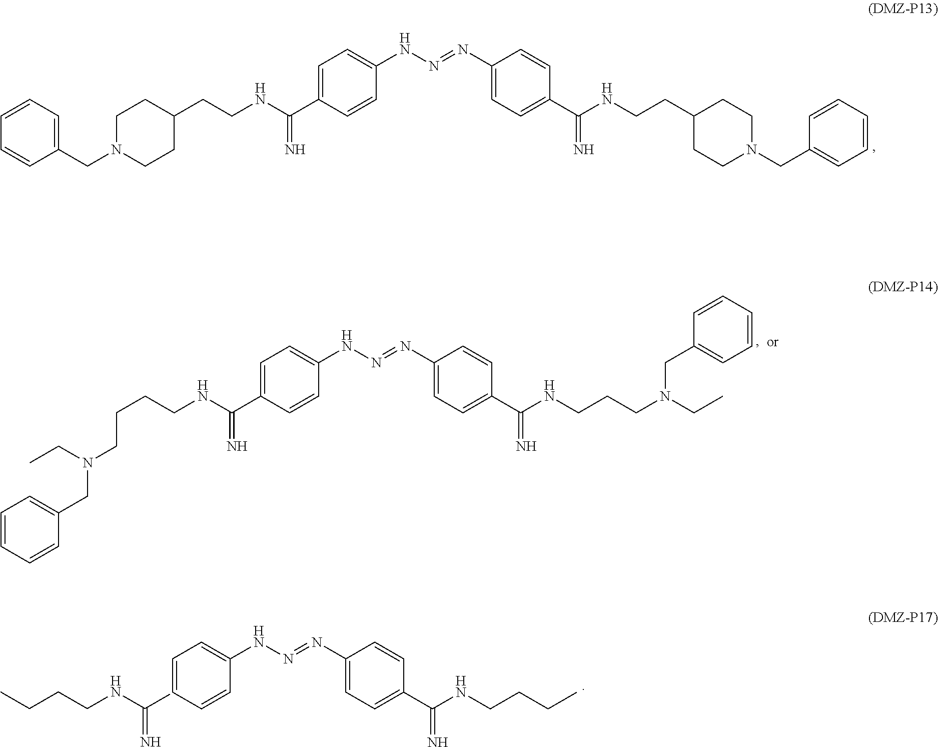

6.-11. (canceled)

12. The compound of claim 3, wherein the compound is

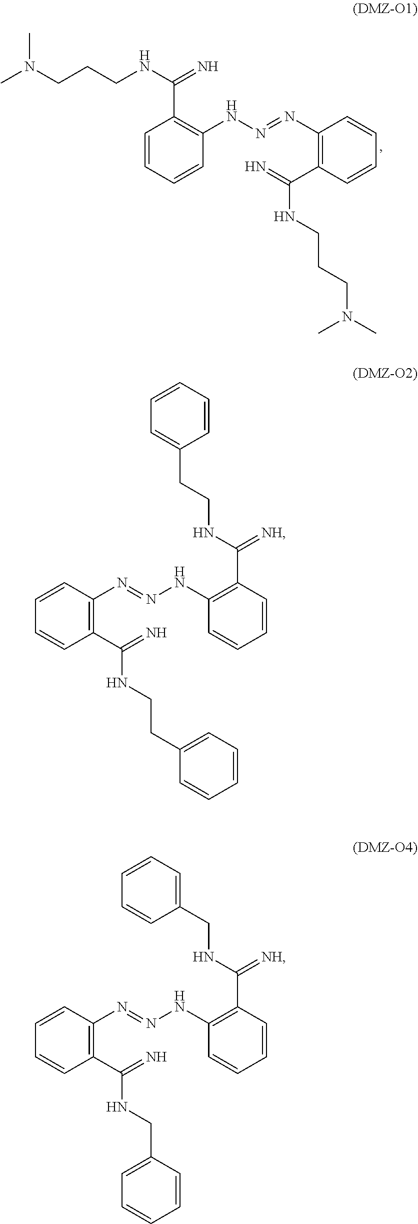

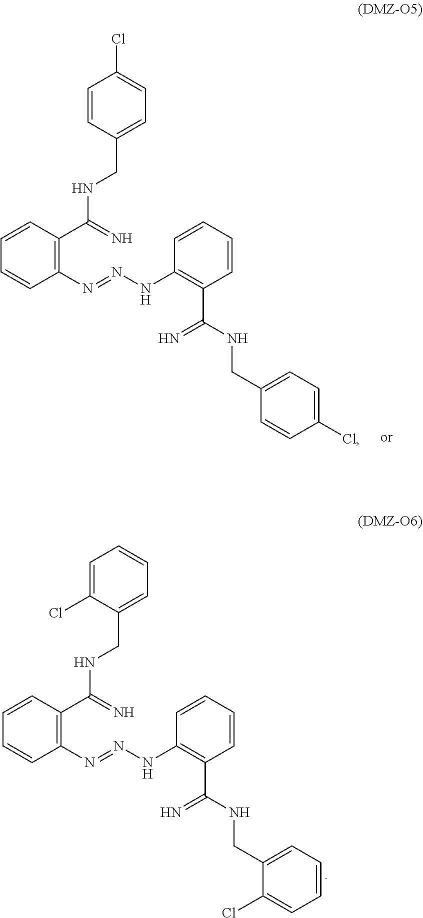 [SMILES: N=C(NCc1ccc(Cl)cc1)c1ccccc1/N=N/Nc1ccccc1C(=N)NCc1ccc(Cl)cc1 ; "or" ; N=C(NCc1ccccc1Cl)c1ccccc1/N=N/Nc1ccccc1C(=N)NCc1ccccc1Cl]

13.-16. (canceled)

17. The compound of claims 3

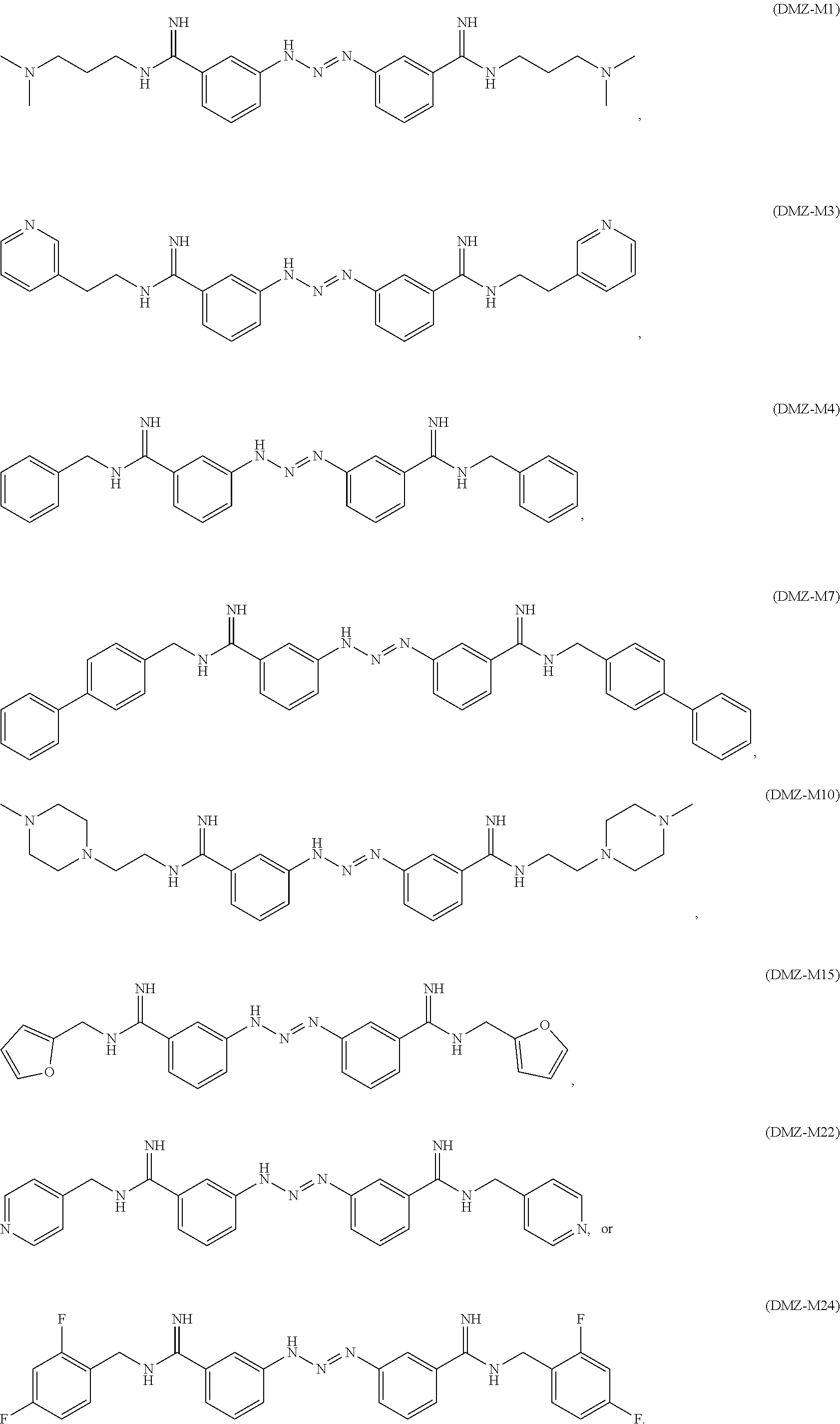

18.-24. (canceled)

25. The compound of claim 3, wherein the compound is

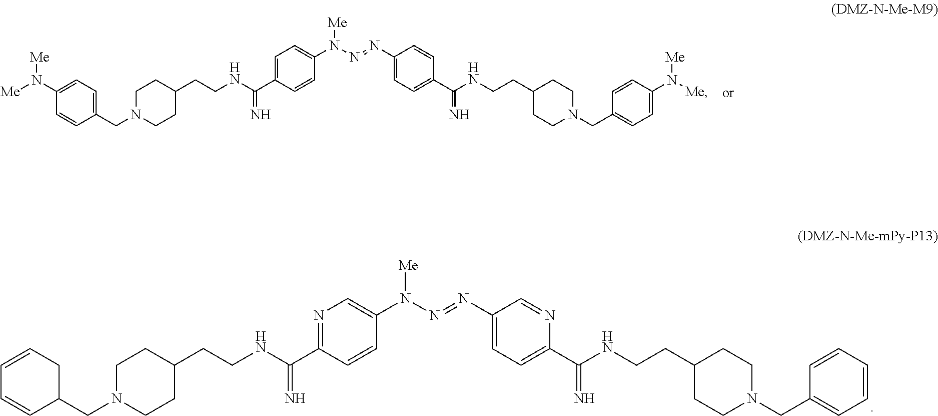 [SMILES: CN(C)c1ccc(CN2CCC(CCNC(=N)c3ccc(/N=N/N(C)c4ccc(C(=N)NCCC5CCN(Cc6ccc(N(C)C)cc6)CC5)cc4)cc3)CC2)cc1 ; CN(/N=N/c1ccc(C(=N)NCCC2CCN(Cc3ccccc3)CC2)nc1)c1ccc(C(=N)NCCC2CCN(CC3C=CC=CC3)CC2)nc1]

26. (canceled)

27. The compound of claim 3, wherein the compound modulates tertiary RNA structure.

28. The compound of claim 27, wherein the compound stabilizes the tertiary RNA structure.

29. The compound of claim 27, wherein the compound destabilizes the tertiary RNA structure.

30. The compound of claim 27, wherein the tertiary RNA structure comprises the MALAT1 triple helix.

31. A composition, comprising: a compound of claim 3, and one or more pharmaceutically acceptable carriers and/or excipients.

32. A method of treating a subject in need thereof, the method comprising:
administering to a subject the composition of claim 31, wherein one or more targeted RNA or targeted RNA structures are modulated.

33. The method of claim 32, wherein the subject has cancer.

34. (canceled)

35. The method of claim 32, wherein administering comprises delivery to one or more of the subject's body systems affected by the targeted RNA or targeted RNA structure, and wherein the one or more body systems affected by the targeted RNA or targeted RNA structure comprise the cardiovascular system, the digestive system, the endocrine system, the lymphatic system, the muscular system, the nervous system, the reproductive system, the respiratory system, the skeletal system, the urinary system, the integumentary system, or any combination thereof.

36. (canceled)

37. The method of claim 32, wherein following the administering step, (i) the subject's symptoms are diminished and/or decreased, (ii) the subject's quality of life is improved and/or enhanced, and/or (iii) one or more of the subject's body systems experience and/or show signs of normal physiology and/or cellular homeostasis.

38.-39. (canceled)

40. The method of claim 32, further comprising administering to the subject one or more therapeutic agents and/or active agents.

41. The method of claim 40, wherein the one or more therapeutic agents and/or active agents comprise an anti-cancer agent.

42. The method of claim 32, further comprising monitoring the subject for adverse effects.

43. The method of claim 42, wherein in the absence of adverse effects, the method further comprises continuing to treat the subject and/or continuing to monitor the subject, or wherein in the presence of adverse effects, the method further comprises modifying one or more steps of the method.

44. (canceled)

45. The method of claim 33, wherein the cancer is prostate cancer.

46. The compound of claim 3, wherein the compound is [chemical structure]


SEQUENCE LISTING (25 sequences)

SEQ ID NO: 1 - DNA, 94 bp, synthetic construct
  source 1..94: mol_type = other DNA
ggaaggtttttcttttcctgagaaaacaacacgtattgttttctcaggttttgctttttggcctttttctagcttaaaaaaaaaaaaagcaaaa

SEQ ID NO: 2 - DNA, 59 bp, synthetic construct
  source 1..59: mol_type = other DNA
gaaattaatacgactcactataggaaggtttttcttttcctgagaaaacaacacgtatt

SEQ ID NO: 3 - DNA, 21 bp, synthetic construct
  source 1..21: mol_type = other DNA
  misc_binding 1: bound_moiety = Tmt 2- OMe modified nucleotide
ttgctttttttttttttaagc

SEQ ID NO: 4 - DNA, 67 bp, synthetic construct
  source 1..67: mol_type = other DNA
ggaaggtttttcttttcctgagaaaacaacacgtattgttttctcaggttttgctttttggcctttt

SEQ ID NO: 5 - DNA, 59 bp, synthetic construct
  source 1..59: mol_type = other DNA
gaaattaatacgactcactataggaagatttttcttttcctgagaaaacaacacgtatt

SEQ ID NO: 6 - DNA, 17 bp, synthetic construct
  source 1..17: mol_type = other DNA
  misc_binding 2: bound_moiety = modified by a sugar moeity
aaaaggccaaaaagcaa

SEQ ID NO: 7 - RNA, 32 nt, synthetic construct
  source 1..32: mol_type = other RNA
cctgagaaaacaacacgtattgttttctcagg

SEQ ID NO: 8 - RNA, 95 nt, synthetic construct
  source 1..95: mol_type = other RNA
  misc_binding 1: bound_moiety = Cy5
tggaaggtttttcttttcctgagaaaacaacacgtattgttttctcaggttttgctttttggcctttttctagcttaaaaaaaaaaaaagcaaaa

SEQ ID NO: 9 - RNA, 8779 nt, Homo sapiens
  source 1..8779: mol_type = mRNA
cgcagcctgcagcccgagacttctgtaaaggactggggccccgcaactggcctctcctgccctcttaagcgcagcgccattttagcaacgcagaagcccggcgccgggaagcctcagctcgcctgaaggcaggtcccctctgacgcctccgggagcccaggtttcccagagtccttgggacgcagcgacgagttgtgctgctatcttagctgtccttataggctggccattccaggtggtggtatttagataaaaccactcaaactctgcagtttggtcttggggtttggaggaaagcttttatttttcttcctgctccggttcagaaggtctgaagctcatacctaaccaggcataacacagaatctgcaaaacaaaaacccctaaaaaagcagacccagagcagtgtaaacacttctgggtgtgtccctgactggctgcccaaggtctctgtgtcttcggagacaaagccattcgcttagttggtctactttaaaaggccacttgaactcgctttccatggcgatttgccttgtgagcactttcaggagagcctggaagctgaaaaacggtagaaaaatttccgtgcgggccgtggggggctggcggcaactggggggccgcagatcagagtgggccactggcagccaacggcccccggggctcaggcggggagcagctctgtggtgtgggattgaggcgttttccaagagtgggttttcacgtttctaagatttcccaagcagacagcccgtgctgctccgatttctcgaacaaaaaagcaaaacgtgtggctgtcttgggagcaagtcgcaggactgcaagcagttgggggagaaagtccgccattttgccacttctcaaccgtccctgcaaggctggggctcagttgcgtaatggaaagtaaagccctgaactatcacactttaatcttccttcaaaaggtggtaaactatacctactgtccctcaagagaacacaagaagtgctttaagaggtattttaaaagttccgggggttttgtgaggtgtttgatgacccgtttaaaatatgatttccatgtttcttttgtctaaagtttgcagctcaaatctttccacacgctagtaatttaagtatttctgcatgtgtagtttgcattcaagttccataagctgttaagaaaaatctagaaaagtaaaactagaacctatttttaaccgaagaactactttttgcctccctcacaaaggcggcggaaggtgatcgaattccggtgatgcgagttgttctccgtctataaatacgcctcgcccgagctgtgcggtaggcattgaggcagccagcgcaggggcttctgctgagggggcaggcggagcttgaggaaaccgcagataagtttttttctctttgaaagatagagattaatacaactacttaaaaaatatagtcaataggttactaagatattgcttagcgttaagtttttaacgtaattttaatagcttaagattttaagagaaaatatgaagacttagaagagtagcatgaggaaggaaaagataaaaggtttctaaaacatgacggaggttgagatgaagcttcttcatggagtaaaaaatgtatttaaaagaaaattgagagaaaggactacagagccccgaattaataccaatagaagggcaatgcttttagattaaaatgaaggtgacttaaacagcttaaagtttagtttaaaagttgtaggtgattaaaataatttgaaggcgatcttttaaaaagagattaaaccgaaggtgattaaaagaccttgaaatccatgacgcagggagaattgcgtcatttaaagcctagttaacgcatttactaaacgcagacgaaaatggaaagattaattgggagtggtaggatgaaacaatttggagaagatagaagtttgaagtggaaaactggaagacagaagtacgggaaggcgaagaaaagaatagagaagatagggaaattagaagataaaaacatacttttagaagaaaaaagataaatttaaacctgaaaagtaggaagcagaagaaaaaagacaagctaggaaacaaaaagctaagggcaaaatgtacaaacttagaagaaaattggaagatagaaacaagatagaaaatgaaaatattgtcaagagtttcagatagaaaatgaaaaacaagctaagacaagtattggagaagtatagaagatagaaaaatataaagccaaaaattggataaaatagcactgaaaaaatgaggaaattattggtaaccaatttattttaaaagcccatcaatttaatttctggtggtgcagaagttagaaggtaaagcttgagaagatgagggtgtttacgtagaccagaaccaatttagaagaatacttgaagctagaaggggaagttggttaaaaatcacatcaaaaagctactaaaaggactggtgtaatttaaaaaaaactaaggcagaaggcttttggaagagttagaagaatttggaaggccttaaatatagtagcttagtttgaaaaatgtgaaggactttcgtaacggaagtaattcaagatcaagagtaattaccaacttaatgtttttgcattggactttgagttaagattattttttaaatcctgaggactagcattaattgacagctgacccaggtgctacacagaagtggattcagtgaatctaggaagacagcagcagacaggattccaggaaccagtgtttgatgaagctaggactgaggagcaagcgagcaagcagcagttcgtggtgaagataggaaaagagtccaggagccagtgcgatttggtgaaggaagctaggaagaaggaaggagcgctaacgatttggtggtgaagctaggaaaaaggattccaggaaggagcgagtgcaatttggtgatgaaggtagcaggcggcttggcttggcaaccacacggaggaggcgagcaggcgttgtgcgtagaggatcctagaccagcatgccagtgtgccaaggccacagggaaagcgagtggttggtaaaaatccgtgaggtcggcaatatgttgtttttctggaacttacttatggtaaccttttatttattttctaatataatgggggagtttcgtactgaggtgtaaagggatttatatggggacgtaggccgatttccgggtgttgtaggtttctctttttcaggcttatactcatgaatcttgtctgaagcttttgagggcagactgccaagtcctggagaaatagtagatggcaagtttgtgggttttttttttttacacgaatttgaggaaaaccaaatgaatttgatagccaaattgagacaatttcagcaaatctgtaagcagtttgtatgtttagttggggtaatgaagtatttcagttttgtgaatagatgacctgtttttacttcctcaccctgaattcgttttgtaaatgtagagtttggatgtgtaactgaggcgggggggagttttcagtatttttttttgtgggggtgggggcaaaatatgttttcagttctttttcccttaggtctgtctagaatcctaaaggcaaatgactcaaggtgtaacagaaaacaagaaaatccaatatcaggataatcagaccaccacaggtttacagtttatagaaactagagcagttctcacgttgaggtctgtggaagagatgtccattggagaaatggctggtagttactcttttttccccccacccccttaatcagactttaaaagtgcttaaccccttaaacttgttattttttacttgaagcattttgggatggtcttaacagggaagagagagggtgggggagaaaatgtttttttctaagattttccacagatgctatagtactattgacaaactgggttagagaaggagtgtaccgctgtgctgttggcacgaacaccttcagggactggagctgcttttatccttggaagagtattcccagttgaagctgaaaagtacagcacagtgcagctttggttcatattcagtcatctcaggagaacttcagaagagcttgagtaggccaaatgttgaagttaagttttccaataatgtgacttcttaaaagttttattaaaggggaggggcaaatattggcaattagttggcagtggcctgttacggttgggattggtggggtgggtttaggtaattgtttagtttatgattgcagataaactcatgccagagaacttaaagtcttagaatggaaaaagtaaagaaatatcaacttccaagttggcaagtaactcccaatgatttagtttttttccccccagtttgaattgggaagctgggggaagttaaatatgagccactgggtgtaccagtgcattaatttgggcaaggaaagtgtcataatttgatactgtatctgttttccttcaaagtatagagcttttggggaaggaaagtattgaactgggggttggtctggcctactgggctgacattaactacaattatgggaaatgcaaaagttgtttggatatggtagtgtgtggttctcttttggaatttttttcaggtgatttaataataatttaaaactactatagaaactgcagagcaaaggaagtggcttaatgatcctgaagggatttcttctgatggtagcttttgtattatcaagtaagattctattttcagttgtgtgtaagcaagtttttttttagtgtaggagaaatacttttccattgtttaactgcaaaacaagatgttaaggtatgcttcaaaaattttgtaaattgtttattttaaacttatctgtttgtaaattgtaactgattaagaattgtgatagttcagcttgaatgtctcttagagggtgggcttttgttgatgagggaggggaaactttttttttttctatagacttttttcagataacatcttctgagtcataaccagcctggcagtatgatggcctagatgcagagaaaacagctccttggtgaattgataagtaaaggcagaaaagattatatgtcatacctccattggggaataagcataaccctgagattcttactactgatgagaacattatctgcatatgccaaaaaattttaagcaaatgaaagctaccaatttaaagttacggaatctaccattttaaagttaattgcttgtcaagctataaccacaaaaataatgaattgatgagaaatacaatgaagaggcaatgtccatctcaaaatactgcttttacaaaagcagaataaaagcgaaaagaaatgaaaatgttacactacattaatcctggaataaaagaagccgaaataaatgagagatgagttgggatcaagtggattgaggaggctgtgctgtgtgccaatgtttcgtttgcctcagacaggtatctcttcgttatcagaagagttgcttcatttcatctgggagcagaaaacagcaggcagctgttaacagataagtttaacttgcatctgcagtattgcatgttagggataagtgcttatttttaagagctgtggagttcttaaatatcaaccatggcactttctcctgaccccttccctaggggatttcaggattgagaaatttttccatcgagcctttttaaaattgtaggacttgttcctgtgggcttcagtgatgggatagtacacttcactcagaggcatttgcatctttaaataatttcttaaaagcctctaaagtgatcagtgccttgatgccaactaaggaaatttgtttagcattgaatctctgaaggctctatgaaaggaatagcatgatgtgctgttagaatcagatgttactgctaaaatttacatgttgtgatgtaaattgtgtagaaaaccattaaatcattcaaaataataaactatttttattagagaatgtatacttttagaaagctgtctccttatttaaataaaatagtgtttgtctgtagttcagtgttggggcaatcttgggggggattcttctctaatctttcagaaactttgtctgcgaacactctttaatggaccagatcaggatttgagcggaagaacgaatgtaactttaaggcaggaaagacaaattttattcttcataaagtgatgagcatataataattccaggcacatggcaatagaggccctctaaataaggaataaataacctcttagacaggtgggagattatgatcagagtaaaaggtaattacacattttatttccagaaagtcaggggtctataaattgacagtgattagagtaatactttttcacatttccaaagtttgcatgttaactttaaatgcttacaatcttagagtggtaggcaatgttttacactattgaccttatatagggaagggagggggtgcctgtggggttttaaagaattttcctttgcagaggcatttcatccttcatgaagccattcaggattttgaattgcatatgagtgcttggctcttccttctgttctagtgagtgtatgagaccttgcagtgagtttatcagcatactcaaaatttttttcctggaatttggagggatgggaggagggggtggggcttacttgttgtagctttttttttttttacagacttcacagagaatgcagttgtcttgacttcaggtctgtctgttctgttggcaagtaaatgcagtactgttctgatcccgctgctattagaatgcattgtgaaacgactggagtatgattaaaagttgtgttccccaatgcttggagtagtgattgttgaaggaaaaaatccagctgagtgataaaggctgagtgttgaggaaatttctgcagttttaagcagtcgtatttgtgattgaagctgagtacattttgctggtgtatttttaggtaaaatgctttttgttcatttctggtggtgggaggggactgaagcctttagtcttttccagatgcaaccttaaaatcagtgacaagaaacattccaaacaagcaacagtcttcaagaaattaaactggcaagtggaaatgtttaaacagttcagtgatctttagtgcattgtttatgtgtgggtttctctctcccctcccttggtcttaattcttacatgcaggaacactcagcagacacacgtatgcgaagggccagagaagccagacccagtaagaaaaaatagcctatttactttaaataaaccaaacattccattttaaatgtggggattgggaaccactagttctttcagatggtattcttcagactatagaaggagcttccagttgaattcaccagtggacaaaatgaggaaaacaggtgaacaagctttttctgtatttacatacaaagtcagatcagttatgggacaatagtattgaatagatttcagctttatgctggagtaactggcatgtgagcaaactgtgttggcgtgggggtggaggggtgaggtgggcgctaagcctttttttaagatttttcaggtacccctcactaaaggcaccgaaggcttaaagtaggacaaccatggagccttcctgtggcaggagagacaacaaagcgctattatcctaaggtcaagagaagtgtcagcctcacctgatttttattagtaatgaggacttgcctcaactccctctttctggagtgaagcatccgaaggaatgcttgaagtacccctgggcttctcttaacatttaagcaagctgtttttatagcagctcttaataataaagcccaaatctcaagcggtgcttgaaggggagggaaagggggaaagcgggcaaccacttttccctagcttttccagaagcctgttaaaagcaaggtctccccacaagcaacttctctgccacatcgccaccccgtgccttttgatctagcacagacccttcacccctcacctcgatgcagccagtagcttggatccttgtgggcatgatccataatcggtttcaaggtaacgatggtgtcgaggtctttggtgggttgaactatgttagaaaaggccattaatttgcctgcaaattgttaacagaagggtattaaaaccacagctaagtagctctattataatacttatccagtgactaaaaccaacttaaaccagtaagtggagaaataacatgttcaagaactgtaatgctgggtgggaacatgtaacttgtagactggagaagataggcatttgagtggctgagagggcttttgggtgggaatgcaaaaattctctgctaagactttttcaggtgaacataacagacttggccaagctagcatcttagcggaagctgatctccaatgctcttcagtagggtcatgaaggtttttcttttcctgagaaaacaacacgtattgttttctcaggttttgctttttggcctttttctagcttaaaaaaaaaaaaagcaaaagatgctggtggttggcactcctggtttccaggacggggttcaaatccctgcggcgtctttgctttgactactaatctgtcttcaggactctttctgtatttctccttttctctgcaggtgctagttcttggagttttggggaggtgggaggtaacagcacaatatctttgaactatatacatccttgatgtataatttgtcaggagcttgacttgattgtatattcatatttacacgagaacctaatataactgccttgtctttttcaggtaatagcctgcagctggtgttttgagaagccctactgctgaaaacttaacaattttgtgtaataaaaatggagaagctctaaattgttgtggttcttttgtgaataaaaaaatcttgattggggaaaaaa

SEQ ID NO: 10 - RNA, 8545 nt, Homo sapiens
  source 1..8545: mol_type = mRNA
cgcagcctgcagcccgagacttctgtaaaggactggggccccgcaactggcctctcctgccctcttaagcgcagcgccattttagcaacgcagaagcccggcgccgggaagcctcagctcgcctgaaggcaggtcccctctgacgcctccgggagcccaggtttcccagagtccttgggacgcagcgacgagttgtgctgctatcttagctgtccttataggctggccattccaggtggtggtatttagataaaaccactcaaactctgcagtttggtcttggggtttggaggaaagcttttatttttcttcctgctccggttcagaaggtctgaagctcatacctaaccaggcataacacagaatctgcaaaacaaaaacccctaaaaaagcagacccagagcagtgtaaacacttctgggtgtgtccctgactggctgcccaaggtctctgtgtcttcggagacaaagccattcgcttagttggtctactttaaaaggccacttgaactcgctttccatggcgatttgccttgtgagcactttcaggagagcctggaagctgaaaaacggtagaaaaatttccgtgcgggccgtggggggctggcggcaactggggggccgcagatcagagtgggccactggcagccaacggcccccggggctcaggcggggagcagctctgtggtgtgggattgaggcgttttccaagagtgggttttcacgtttctaagatttcccaagcagacagcccgtgctgctccgatttctcgaacaaaaaagcaaaacgtgtggctgtcttgggagcaagtcgcaggactgcaagcagttgggggagaaagtccgccattttgccacttctcaaccgtccctgcaaggctggggctcagttgcgtaatggaaagtaaagccctgaactatcacactttaatcttccttcaaaaggtggtaaactatacctactgtccctcaagagaacacaagaagtgctttaagaggcggcggaaggtgatcgaattccggtgatgcgagttgttctccgtctataaatacgcctcgcccgagctgtgcggtaggcattgaggcagccagcgcaggggcttctgctgagggggcaggcggagcttgaggaaaccgcagataagtttttttctctttgaaagatagagattaatacaactacttaaaaaatatagtcaataggttactaagatattgcttagcgttaagtttttaacgtaattttaatagcttaagattttaagagaaaatatgaagacttagaagagtagcatgaggaaggaaaagataaaaggtttctaaaacatgacggaggttgagatgaagcttcttcatggagtaaaaaatgtatttaaaagaaaattgagagaaaggactacagagccccgaattaataccaatagaagggcaatgcttttagattaaaatgaaggtgacttaaacagcttaaagtttagtttaaaagttgtaggtgattaaaataatttgaaggcgatcttttaaaaagagattaaaccgaaggtgattaaaagaccttgaaatccatgacgcagggagaattgcgtcatttaaagcctagttaacgcatttactaaacgcagacgaaaatggaaagattaattgggagtggtaggatgaaacaatttggagaagatagaagtttgaagtggaaaactggaagacagaagtacgggaaggcgaagaaaagaatagagaagatagggaaattagaagataaaaacatacttttagaagaaaaaagataaatttaaacctgaaaagtaggaagcagaagaaaaaagacaagctaggaaacaaaaagctaagggcaaaatgtacaaacttagaagaaaattggaagatagaaacaagatagaaaatgaaaatattgtcaagagtttcagatagaaaatgaaaaacaagctaagacaagtattggagaagtatagaagatagaaaaatataaagccaaaaattggataaaatagcactgaaaaaatgaggaaattattggtaaccaatttattttaaaagcccatcaatttaatttctggtggtgcagaagttagaaggtaaagcttgagaagatgagggtgtttacgtagaccagaaccaatttagaagaatacttgaagctagaaggggaagttggttaaaaatcacatcaaaaagctactaaaaggactggtgtaatttaaaaaaaactaaggcagaaggcttttggaagagttagaagaatttggaaggccttaaatatagtagcttagtttgaaaaatgtgaaggactttcgtaacggaagtaattcaagatcaagagtaattaccaacttaatgtttttgcattggactttgagttaagattattttttaaatcctgaggactagcattaattgacagctgacccaggtgctacacagaagtggattcagtgaatctaggaagacagcagcagacaggattccaggaaccagtgtttgatgaagctaggactgaggagcaagcgagcaagcagcagttcgtggtgaagataggaaaagagtccaggagccagtgcgatttggtgaaggaagctaggaagaaggaaggagcgctaacgatttggtggtgaagctaggaaaaaggattccaggaaggagcgagtgcaatttggtgatgaaggtagcaggcggcttggcttggcaaccacacggaggaggcgagcaggcgttgtgcgtagaggatcctagaccagcatgccagtgtgccaaggccacagggaaagcgagtggttggtaaaaatccgtgaggtcggcaatatgttgtttttctggaacttacttatggtaaccttttatttattttctaatataatgggggagtttcgtactgaggtgtaaagggatttatatggggacgtaggccgatttccgggtgttgtaggtttctctttttcaggcttatactcatgaatcttgtctgaagcttttgagggcagactgccaagtcctggagaaatagtagatggcaagtttgtgggttttttttttttacacgaatttgaggaaaaccaaatgaatttgatagccaaattgagacaatttcagcaaatctgtaagcagtttgtatgtttagttggggtaatgaagtatttcagttttgtgaatagatgacctgtttttacttcctcaccctgaattcgttttgtaaatgtagagtttggatgtgtaactgaggcgggggggagttttcagtatttttttttgtgggggtgggggcaaaatatgttttcagttctttttcccttaggtctgtctagaatcctaaaggcaaatgactcaaggtgtaacagaaaacaagaaaatccaatatcaggataatcagaccaccacaggtttacagtttatagaaactagagcagttctcacgttgaggtctgtggaagagatgtccattggagaaatggctggtagttactcttttttccccccacccccttaatcagactttaaaagtgcttaaccccttaaacttgttattttttacttgaagcattttgggatggtcttaacagggaagagagagggtgggggagaaaatgtttttttctaagattttccacagatgctatagtactattgacaaactgggttagagaaggagtgtaccgctgtgctgttggcacgaacaccttcagggactggagctgcttttatccttggaagagtattcccagttgaagctgaaaagtacagcacagtgcagctttggttcatattcagtcatctcaggagaacttcagaagagcttgagtaggccaaatgttgaagttaagttttccaataatgtgacttcttaaaagttttattaaaggggaggggcaaatattggcaattagttggcagtggcctgttacggttgggattggtggggtgggtttaggtaattgtttagtttatgattgcagataaactcatgccagagaacttaaagtcttagaatggaaaaagtaaagaaatatcaacttccaagttggcaagtaactcccaatgatttagtttttttccccccagtttgaattgggaagctgggggaagttaaatatgagccactgggtgtaccagtgcattaatttgggcaaggaaagtgtcataatttgatactgtatctgttttccttcaaagtatagagcttttggggaaggaaagtattgaactgggggttggtctggcctactgggctgacattaactacaattatgggaaatgcaaaagttgtttggatatggtagtgtgtggttctcttttggaatttttttcaggtgatttaataataatttaaaactactatagaaactgcagagcaaaggaagtggcttaatgatcctgaagggatttcttctgatggtagcttttgtattatcaagtaagattctattttcagttgtgtgtaagcaagtttttttttagtgtaggagaaatacttttccattgtttaactgcaaaacaagatgttaaggtatgcttcaaaaattttgtaaattgtttattttaaacttatctgtttgtaaattgtaactgattaagaattgtgatagttcagcttgaatgtctcttagagggtgggcttttgttgatgagggaggggaaactttttttttttctatagacttttttcagataacatcttctgagtcataaccagcctggcagtatgatggcctagatgcagagaaaacagctccttggtgaattgataagtaaaggcagaaaagattatatgtcatacctccattggggaataagcataaccctgagattcttactactgatgagaacattatctgcatatgccaaaaaattttaagcaaatgaaagctaccaatttaaagttacggaatctaccattttaaagttaattgcttgtcaagctataaccacaaaaataatgaattgatgagaaatacaatgaagaggcaatgtccatctcaaaatactgcttttacaaaagcagaataaaagcgaaaagaaatgaaaatgttacactacattaatcctggaataaaagaagccgaaataaatgagagatgagttgggatcaagtggattgaggaggctgtgctgtgtgccaatgtttcgtttgcctcagacaggtatctcttcgttatcagaagagttgcttcatttcatctgggagcagaaaacagcaggcagctgttaacagataagtttaacttgcatctgcagtattgcatgttagggataagtgcttatttttaagagctgtggagttcttaaatatcaaccatggcactttctcctgaccccttccctaggggatttcaggattgagaaatttttccatcgagcctttttaaaattgtaggacttgttcctgtgggcttcagtgatgggatagtacacttcactcagaggcatttgcatctttaaataatttcttaaaagcctctaaagtgatcagtgccttgatgccaactaaggaaatttgtttagcattgaatctctgaaggctctatgaaaggaatagcatgatgtgctgttagaatcagatgttactgctaaaatttacatgttgtgatgtaaattgtgtagaaaaccattaaatcattcaaaataataaactatttttattagagaatgtatacttttagaaagctgtctccttatttaaataaaatagtgtttgtctgtagttcagtgttggggcaatcttgggggggattcttctctaatctttcagaaactttgtctgcgaacactctttaatggaccagatcaggatttgagcggaagaacgaatgtaactttaaggcaggaaagacaaattttattcttcataaagtgatgagcatataataattccaggcacatggcaatagaggccctctaaataaggaataaataacctcttagacaggtgggagattatgatcagagtaaaaggtaattacacattttatttccagaaagtcaggggtctataaattgacagtgattagagtaatactttttcacatttccaaagtttgcatgttaactttaaatgcttacaatcttagagtggtaggcaatgttttacactattgaccttatatagggaagggagggggtgcctgtggggttttaaagaattttcctttgcagaggcatttcatccttcatgaagccattcaggattttgaattgcatatgagtgcttggctcttccttctgttctagtgagtgtatgagaccttgcagtgagtttatcagcatactcaaaatttttttcctggaatttggagggatgggaggagggggtggggcttacttgttgtagctttttttttttttacagacttcacagagaatgcagttgtcttgacttcaggtctgtctgttctgttggcaagtaaatgcagtactgttctgatcccgctgctattagaatgcattgtgaaacgactggagtatgattaaaagttgtgttccccaatgcttggagtagtgattgttgaaggaaaaaatccagctgagtgataaaggctgagtgttgaggaaatttctgcagttttaagcagtcgtatttgtgattgaagctgagtacattttgctggtgtatttttaggtaaaatgctttttgttcatttctggtggtgggaggggactgaagcctttagtcttttccagatgcaaccttaaaatcagtgacaagaaacattccaaacaagcaacagtcttcaagaaattaaactggcaagtggaaatgtttaaacagttcagtgatctttagtgcattgtttatgtgtgggtttctctctcccctcccttggtcttaattcttacatgcaggaacactcagcagacacacgtatgcgaagggccagagaagccagacccagtaagaaaaaatagcctatttactttaaataaaccaaacattccattttaaatgtggggattgggaaccactagttctttcagatggtattcttcagactatagaaggagcttccagttgaattcaccagtggacaaaatgaggaaaacaggtgaacaagctttttctgtatttacatacaaagtcagatcagttatgggacaatagtattgaatagatttcagctttatgctggagtaactggcatgtgagcaaactgtgttggcgtgggggtggaggggtgaggtgggcgctaagcctttttttaagatttttcaggtacccctcactaaaggcaccgaaggcttaaagtaggacaaccatggagccttcctgtggcaggagagacaacaaagcgctattatcctaaggtcaagagaagtgtcagcctcacctgatttttattagtaatgaggacttgcctcaactccctctttctggagtgaagcatccgaaggaatgcttgaagtacccctgggcttctcttaacatttaagcaagctgtttttatagcagctcttaataataaagcccaaatctcaagcggtgcttgaaggggagggaaagggggaaagcgggcaaccacttttccctagcttttccagaagcctgttaaaagcaaggtctccccacaagcaacttctctgccacatcgccaccccgtgccttttgatctagcacagacccttcacccctcacctcgatgcagccagtagcttggatccttgtgggcatgatccataatcggtttcaaggtaacgatggtgtcgaggtctttggtgggttgaactatgttagaaaaggccattaatttgcctgcaaattgttaacagaagggtattaaaaccacagctaagtagctctattataatacttatccagtgactaaaaccaacttaaaccagtaagtggagaaataacatgttcaagaactgtaatgctgggtgggaacatgtaacttgtagactggagaagataggcatttgagtggctgagagggcttttgggtgggaatgcaaaaattctctgctaagactttttcaggtgaacataacagacttggccaagctagcatcttagcggaagctgatctccaatgctcttcagtagggtcatgaaggtttttcttttcctgagaaaacaacacgtattgttttctcaggttttgctttttggcctttttctagcttaaaaaaaaaaaaagcaaaagatgctggtggttggcactcctggtttccaggacggggttcaaatccctgcggcgtctttgctttgactactaatctgtcttcaggactctttctgtatttctccttttctctgcaggtgctagttcttggagttttggggaggtgggaggtaacagcacaatatctttgaactatatacatccttgatgtataatttgtcaggagcttgacttgattgtatattcatatttacacgagaacctaatataactgccttgtctttttcaggtaatagcctgcagctggtgttttgagaagccctactgctgaaaacttaacaattttgtgtaataaaaatggagaagctctaaattgttgtggttcttttgtgaataaaaaaatcttgattggggaaaaaa

SEQ ID NO: 11 - RNA, 8302 nt, Homo sapiens
  source 1..8302: mol_type = mRNA
cgcagcctgcagcccgagacttctgtaaaggactggggccccgcaactggcctctcctgccctcttaagcgcagcgccattttagcaacgcagaagcccggcgccgggaagcctcagctcgcctgaaggcaggtcccctctgacgcctccgggagcccaggtttcccagagtccttgggacgcagcgacgagttgtgctgctatcttagctgtccttataggctggccattccaggtggtggtatttagataaaaccactcaaactctgcagtttggtcttggggtttggaggaaagcttttatttttcttcctgctccggttcagaaggtctgaagctcatacctaaccaggcataacacagaatctgcaaaacaaaaacccctaaaaaagcagacccagagcagtgtaaacacttctgggtgtgtccctgactggctgcccaaggtctctgtgtcttcggagacaaagccattcgcttagttggtctactttaaaaggccacttgaactcgctttccatggcgatttgccttgtgagcactttcaggagagcctggaagctgaaaaacggtagaaaaatttccgtgcgggccgtggggggctggcggcaactggggggccgcagatcagagtgggccactggcagccaacggcccccggggctcaggcggggagcagctctgtggtgtgggattgaggcgttttccaagagtgggttttcacgtttctaagatttcccaagcagacagcccgtgctgctccgatttctcgaacaaaaaagcaaaacgtgtggctgtcttgggagcaagtcgcaggactgcaagcagttgggggagaaagtccgccattttgccacttctcaaccgtccctgcaaggctggggctcagttgcgtaatggaaagtaaagccctgaactatcacactttaatcttccttcaaaaggtggtaaactatacctactgtccctcaagagaacacaagaagtgctttaagaggcggcggaaggtgatcgaattccggtgatgcgagttgttctccgtctataaatacgcctcgcccgagctgtgcggtaggcattgaggcagccagcgcaggggcttctgctgagggggcaggcggagcttgaggaaaccgcagataagtttttttctctttgaaagatagagattaatacaactacttaaaaaatatagtcaataggttactaagatattgcttagcgttaagtttttaacgtaattttaatagcttaagattttaagagaaaatatgaagacttagaagagtagcatgaggaaggaaaagataaaaggtttctaaaacatgacggaggttgagatgaagcttcttcatggagtaaaaaatgtatttaaaagaaaattgagagaaaggactacagagccccgaattaataccaatagaagggcaatgcttttagattaaaatgaaggtgacttaaacagcttaaagtttagtttaaaagttgtaggtgattaaaataatttgaaggcgatcttttaaaaagagattaaaccgaaggtgattaaaagaccttgaaatccatgacgcagggagaattgcgtcatttaaagcctagttaacgcatttactaaacgcagacgaaaatggaaagattaattgggagtggtaggatgaaacaatttggagaagatagaagtttgaagtggaaaactggaagacagaagtacgggaaggcgaagaaaagaatagagaagatagggaaattagaagataaaaacatacttttagaagaaaaaagataaatttaaacctgaaaagtaggaagcagaagaaaaaagacaagctaggaaacaaaaagctaagggcaaaatgtacaaacttagaagaaaattggaagatagaaacaagatagaaaatgaaaatattgtcaagagtttcagatagaaaatgaaaaacaagctaagacaagtattggagaagtatagaagatagaaaaatataaagccaaaaattggataaaatagcactgaaaaaatgaggaaattattggtaaccaatttattttaaaagcccatcaatttaatttctggtggtgcagaagttagaaggtaaagcttgagaagatgagggtgtttacgtagaccagaaccaatttagaagaatacttgaagctagaaggggaagttggttaaaaatcacatcaaaaagctactaaaaggactggtgtaatttaaaaaaaactaaggcagaaggcttttggaagagttagaagaatttggaaggccttaaatatagtagcttagtttgaaaaatgtgaaggactttcgtaacggaagtaattcaagatcaagagtaattaccaacttaatgtttttgcattggactttgagttaagattattttttaaatcctgaggactagcattaattgacagctgacccaggtgctacacagaagtggattcagtgaatctaggaagacagcagcagacaggattccaggaaccagtgtttgatgaagctaggactgaggagcaagcgagcaagcagcagttcgtggtgaagataggaaaagagtccaggagccagtgcgatttggtgaaggaagctaggaagaaggaaggagcgctaacgatttggtggtgaagctaggaaaaaggattccaggaaggagcgagtgcaatttggtgatgaaggtagcaggcggcttggcttggcaaccacacggaggaggcgagcaggcgttgtgcgtagaggatcctagaccagcatgccagtgtgccaaggccacagggaaagcgagtggttggtaaaaatccgtgaggtcggcaatatgttgtttttctggaacttacttatggtaaccttttatttattttctaatataatgggggagtttcgtactgaggtgtaaagggatttatatggggacgtaggccgatttccgggtgttgtaggtttctctttttcaggcttatactcatgaatcttgtctgaagcttttgagggcagactgccaagtcctggagaaatagtagatggcaagtttgtgggttttttttttttacacgaatttgaggaaaaccaaatgaatttgatagccaaattgagacaatttcagcaaatctgtaagcagtttgtatgtttagttggggtaatgaagtatttcagttttgtgaatagatgacctgtttttacttcctcaccctgaattcgttttgtaaatgtagagtttggatgtgtaactgaggcgggggggagttttcagtatttttttttgtgggggtgggggcaaaatatgttttcagttctttttcccttaggtctgtctagaatcctaaaggcaaatgactcaaggtgtaacagaaaacaagaaaatccaatatcaggataatcagaccaccacaggtttacagtttatagaaactagagcagttctcacgttgaggtctgtggaagagatgtccattggagaaatggctggtagttactcttttttccccccacccccttaatcagactttaaaagtgcttaaccccttaaacttgttattttttacttgaagcattttgggatggtcttaacagggaagagagagggtgggggagaaaatgtttttttctaagattttccacagatgctatagtactattgacaaactgggttagagaaggagtgtaccgctgtgctgttggcacgaacaccttcagggactggagctgcttttatccttggaagagtattcccagttgaagctgaaaagtacagcacagtgcagctttggttcatattcagtcatctcaggagaacttcagaagagcttgagtaggccaaatgttgaagttaagttttccaataatgtgacttcttaaaagttttattaaaggggaggggcaaatattggcaattagttggcagtggcctgttacggttgggattggtggggtgggtttaggtaattgtttagtttatgattgcagataaactcatgccagagaacttaaagtcttagaatggaaaaagtaaagaaatatcaacttccaagttggcaagtaactcccaatgatttagtttttttccccccagtttgaattgggaagctgggggaagttaaatatgagccactgggtgtaccagtgcattaatttgggcaaggaaagtgtcataatttgatactgtatctgttttccttcaaagtatagagcttttggggaaggaaagtattgaactgggggttggtctggcctactgggctgacattaactacaattatgggaaatgcaaaagttgtttggatatggtagtgtgtggttctcttttggaatttttttcaggtgatttaataataatttaaaactactatagaaactgcagagcaaaggaagtggcttaatgatcctgaagggatttcttctgatggtagcttttgtattatcaaacttttttcagataacatcttctgagtcataaccagcctggcagtatgatggcctagatgcagagaaaacagctccttggtgaattgataagtaaaggcagaaaagattatatgtcatacctccattggggaataagcataaccctgagattcttactactgatgagaacattatctgcatatgccaaaaaattttaagcaaatgaaagctaccaatttaaagttacggaatctaccattttaaagttaattgcttgtcaagctataaccacaaaaataatgaattgatgagaaatacaatgaagaggcaatgtccatctcaaaatactgcttttacaaaagcagaataaaagcgaaaagaaatgaaaatgttacactacattaatcctggaataaaagaagccgaaataaatgagagatgagttgggatcaagtggattgaggaggctgtgctgtgtgccaatgtttcgtttgcctcagacaggtatctcttcgttatcagaagagttgcttcatttcatctgggagcagaaaacagcaggcagctgttaacagataagtttaacttgcatctgcagtattgcatgttagggataagtgcttatttttaagagctgtggagttcttaaatatcaaccatggcactttctcctgaccccttccctaggggatttcaggattgagaaatttttccatcgagcctttttaaaattgtaggacttgttcctgtgggcttcagtgatgggatagtacacttcactcagaggcatttgcatctttaaataatttcttaaaagcctctaaagtgatcagtgccttgatgccaactaaggaaatttgtttagcattgaatctctgaaggctctatgaaaggaatagcatgatgtgctgttagaatcagatgttactgctaaaatttacatgttgtgatgtaaattgtgtagaaaaccattaaatcattcaaaataataaactatttttattagagaatgtatacttttagaaagctgtctccttatttaaataaaatagtgtttgtctgtagttcagtgttggggcaatcttgggggggattcttctctaatctttcagaaactttgtctgcgaacactctttaatggaccagatcaggatttgagcggaagaacgaatgtaactttaaggcaggaaagacaaattttattcttcataaagtgatgagcatataataattccaggcacatggcaatagaggccctctaaataaggaataaataacctcttagacaggtgggagattatgatcagagtaaaaggtaattacacattttatttccagaaagtcaggggtctataaattgacagtgattagagtaatactttttcacatttccaaagtttgcatgttaactttaaatgcttacaatcttagagtggtaggcaatgttttacactattgaccttatatagggaagggagggggtgcctgtggggttttaaagaattttcctttgcagaggcatttcatccttcatgaagccattcaggattttgaattgcatatgagtgcttggctcttccttctgttctagtgagtgtatgagaccttgcagtgagtttatcagcatactcaaaatttttttcctggaatttggagggatgggaggagggggtggggcttacttgttgtagctttttttttttttacagacttcacagagaatgcagttgtcttgacttcaggtctgtctgttctgttggcaagtaaatgcagtactgttctgatcccgctgctattagaatgcattgtgaaacgactggagtatgattaaaagttgtgttccccaatgcttggagtagtgattgttgaaggaaaaaatccagctgagtgataaaggctgagtgttgaggaaatttctgcagttttaagcagtcgtatttgtgattgaagctgagtacattttgctggtgtatttttaggtaaaatgctttttgttcatttctggtggtgggaggggactgaagcctttagtcttttccagatgcaaccttaaaatcagtgacaagaaacattccaaacaagcaacagtcttcaagaaattaaactggcaagtggaaatgtttaaacagttcagtgatctttagtgcattgtttatgtgtgggtttctctctcccctcccttggtcttaattcttacatgcaggaacactcagcagacacacgtatgcgaagggccagagaagccagacccagtaagaaaaaatagcctatttactttaaataaaccaaacattccattttaaatgtggggattgggaaccactagttctttcagatggtattcttcagactatagaaggagcttccagttgaattcaccagtggacaaaatgaggaaaacaggtgaacaagctttttctgtatttacatacaaagtcagatcagttatgggacaatagtattgaatagatttcagctttatgctggagtaactggcatgtgagcaaactgtgttggcgtgggggtggaggggtgaggtgggcgctaagcctttttttaagatttttcaggtacccctcactaaaggcaccgaaggcttaaagtaggacaaccatggagccttcctgtggcaggagagacaacaaagcgctattatcctaaggtcaagagaagtgtcagcctcacctgatttttattagtaatgaggacttgcctcaactccctctttctggagtgaagcatccgaaggaatgcttgaagtacccctgggcttctcttaacatttaagcaagctgtttttatagcagctcttaataataaagcccaaatctcaagcggtgcttgaaggggagggaaagggggaaagcgggcaaccacttttccctagcttttccagaagcctgttaaaagcaaggtctccccacaagcaacttctctgccacatcgccaccccgtgccttttgatctagcacagacccttcacccctcacctcgatgcagccagtagcttggatccttgtgggcatgatccataatcggtttcaaggtaacgatggtgtcgaggtctttggtgggttgaactatgttagaaaaggccattaatttgcctgcaaattgttaacagaagggtattaaaaccacagctaagtagctctattataatacttatccagtgactaaaaccaacttaaaccagtaagtggagaaataacatgttcaagaactgtaatgctgggtgggaacatgtaacttgtagactggagaagataggcatttgagtggctgagagggcttttgggtgggaatgcaaaaattctctgctaagactttttcaggtgaacataacagacttggccaagctagcatcttagcggaagctgatctccaatgctcttcagtagggtcatgaaggtttttcttttcctgagaaaacaacacgtattgttttctcaggttttgctttttggcctttttctagcttaaaaaaaaaaaaagcaaaagatgctggtggttggcactcctggtttccaggacggggttcaaatccctgcggcgtctttgctttgactactaatctgtcttcaggactctttctgtatttctccttttctctgcaggtgctagttcttggagttttggggaggtgggaggtaacagcacaatatctttgaactatatacatccttgatgtataatttgtcaggagcttgacttgattgtatattcatatttacacgagaacctaatataactgccttgtctttttcaggtaatagcctgcagctggtgttttgagaagccctactgctgaaaacttaacaattttgtgtaataaaaatggagaagctctaaattgttgtggttcttttgtgaataaaaaaatcttgattggggaaaaaa

SEQ ID NO: 12 - RNA, 3756 nt, Homo sapiens
  source 1..3756: mol_type = mRNA
ggagttagcgacagggagggatgcgcgcctgggtgtagttgtgggggaggaagtggctagctcagggcttcaggggacagacagggagagatgactgagttagatgagacgagggggcgggctgggggtgcgagaaggaagcttggcaaggagactaggtctagggggaccacagtggggcaggctgcatggaaaatatccgcagggtcccccaggcagaacagccacgctccaggccaggctgtccctactgcctggtggagggggaacttgacctctgggagggcgccgctcttgcatagctgagcgagcccgggtgcgctggtctgtgtggaaggaggaaggcagggagaggtagaaggggtggaggagtcaggaggaataggccgcagcagccctggaaatgatcaggaaggcaggcagtgggtgcagggctgcaggagggccgggagggctaatcttcaacttgtccatgccagcagcccctttttttccagaccaagggctgtgaacccgcctggggatgaggcctggtcttgtggaactgaacttagctcgacggggctgaccgctctggcccagggtggtatgtaattttcgctcggcctgggacggggcccaggccgggcccagcctggtggagcgtccaggtctgggtgcgaagccaggcccctgggcggaggtgaggggtggtctgaggagtgatgtggagttaaggcgccatcctcaccggtgactggtgcggcacctagcatgtttgacaggcggggactgcgaggcacgctgctcgggtgttggggacaacattgaccaacgctttattttccaggtggcagtgctccttttggacttttctctaggtttggcgctaaactcttcttgtgagctcactccaccccttcttcctccctttaacttatccattcacttaaaacattacctggtcatctggtaagcccgggacagtaagccgagtggctgttggagtcggtattgttggtaatggtggaggaagagaggccttcccgctgaggctggggtggggcggatcggtgttgcttgcctgcagagagggtggggagtgaatgtgcacccttgggtgggcctgcagccatccagctgaaagttacaaaaatgcttcatggaccgtggtttgttactatagtgttcctcatggcgagcagatggaaccgggagacatggagtccctggccagtgtgagtcctagcattgcaggaggggagaccctggaggagagagcccgcctcaattgatgcctgcagattgaatttccagaggcttaggaggaggaagttctccaatgttctgtttccaggccttgctcaggaagccctgtattcaggaggctaccatttaaagtttgcagatgagcttatggggggcaatcttaaaaagtccacagcagatgcatccggctcgaggggccatcagctttgaataaatgcttgttccagagcccatgaatgccagcaggcacccctcctttcctggggtaaaggttttcagatgctgcatcttctaaattgagcctccggtcatactagttttgtgcttggaaccttgcttcaagaagatccctaagctgtagaacattttaacgttgatgccacaacgcagattgatgccttgtagatggagcttgcagatggagccccgtgacctctcacctacccacctgtttgcctgccttcttgtgcgtttctcggagaagttcttagcctgatgaaataacttggggcgttgaagagctgtttaattttaaatgccttagactggggatatattagaggaagcagattgtcaaattaagggtgtcattgtgttgtgctaaacgctgggagggtacaagttggtcattcctaaatctgtgtgtgagaaatggcaggtctagtttgggcattgtgattgcattgcagattactaggagaagggaatggtgggtacaccggtagtgctcttttgttcttgcttcgtttttttaaacttgaactttacttcgttagatttcataatactttcttggcattctagtaagaggaccctgaggtgggagttgtgggggacggggagaaggggacagcttggcaccggtcccgtgggcgttgcagtgtgggggatgggggtatgcagcttggcactggtactgggagggatgagggtgaagaaggggagagggttggttagagatacagtgtgggtggtgggggtggtaggaaatgcaggttgaagggaattctctggggctttggggaatttagtgcgtgggtgagccaagaaaatactaattaataatagtaagttgttagtgttggttaagttgttgcttggaagtgagaagttgcttagaaactttccaaagtgcttagaactttaagtgcaaacagacaaactaacaaacaaaaattgttttgctttgctacaaggtggggaagactgaagaagtgttaactgaaaacaggtgacacagagtcaccagttttccgagaaccaaagggaggggtgtgtgatgccatctcacaggcaggggaaatgtctttaccagcttcctcctggtggccaagacagcctgtttcagagggttgttttgtttggggtgtgggtgttatcaagtgaattagtcacttgaaagatgggcgtcagacttgcatacgcagcagatcagcatccttcgctgccccttagcaacttaggtggttgatttgaaactgtgaaggtgtgattttttcaggagctggaagtcttagaaaagccttgtaaatgcctatattgtgggcttttaacgtatttaagggaccacttaagacgagattagatgggctcttctggatttgttcctcatttgtcacaggtgtcttgtgattgaaaatcatgagcgaagtgaaattgcattgaatttcaagggaatttagtatgtaaatcgtgccttagaaacacatctgttgtcttttctgtgtttggtcgatattaataatggcaaaatttttgcctatctagtatcttcaaattgtagtctttgtaacaaccaaataaccttttgtggtcactgtaaaattaatatttggtagacagaatccatgtacctttgctaaggttagaatgaataatttattgtatttttaatttgaatgtttgtgctttttaaatgagccaagactagaggggaaactatcacctaaaatcagtttggaaaacaagacctaaaaagggaaggggatggggattgtggggagagagtgggcgaggtgcctttactacatgtgtgatctgaaaaccctgcttggttctgagctgcgtctattgaattggtaaagtaataccaatggctttttatcatttccttcttccctttaagtttcacttgaaattttaaaaatcatggttatttttatcgttgggatctttctgtcttctgggttccattttttaaatgtttaaaaatatgttgacatggtagttcagttcttaaccaatgacttggggatgatgcaaacaattactgtcgttgggatttagagtgtattagtcacgcatgtatggggaagtagtctcgggtatgctgttgtgaaattgaaactgtaaaagtagatggttgaaagtactggtatgttgctctgtatggtaagaactaattctgttacgtcatgtacataattactaatcacttttcttcccctttacagcacaaataaagtttgagttctaaactcattagaaaaaaaaaaaaaaaaaaaaaaa

SEQ ID NO: 13 - RNA, 22743 nt, Homo sapiens
  source 1..22743: mol_type = mRNA
ggagttagcgacagggagggatgcgcgcctgggtgtagttgtgggggaggaagtggctagctcagggcttcaggggacagacagggagagatgactgagttagatgagacgagggggcgggctgggggtgcgagaaggaagcttggcaaggagactaggtctagggggaccacagtggggcaggctgcatggaaaatatccgcagggtcccccaggcagaacagccacgctccaggccaggctgtccctactgcctggtggagggggaacttgacctctgggagggcgccgctcttgcatagctgagcgagcccgggtgcgctggtctgtgtggaaggaggaaggcagggagaggtagaaggggtggaggagtcaggaggaataggccgcagcagccctggaaatgatcaggaaggcaggcagtgggtgcagggctgcaggagggccgggagggctaatcttcaacttgtccatgccagcagcccctttttttccagaccaagggctgtgaacccgcctggggatgaggcctggtcttgtggaactgaacttagctcgacggggctgaccgctctggcccagggtggtatgtaattttcgctcggcctgggacggggcccaggccgggcccagcctggtggagcgtccaggtctgggtgcgaagccaggcccctgggcggaggtgaggggtggtctgaggagtgatgtggagttaaggcgccatcctcaccggtgactggtgcggcacctagcatgtttgacaggcggggactgcgaggcacgctgctcgggtgttggggacaacattgaccaacgctttattttccaggtggcagtgctccttttggacttttctctaggtttggcgctaaactcttcttgtgagctcactccaccccttcttcctccctttaacttatccattcacttaaaacattacctggtcatctggtaagcccgggacagtaagccgagtggctgttggagtcggtattgttggtaatggtggaggaagagaggccttcccgctgaggctggggtggggcggatcggtgttgcttgcctgcagagagggtggggagtgaatgtgcacccttgggtgggcctgcagccatccagctgaaagttacaaaaatgcttcatggaccgtggtttgttactatagtgttcctcatggcgagcagatggaaccgggagacatggagtccctggccagtgtgagtcctagcattgcaggaggggagaccctggaggagagagcccgcctcaattgatgcctgcagattgaatttccagaggcttaggaggaggaagttctccaatgttctgtttccaggccttgctcaggaagccctgtattcaggaggctaccatttaaagtttgcagatgagcttatggggggcaatcttaaaaagtccacagcagatgcatccggctcgaggggccatcagctttgaataaatgcttgttccagagcccatgaatgccagcaggcacccctcctttcctggggtaaaggttttcagatgctgcatcttctaaattgagcctccggtcatactagttttgtgcttggaaccttgcttcaagaagatccctaagctgtagaacattttaacgttgatgccacaacgcagattgatgccttgtagatggagcttgcagatggagccccgtgacctctcacctacccacctgtttgcctgccttcttgtgcgtttctcggagaagttcttagcctgatgaaataacttggggcgttgaagagctgtttaattttaaatgccttagactggggatatattagaggaagcagattgtcaaattaagggtgtcattgtgttgtgctaaacgctgggagggtacaagttggtcattcctaaatctgtgtgtgagaaatggcaggtctagtttgggcattgtgattgcattgcagattactaggagaagggaatggtgggtacaccggtagtgctcttttgttcttgcttcgtttttttaaacttgaactttacttcgttagatttcataatactttcttggcattctagtaagaggaccctgaggtgggagttgtgggggacggggagaaggggacagcttggcaccggtcccgtgggcgttgcagtgtgggggatgggggtatgcagcttggcactggtactgggagggatgagggtgaagaaggggagagggttggttagagatacagtgtgggtggtgggggtggtaggaaatgcaggttgaagggaattctctggggctttggggaatttagtgcgtgggtgagccaagaaaatactaattaataatagtaagttgttagtgttggttaagttgttgcttggaagtgagaagttgcttagaaactttccaaagtgcttagaactttaagtgcaaacagacaaactaacaaacaaaaattgttttgctttgctacaaggtggggaagactgaagaagtgttaactgaaaacaggtgacacagagtcaccagttttccgagaaccaaagggaggggtgtgtgatgccatctcacaggcaggggaaatgtctttaccagcttcctcctggtggccaagacagcctgtttcagagggttgttttgtttggggtgtgggtgttatcaagtgaattagtcacttgaaagatgggcgtcagacttgcatacgcagcagatcagcatccttcgctgccccttagcaacttaggtggttgatttgaaactgtgaaggtgtgattttttcaggagctggaagtcttagaaaagccttgtaaatgcctatattgtgggcttttaacgtatttaagggaccacttaagacgagattagatgggctcttctggatttgttcctcatttgtcacaggtgtcttgtgattgaaaatcatgagcgaagtgaaattgcattgaatttcaagggaatttagtatgtaaatcgtgccttagaaacacatctgttgtcttttctgtgtttggtcgatattaataatggcaaaatttttgcctatctagtatcttcaaattgtagtctttgtaacaaccaaataaccttttgtggtcactgtaaaattaatatttggtagacagaatccatgtacctttgctaaggttagaatgaataatttattgtatttttaatttgaatgtttgtgctttttaaatgagccaagactagaggggaaactatcacctaaaatcagtttggaaaacaagacctaaaaagggaaggggatggggattgtggggagagagtgggcgaggtgcctttactacatgtgtgatctgaaaaccctgcttggttctgagctgcgtctattgaattggtaaagtaataccaatggctttttatcatttccttcttccctttaagtttcacttgaaattttaaaaatcatggttatttttatcgttgggatctttctgtcttctgggttccattttttaaatgtttaaaaatatgttgacatggtagttcagttcttaaccaatgacttggggatgatgcaaacaattactgtcgttgggatttagagtgtattagtcacgcatgtatggggaagtagtctcgggtatgctgttgtgaaattgaaactgtaaaagtagatggttgaaagtactggtatgttgctctgtatggtaagaactaattctgttacgtcatgtacataattactaatcacttttcttcccctttacagcacaaataaagtttgagttctaaactcattagaattgttgtattgctatgttacatttctcgacccctatcacattgccttcataacgactttggatgtatcttcatattgtagatttaggtctagatttgctagctccaagtaattaaggccatgtaggagagcatggtaaccacagatagaactggtattatcccaagtggtctgcagactgctgagtggggatgggatctgctctctgttgagagttggtaatcattggtttgaaatgtgatgaaaccactcaagccaatgaaggtgggtgtgtaggtggggagtactttgccataatattttaaaacattacctggttagagttctaagtggtacttatttttgtttggttaggggaaagcctgaataaaaacagaaatggacacataatatgcatattccatagtctttgggaggctggaatgtgcctgggatttgggtctaagtgtatgcgtaattcttacctcactaaagaatttgccttgtttttttccttttggtgagtgactaaaacgtctgggcttccctgtgtgcgtgctacagtaagcaagcagaggctgtgcaaaggtgtgagcaggatcacgtggaatctggaggatacatcttggcttgcaaactgcctctgtctcctgggtgggactgttctgtccttgcactgctgttctgtgttacctcttggggtgtaaggttttgcttacaggagacaaactttgggcgtagaatggaagccactgccagcctctgtgctgagaaggaaggtgcttgtttcaaagggagcagcaagggaggcttgttctactcacctgggcctgtttgcctgagaaggggagataagggctgaactgggactagccagggggaccaacacaaatggtgggggatcatgacctgaaggattctttccttcccatgagctgcagggctggttgccgtccttgcaactgtgtcttatttgcctgtgccgttatatcttggtgacccctccacgtgtacactactgacaaacgggtggagtgctggggagaagtcactgtgccgcccacctagtaaaccttctgtctgtgctcatggcatctccaagatggggcactgctgtgtgcagaatccagggtcctctttctgcttgcaactcctttccctggatgccccagaaacaatccaggcctcctttcctatcttacccctttgctttgctttttaccccagcacctctataaccgccttctcttcttttcagaactccttgtttctcgtcctgttttttatgattacaaaactcttgcttccaccctggaagataactgctatagatgcctgtatgtaaatggtgctgtctccagcaactggcatgctgaagaagaattgattcacggggtataaatgttggggattggaagtggggatgaaatggcacttgttgatacaggagcagagaggtgaggccgactgctgaagacagctcgccaccctccttgcctccactccaatccaggggctggggccacattctttgccttcatttatcctcagatcaggtgagatcgacaggaggtgttgatggcagtgccagcaattattgctaatccgtttgcatccttatgcatagatctgaattcagactttgtgaatttccagaggtgtgggtaatataatagaattcagtgagtgggcatggctgatcttgtgcaaattaaaagttatggggcataagaatagcaaaagttgaacttcttttaaaaaggaaagtaccctgagagccagtattggttgaggctcttcagtatgcccaggttggcagcactgagaaccgcaggaacggcctgttgttacaaaaaggagattgactcagctgcccttggtgcatctgactgactatgactgctgagagattccaaggacccttaatgccagggctaacctctccatgtgcagtgagacctctggaggaagtgtcatcctctggctttgtgtggtactcattatggtgcagtgcgggcatgaaatgaagacacccaaataggcttacagatacgatatgttttaaatgttcgtatttaacaaaaacatactgacactgtttggaaatggcaacaggaagatagcaaaatgaatactaacattacgaaaagatgaacaggtacatgttccaaggcaggtggctgtgaacttcctctgagtgaaggcatcccctccagcacctttcagcctgctagttaggacgacccgccgccaccctccaggacctccagccctgcactgcctttcctctcttttaaataattcttcattgagttctaatatgtaaaaaaaaaaagtttactgtaaagtttgcaaataaggaaattttttttaaaagtcctcagtaatcttaccagtaacaattgttatgggcacatttgcttttggaagatttcttttgtatgcatgggataagtacatttttaaacaaaaatgggattatgccataaattctattttgtgactttaatatatagtgaacaccttttttaatgatgacaggatgttcccttgcatggctgtatcaatttaaacaatcttgtttcaatgggcatacagggtattttctagtttttttttcctcttagaaaataatacttgcgatgactttccttgtagctcagactttttcacgtctgttgttatctctttgggaatgctgaatacatacatttcgagaaggaaatgactgttaaactcttaagacttcaggttcatattgctaaactgcccagcagggagggattttttcaattagtgttctcactggtgaggcaaacctgatgccttcccctcttcctcagaaccggctttatcacattgaaaacctttgctcctccgacggatcgagtctgctttccctctggatgtgagcattgctttgtctgctggtgactgaacatctctaccttgtgtcaattggccatttgtggtgtgtgtgtgtgtgcgtgtgtgtgtgtgtgtgtgtgtatgattttctaattcctagtcatttttctattgattgttttgcaaaagccatttacatcttaaggatattgataatcttttgttatatttgatgcaaatatttttttccagtttataggttgccttttaattttgtgtttcaggtagataaaagttaaacgattttcttaggttagtttatcactgtggtttctgaacttgttatgtgtagatcttttccaccccaagagtacataaatattaatccatactttcttatggaacttgtatggtttcgttttttacatttaaaccttcttccccgtggtgtgtgttgtggaatctgtgtttgtgtgaggaggggcatggtgctctcagaacccacctcctgtggccagagagccctgtcctgtgagggtggttgtcacagtggcagggttcaattcagaagaccttgagggcaggctgatgtttcctgaatgggcccctggttgttgcttgtccctgactctccatttccccatctgagtggatttggacctaatagggcactggagctggttcgaatcctgactggactacttggcaactttatgtctgggagcaagttacttaacctccccaagcctgtgtctgtgaaatgcgggtaaatgaatgtagatgtttggcagcagctactccttgttgagctctcacagtgaactctcctgcctctgccctccttccccgcctcccctggtgcctagcgtcaggtctagccacttcctcctgggcccctctcccttttctgtggctggctgcctgcccgcctggcgctggacctttcatgtaacgggaatcagcatgtatattctggtctggtctgtttctacacttaattttgtttccagtagtatttccctgtaccggcagagttcacaaacacatttgaagaggctttttctcaggattcttaaccttcccaaaggaagtcccatggatgggtttctagaagtctataaatgctctgaaattgtatttttctgtggaaagcataactttcatctgcttgttcgtgctcaaaaaagatcatgaatgaatgattgcatgattttatgccattgtgcttatactaaaggatatgtagcccatctcttgagctgttaaactgttttgactactttaaatcgtgcagctgtgagcatctctgtaaatttagtgtacacatgtatcccctggagtggcattgcctcggcagtgagcacttatggttttataactctcttcacagactcaaatgactccagaaagctacacttcctgttgtgagtatatgatatccatttccctacatagccactaacatcaggtttttacaattttatttatttcttgctactttaagaaatttttgtggtgaaatacatataatagaagttgactatctgaatcatttttaagtatacattcagtagtgttaagtatgtcgccattgttgtacaaccaatctccagaactttttcatcttgcaaaacaaactctgtacccattaaataacattaaacattccattccctccagcctcagcaaccccattctactttctgtttctgtgagtttgactattccaagcacttcatatcagttaaatcatgaagtatttgtctgtctgtgactggcttatttctctgagcacagtgtcctcgagatgcgtctatgttgtagcatatgtcagaatttccttcctttttaaaagatccaaataatattcttattttatatcttttttttatccattcatccattagtggacacttgggttgcttttggctattgtaaataatggtgctatgtacaaatatctatattattgtatttacaagtataatgctgtaatgtacacacatctttttgagatcctaccttcagttcttttgagtatatagccagaagtggtattactaaatcttacgatatttctatttttaatttattgaggaaccactgtagtttttcatagcaactgcaccattttacgttctcaccaagagtgcacaagggttccgaggttcccacatcctccccaacacttgttattttctgctttttttagattgcagccatcatagtgggtgtgaggtgacatttcattgtggttttgatttgcatttccctaatgaggagtgatgctgagcatcttttcatatgcttactggtcatttgtatgttgtctttggaaaaatgtctattcaagtcctttgactattttaaaaattgggttattagagttatcgttgttgttgacttgtaggagtttctttctatattctggatattaatcccctatcagatatatgatttgcaaatatcttctcttattccataaggttactttttcactttgttgattgtgttctttgatgtatagaagtttttagttttgaaatagtctaatttatctgtttttacttttgtggtctgtgcttttggtgtcatatccaagaaatccttgccaaatccaacgttataaggtacttttaaggtattttagttgtcttagtctatatttctgtactcacctttctttatccactcatcagttgatgggcatgtaggttggttccatatctttgcaattctgaattgtgctatgatcaggtgtctttttagtataatgatttactctcctttgggtagatacccagtagtgggattgctggatcgaatggtttttataattttctattttaccacagtttctctctgcatttttcctctttgaccactaaccatgtgaaattctcatattgacctttataatgatcatgaactcttagtatcattgggaaggccacatttgccacttatgattgtaaaccttatcctccatttttcctgttattgttggtgcaaaaagcacctattataccaggactttaaaaatcagtctgataagtctttgataagtctaataataataactgataagtccattgaatttgcttctgattactttttctttagtagctaaacatgtatgtactcctatgattacaatgaacactcctctccatttaaattaattatttacattgatgaaatagcaaaatgttaatgactaaatactgtcttggttttttcgttccaggtcagtcaatattaacttcttataattttcttttttttctttatgtgtgtgtgtgtgtgtatttttttttttttaatttcaatggcttttggggtacaaatggcttttggtcatatagatgaattctacagtagtgaagtctgagattttactgcaccggtcacctgagtagtgtacattgtacccaatatgtggttttttataccttgcccccctcttaccctccccactttgagtctctagtgtccattatgtcactctgtatacctttttgtacccataagttagctctcacttataagtgagaacacacagtatttggttttccattcctgagttgcttcacttagaataatatcctccagctccatccaaaattgctgcaaaaaaaaaaaaaaccacaaacattattttgttcttttttattgctaagtcatattccatggtgtagagataccacattttatttatccactcactggttgatgggttggttccacatctttgcaattgtgacttgtactgccatcaagtgtctttctggtataatgacttcttttcctttgggtagatacccaggagtgggattgctagatcaaatggttcttaacattttctctctggatctatttctggaaattttaggctccagtttttgttgttgttgttaataaaatgcaatggaatgtaatgatcatcacttttcattatgctttaaaatctggtaaatggaggctagaacactcctgtaaggcaagaatattctctctgttggaactcaaatacacagaactgggtaaatctcaatcttaatctttgattcaggacacaacatggctctcttttacttgctttctttaattgttttttaataatgtggtaagcatttctgaatctcctatccaatacaaaaactaggacaatacagacagtaactcctatggttacaatgaacactcctctccacttaaattaattatttacactgatgaaattgaaatagcaaaattttaatgactaaatactgtctttgattttttgttccaggtctgtcaatattaacttcttataattttctttttttttctttatgtgtgtgtgtgtgtgtgtatatatatatatttaatttcaatggcttttggggtacaaatggcttttggtcatatatatgagttctacagtagtgaagtctgagattttactacaccttccacttatgtggtcccacaccacccgcctcccctgccgcctcctgccaccccctaggccaaggtaataatcatcctgaatcctgggtttatctctcacttgctttcttttcatataattttgcaaaagaatctgatctaaatgtgtttttcagagtatatatttatattttagctgttcttagagaaaatttattattttgcatgtaatcttatggaacattctcatttaataccatggtaagattcagcccttgcccaggggatagttcatttagtttgtttactggatagagctcatcatgtgactatacctcagttagtttatcagttctcccatccatggtgactaggttgcctctcagcctctcaacaacactgtttctcagtgtccttgtagaagtgatatgtgggtgttttctccttacacagagttgaaaggtgacgacaacaacgttggcactaccaatcccccaccctccagaggggtaaccagtgttaccagtttgctgtgtttcctgctacacctcgccttattcacttccatttgtatctgaaaaacgtgttgcatggtttcttttctatagaagtggtaaaatgctattgtgtcctgtacattattgattactttttttcatttaacagtagggagatgcctgggagtacacagagaactgccctcattgttttcaacttctgcactgtatgtctgtgagtttagccattctgctgttaatggaaatttacagtattctaatcttttgatattacaaacagttctgtgcgatcatcgtcatacacaaccccttgtgcacaatgcatgagtgtttctcagggtaggtaccaagaagtgaaattcctgggtcatagggcgtgagtccgacatttttctccattctgccctgttgccctccagagtgggtgtccagctttgcatacctaagtatgagagtatctgttgttcatatcctctacgacgctccatatatgaaacttaagtttctgctagttgccatctttgatctatcatgtatgcagtgacctactaagactgtaattggtacagtagattcttgtcatctgtgtgtgaatttagcattcatgggcttaatgctgacaaggcccccagggtccaagacatataatcatgtataattttgtcaaggtataattttttaaattgcttttgtcatgtgtctgctggtgatgcccaacccagtgctctgcacccaggtcacactgtggctttgtcctctgcttatgcctgcattgcagcaactgtcctgaagagaccaaaattatgcagatttaggtaagtccatggctaatgttattatattatgtgctattgtaatggatggggctgtggagtgtatgaatttataaatcactggtcttgtaattaaaattcaaacactatagaaaaaggccatgtagaagataaaagttcctctataatcccggacccctaagataactactaatgacaacttcatttatattccttcagacattttctggctgtggatgtactaaaatgtatcctattattctctgccctaaaatggaatcatacaaggtgtactgttatttttatggctctataacatgtcatattgtacgtgttggtatggtcattttaaccatttttctagtgatggctttgaggttatttgcagtttcctagccatctcaaagtgtgctgcggggatctcttttgcatccctctgggtgcagagctgaggcacccagaggcagtgtccagaggaggcagcatctgtaggtgtcttcacctgctctggctcttggcacatctggttggtgacactgttttgtgagatgggttgaaagcacgtgctgccaaaatagaataatgttggtcctctcctcatgtgccgtggaactggggtaaaactgcgtagtggctgcagctgcctgtccataccggaatcgagtataacacggtgcctggcttagcacaaaacagtagtgggtcctgcaggccccagagtctaattcctggtattctttcccctacacagattaaataaaccaaaaacaaactattctaggaaagcgtctgtgacatttgtaaaaagtggtatttaatgatcttttattcacttgtctgtttagtttgttgaaatcttaagtggcatcctggtctgggaaggagtgctgtctgcgcctgccctccgctgggcacagcgtggctgcttcaggggctaagcacacactttctgtcttctaaagggccgccacatgccaggagctcaggtgtgagcccggctctggctcttacctcatagggtcactcataggggcacagggagcagaacattgtacacagcgaggcaccacccggcttggcatctgcctcggtggacttactacctctagaaggaaatacctgagttcctctggcctcagctcctagagtgactggtgtgctgtccctgttactcttctgtcaaggtgacaactgtgtgacccatcatctgtgtgtcaaagcaaggccctgcctgggcctctgctcctgtgctgaccccaaaggcaaatgctttgctagtttccttccagttaatttcacctatgaatagatgtgtgaaaactgttcaaagccatacctgcacatgtttgaacttcaaaccctgtgggtgattcagtggcatctttctctaacccccagcctcccttcccacagaggccaccgtcatggccagttgctgcagtttctttccagagaacctgtgtatgtgtaaagctgtacaggcgtgggtacaccacacagcctgtcttgcactgtggactgttgagttactagtacatctaggtaagcaccgcatatctgtattcatgtctgccttggtcttttcaacatctgtgtggtagccgtgtttgaattacccattccctttttggggaaccattaagttgtttcagcaatttttactgtagataaggctataccgcatatctgtgtacatgggtttttatgtacatgggcaagtatatctgtgagagaaaagtttcctcaggaggaattctgggcacagcatgtgtaaatttctaaatatgatggacacccccagcttccacctcaaggaggttggtcccattgacatttccccacaccttcacccaggctgtgcccttaaacttggttatttgtcaatgtgagaagtggaaaatagtatttaattgtagtttggatttgtatttctattgggttgtatacttactgattaataataagagctctttacatattaaggaaattaacccttttcaaatacattcctatttctcactaatctttaagttttattgtaatattttgctctttagtttatatatatatgtatatatatatatatgtatatatatatatatacatatatatatacatatatatatactaattttcttttatggttcctggattttgtgagtagtttgaaaaggctaatccagctgaagattttgttgttgttgttaaaccccatgttttctcctaactctttttatttttattttggaggactctatctagacttaattttagcataacaagtgacagggttagttagcctgttgtccttacaccattttctggctaatacagctattaactattgatctgtctattcacgtgccagttcctaatggttttacatagtgtaatctgcacttcaaaatagcgaagggaagccctacctcattattctacttttccagaattctcctggctattccaggctgcatgtttaccttaaccttccctgtgatgtcttcatgccgttgtcttcttatgcaagaataaggtacgtctttccatccactcacgtctatttaatttgactttgcattacacagaaagctggtcttggtctgtctacctcggcatctagttgtcctcactgccccctagccgaccccaccccatctgactgactaccccatcacagagtacttttatttacgttttgctctgcctaatggttacttgatactgtcacgccgacagtgtccagttcagtggtctttgcagttgaaatgctcccgtacacactgtcttgttaaaaatgccagtaagttcatacaaacccagcttgcacccaaggtcacattcagagagcgtagggctgggatgggttgttttccaagcttctgccactgtgtggctagctcttcccactgggaagttctgtgtacccggaatgtcggagtggagtcctgttctagtgtccagcacctgaccctgtgcccaacccctcaacagcctattcctgctgtccacagcctgctggaactttttacaaaatatgttgccatgctggaccctgggcactggacataagccccctggcagcctttttcatgtcacccaaaggggtaattgtcctactggtggtctgtaagatgagttagggtgacttgctaatagacattgtaaatcttaatatttatgtatgtattttattattaccggttttccatttatgatggtaatattgtttcttctaagaatatttatttttccttctaaatattgagataaaattcatgcttttgaaatgttctattcagtggcttttagtatatttgctatgttgtgcaaccatcgacactatccatttctagaactttttcgtcatcccaaacagacgctctgtattcataaaaaaataacttcctacctgtctctccccctagtctttggtaacctttgttatactggtaaactttgttgtgctctctgtctgtgtgaatttgcctattctaggggcctcatataagtgtaatcatacagtatttgtctttttgggtctgtctgatttcacttagcgggttttcagggttcattcatgttgcagcatataacagtactgcgttcctttttctggctgaataatattccactgtatggatagaccccattttgtttattcacacatcatttggacatttggattatttctggtttttggctattatgaacaatggtgctatgaacagttgcgtacaagtttttgtgtgaacatatgttttcaattctctcattatatacctaggagtagaattactgggtcatatggtaactgtatatttttgaggaactgccaaactattttcccacgtccatgcaccatttcacattcccaccagtaagtaagagggttccaatttctgcgcattcttgccaacactagttattatctgactttctggttataatcattctaatgagtgtgaagtagcctctggtgtcatttggatttgcatttctctgatgagtgatgctatcaagcacctttgctggtgctgttggccatatgtgtatgttccctggagaagtgtctgtgctgagccttggcccactttttaattaggcgtttgtctttttattactgagttgtaagagttctttatatattctggattctagacccttatcagatacatggtttgcaaatattttctcccattctgtgggttgtgttttcactttatcgataatgtccttagacatataataaatttgtattttaaaagtgacttgatttggctgtgcaaggtggctcacgcttgtaatcccagcactttgggagactgaggtgggtggatcatatgaggaggctaggagttcgaggtcagcctggccagcatagcgaaaacttgtctctactaaaaatacaaaaattagtcaggcatggtggtgcacgtctgtaataccagcttctcaggaggctgaggcacgaggatcacttgaacccaggaggaggaggttgcagtgagctgagatcatgccagggcaacagaatgagactttgtttaaaaaaaaaaaaaagtgacttgatttaagggaaaaaatgactggctatattcagtcagatatggcaaaaagtctcaaggtgttaatgtgaatgattaaggtcttggggggggtgtcccctatcagactacaggtgtttagaggcacagaaaaaggtgcagttgggttcttaatgtgaaatgatgagaagcacaactccagtgtgtctctttgtgtagaatgtcagcagacaccccctgctagatgtgctggatcatgggaaagcatttccatttgttactagattgttcagaagttttaatttatgatgggtgtggtggctcatgcctgtagtcccagcactgtgggaggctgaggcaggaggatcatctgaggccaagagttcaagatcagcctgggcaacatagtgataccctatctcttaaaaaagaagaagtttttaaatttgaaataataataggtactggatttatgcaaatgtcttttctgcgtcttttgagatgagtatcaggtttttttttttccttttatcatctgatgatgaacttaatgtttccatttgtattaatggaatactaagtccctctgtgatttctgaaccaagctattcctaggcctgagttttattttgttgacacagaaataaattagaaggccaagcgtggtggcatgtgcctgtagtcctagttgctgaggtaagaggattgcttgagcccaggagttcaaggctgcagcaagctttgattgcgccactgcactccagccttggcgacagactaagacgctgtctcaaaaaaaaacaaaaacgacaaaaaaaaaacaaaacagaaaaaataaactaaggcaatgacagtccctggcaaatgctgggagggaggcagcagtggtcagggaaggtaaccctgaagcaggacttgtaaagcaaataagattgggaggccaaggtgggtggatcacgaggtcaggagttcgagaccagcctggccaacatagtgaaaccccgtctttactaaaaatacaaaaaaattagccaggtgtggtggtgggtgcctgtagtcccagctacttgggaggctgaggcaggagaatctcgaacccaggaggcggaggttacagtcagctgagaccgcaccattgcactccagcctgggtgacagagcaagattccgtctcaaaaaaaaaaaaaaaaaaaaaaccaagaagaaaaggaatgaattagaacttcttctgcttggacttaagggcatcatcaggcaggttttgggtaggatagcaggggaggcagagacatagtcggggtcagtggtcatgagtgtggctttgagcccaaaaacttggtttctgttccctactttgccactcagtagtgcatgactttggccaaatttcttaaattcatgaagcaagtttccgggtgaatgaaatggggataaaaatagtgttcaaacctatccgttggtttgtgtgaaactgaaatgaatagtatcgtgcaggtacttgtgagcaaggggagctgctgtttcctgtccctttatgatgggaaatatctagacaagttcccaaccctctgcactgcaggctgcatggcacggagggtcttgtaacaccagctggggctggccttcttttaggagcttcagtggttctgaaaacttttatttgtttgtttgttttagtagatgtggggtctttctgtgttgcccggactggtctcaaacttctggactcaagtgatcctcccccgctcaacctcccaaagtgttgggattacaggtgtgagccactgtgcccagccttgaaaactttttcaggttcttccagggttactgggctattaaatatttctatttcattataagtcagtttttcaaagttatattatcttaattaccttttttatatgtattagtgtagagtagcattttatattttgatatcctccttatgcatagtttttcactttttattcctagtttttcgtttttaataagactttcaagaaatttattttattggccttttgaaaaaagcagctttagataaagtaagcagttctgctttcattttataatttatttctacttttgtttcattaatcttttcctccggcatgccttggattttgttgtgttactctttttctagaggctcgcattgtgtgtctggttcacttatgatcacgcttgcctacttttaagaatggaagaggggaggtggagggtggctgcacagtcgagggtgtgaggcagtcttgctctagccccaccatgccctcagcccgctgtggccacgctggttcctcaattgctggggcgtgcagtgtctgtaagggaggctactgatgccatccgaggaagatgtaaggtttcgtgtgggcagcgagagcctagcaggcatgtggggtgcccagcaaagggtaacagtggacagttgttgcctcattccacagagttttgattttttttttttttttaatggtcactccatcaacatcccccatggccagagcctgagctggtccccagagacacaggcattcagctgacagcctcgccttcacgctgctgctgttctcatgggggacaggcctcaggtggcaatgcacaaatcattagttaagggcagttgtgacagttaccaaggagtgtagtcccccgccccccgcccagtgaaaacagccctaaccaggggtggggacctttgggctctgacccgaagggtaggagaagctggaaggacagcattcctgtctgcgaaggcaggagcaaagctgccaggctatgaaggaaatggctggagcctgaagtcatgcaagctggggctggcagggacagggccaacttccaggcctgggggccaccatgaggattcaggacgtgacccccagggcacatgaaggccttccatctgtatttaagaaaagactttatcagacgagtatggtggctcacgcctgaatcttagcactttgggaggctgaggcaggtggatcacgaggtcaggagttcaataccagcctggccaatatggtaaaaccccatctctactaaaactacaaaaattagccaggcatggtggcgcacgcctgtagtcccagctactcgggaggctgaggcagaagaatcacttgaacccgggaggtggaggttacagtgagccaagatcgcgccactacactccagcctgggtgacagagtgagactccgtctcaaaaaaaccaaaagactttatcttatttcctatatgtttgtggtttcagtcctgatgtataatttgaccctagttagaatggttatctgaggaagtggcctgtacgatttctgcttttttaaatgtgtggctccctttcttcattgattaacgtatgattatttttataaatgttccatggcagtgggaagggattctctgtcacattccacatctggatcagttcctccccattttgttggtcaaatccgatctgccatatcctgtgtaatgacaagtgagttgcattctcaccgtcactcctggggtctctccgcttcccctgagctggctcagcagtctgctccatgtgttttgatgcagggtgacccattggtattcccgacactaacgcccccgtctgtggactgcttgctgcttgggcttcactgtgtctggtgttgacagtgcagacctaaaggtgtgcacacatgtgcacacacactccgctgtcttcttgtttgcactggacttaaatatctatgagggttattttcaactgctgaatttggaatgatttttatatcttttctgctttctgcccatgtacatgtgtttattttacactgttgtgattggtagttactatgtggggacacaattacttgggctgaaataatccacctgttgtggttggggtcctctggggcattccagggtgagaggttgtcactgccacctgggccatgtgggccggcaccagcattttgtggttacgaattctacagtcacaaatatctttgggcaaatccccttctatacctcaaggcagcttttggtttgcaaccccactggccagagggaagggccagtcacttggctctctcactgccctgcgccccagatggttctagggctgctgttttcccttggccctgccaacaccactgtttttacttctgctcattggctgagtgcagtggttcctggaagccagtggcacgtttccccgcgtagctcgcttatcccacagcacacacccaagggttctgttgctaacacgctgaattaattctttgctcatcttacagagtgtgttttgactgcccccatttctgaggccttgtaaggccagagctttgttgcttcatcggcaggttgggacttagatggccgtgaatgtttcctctctgctgctgcagtaagtaagtgcccgcaccatagtgtgtttggaggctgaagttgaagcgaggctgtgaggggagatggacgtgtgaggagggatgatggggcttgagcaaagtgggggagggggcaaaggcagttggcccaacacattccccacccctttgagaggtctgaggcctgcagacctggctcggagcccacctggtagtcctcagactgtgtgtgtgtgtgtgtgtgtgtgtgtgtgtgtgtgtgtgtgtgtgtgtgtgtgtgtgtaaaagagagaagttgtggagaaatggggggctgattctgctcagattcatcaggatgagtagaaggcacccagctctcaccctggcctgacatgtgtgtccctgagcaggttacagtcctctctgagcctctgcttcccatctggaccctgctgggcagggcttctgagctccttagcactagcaggaggggctccaggggccctccctccatggcagccaggacaggactctcaaatgaggacagcagagctcgtggggggctcccacggacccgccgtgggcccaggggaggcagagcctgagccaacagcagtggtgctgtggaccgtggatcctgagggtggcctggggcaagtaccggctgagggtccaggtgggctttgtgtacctttgggtcctggggccctggtgacttggactccaggttagagtcaagtgacaggagaaaggctggtggggccctgtgcttccgacttcatttcgagtgatggcagttcccaggaaggaatccacagctgacggtggctgacagatcagagaatggaaggcgaggcaggcgggcgtctgcgtgacctcaggtgcttggggcccagcagacccagagaaccatttccactaggccagggtgccggaagtgtccacaggtcttagattccctgttcagatgaaaagatttgtgcctttaatgataaaagtgatctgcatagagtcaaaaattcaagccatgggtataaaatgcaagtaaaatccctgccctcacctatcccaccctactacacagagatgtcctctcgagtttcctagactcactctggaaatttctgtatacacacagaagcttgtgcctctgctcgtgaaggcagagggagggagagctgaagggccagcaccttctcacctgtgggccccctcagtgctcggtcccagagcatgcaggactgtgcctcgtgttcagtttgctggtctgacttcatgctccttgggcaggatatgcatgtgccatgctaggagacatgtggatgtgaagctgggggacaatgtcccctggctatgcctttacaagggaagtaaggaaggtaggaggtgagcctgggagggagggagggaggcgcggagccgccgcaggtgtttcttttactgagtgcagcccatggccgcactcaggttttgcttttcaccttcccatctgtgaaagagtgagcaggaaaaagcaaaa

SEQ ID NO: 14 - RNA, 7253 nt, Homo sapiens
  source 1..7253: mol_type = mRNA
ggaaagaagtggaataattaggaacctagggtggggtagggtagcaggacatttcaaacattaatgagcatatgagattccaggtcttgttaaaatgcaaattctgattcagctggtaggtgaggtctgagattgtgcatttctaacaagcactcagataatcttaaggctgttggccccagggtcacacttatagtgattttctagaacccagttggggaagtgaatcttgggcaggagaaatacacacctcttgcattgagtttggagatctcatctgatataactttttaagaaagaaaaataattttccaaatatccaattgataagctttcccactaagtggctttcccactaagtggctgcgttatgaaaattgcttcactttgaaacttctggtcttggtaatatagaatttctgtgttctcacagtgcttgattgagaatatgatattgagattatggcataaaatatagtggctgtacaaaaaaaaatacattattaggatctctaacaattatgtaaaagtcattgcttcatgggtagagctcaaactttggtgtgagacctggttttattcttggcacttactctgagttgtcttaggcaaattaataccttaagcaaaaatattctcatgtacattttacatgagaattataaatgaagtacataaagtccagcagtcacaaatgttatctattattaccatcgtcctaagactgcaatcagctatagtgaaagtagtctcaaagattgtttcataaatcatcagattcacctaattttctaaagaatttaaataaggagatggaatgaatagattgcattttgtttccatgcacaggggaactgtgcatatttcttctgtgactcggaaatggtttaacttttaaaaatcccaaaatagctgaagttagcagacatgcaatttaccaaggatgattggaatttttatctttcctgtaataatactatacccaagcacactgctcatgaggaaaacatttttatgtgaatcttttactcttgggggcaaagaatgctgtttttctttttgataactatgtttatagaatctaaatcaccctgagcaattatttcaacatctaaagttattattaccattcatgtttcatttatagctatttgaattttgatgaatttcaatatggtgctacagtgatagggcaagtgcaaataagttcaatatatgggtacggtctaaagctattttaatttttttattacaactgctatgaagaaaattaggatatgccatattttcacgttttacagttggatgtcctatgatgttctcttccagagaacagagctcggagctctggaaatttggaggcaactgatatgtgctcatgtctgcatctgtgtgggttggctgtatctcagggacagagtctgcagcaaaaaagatataattttgaggactgaacaaaattcaggaaggactattctcattaaggcagtaacagagaagaattttgaaacaaaagattttcgagcctctctagaaaatggtgttctgctgtgtgatttgattaataagcttaaacctggcgtcattaagaagatcaatagactgtctacaccaatagcaggattggataatataaacgttttcttgaaagcttgtgaacagattggattgaaagaagcccagcttttccatcctggagatctacaggatttatcaaatcgagtcactgtcaagcaagaagagactgacaggagagtgaaaaatgttttgataacattgtactggctgggaagaaaagcacaaagcaacccgtactataatggtccccatcttaatttgaaagcgtttgagaatcttttaggacaagcactgacgaaggcactcgaagactccagcttcctgaaaagaagtggcagggacagtggctacggtgacatctggtgtcctgaacgtggagaatttcttgctcctccaaggcaccataagagagaagattcctttgaaagcttggactctttgggctcgaggtcattgacaagctgctcctctgatatcacgttgagaggggggcgtgaaggttttgaaagtgacacagattcggaatttacatttaagatgcaggattataataaagatgatatgtcgtatcgaaggatttcggctgttgagccaaagactgcgttacccttcaatcgttttttacccaacaaaagtagacagccatcctatgtaccagcacctctgagaaagaaaaagccagacaaacatgaggataacagaagaagttgggcaagcccggtttatacagaagcagatggaacattttcaagactctttcaaaagatttatggtgagaatgggagtaagtccatgagtgatgtcagcgcagaagatgttcaaaacttgcgtcagctgcgttacgaggagatgcagaaaataaaatcacaattaaaagaacaagatcagaaatggcaggatgaccttgcaaaatggaaagatcgtcgaaaaagttacacttcagatctgcagaagaaaaaagaagagagagaagaaattgaaaagcaggcacttgagaagtctaagagaagctctaagacgtttaaggaaatgctgcaggacagggaatcccaaaatcaaaagtctacagttccgtcaagaaggagaatgtattcttttgatgatgtgctggaggaaggaaagcgaccccctacaatgactgtgtcagaagcaagttaccagagtgagagagtagaagagaagggagcaacttatccttcagaaattcccaaagaagattctaccacttttgcaaaaagagaggaccgtgtaacaactgaaattcagcttccttctcaaagtcctgtggaagaacaaagcccagcctctttgtcttctctgcgttcacggagcacacaaatggaatcaactcgtgtttcagcttctctccccagaagttaccggaaaactgatacagtcaggttaacatctgtggtcacaccaagaccctttggctctcagacaaggggaatctcatcactccccagatcttacacgatggatgatgcttggaagtataatggagatgttgaagacattaagagaactccaaacaatgtggtcagcacccctgcaccaagcccggacgcaagccaactggcttcaagcttatctagccagaaagaggtagcagcaacagaagaagatgtgacaaggctgccctctcctacatcccccttctcatctctttcccaagaccaggctgccacttctaaagccacattgtcttccacatctggtcttgatttaatgtctgaatctggagaaggggaaatctccccacaaagagaagtctcaagatcccaggatcagttcagtgatatgagaatcagcataaaccagacgcctgggaagagtcttgactttgggtttacaataaaatgggatattcctgggatcttcgtagcatcagttgaagcaggtagcccagcagaattttctcagctacaagtagatgatgaaattattgctattaacaacaccaagttttcatataacgattcaaaagagtgggaggaagccatggctaaggctcaagaaactggacacctagtgatggatgtgaggcgctatggaaaggctggttcacctgaaacaaagtggattgatgcaacttctggaatttacaactcagaaaaatcttcaaatctatctgtaacaactgatttctccgaaagccttcagagttctaatattgaatccaaagaaatcaatggaattcatgatgaaagcaatgcttttgaatcaaaagcatctgaatccatttctttgaaaaacttaaaaaggcgatcacaattttttgaacaaggaagctctgattcggtggttcctgatcttccagttccaaccatcagtgccccgagtcgctgggtgtgggatcaagaggaggagcggaagcggcaggagaggtggcagaaggagcaggaccgcctactgcaggaaaaatatcaacgtgagcaggagaaactgagggaagagtggcaaagggccaaacaggaggcagagagagagaattccaagtacttggatgaggaactgatggtcctaagctcaaacagcatgtctctgaccacacgggagccctctcttgccacctgggaagctacctggagtgaagggtccaagtcttcagacagagaaggaacccgagcaggagaagaggagaggagacagccacaagaggaagttgttcatgaggaccaaggaaagaagccgcaggatcagcttgttattgagagagagaggaaatgggagcaacagcttcaggaagagcaagagcaaaagcggcttcaggctgaggctgaggagcagaagcgtcctgcggaggagcagaagcgccaggcagagatagagcgggaaacatcagtcagaatataccagtacaggaggcctgttgattcctatgatataccaaagacagaagaagcatcttcaggttttcttcctggtgacaggaataaatccagatctactactgaactggatgattactccacaaataaaaatggaaacaataaatatttagaccaaattgggaacatgacctcttcacagaggagatccaagaaagaacaagtaccatcaggagcagaattggagaggcaacaaatccttcaggaaatgaggaagagaacaccccttcacaatgacaacagctggatccgacagcgcagtgccagtgtcaacaaagagcctgttagtcttcctgggatcatgagaagaggcgaatctttagataacctggactccccccgatccaattcttggagacagcctccttggctcaatcagcccacaggattctatgcttcttcctctgtgcaagactttagtcgcccaccacctcagctggtgtccacatcaaaccgtgcctacatgcggaacccctcctccagcgtgcccccaccttcagctggctccgtgaagacctccaccacaggtgtggccaccacacagtcccccaccccgagaagccattccccttcagcttcacagtcaggctctcagctgcgtaacaggtcagtcagtgggaagcgcatatgctcctactgcaataacattctgggcaaaggagccgccatgatcatcgagtccctgggtctttgttatcatttgcattgttttaagtgtgttgcctgtgagtgtgacctcggaggctcttcctcaggagctgaagtcaggatcagaaaccaccaactgtactgcaacgactgctatctcagattcaaatctggacggccaaccgccatgtgatgtaagcctccatacgaaagcactgttgcagatagaagaagaggtggttgctgctcatgtagatctataaatatgtgttgtatgtcttttttgctttttttttaaaaaaaagaataactttttttgcctctttagattacatagaagcattgtagtcttggtagaaccagtatttttgttgtttatttataaggtaattgtgtgtggggaaaagtgcagtatttacctgttgaattcagcatcttgagagcacaagggaaaaaataagaacctacgaatatttttgaggcagataatgatctagtttgactttctagttagtggtgttttgaagagggtattttattgttttttaaaaaaaggttcttaaacattatttgaaatagttaatataaatacataattgcatttgctctgtttattgtaatgtattctaaattaatgcagaaccatatggaaaatttcattaaaatctatccccaaatgtgctttctgtatccttccttctacctattattctgatttttaaaaatgcagttaatgtaccatttatttgcttgatgaagggagctctattttctttaccagaaatgttgctaagtaattcccaatagaaagctgcttattttcattaatgaaaaataaccatggtttgtatactagaagtcttcttcagaaactggtgagcctttctgttcaattgcatttgtaaataaacttgctgatgcatttaacgagtgggtcgtctttttcttaggtgtatgtgtctgacctcaggccttttagccatatttcagtatgtggccttttttgatgttatgttttatccagtagctttactaaggtataattgatgtaataaactgcatatatttaaagtgtatactttgacaaattttgacatggtgtataccttcgaaactatgccacagtctggatgtgtttactgaaacattttaataaggaagtttatttttgataaagttatgtttttggatacaatatatttgtatggtgagagtgatgaattgttggatcatttgaataaaatcttttactaaccccatgataaaaggagaagacaacagtgagcttagaatatctataaagcaaaaaatgtagtctcttgtttaaaaaatctggagcgggaatgcaaggatacaaaactttagcatgctttgagcaaaaatttaaacttactggaatcttttataataatgtaagtggaatggaggattctaggaactgagaactgtattggaataggttcaaaatatgtaagaaatgctaatgtgggagataaaaattttatttagtacttattctgattattattaaagtaataatgtgttccttgaggataacttgtcaaatgccccaaagcataaagaatataattctgaatcccaaattccaaagacaagaactctgtgtttgaattcattctgcatataattatttataagtatagattgtgaatttttccatgttcttaaaattatttttatcttttttcatggttgcatagtgctccattgtttggccttggtaatatttagttgataattccattactgtgtatttttcacttgtttctaagatcaaacattttaatatgtgcatgttatatataaatatgtaaattctgtgatactctatgatcatctctttctttatattattttcatagacatgaaatagttgctcagagattatgcattttaagacactcatagtatatattgccaaagtggtttccagaaaggcactgctggcttcgactcctataagcagcacgtgggcttgttcatctcactgcatgtttatgaagatacagttcttttgccttgttctctgcctgatgtgtatgcagaggcagccctcaatatgcagtggttgaataaatgaatgaagaaaccactatcaaaaaaaaaaaaaaaaaa

SEQ ID NO: 15 - RNA, 6729 nt, Homo sapiens
  source 1..6729: mol_type = mRNA
attccacagtcgttctgtaatttgtaatcatagaaacagaatgttcctcctcttatttcttccctgccaacagtttctgggtgggtgaatgtcagtagctggagttatttttaaagaggagaggtgaggcttgccgtgcaaagctggaaagagggcgtttgtttgcagagcagagctgacatcaaagtgtagattactgctcagtggctaggcacttgtcctgtaacaggataatataaacgttttcttgaaagcttgtgaacagattggattgaaagaagcccagcttttccatcctggagatctacaggatttatcaaatcgagtcactgtcaagcaagaagagactgacaggagagtgaaaaatgttttgataacattgtactggctgggaagaaaagcacaaagcaacccgtactataatggtccccatcttaatttgaaagcgtttgagaatcttttaggacaagcactgacgaaggcactcgaagactccagcttcctgaaaagaagtggcagggacagtggctacggtgacatctggtgtcctgaacgtggagaatttcttgctcctccaaggcaccataagagagaagattcctttgaaagcttggactctttgggctcgaggtcattgacaagctgctcctctgatatcacgttgagaggggggcgtgaaggttttgaaagtgacacagattcggaatttacatttaagatgcaggattataataaagatgatatgtcgtatcgaaggatttcggctgttgagccaaagactgcgttacccttcaatcgttttttacccaacaaaagtagacagccatcctatgtaccagcacctctgagaaagaaaaagccagacaaacatgaggataacagaagaagttgggcaagcccggtttatacagaagcagatggaacattttcaagtaatcagaggaggatttggggcaccaatgtggagaactggccaactgtacaaggaacttcaaagtcctcttgttatttggaagaggaaaaagcaaagacaagaagcatacccaacattgtaaaggatgatctttatgtgcgcaagctcagtccagtcatgccaaacccagggaatgcttttgatcagtttcttcccaaatgttggaccccagaagatgtgaactggaaaagaataaaaagggaaacttataagccatggtataaagaatttcagggattcagtcagtttttactgcttcaggccctccaaacatactctgatgacatcttgtcttctgaaacacataccaaaattgatcccacttctggcccaaggctcataacccgcaggaagaatctctcttatgcaccaggctatagaagagatgacctcgagatggcagccctggatcctgacttagagaatgatgatttctttgtcagaaagactggggctttccatgcaaatccatatgttctccgagcttttgaagactttagaaagttctctgagcaagatgattctgtagagcgagatataattttacagtgtagagaaggtgaacttgtacttccggatttggaaaaagatgatatgattgttcgccgaattccagcacagaagaaagaagtgccgctgtctggggccccagatagataccacccagtcccttttcccgaaccctggactcttcctccagaaattcaagcaaaatttctctgtgtacttgaaaggacatgcccatccaaagaaaaaagtaatagctgtagaatattagttccttcatatcggcagaagaaagatgacatgctgacacgtaagattcagtcctggaaactgggaactaccgtgcctcccatcagtttcacccctggcccctgcagtgaggctgacttgaagagatgggaggccatccgggaggccagcagacttaggcacaagaaaaggctgatggtggagagactctttcaaaagatttatggtgagaatgggagtaagtccatgagtgatgtcagcgcagaagatgttcaaaacttgcgtcagctgcgttacgaggagatgcagaaaataaaatcacaattaaaagaacaagatcagaaatggcaggatgaccttgcaaaatggaaagatcgtcgaaaaagttacacttcagatctgcagaagaaaaaagaagagagagaagaaattgaaaagcaggcacttgagaagtctaagagaagctctaagacgtttaaggaaatgctgcaggacagggaatcccaaaatcaaaagtctacagttccgtcaagaaggagaatgtattcttttgatgatgtgctggaggaaggaaagcgaccccctacaatgactgtgtcagaagcaagttaccagagtgagagagtagaagagaagggagcaacttatccttcagaaattcccaaagaagattctaccacttttgcaaaaagagaggaccgtgtaacaactgaaattcagcttccttctcaaagtcctgtggaagaacaaagcccagcctctttgtcttctctgcgttcacggagcacacaaatggaatcaactcgtgtttcagcttctctccccagaagttaccggaaaactgatacagtcaggttaacatctgtggtcacaccaagaccctttggctctcagacaaggggaatctcatcactccccagatcttacacgatggatgatgcttggaagtataatggagatgttgaagacattaagagaactccaaacaatgtggtcagcacccctgcaccaagcccggacgcaagccaactggcttcaagcttatctagccagaaagaggtagcagcaacagaagaagatgtgacaaggctgccctctcctacatcccccttctcatctctttcccaagaccaggctgccacttctaaagccacattgtcttccacatctggtcttgatttaatgtctgaatctggagaaggggaaatctccccacaaagagaagtctcaagatcccaggatcagttcagtgatatgagaatcagcataaaccagacgcctgggaagagtcttgactttgggtttacaataaaatgggatattcctgggatcttcgtagcatcagttgaagcaggtagcccagcagaattttctcagctacaagtagatgatgaaattattgctattaacaacaccaagttttcatataacgattcaaaagagtgggaggaagccatggctaaggctcaagaaactggacacctagtgatggatgtgaggcgctatggaaaggctggttcacctgaaacaaagtggattgatgcaacttctggaatttacaactcagaaaaatcttcaaatctatctgtaacaactgatttctccgaaagccttcagagttctaatattgaatccaaagaaatcaatggaattcatgatgaaagcaatgcttttgaatcaaaagcatctgaatccatttctttgaaaaacttaaaaaggcgatcacaattttttgaacaaggaagctctgattcggtggttcctgatcttccagttccaaccatcagtgccccgagtcgctgggtgtgggatcaagaggaggagcggaagcggcaggagaggtggcagaaggagcaggaccgcctactgcaggaaaaatatcaacgtgagcaggagaaactgagggaagagtggcaaagggccaaacaggaggcagagagagagaattccaagtacttggatgaggaactgatggtcctaagctcaaacagcatgtctctgaccacacgggagccctctcttgccacctgggaagctacctggagtgaagggtccaagtcttcagacagagaaggaacccgagcaggagaagaggagaggagacagccacaagaggaagttgttcatgaggaccaaggaaagaagccgcaggatcagcttgttattgagagagagaggaaatgggagcaacagcttcaggaagagcaagagcaaaagcggcttcaggctgaggctgaggagcagaagcgtcctgcggaggagcagaagcgccaggcagagatagagcgggaaacatcagtcagaatataccagtacaggaggcctgttgattcctatgatataccaaagacagaagaagcatcttcaggttttcttcctggtgacaggaataaatccagatctactactgaactggatgattactccacaaataaaaatggaaacaataaatatttagaccaaattgggaacatgacctcttcacagaggagatccaagaaagaacaagtaccatcaggagcagaattggagaggcaacaaatccttcaggaaatgaggaagagaacaccccttcacaatgacaacagctggatccgacagcgcagtgccagtgtcaacaaagagcctgttagtcttcctgggatcatgagaagaggcgaatctttagataacctggactccccccgatccaattcttggagacagcctccttggctcaatcagcccacaggattctatgcttcttcctctgtgcaagactttagtcgcccaccacctcagctggtgtccacatcaaaccgtgcctacatgcggaacccctcctccagcgtgcccccaccttcagctggctccgtgaagacctccaccacaggtgtggccaccacacagtcccccaccccgagaagccattccccttcagcttcacagtcaggctctcagctgcgtaacagtgtgttgcctgtgagtgtgacctcggaggctcttcctcaggagctgaagtcaggatcagaaaccaccaactgtactgcaacgactgctatctcagattcaaatctggacggccaaccgccatgtgatgtaagcctccatacgaaagcactgttgcagatagaagaagaggtggttgctgctcatgtagatctataaatatgtgttgtatgtcttttttgctttttttttaaaaaaaagaataactttttttgcctctttagattacatagaagcattgtagtcttggtagaaccagtatttttgttgtttatttataaggtaattgtgtgtggggaaaagtgcagtatttacctgttgaattcagcatcttgagagcacaagggaaaaaataagaacctacgaatatttttgaggcagataatgatctagtttgactttctagttagtggtgttttgaagagggtattttattgttttttaaaaaaaggttcttaaacattatttgaaatagttaatataaatacataattgcatttgctctgtttattgtaatgtattctaaattaatgcagaaccatatggaaaatttcattaaaatctatccccaaatgtgctttctgtatccttccttctacctattattctgatttttaaaaatgcagttaatgtaccatttatttgcttgatgaagggagctctattttctttaccagaaatgttgctaagtaattcccaatagaaagctgcttattttcattaatgaaaaataaccatggtttgtatactagaagtcttcttcagaaactggtgagcctttctgttcaattgcatttgtaaataaacttgctgatgcatttaacgagtgggtcgtctttttcttaggtgtatgtgtctgacctcaggccttttagccatatttcagtatgtggccttttttgatgttatgttttatccagtagctttactaaggtataattgatgtaataaactgcatatatttaaagtgtatactttgacaaattttgacatggtgtataccttcgaaactatgccacagtctggatgtgtttactgaaacattttaataaggaagtttatttttgataaagttatgtttttggatacaatatatttgtatggtgagagtgatgaattgttggatcatttgaataaaatcttttactaaccccatgataaaaggagaagacaacagtgagcttagaatatctataaagcaaaaaatgtagtctcttgtttaaaaaatctggagcgggaatgcaaggatacaaaactttagcatgctttgagcaaaaatttaaacttactggaatcttttataataatgtaagtggaatggaggattctaggaactgagaactgtattggaataggttcaaaatatgtaagaaatgctaatgtgggagataaaaattttatttagtacttattctgattattattaaagtaataatgtgttccttgaggataacttgtcaaatgccccaaagcataaagaatataattctgaatcccaaattccaaagacaagaactctgtgtttgaattcattctgcatataattatttataagtatagattgtgaatttttccatgttcttaaaattatttttatcttttttcatggttgcatagtgctccattgtttggccttggtaatatttagttgataattccattactgtgtatttttcacttgtttctaagatcaaacattttaatatgtgcatgttatatataaatatgtaaattctgtgatactctatgatcatctctttctttatattattttcatagacatgaaatagttgctcagagattatgcattttaagacactcatagtatatattgccaaagtggtttccagaaaggcactgctggcttcgactcctataagcagcacgtgggcttgttcatctcactgcatgtttatgaagatacagttcttttgccttgttctctgcctgatgtgtatgcagaggcagccctcaatatgcagtggttgaataaatgaatgaagaaaccacta

SEQ ID NO: 16 - RNA, 7147 nt, Homo sapiens
  source 1..7147: mol_type = mRNA
aaactgtcgtcggggctggtgccgcgcgctgccgctgggcacccgcttcgcttcccgcgtcctgcctgactgcccgggtccccgcgggccttgggtcgctttcaggagtttagagaaagccaggtcttcacgttcgtgtaggttcgagaccttaacgaactgcagagcgcaacaaagggaactagagccccggcgccttcgcagccggagcggaagccggagttgtgggaggcccgcgtgccctccccgcccgtggggcccagacgcgcggggacaacccctcccctccacgcatcccgagtctctctctccctcccttgtcctagccatggaagggctggaggaggcagaggccaactgctccgtggcgttcgctgaggctcagagatgggtggaggcagtaacagagaagaattttgaaacaaaagattttcgagcctctctagaaaatggtgttctgctgtgtgatttgattaataagcttaaacctggcgtcattaagaagatcaatagactgtctacaccaatagcaggattggataatataaacgttttcttgaaagcttgtgaacagattggattgaaagaagcccagcttttccatcctggagatctacaggatttatcaaatcgagtcactgtcaagcaagaagagactgacaggagagtgaaaaatgttttgataacattgtactggctgggaagaaaagcacaaagcaacccgtactataatggtccccatcttaatttgaaagcgtttgagaatcttttaggacaagcactgacgaaggcactcgaagactccagcttcctgaaaagaagtggcagggacagtggctacggtgacatctggtgtcctgaacgtggagaatttcttgctcctccaaggcaccataagagagaagattcctttgaaagcttggactctttgggctcgaggtcattgacaagctgctcctctgatatcacgttgagaggggggcgtgaaggttttgaaagtgacacagattcggaatttacatttaagatgcaggattataataaagatgatatgtcgtatcgaaggatttcggctgttgagccaaagactgcgttacccttcaatcgttttttacccaacaaaagtagacagccatcctatgtaccagcacctctgagaaagaaaaagccagacaaacatgaggataacagaagaagttgggcaagcccggtttatacagaagcagatggaacattttcaagtaatcagaggaggatttggggcaccaatgtggagaactggccaactgtacaaggaacttcaaagtcctcttgttatttggaagaggaaaaagcaaagacaagaagcatacccaacattgtaaaggatgatctttatgtgcgcaagctcagtccagtcatgccaaacccagggaatgcttttgatcagtttcttcccaaatgttggaccccagaagatgtgaactggaaaagaataaaaagggaaacttataagccatggtataaagaatttcagggattcagtcagtttttactgcttcaggccctccaaacatactctgatgacatcttgtcttctgaaacacataccaaaattgatcccacttctggcccaaggctcataacccgcaggaagaatctctcttatgcaccaggctatagaagagatgacctcgagatggcagccctggatcctgacttagagaatgatgatttctttgtcagaaagactggggctttccatgcaaatccatatgttctccgagcttttgaagactttagaaagttctctgagcaagatgattctgtagagcgagatataattttacagtgtagagaaggtgaacttgtacttccggatttggaaaaagatgatatgattgttcgccgaattccagcacagaagaaagaagtgccgctgtctggggccccagatagataccacccagtcccttttcccgaaccctggactcttcctccagaaattcaagcaaaatttctctgtgtacttgaaaggacatgcccatccaaagaaaaaagtaatagctgtagaatattagttccttcatatcggcagaagaaagatgacatgctgacacgtaagattcagtcctggaaactgggaactaccgtgcctcccatcagtttcacccctggcccctgcagtgaggctgacttgaagagatgggaggccatccgggaggccagcagacttaggcacaagaaaaggctgatggtggagagactctttcaaaagatttatggtgagaatgggagtaagtccatgagtgatgtcagcgcagaagatgttcaaaacttgcgtcagctgcgttacgaggagatgcagaaaataaaatcacaattaaaagaacaagatcagaaatggcaggatgaccttgcaaaatggaaagatcgtcgaaaaagttacacttcagatctgcagaagaaaaaagaagagagagaagaaattgaaaagcaggcacttgagaagtctaagagaagctctaagacgtttaaggaaatgctgcaggacagggaatcccaaaatcaaaagtctacagttccgtcaagaaggagaatgtattcttttgatgatgtgctggaggaaggaaagcgaccccctacaatgactgtgtcagaagcaagttaccagagtgagagagtagaagagaagggagcaacttatccttcagaaattcccaaagaagattctaccacttttgcaaaaagagaggaccgtgtaacaactgaaattcagcttccttctcaaagtcctgtggaagaacaaagcccagcctctttgtcttctctgcgttcacggagcacacaaatggaatcaactcgtgtttcagcttctctccccagaagttaccggaaaactgatacagtcaggttaacatctgtggtcacaccaagaccctttggctctcagacaaggggaatctcatcactccccagatcttacacgatggatgatgcttggaagtataatggagatgttgaagacattaagagaactccaaacaatgtggtcagcacccctgcaccaagcccggacgcaagccaactggcttcaagcttatctagccagaaagaggtagcagcaacagaagaagatgtgacaaggctgccctctcctacatcccccttctcatctctttcccaagaccaggctgccacttctaaagccacattgtcttccacatctggtcttgatttaatgtctgaatctggagaaggggaaatctccccacaaagagaagtctcaagatcccaggatcagttcagtgatatgagaatcagcataaaccagacgcctgggaagagtcttgactttgggtttacaataaaatgggatattcctgggatcttcgtagcatcagttgaagcaggtagcccagcagaattttctcagctacaagtagatgatgaaattattgctattaacaacaccaagttttcatataacgattcaaaagagtgggaggaagccatggctaaggctcaagaaactggacacctagtgatggatgtgaggcgctatggaaaggctggttcacctgaaacaaagtggattgatgcaacttctggaatttacaactcagaaaaatcttcaaatctatctgtaacaactgatttctccgaaagccttcagagttctaatattgaatccaaagaaatcaatggaattcatgatgaaagcaatgcttttgaatcaaaagcatctgaatccatttctttgaaaaacttaaaaaggcgatcacaattttttgaacaaggaagctctgattcggtggttcctgatcttccagttccaaccatcagtgccccgagtcgctgggtgtgggatcaagaggaggagcggaagcggcaggagaggtggcagaaggagcaggaccgcctactgcaggaaaaatatcaacgtgagcaggagaaactgagggaagagtggcaaagggccaaacaggaggcagagagagagaattccaagtacttggatgaggaactgatggtcctaagctcaaacagcatgtctctgaccacacgggagccctctcttgccacctgggaagctacctggagtgaagggtccaagtcttcagacagagaaggaacccgagcaggagaagaggagaggagacagccacaagaggaagttgttcatgaggaccaaggaaagaagccgcaggatcagcttgttattgagagagagaggaaatgggagcaacagcttcaggaagagcaagagcaaaagcggcttcaggctgaggctgaggagcagaagcgtcctgcggaggagcagaagcgccaggcagagatagagcgggaaacatcagtcagaatataccagtacaggaggcctgttgattcctatgatataccaaagacagaagaagcatcttcaggttttcttcctggtgacaggaataaatccagatctactactgaactggatgattactccacaaataaaaatggaaacaataaatatttagaccaaattgggaacatgacctcttcacagaggagatccaagaaagaacaagtaccatcaggagcagaattggagaggcaacaaatccttcaggaaatgaggaagagaacaccccttcacaatgacaacagctggatccgacagcgcagtgccagtgtcaacaaagagcctgttagtcttcctgggatcatgagaagaggcgaatctttagataacctggactccccccgatccaattcttggagacagcctccttggctcaatcagcccacaggattctatgcttcttcctctgtgcaagactttagtcgcccaccacctcagctggtgtccacatcaaaccgtgcctacatgcggaacccctcctccagcgtgcccccaccttcagctggctccgtgaagacctccaccacaggtgtggccaccacacagtcccccaccccgagaagccattccccttcagcttcacagtcaggctctcagctgcgtaacaggtcagtcagtgggaagcgcatatgctcctactgcaataacattctgggcaaaggagccgccatgatcatcgagtccctgggtctttgttatcatttgcattgttttaagtgtgttgcctgtgagtgtgacctcggaggctcttcctcaggagctgaagtcaggatcagaaaccaccaactgtactgcaacgactgctatctcagattcaaatctggacggccaaccgccatgtgatgtaagcctccatacgaaagcactgttgcagatagaagaagaggtggttgctgctcatgtagatctataaatatgtgttgtatgtcttttttgctttttttttaaaaaaaagaataactttttttgcctctttagattacatagaagcattgtagtcttggtagaaccagtatttttgttgtttatttataaggtaattgtgtgtggggaaaagtgcagtatttacctgttgaattcagcatcttgagagcacaagggaaaaaataagaacctacgaatatttttgaggcagataatgatctagtttgactttctagttagtggtgttttgaagagggtattttattgttttttaaaaaaaggttcttaaacattatttgaaatagttaatataaatacataattgcatttgctctgtttattgtaatgtattctaaattaatgcagaaccatatggaaaatttcattaaaatctatccccaaatgtgctttctgtatccttccttctacctattattctgatttttaaaaatgcagttaatgtaccatttatttgcttgatgaagggagctctattttctttaccagaaatgttgctaagtaattcccaatagaaagctgcttattttcattaatgaaaaataaccatggtttgtatactagaagtcttcttcagaaactggtgagcctttctgttcaattgcatttgtaaataaacttgctgatgcatttaacgagtgggtcgtctttttcttaggtgtatgtgtctgacctcaggccttttagccatatttcagtatgtggccttttttgatgttatgttttatccagtagctttactaaggtataattgatgtaataaactgcatatatttaaagtgtatactttgacaaattttgacatggtgtataccttcgaaactatgccacagtctggatgtgtttactgaaacattttaataaggaagtttatttttgataaagttatgtttttggatacaatatatttgtatggtgagagtgatgaattgttggatcatttgaataaaatcttttactaaccccatgataaaaggagaagacaacagtgagcttagaatatctataaagcaaaaaatgtagtctcttgtttaaaaaatctggagcgggaatgcaaggatacaaaactttagcatgctttgagcaaaaatttaaacttactggaatcttttataataatgtaagtggaatggaggattctaggaactgagaactgtattggaataggttcaaaatatgtaagaaatgctaatgtgggagataaaaattttatttagtacttattctgattattattaaagtaataatgtgttccttgaggataacttgtcaaatgccccaaagcataaagaatataattctgaatcccaaattccaaagacaagaactctgtgtttgaattcattctgcatataattatttataagtatagattgtgaatttttccatgttcttaaaattatttttatcttttttcatggttgcatagtgctccattgtttggccttggtaatatttagttgataattccattactgtgtatttttcacttgtttctaagatcaaacattttaatatgtgcatgttatatataaatatgtaaattctgtgatactctatgatcatctctttctttatattattttcatagacatgaaatagttgctcagagattatgcattttaagacactcatagtatatattgccaaagtggtttccagaaaggcactgctggcttcgactcctataagcagcacgtgggcttgttcatctcactgcatgtttatgaagatacagttcttttgccttgttctctgcctgatgtgtatgcagaggcagccctcaatatgcagtggttgaataaatgaatgaagaaaccacta

SEQ ID NO: 17 - protein, 1349 aa, Homo sapiens
MKKIRICHIFTFYSWMSYDVLFQRTELGALEIWRQLICAHVCICVGWLYLRDRVCSKKDIILRTEQNSGRTILIKAVTEKNFETKDFRASLENGVLLCDLINKLKPGVIKKINRLSTPIAGLDNINVFLKACEQIGLKEAQLFHPGDLQDLSNRVTVKQEETDRRVKNVLITLYWLGRKAQSNPYYNGPHLNLKAFENLLGQALTKALEDSSFLKRSGRDSGYGDIWCPERGEFLAPPRHHKREDSFESLDSLGSRSLTSCSSDITLRGGREGFESDTDSEFTFKMQDYNKDDMSYRRISAVEPKTALPFNRFLPNKSRQPSYVPAPLRKKKPDKHEDNRRSWASPVYTEADGTFSRLFQKIYGENGSKSMSDVSAEDVQNLRQLRYEEMQKIKSQLKEQDQKWQDDLAKWKDRRKSYTSDLQKKKEEREEIEKQALEKSKRSSKTFKEMLQDRESQNQKSTVPSRRRMYSFDDVLEEGKRPPTMTVSEASYQSERVEEKGATYPSEIPKEDSTTFAKREDRVTTEIQLPSQSPVEEQSPASLSSLRSRSTQMESTRVSASLPRSYRKTDTVRLTSVVTPRPFGSQTRGISSLPRSYTMDDAWKYNGDVEDIKRTPNNVVSTPAPSPDASQLASSLSSQKEVAATEEDVTRLPSPTSPFSSLSQDQAATSKATLSSTSGLDLMSESGEGEISPQREVSRSQDQFSDMRISINQTPGKSLDFGFTIKWDIPGIFVASVEAGSPAEFSQLQVDDEIIAINNTKFSYNDSKEWEEAMAKAQETGHLVMDVRRYGKAGSPETKWIDATSGIYNSEKSSNLSVTTDFSESLQSSNIESKEINGIHDESNAFESKASESISLKNLKRRSQFFEQGSSDSVVPDLPVPTISAPSRWVWDQEEERKRQERWQKEQDRLLQEKYQREQEKLREEWQRAKQEAERENSKYLDEELMVLSSNSMSLTTREPSLATWEATWSEGSKSSDREGTRAGEEERRQPQEEVVHEDQGKKPQDQLVIERERKWEQQLQEEQEQKRLQAEAEEQKRPAEEQKRQAEIERETSVRIYQYRRPVDSYDIPKTEEASSGFLPGDRNKSRSTTELDDYSTNKNGNNKYLDQIGNMTSSQRRSKKEQVPSGAELERQQILQEMRKRTPLHNDNSWIRQRSASVNKEPVSLPGIMRRGESLDNLDSPRSNSWRQPPWLNQPTGFYASSSVQDFSRPPPQLVSTSNRAYMRNPSSSVPPPSAGSVKTSTTGVATTQSPTPRSHSPSASQSGSQLRNRSVSGKRICSYCNNILGKGAAMIIESLGLCYHLHCFKCVACECDLGGSSSGAEVRIRNHQLYCNDCYLRFKSGRPTAM

SEQ ID NO: 18 - protein, 1385 aa, Homo sapiens
MQDYNKDDMSYRRISAVEPKTALPFNRFLPNKSRQPSYVPAPLRKKKPDKHEDNRRSWASPVYTEADGTFSSNQRRIWGTNVENWPTVQGTSKSSCYLEEEKAKTRSIPNIVKDDLYVRKLSPVMPNPGNAFDQFLPKCWTPEDVNWKRIKRETYKPWYKEFQGFSQFLLLQALQTYSDDILSSETHTKIDPTSGPRLITRRKNLSYAPGYRRDDLEMAALDPDLENDDFFVRKTGAFHANPYVLRAFEDFRKFSEQDDSVERDIILQCREGELVLPDLEKDDMIVRRIPAQKKEVPLSGAPDRYHPVPFPEPWTLPPEIQAKFLCVLERTCPSKEKSNSCRILVPSYRQKKDDMLTRKIQSWKLGTTVPPISFTPGPCSEADLKRWEAIREASRLRHKKRLMVERLFQKIYGENGSKSMSDVSAEDVQNLRQLRYEEMQKIKSQLKEQDQKWQDDLAKWKDRRKSYTSDLQKKKEEREEIEKQALEKSKRSSKTFKEMLQDRESQNQKSTVPSRRRMYSFDDVLEEGKRPPTMTVSEASYQSERVEEKGATYPSEIPKEDSTTFAKREDRVTTEIQLPSQSPVEEQSPASLSSLRSRSTQMESTRVSASLPRSYRKTDTVRLTSVVTPRPFGSQTRGISSLPRSYTMDDAWKYNGDVEDIKRTPNNVVSTPAPSPDASQLASSLSSQKEVAATEEDVTRLPSPTSPFSSLSQDQAATSKATLSSTSGLDLMSESGEGEISPQREVSRSQDQFSDMRISINQTPGKSLDFGFTIKWDIPGIFVASVEAGSPAEFSQLQVDDEIIAINNTKFSYNDSKEWEEAMAKAQETGHLVMDVRRYGKAGSPETKWIDATSGIYNSEKSSNLSVTTDFSESLQSSNIESKEINGIHDESNAFESKASESISLKNLKRRSQFFEQGSSDSVVPDLPVPTISAPSRWVWDQEEERKRQERWQKEQDRLLQEKYQREQEKLREEWQRAKQEAERENSKYLDEELMVLSSNSMSLTTREPSLATWEATWSEGSKSSDREGTRAGEEERRQPQEEVVHEDQGKKPQDQLVIERERKWEQQLQEEQEQKRLQAEAEEQKRPAEEQKRQAEIERETSVRIYQYRRPVDSYDIPKTEEASSGFLPGDRNKSRSTTELDDYSTNKNGNNKYLDQIGNMTSSQRRSKKEQVPSGAELERQQILQEMRKRTPLHNDNSWIRQRSASVNKEPVSLPGIMRRGESLDNLDSPRSNSWRQPPWLNQPTGFYASSSVQDFSRPPPQLVSTSNRAYMRNPSSSVPPPSAGSVKTSTTGVATTQSPTPRSHSPSASQSGSQLRNSVLPVSVTSEALPQELKSGSETTNCTATTAISDSNLDGQPPCDVSLHTKALLQIEEEVVAAHVDL

SEQ ID NO: 19 - protein, 1631 aa, Homo sapiens
MEGLEEAEANCSVAFAEAQRWVEAVTEKNFETKDFRASLENGVLLCDLINKLKPGVIKKINRLSTPIAGLDNINVFLKACEQIGLKEAQLFHPGDLQDLSNRVTVKQEETDRRVKNVLITLYWLGRKAQSNPYYNGPHLNLKAFENLLGQALTKALEDSSFLKRSGRDSGYGDIWCPERGEFLAPPRHHKREDSFESLDSLGSRSLTSCSSDITLRGGREGFESDTDSEFTFKMQDYNKDDMSYRRISAVEPKTALPFNRFLPNKSRQPSYVPAPLRKKKPDKHEDNRRSWASPVYTEADGTFSSNQRRIWGTNVENWPTVQGTSKSSCYLEEEKAKTRSIPNIVKDDLYVRKLSPVMPNPGNAFDQFLPKCWTPEDVNWKRIKRETYKPWYKEFQGFSQFLLLQALQTYSDDILSSETHTKIDPTSGPRLITRRKNLSYAPGYRRDDLEMAALDPDLENDDFFVRKTGAFHANPYVLRAFEDFRKFSEQDDSVERDIILQCREGELVLPDLEKDDMIVRRIPAQKKEVPLSGAPDRYHPVPFPEPWTLPPEIQAKFLCVLERTCPSKEKSNSCRILVPSYRQKKDDMLTRKIQSWKLGTTVPPISFTPGPCSEADLKRWEAIREASRLRHKKRLMVERLFQKIYGENGSKSMSDVSAEDVQNLRQLRYEEMQKIKSQLKEQDQKWQDDLAKWKDRRKSYTSDLQKKKEEREEIEKQALEKSKRSSKTFKEMLQDRESQNQKSTVPSRRRMYSFDDVLEEGKRPPTMTVSEASYQSERVEEKGATYPSEIPKEDSTTFAKREDRVTTEIQLPSQSPVEEQSPASLSSLRSRSTQMESTRVSASLPRSYRKTDTVRLTSVVTPRPFGSQTRGISSLPRSYTMDDAWKYNGDVEDIKRTPNNVVSTPAPSPDASQLASSLSSQKEVAATEEDVTRLPSPTSPFSSLSQDQAATSKATLSSTSGLDLMSESGEGEISPQREVSRSQDQFSDMRISINQTPGKSLDFGFTIKWDIPGIFVASVEAGSPAEFSQLQVDDEIIAINNTKFSYNDSKEWEEAMAKAQETGHLVMDVRRYGKAGSPETKWIDATSGIYNSEKSSNLSVTTDFSESLQSSNIESKEINGIHDESNAFESKASESISLKNLKRRSQFFEQGSSDSVVPDLPVPTISAPSRWVWDQEEERKRQERWQKEQDRLLQEKYQREQEKLREEWQRAKQEAERENSKYLDEELMVLSSNSMSLTTREPSLATWEATWSEGSKSSDREGTRAGEEERRQPQEEVVHEDQGKKPQDQLVIERERKWEQQLQEEQEQKRLQAEAEEQKRPAEEQKRQAEIERETSVRIYQYRRPVDSYDIPKTEEASSGFLPGDRNKSRSTTELDDYSTNKNGNNKYLDQIGNMTSSQRRSKKEQVPSGAELERQQILQEMRKRTPLHNDNSWIRQRSASVNKEPVSLPGIMRRGESLDNLDSPRSNSWRQPPWLNQPTGFYASSSVQDFSRPPPQLVSTSNRAYMRNPSSSVPPPSAGSVKTSTTGVATTQSPTPRSHSPSASQSGSQLRNRSVSGKRICSYCNNILGKGAAMIIESLGLCYHLHCFKCVACECDLGGSSSGAEVRIRNHQLYCNDCYLRFKSGRPTAM

SEQ ID NO: 20 - RNA, 1436 nt, Homo sapiens
  source 1..1436: mol_type = mRNA
gcttttatgagctgtaacactcaccgcgaaggtccgcagcttcactcctgaagccagcgagaccacgagcctactgggaggaacgaacaactcccgacgcgccgccttaagagctgtaacactcaccgcgaaggtctgcagcttcactcctgagccagcgagaccacgaacccaccagaaggaaaaaactccgaacacatctgaacatcagaagcaacaaactccggacacgccgcctttaagaactgtaacactcactgcgagggtccgcggcttcattcttgaagtgagtgagaccaagaacccaccagttctggacacaatttcaagtcctcaggtgccatcaatattctgaaaatggcagtgatttttattcaacctgtataaggcactttcaccatgtacctggaagcaacatctacatctttttcagcaatctagatgctggggacacaaggtccaccttccaggaatatggccatgacaccagaaatcacaaacatgatgagaatggaatgactggggaagaagtgccagatgcttcacttgtaaatgaagacccagcctctggggatgcagataccacctccctgaagaagctgaatatctgcagataagtggagttcaccaatgatgaggagcgggatggagaaaggaggtagggagagtcatccaaggaacatgagcaacatgttaaaagccaagtggtttaatttctggagatggtgaacccaagaggctctgctgggagacaacaaaaataatgaagaattgaaccagagtccggtgaatatcagcactgggaccagttagcagaggaaaaggaaagaataaaagcgaaaagaatgaagagtcatatgattaccaacttttcctttttcatataaattgagtgtatatgggtctggaacaacctgaatttccatcaagtcctggctaacctcattatgtcctatgaatatttttgactaatcccactttacattaatctgtattgtgaatgtggatattgaattatatttctttgtaatcccattatccaaaatccagttcagagactattagttaccaatgttcactgtgaaggaaaaaaaaaaaaaaaaagctcagaggataaacatgtgatatggtttggctgtgtccccacccaaatatcatcttgaattgtagctcccataattcccacgtgttgtgggagggacccggtgggagataattgtatcatgggggtggttcccccatactattctcatagtagtgaataagtctcacaaaatctgatggttttatgagggaaaacccctttcacctggttctcattctcttctctggtctgtcgtcatgtaagacatgcctttcaccttctccaccatgactgtgaggcctccccagccacgtggaactgtgagcccattaaacctctttcacttataaat

SEQ ID NO: 21 - RNA, 1367 nt, Homo sapiens
  source 1..1367: mol_type = mRNA
gcttttatgagctgtaacactcaccgcgaaggtccgcagcttcactcctgaagccagcgagaccacgagcctactgggaggaacgaacaactcccgacgcgccgccttaagagctgtaacactcaccgcgaaggtctgcagcttcactcctgagccagcgagaccacgaacccaccagaaggaaaaaactccgaacacatctgaacatcagaagcaacaaactccggacacgccgcctttaagaactgtaacactcactgcgagggtccgcggcttcattcttgaagtgagtgagaccaagaacccaccagttctggacacaatttcaagtcctcaggtgccatcaatattctgaaaatggcagtgatttttattcaacctgtataaggcactttcaccatgtacctggaagcaacatctacatctttttcagcaatctagatgctggggacacaaggtccaccttccaggaatatggccatgacaccagaaatcacaaacatgatgagaatggaatgactggggaagaagtgccagatgcttcacttgtaaatgaagacccagcctctggggatgcagataccacctccctgaagaagctgaatatctgcagataagtggagttcaccaatgatgaggagcgggatggagaaaggaggtagggagagtcatccaaggaacatgagcaacatgttaaaagaattgaaccagagtccggtgaatatcagcactgggaccagttagcagaggaaaaggaaagaataaaagcgaaaagaatgaagagtcatatgattaccaacttttcctttttcatataaattgagtgtatatgggtctggaacaacctgaatttccatcaagtcctggctaacctcattatgtcctatgaatatttttgactaatcccactttacattaatctgtattgtgaatgtggatattgaattatatttctttgtaatcccattatccaaaatccagttcagagactattagttaccaatgttcactgtgaaggaaaaaaaaaaaaaaaaagctcagaggataaacatgtgatatggtttggctgtgtccccacccaaatatcatcttgaattgtagctcccataattcccacgtgttgtgggagggacccggtgggagataattgtatcatgggggtggttcccccatactattctcatagtagtgaataagtctcacaaaatctgatggttttatgagggaaaacccctttcacctggttctcattctcttctctggtctgtcgtcatgtaagacatgcctttcaccttctccaccatgactgtgaggcctccccagccacgtggaactgtgagcccattaaacctctttcacttataaat

SEQ ID NO: 22 - RNA, 1100 nt, Homo sapiens
  source 1..1100: mol_type = mRNA
gcttttatgagctgtaacactcaccgcgaaggtccgcagcttcactcctgaagccagcgagaccacgagcctactgggaggaacgaacaactcccgacgcgccgccttaagagctgtaacactcaccgcgaaggtctgcagcttcactcctgagccagcgagaccacgaacccaccagaaggaaaaaactccgaacacatctgaacatcagaagcaacaaactccggacacgccgcctttaagaactgtaacactcactgcgagggtccgcggcttcattcttgaagtgagtgagaccaagaacccaccagttctggacacaatttcaagtcctcaggtgccatcaatattctgaaaatggcagtgatttttattcaacctgtataaggcactttcaccatgtacctggaagcaacatctacatctttttcagaattgaaccagagtccggtgaatatcagcactgggaccagttagcagaggaaaaggaaagaataaaagcgaaaagaatgaagagtcatatgattaccaacttttcctttttcatataaattgagtgtatatgggtctggaacaacctgaatttccatcaagtcctggctaacctcattatgtcctatgaatatttttgactaatcccactttacattaatctgtattgtgaatgtggatattgaattatatttctttgtaatcccattatccaaaatccagttcagagactattagttaccaatgttcactgtgaaggaaaaaaaaaaaaaaaaagctcagaggataaacatgtgatatggtttggctgtgtccccacccaaatatcatcttgaattgtagctcccataattcccacgtgttgtgggagggacccggtgggagataattgtatcatgggggtggttcccccatactattctcatagtagtgaataagtctcacaaaatctgatggttttatgagggaaaacccctttcacctggttctcattctcttctctggtctgtcgtcatgtaagacatgcctttcaccttctccaccatgactgtgaggcctccccagccacgtggaactgtgagcccattaaacctctttcacttataaat

SEQ ID NO: 23 - RNA, 1370 nt, Homo sapiens
  source 1..1370: mol_type = mRNA
gcttttatgagctgtaacactcaccgcgaaggtccgcagcttcactcctgaagccagcgagaccacgagcctactgggaggaacgaacaactcccgacgcgccgccttaagagctgtaacactcaccgcgaaggtctgcagcttcactcctgagccagcgagaccacgaacccaccagaaggaaaaaactccgaacacatctgaacatcagaagcaacaaactccggacacgccgcctttaagaactgtaacactcactgcgagggtccgcggcttcattcttgaagtgagtgagaccaagaacccaccagttctggacacaatttcaagtcctcaggtgccatcaatattctgaaaatggcagtgatttttattcaacctgtataaggcactttcaccatgtacctggaagcaacatctacatctttttcagtttcttctacgccaggtgtgtgcttagctccatgacaaaaggtgacagcttattctgcagcacacacacatcatcaaagtgggaggtggtgagactggcacactgacagtctgtcctagcagatttcagctcacactgcaatctagatgctggggacacaaggtccaccttccaggaatatggccatgacaccagaaatcacaaacatgatgagaatggaatgactggggaagaagtgccagatgcttcacttgtaaatgaagacccagcctctggggatgcagataccacctccctgaagaagctgaatatctgcagataagtggagttcaccaatgatgaggagcgggatggagaaaggaggtagggagagtcatccaaggaacatgagcaacatgttaaaagccaagtggtttaatttctggagatggtgaacccaagaggctctgctgggagacaacaaaaataatgaagaattgaaccagagtccggtgaatatcagcactgggaccagttagcagaggaaaaggaaagaataaaagcgaaaagaatgaagagtcatatgattaccaacttttcctttttcatataaattgagtgtatatgggtctggaacaacctgaatttccatcaagtcctggctaacctcattatgtcctatgaatatttttgactaatcccactttacattaatctgtattgtgaatgtggatattgaattatatttctttgtaatcccattatccaaaatccagttcagagactattagttaccaatgttcactgtgaaggaaaaaaaaaaaaaaaaagctcagaggataaacatgtgatatggtttggctgtgtccccacccaaatatcatcttgaattgtagctcccataattcccacgtgttgtgggagggacccggtgggagataattgtatcatgggggtg

SEQ ID NO: 24 - RNA, 3812 nt, Homo sapiens
  source 1..3812: mol_type = mRNA
gcggagagaaccctctgttttcccccactctctctccacctcctcctgccttccccaccccgagtgcggagccagagatcaaaagatgaaaaggcagtcaggtcttcagtagccaaaaaacaaaacaaacaaaaacaaaaaagccgaaataaaagaaaaagataataactcagttcttatttgcacctacttcagtggacactgaatttggaaggtggaggattttgtttttttcttttaagatctgggcatcttttgaatctacccttcaagtattaagagacagactgtgagcctagcagggcagatcttgtccaccgtgtgtcttcttctgcacgagactttgaggctgtcagagcgctttttgcgtggttgctcccgcaagtttccttctctggagcttcccgcaggtgggcagctagctgcagcgactaccgcatcatcacagcctgttgaactcttctgagcaagagaaggggaggcggggtaagggaagtaggtggaagattcagccaagctcaaggatggaagtgcagttagggctgggaagggtctaccctcggccgccgtccaagacctaccgaggagctttccagaatctgttccagagcgtgcgcgaagtgatccagaacccgggccccaggcacccagaggccgcgagcgcagcacctcccggcgccagtttgctgctgctgcagcagcagcagcagcagcagcagcagcagcagcagcagcagcagcagcagcagcagcagcagcagcaagagactagccccaggcagcagcagcagcagcagggtgaggatggttctccccaagcccatcgtagaggccccacaggctacctggtcctggatgaggaacagcaaccttcacagccgcagtcggccctggagtgccaccccgagagaggttgcgtcccagagcctggagccgccgtggccgccagcaaggggctgccgcagcagctgccagcacctccggacgaggatgactcagctgccccatccacgttgtccctgctgggccccactttccccggcttaagcagctgctccgctgaccttaaagacatcctgagcgaggccagcaccatgcaactccttcagcaacagcagcaggaagcagtatccgaaggcagcagcagcgggagagcgagggaggcctcgggggctcccacttcctccaaggacaattacttagggggcacttcgaccatttctgacaacgccaaggagttgtgtaaggcagtgtcggtgtccatgggcctgggtgtggaggcgttggagcatctgagtccaggggaacagcttcggggggattgcatgtacgccccacttttgggagttccacccgctgtgcgtcccactccttgtgccccattggccgaatgcaaaggttctctgctagacgacagcgcaggcaagagcactgaagatactgctgagtattcccctttcaagggaggttacaccaaagggctagaaggcgagagcctaggctgctctggcagcgctgcagcagggagctccgggacacttgaactgccgtctaccctgtctctctacaagtccggagcactggacgaggcagctgcgtaccagagtcgcgactactacaactttccactggctctggccggaccgccgccccctccgccgcctccccatccccacgctcgcatcaagctggagaacccgctggactacggcagcgcctgggcggctgcggcggcgcagtgccgctatggggacctggcgagcctgcatggcgcgggtgcagcgggacccggttctgggtcaccctcagccgccgcttcctcatcctggcacactctcttcacagccgaagaaggccagttgtatggaccgtgtggtggtggtgggggtggtggcggcggcggcggcggcggcggcggcggcggcggcggcggcggcggcggcgaggcgggagctgtagccccctacggctacactcggccccctcaggggctggcgggccaggaaagcgacttcaccgcacctgatgtgtggtaccctggcggcatggtgagcagagtgccctatcccagtcccacttgtgtcaaaagcgaaatgggcccctggatggatagctactccggaccttacggggacatgcgtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctgggagaaaaattccgggttggcaattgcaagcatctcaaaatgaccagaccctgaagaaaggctgacttgcctcattcaaaatgagggctctagagggctctagtggatagtctggagaaacctggcgtctgaggcttaggagcttaggtttttgctcctcaacacagactttgacgttggggttgggggctactctcttgattgctgactccctccagcgggaccaatagtgttttcctacctcacagggatgttgtgaggacgggctgtagaagtaatagtggttaccattcatgtagttgtgagtatcatgattattgtttcctgtaatgtggcttggcattggcaaagtgctttttgattgttcttgatcacatatgatgggggccaggcactgactcaggcggatgcagtgaagctctggctcagtcgcttgcttttcgtggtgtgctgccaggaagaaactttgctgatgggactcaaggtgtcaccttggacaagaagcaactgtgtctgtctgaggttcctgtggccatctttatttgtgtattaggcaattcgtatttcccccttaggttctagccttctggatcccagccagtgacctagatcttagcctcaggccctgtcactgagctgaaggtagtagctgatccacagaagttcagtaaacaaggaccagatttctgcttctccaggagaagaagccagccaacccctctcttcaaacacactgagagactacagtccgactttccctcttacatctagccttactgtagccacactccttgattgctctctcacatcacatgcttctcttcatcagttgtaagcctctcattcttctcccaagccagactcaaatattgtattgatgtcaaagaagaatcacttagagtttggaatatcttgttctctctctgctccatagcttccatattgacaccagtttctttctagtggagaagtggagtctgtgaagccagggaaacacacatgtgagagtcagaaggactctccctgacttgcctggggcctgtctttcccaccttctccagtctgtctaaacacacacacacacacacacacacacacacacacgctctctctctctctccccccccaacacacacacactctctctctctctcacacacacacacatacacacacacttctttctctttcccctgactcagcaacattctggagaaaagccaaggaaggacttcaggaggggagtttcccccttctcagggcagaattttaatctccagaccaacaagaagttccctaatgtggattgaaaggctaatgaggtttatttttaactactttctatttgtttgaatgttgcatatttctactagtgaaattttcccttaataaagccattaatacacc

SEQ ID NO: 25 - protein, 644 aa, Homo sapiens
MEVQLGLGRVYPRPPSKTYRGAFQNLFQSVREVIQNPGPRHPEAASAAPPGASLLLLQQQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGEKFRVGNCKHLKMTRP